SEQUENCE LISTING (495 sequences)

SEQ ID NO: 1 - protein, 666 aa, synthetic construct
  REGION 1..666: note = Description of Artificial Sequence: Synthetic polypeptide
MDAMKRGLCCVLLLCGAVFVSPSQEIHARFRRGARAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPWSTQLLLNGSLAEEEVMIRSENITNNAKNILVQFNTPVQINCTRPNNNTRKSIRIGPGQAFYATGDIIGDIRQAHCNVSKATWNETLGKWKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKWKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 2 - protein, 659 aa, synthetic construct
  REGION 1..659: note = Description of Artificial Sequence: Synthetic polypeptide
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYFGDIIGDIRMAHCNVSKATWNETLGKVVKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 3 - DNA, 1980 bp, synthetic construct
  misc_feature 1..1980: note = Description of Artificial Sequence: Synthetic polynucleotide
  source 1..1980: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaaggataagaagtttaatgggacaggcccttgccctagcgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtaacgtgagcaaggccacctggaacgagaccctgggcaaggtggtcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 4 - protein, 688 aa, synthetic construct
  REGION 1..688: note = Description of Artificial Sequence: Synthetic polypeptide
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYFGDIIGDIRMAHCNVSKATWNETLGKVVKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 5 - DNA, 2067 bp, synthetic construct
  misc_feature 1..2067: note = Description of Artificial Sequence: Synthetic polynucleotide
  source 1..2067: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaaggataagaagtttaatgggacaggcccttgccctagcgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtaacgtgagcaaggccacctggaacgagaccctgggcaaggtggtcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgacggtggaggcggttcaggcggcggatctggcggtgggagcggttcgggagcccatatagtgatggttgatgcctataaaccgaccaagtga

SEQ ID NO: 6 - protein, 659 aa, synthetic construct
  REGION 1..659: note = Description of Artificial Sequence: Synthetic polypeptide
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKNKTFNGTGPCPNVSTVQCTHGIKPVVSTQLLLNGSLAEEEVIIRSENITNNAKNILVQLNTSVQINCTRPNNNTVKSIRIGPGQAFYYFGDIIGDIRMAHCNVSKATWNETLGNVSKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 7 - DNA, 1980 bp, synthetic construct
  misc_feature 1..1980: note = Description of Artificial Sequence: Synthetic polynucleotide
  source 1..1980: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaagaataagacgtttaatgggacaggcccttgccctaacgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacaagtgtgcagatcaactgcacccggcccaacaacaacaccgtgaagtccattagaattggtccgggacaggcattttactacttcggagatataataggcgatatcagaatggcgcactgtaacgtgagcaaggccacctggaacgagaccctgggcaatgtgagcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 8 - protein, 688 aa, synthetic construct
  REGION 1..688: note = Description of Artificial Sequence: Synthetic polypeptide
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKNKTFNGTGPCPNVSTVQCTHGIKPVVSTQLLLNGSLAEEEVIIRSENITNNAKNILVQLNTSVQINCTRPNNNTVKSIRIGPGQAFYYFGDIIGDIRMAHCNVSKATWNETLGNVSKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 9 - DNA, 2067 bp, synthetic construct
  misc_feature 1..2067: note = Description of Artificial Sequence: Synthetic polynucleotide
  source 1..2067: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaagaataagacgtttaatgggacaggcccttgccctaacgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacaagtgtgcagatcaactgcacccggcccaacaacaacaccgtgaagtccattagaattggtccgggacaggcattttactacttcggagatataataggcgatatcagaatggcgcactgtaacgtgagcaaggccacctggaacgagaccctgggcaatgtgagcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgacggtggaggcggttcaggcggcggatctggcggtgggagcggttcgggagcccatatagtgatggttgatgcctataaaccgaccaagtga

SEQ ID NO: 10 - protein, 45 aa, Human immunodeficiency virus
KGKGKGKGKGCTRPNNNTRKSIRIGPGQTFYATGDIIGDIRQAHC

SEQ ID NO: 11 - protein, 659 aa, synthetic construct
  REGION 1..659: note = Description of Artificial Sequence: Synthetic polypeptide
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKNKTFNGTGPCPNVSTVQCTHGIKPVVSTQLLLNGSLAEEEVIIRSENITNNAKNILVQLNTPVQINCTRPNNNTVKSIRIGPGQAFYYFGDIIGDIRMAHCNVSKATWNETLGNVSKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 12 - DNA, 1980 bp, synthetic construct
  misc_feature 1..1980: note = Description of Artificial Sequence: Synthetic polynucleotide
  source 1..1980: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaagaataagacgtttaatgggacaggcccttgccctaacgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtaacgtgagcaaggccacctggaacgagaccctgggcaatgtgagcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgactga

SEQ ID NO: 13 - protein, 688 aa, synthetic construct
  REGION 1..688: note = Description of Artificial Sequence: Synthetic polypeptide
MDAMKRGLCCVLLLCGAVFVSPAGAGSNLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNYAPNLLSNMRGELKQCSFNMTTELRDKKQKVYSLFYRLDVVQINENQGNRSNNSNKEYRLINCNTSAITQACPKVSFEPIPIHYCAPAGFAILKCKNKTFNGTGPCPNVSTVQCTHGIKPVVSTQLLLNGSLAEEEVIIRSENITNNAKNILVQLNTPVQINCTRPNNNTVKSIRIGPGQAFYYFGDIIGDIRMAHCNVSKATWNETLGNVSKQLRKHFGNNTIIRFAQSSGGDLEVTTHSFNCGGEFFYCNTSGLFNSTWISNTSVQGSNSTGSNDSIVLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 14 - DNA, 2067 bp, synthetic construct
  misc_feature 1..2067: note = Description of Artificial Sequence: Synthetic polynucleotide
  source 1..2067: mol_type = other DNA
atggacgccatgaagaggggactttgctgtgttcttctgctgtgtggcgccgtgtttgttagccccgctggggccggatccaacctgtgggtcactgtgtattatggtgtgccagtgtggaaggatgcagagacaacactcttttgcgcctccgacgctaaagcatacgaaacggagaagcacaacgtgtgggcgacccatgcctgtgtccctacagaccctaaccctcaggaaattcatcttgaaaatgtcacagaagagtttaacatgtggaaaaacaacatggtggaacagatgcacgaggatatcatttccctgtgggaccagagtctgaaaccatgtgtcaaacttactcctctgtgcgtgactctccagtgtacaaactacgcacccaaccttttgagtaatatgcggggcgagctcaagcagtgcagtttcaatatgacaaccgaattgagagacaaaaaacagaaagtatactccctcttctaccggctggacgtggtgcagatcaatgagaaccaaggaaatagaagcaacaacagtaacaaggaataccggctcataaattgcaataccagcgctattacgcaggcttgccctaaggtgagctttgagccaatcccgatacattattgtgccccggcaggcttcgctatactgaaatgcaagaataagacgtttaatgggacaggcccttgccctaacgtttcaacggtccaatgtacccacgggatcaagcccgtagtgtctacacagctcctgctgaacggcagcctggccgaagaggaggtcataattaggagcgagaacataactaacaacgctaaaaacattctcgtccagctcaatacacctgtgcagatcaactgcacccggcccaacaacaacaccgtgaagtccattagaattggtccgggacaggcattttactacttcggagatataataggcgatatcagaatggcgcactgtaacgtgagcaaggccacctggaacgagaccctgggcaatgtgagcaaacagttgcgcaagcactttgggaacaacaccattattcggtttgcccagtcttccggcggcgaccttgaagtgaccactcatagcttcaactgtggaggggagtttttctattgcaatacatcaggcctgttcaactctacatggatctcaaataccagtgtccaggggtcaaattccaccggtagcaacgacagcatcgtcttgccttgtcgaatcaagcagatcattaatatgtggcagaggattggtcaggccatgtacgcacctccaatacagggagtcattcggtgcgtcagcaatattactggattgatcctcaccagagatggcgggagtaccaatagcactaccgaaactttccgcccaggaggaggcgacatgcgggataattggagatcagagctgtataagtataaggtggtgaaaattgaacccctgggagtggcgccaactagatgtaaacggcgagtggttggccggagacggcggcggagagcagtggggattggcgctgtctcactcggtttcctgggtgctgccggcagtacaatgggcgccgccagcatgacgctcacagtgcaggcccggaatcttcttagcggaattgtgcaacaacaaagcaatctgttgagagccccggaaccgcagcaacatctgttgaaggacacacattggggcatcaagcagctgcaagctcgggttctggctgttgagcattacctgagagaccaacagctgctgggcatatggggatgctcaggaaaactgatctgctgcaccaatgtcccatggaacagctcatggtcaaacaggaacctgagcgagatctgggataacatgacctggttgcagtgggacaaagaaattagcaattacacacagatcatctacggcctcctggaggaaagccagaatcagcaggagaaaaatgagcaggatctgcttgcccttgacggtggaggcggttcaggcggcggatctggcggtgggagcggttcgggagcccatatagtgatggttgatgcctataaaccgaccaagtga

SEQ ID NO: 15 - protein, 4 aa, Human immunodeficiency virus
GDIR

SEQ ID NO: 16 - protein, 4 aa, synthetic construct
  REGION 1..4: note = Description of Artificial Sequence: Synthetic peptide
GAIA

SEQ ID NO: 17 - protein, 4 aa, Human immunodeficiency virus
REKR

SEQ ID NO: 18 - protein, 6 aa, synthetic construct
  REGION 1..6: note = Description of Artificial Sequence: Synthetic peptide
RRRRRR

SEQ ID NO: 19 - protein, 6 aa, synthetic construct
  REGION 1..6: note = Description of Artificial Sequence: Synthetic 6xHis tag
HHHHHH

SEQ ID NO: 20 - protein, 6 aa, synthetic construct
  REGION 1..6: note = Description of Artificial Sequence: Synthetic peptide
  MOD_RES 2..3: note = Any amino acid
QXXDSS

SEQ ID NO: 21 - protein, 4 aa, synthetic construct
  REGION 1..4: note = Description of Artificial Sequence: Synthetic peptide
SYAG

SEQ ID NO: 22 - protein, 11 aa, synthetic construct
  REGION 1..11: note = Description of Artificial Sequence: Synthetic peptide
ASGDELAWFAY

SEQ ID NO: 23 - protein, 11 aa, synthetic construct
  REGION 1..11: note = Description of Artificial Sequence: Synthetic peptide
ASGDELACFAY

SEQ ID NO: 24 - protein, 11 aa, synthetic construct
  REGION 1..11: note = Description of Artificial Sequence: Synthetic peptide
ANGDALAWFAY

SEQ ID NO: 25 - protein, 11 aa, synthetic construct
  REGION 1..11: note = Description of Artificial Sequence: Synthetic peptide
ACGDELAWFAY

SEQ ID NO: 26 - protein, 11 aa, synthetic construct
  REGION 1..11: note = Description of Artificial Sequence: Synthetic peptide
AGGDELAWFAY

SEQ ID NO: 27 - protein, 14 aa, synthetic construct
  REGION 1..14: note = Description of Artificial Sequence: Synthetic peptide
VRGEVYYDYDGFAY

SEQ ID NO: 28 - protein, 14 aa, synthetic construct
  REGION 1..14: note = Description of Artificial Sequence: Synthetic peptide
ARGEVYYDYDGFAY

SEQ ID NO: 29 - protein, 16 aa, synthetic construct
  REGION 1..16: note = Description of Artificial Sequence: Synthetic peptide
ARIRSDYDVGWWYFDV

SEQ ID NO: 30 - protein, 11 aa, synthetic construct
  REGION 1..11: note = Description of Artificial Sequence: Synthetic peptide
ARYYYGHYFDY

SEQ ID NO: 31 - protein, 10 aa, synthetic construct
  REGION 1..10: note = Description of Artificial Sequence: Synthetic peptide
VRSGIYYFDY

SEQ ID NO: 32 - protein, 11 aa, synthetic construct
  REGION 1..11: note = Description of Artificial Sequence: Synthetic peptide
ARYLLLRPFDY

SEQ ID NO: 33 - protein, 12 aa, synthetic construct
  REGION 1..12: note = Description of Artificial Sequence: Synthetic peptide
ARAGTTGYVMDY

SEQ ID NO: 34 - protein, 11 aa, synthetic construct
  REGION 1..11: note = Description of Artificial Sequence: Synthetic peptide
AIASYYYTLDY

SEQ ID NO: 35 - protein, 12 aa, synthetic construct
  REGION 1..12: note = Description of Artificial Sequence: Synthetic peptide
ARRGAAQAPFAY

SEQ ID NO: 36 - protein, 11 aa, synthetic construct
  REGION 1..11: note = Description of Artificial Sequence: Synthetic peptide
VRSELGPAFAY

SEQ ID NO: 37 - protein, 12 aa, synthetic construct
  REGION 1..12: note = Description of Artificial Sequence: Synthetic peptide
ARRGYGYGAMDY

SEQ ID NO: 38 - protein, 13 aa, synthetic construct
  REGION 1..13: note = Description of Artificial Sequence: Synthetic peptide
ARAYSNYVPWFAY

SEQ ID NO: 39 - protein, 10 aa, synthetic construct
  REGION 1..10: note = Description of Artificial Sequence: Synthetic peptide
ARREYGFFDY

SEQ ID NO: 40 - protein, 14 aa, synthetic construct
  REGION 1..14: note = Description of Artificial Sequence: Synthetic peptide
ARHGRLTGTGAMDY

SEQ ID NO: 41 - protein, 11 aa, synthetic construct
  REGION 1..11: note = Description of Artificial Sequence: Synthetic peptide
ARHGAGNALDY

SEQ ID NO: 42 - protein, 11 aa, synthetic construct
  REGION 1..11: note = Description of Artificial Sequence: Synthetic peptide
ARHGAGNAMDY

SEQ ID NO: 43 - protein, 9 aa, synthetic construct
  REGION 1..9: note = Description of Artificial Sequence: Synthetic peptide
QVEVTMWTT

SEQ ID NO: 44 - protein, 9 aa, synthetic construct
  REGION 1..9: note = Description of Artificial Sequence: Synthetic peptide
ASGRNYVDY

SEQ ID NO: 45 - protein, 9 aa, synthetic construct
  REGION 1..9: note = Description of Artificial Sequence: Synthetic peptide
ASGPNYFDY

SEQ ID NO: 46 - protein, 15 aa, synthetic construct
  REGION 1..15: note = Description of Artificial Sequence: Synthetic peptide
ARHGHYYGSSYGMDY

SEQ ID NO: 47 - protein, 12 aa, synthetic construct
  REGION 1..12: note = Description of Artificial Sequence: Synthetic peptide
ARDDGGYWYFDV

SEQ ID NO: 48 - protein, 12 aa, synthetic construct
  REGION 1..12: note = Description of Artificial Sequence: Synthetic peptide
ANIPKDRLCYGP

SEQ ID NO: 49 - protein, 12 aa, synthetic construct
  REGION 1..12: note = Description of Artificial Sequence: Synthetic peptide
ARHEEDGYWFAY

SEQ ID NO: 50 - protein, 9 aa, synthetic construct
  REGION 1..9: note = Description of Artificial Sequence: Synthetic peptide
LQYDEFPYT

SEQ ID NO: 51 - protein, 9 aa, synthetic construct
  REGION 1..9: note = Description of Artificial Sequence: Synthetic peptide
LHYDDFPYT

SEQ ID NO: 52 - protein, 9 aa, synthetic construct
  REGION 1..9: note = Description of Artificial Sequence: Synthetic peptide
LQYDEFPFT

SEQ ID NO: 53 - protein, 9 aa, synthetic construct
  REGION 1..9: note = Description of Artificial Sequence: Synthetic peptide
LRYDDFPYT

SEQ ID NO: 54 - protein, 9 aa, synthetic construct
  REGION 1..9: note = Description of Artificial Sequence: Synthetic peptide
IQYDEFPYT

SEQ ID NO: 55 - protein, 9 aa, synthetic construct
  REGION 1..9: note = Description of Artificial Sequence: Synthetic peptide
LHYDEFPYT

SEQ ID NO: 56 - protein, 9 aa, synthetic construct
  REGION 1..9: note = Description of Artificial Sequence: Synthetic peptide
LHYDDLPYT

SEQ ID NO: 57 - protein, 9 aa, synthetic construct
  REGION 1..9: note = Description of Artificial Sequence: Synthetic peptide
LQYDEFPHT

SEQ ID NO: 58 - protein, 9 aa, synthetic construct
  REGION 1..9: note = Description of Artificial Sequence: Synthetic peptide
LQYDESPYT

SEQ ID NO: 59 - protein, 9 aa, synthetic construct
  REGION 1..9: note = Description of Artificial Sequence: Synthetic peptide
LQYDEFPCT

SEQ ID NO: 60 - protein, 9 aa, synthetic construct
  REGION 1..9: note = Description of Artificial Sequence: Synthetic peptide
LQYDDFPHT

SEQ ID NO: 61 - protein, 9 aa, synthetic construct
  REGION 1..9: note = Description of Artificial Sequence: Synthetic peptide
QQSNEDPYT

SEQ ID NO: 62 - protein, 9 aa, synthetic construct
  REGION 1..9: note = Description of Artificial Sequence: Synthetic peptide
QQSNVDPYT

SEQ ID NO: 63 - protein, 9 aa, synthetic construct
  REGION 1..9: note = Description of Artificial Sequence: Synthetic peptide
QQSHEDPYT

SEQ ID NO: 64 - protein, 9 aa, synthetic construct
  REGION 1..9: note = Description of Artificial Sequence: Synthetic peptide
QHSNEDPYT

SEQ ID NO: 65 - protein, 10 aa, synthetic construct
  REGION 1..10: note = Description of Artificial Sequence: Synthetic peptide
QQSNEDPPWT

SEQ ID NO: 66 - protein, 10 aa, synthetic construct
  REGION 1..10: note = Description of Artificial Sequence: Synthetic peptide
QQGNEDPPWT

SEQ ID NO: 67 - protein, 10 aa, synthetic construct
  REGION 1..10: note = Description of Artificial Sequence: Synthetic peptide
HQSNEDPPWT

SEQ ID NO: 68 - protein, 10 aa, synthetic construct
  REGION 1..10: note = Description of Artificial Sequence: Synthetic peptide
QQINEDPPWT

SEQ ID NO: 69 - protein, 10 aa, synthetic construct
  REGION 1..10: note = Description of Artificial Sequence: Synthetic peptide
QQSYEDPPWT

SEQ ID NO: 70 - protein, 9 aa, synthetic construct
  REGION 1..9: note = Description of Artificial Sequence: Synthetic peptide
QQSNEDPWT

SEQ ID NO: 71 - protein, 9 aa, synthetic construct
  REGION 1..9: note = Description of Artificial Sequence: Synthetic peptide
QQNNEDPWT

SEQ ID NO: 72 - protein, 9 aa, synthetic construct
  REGION 1..9: note = Description of Artificial Sequence: Synthetic peptide
LQYDEFTFT

SEQ ID NO: 73 - protein, 9 aa, synthetic construct
  REGION 1..9: note = Description of Artificial Sequence: Synthetic peptide
QQYDSYPYT

SEQ ID NO: 74 - protein, 9 aa, synthetic construct
  REGION 1..9: note = Description of Artificial Sequence: Synthetic peptide
QQYNNYPYT

SEQ ID NO: 75 - protein, 9 aa, synthetic construct
  REGION 1..9: note = Description of Artificial Sequence: Synthetic peptide
QQYNSYPYT

SEQ ID NO: 76 - protein, 9 aa, synthetic construct
  REGION 1..9: note = Description of Artificial Sequence: Synthetic peptide
QQYNTYPYT

SEQ ID NO: 77 - protein, 9 aa, synthetic construct
  REGION 1..9: note = Description of Artificial Sequence: Synthetic peptide
QQYNIYPYT

SEQ ID NO: 78 - protein, 9 aa, synthetic construct
  REGION 1..9: note = Description of Artificial Sequence: Synthetic peptide
QQSNEDPFT

SEQ ID NO: 79 - protein, 9 aa, synthetic construct
  REGION 1..9: note = Description of Artificial Sequence: Synthetic peptide
LQYDEYMYT

SEQ ID NO: 80 - protein, 9 aa, synthetic construct
  REGION 1..9: note = Description of Artificial Sequence: Synthetic peptide
QQGNTLPWT

SEQ ID NO: 81 - protein, 9 aa, synthetic construct
  REGION 1..9: note = Description of Artificial Sequence: Synthetic peptide
QQGNTIPWT

SEQ ID NO: 82 - protein, 9 aa, synthetic construct
  REGION 1..9: note = Description of Artificial Sequence: Synthetic peptide
QQGNTLPRT

SEQ ID NO: 83 - protein, 8 aa, synthetic construct
  REGION 1..8: note = Description of Artificial Sequence: Synthetic peptide
SQSTHVPT

SEQ ID NO: 84 - protein, 9 aa, synthetic construct
  REGION 1..9: note = Description of Artificial Sequence: Synthetic peptide
VQYVQFPLT

SEQ ID NO: 85 - protein, 9 aa, synthetic construct
  REGION 1..9: note = Description of Artificial Sequence: Synthetic peptide
VQYAQFPLT

SEQ ID NO: 86 - protein, 9 aa, synthetic construct
  REGION 1..9: note = Description of Artificial Sequence: Synthetic peptide
FQGSHVPWT

SEQ ID NO: 87 - protein, 9 aa, synthetic construct
  REGION 1..9: note = Description of Artificial Sequence: Synthetic peptide
VQYAQFPFT

SEQ ID NO: 88 - protein, 9 aa, synthetic construct
  REGION 1..9: note = Description of Artificial Sequence: Synthetic peptide
LQHGESPYT

SEQ ID NO: 89 - protein, 9 aa, synthetic construct
  REGION 1..9: note = Description of Artificial Sequence: Synthetic peptide
QQFTSSPYT

SEQ ID NO: 90 - protein, 9 aa, synthetic construct
  REGION 1..9: note = Description of Artificial Sequence: Synthetic peptide
QQGNTLPYT

SEQ ID NO: 91 - protein, 9 aa, synthetic construct
  REGION 1..9: note = Description of Artificial Sequence: Synthetic peptide
LQYDEFRTT

SEQ ID NO: 92 - protein, 9 aa, synthetic construct
  REGION 1..9: note = Description of Artificial Sequence: Synthetic peptide
QQYNSYPFT

SEQ ID NO: 93 - protein, 9 aa, synthetic construct
  REGION 1..9: note = Description of Artificial Sequence: Synthetic peptide
QQCSGYPLT

SEQ ID NO: 94 - protein, 9 aa, synthetic construct
  REGION 1..9: note = Description of Artificial Sequence: Synthetic peptide
QQYSKLPWT

SEQ ID NO: 95 - protein, 10 aa, synthetic construct
  REGION 1..10: note = Description of Artificial Sequence: Synthetic peptide
LQYDEFPPFT

SEQ ID NO: 96 - protein, 13 aa, synthetic construct
  REGION 1..13: note = Description of Artificial Sequence: Synthetic peptide
ARHSRTGTGAMDY

SEQ ID NO: 97 - protein, 14 aa, synthetic construct
  REGION 1..14: note = Description of Artificial Sequence: Synthetic peptide
ARPYYYGSSPYFDY

SEQ ID NO: 98 - protein, 8 aa, synthetic construct
  REGION 1..8: note = Description of Artificial Sequence: Synthetic peptide
ARSIVPDY

SEQ ID NO: 99 - protein, 10 aa, synthetic construct
  REGION 1..10: note = Description of Artificial Sequence: Synthetic peptide
ASLYGNAFDY

SEQ ID NO: 100 - protein, 9 aa, synthetic construct
  REGION 1..9: note = Description of Artificial Sequence: Synthetic peptide
ASGGNYFDY

SEQ ID NO: 101 - protein, 11 aa, synthetic construct
  REGION 1..11: note = Description of Artificial Sequence: Synthetic peptide
ARHVGDHAMDY

SEQ ID NO: 102 - protein, 14 aa, synthetic construct
  REGION 1..14: note = Description of Artificial Sequence: Synthetic peptide
ARPYYYGSSPNFDY

SEQ ID NO: 103 - protein, 9 aa, synthetic construct
  REGION 1..9: note = Description of Artificial Sequence: Synthetic peptide
GTGKNYFDH

SEQ ID NO: 104 - protein, 10 aa, synthetic construct
  REGION 1..10: note = Description of Artificial Sequence: Synthetic peptide
ATNYGAWFPY

SEQ ID NO: 105 - protein, 14 aa, synthetic construct
  REGION 1..14: note = Description of Artificial Sequence: Synthetic peptide
ARHGITTVGVAMDY

SEQ ID NO: 106 - protein, 11 aa, synthetic construct
  REGION 1..11: note = Description of Artificial Sequence: Synthetic peptide
ANIPKDRLCYG

SEQ ID NO: 107 - protein, 13 aa, synthetic construct
  REGION 1..13: note = Description of Artificial Sequence: Synthetic peptide
ARHEGNYLYAMDY

SEQ ID NO: 108 - protein, 15 aa, synthetic construct
  REGION 1..15: note = Description of Artificial Sequence: Synthetic peptide
ARPPFITVVANYFDY

SEQ ID NO: 109 - protein, 9 aa, synthetic construct
  REGION 1..9: note = Description of Artificial Sequence: Synthetic peptide
QQRSSYPPT

SEQ ID NO: 110 - protein, 11 aa, synthetic construct
  REGION 1..11: note = Description of Artificial Sequence: Synthetic peptide
ARVVNYGPLDY

SEQ ID NO: 111 - protein, 11 aa, synthetic construct
  REGION 1..11: note = Description of Artificial Sequence: Synthetic peptide
ARVVKNGPLDY

SEQ ID NO: 112 - protein, 11 aa, synthetic construct
  REGION 1..11: note = Description of Artificial Sequence: Synthetic peptide
ARVVKYGPLDY

SEQ ID NO: 113 - protein, 11 aa, synthetic construct
  REGION 1..11: note = Description of Artificial Sequence: Synthetic peptide
ARLVRYGPLDY

SEQ ID NO: 114 - protein, 11 aa, synthetic construct
  REGION 1..11: note = Description of Artificial Sequence: Synthetic peptide
ARIVKYGPLDF

SEQ ID NO: 115 - protein, 11 aa, synthetic construct
  REGION 1..11: note = Description of Artificial Sequence: Synthetic peptide
ARGSRIAPFDY

SEQ ID NO: 116 - protein, 11 aa, synthetic construct
  REGION 1..11: note = Description of Artificial Sequence: Synthetic peptide
ARGSRIAPFDH

SEQ ID NO: 117 - protein, 11 aa, synthetic construct
  REGION 1..11: note = Description of Artificial Sequence: Synthetic peptide
SRYQARGPIDS

SEQ ID NO: 118 - protein, 11 aa, synthetic construct
  REGION 1..11: note = Description of Artificial Sequence: Synthetic peptide
ARDQARGPIDY

SEQ ID NO: 119 - protein, 11 aa, synthetic construct
  REGION 1..11: note = Description of Artificial Sequence: Synthetic peptide
ARNQARGPIDY

SEQ ID NO: 120 - protein, 11 aa, synthetic construct
  REGION 1..11: note = Description of Artificial Sequence: Synthetic peptide
ARDNRIGPFDY

SEQ ID NO: 121 - protein, 11 aa, synthetic construct
  REGION 1..11: note = Description of Artificial Sequence: Synthetic peptide
ARDKRIGPFDY

SEQ ID NO: 122 - protein, 11 aa, synthetic construct
  REGION 1..11: note = Description of Artificial Sequence: Synthetic peptide
AKKRRQLENDY

SEQ ID NO: 123 - protein, 11 aa, synthetic construct
  REGION 1..11: note = Description of Artificial Sequence: Synthetic peptide
VKKRRQLENDY

SEQ ID NO: 124 - protein, 11 aa, synthetic construct
  REGION 1..11: note = Description of Artificial Sequence: Synthetic peptide
ASRIAGGPFDY

SEQ ID NO: 125 - protein, 11 aa, synthetic construct
  REGION 1..11: note = Description of Artificial Sequence: Synthetic peptide
ASRIAGGPFDF

SEQ ID NO: 126 - protein, 11 aa, synthetic construct
  REGION 1..11: note = Description of Artificial Sequence: Synthetic peptide
ASLIAAGPFDY

SEQ ID NO: 127 - protein, 11 aa, synthetic construct
  REGION 1..11: note = Description of Artificial Sequence: Synthetic peptide
ASRIRGGPFDY

SEQ ID NO: 128 - protein, 11 aa, synthetic construct
  REGION 1..11: note = Description of Artificial Sequence: Synthetic peptide
ARDIVVGPIDY

SEQ ID NO: 129 - protein, 11 aa, synthetic construct
  REGION 1..11: note = Description of Artificial Sequence: Synthetic peptide
ARDIVIGPIDY

SEQ ID NO: 130 - protein, 11 aa, synthetic construct
  REGION 1..11: note = Description of Artificial Sequence: Synthetic peptide
ATVGRLAPFDY

SEQ ID NO: 131 - protein, 11 aa, synthetic construct
  REGION 1..11: note = Description of Artificial Sequence: Synthetic peptide
ARVGRVVPFDY

SEQ ID NO: 132 - protein, 11 aa, synthetic construct
  REGION 1..11: note = Description of Artificial Sequence: Synthetic peptide
ARVGRVAPFDY

SEQ ID NO: 133 - protein, 16 aa, synthetic construct
  REGION 1..16: note = Description of Artificial Sequence: Synthetic peptide
AKSPWGQSSSFEYFEF

SEQ ID NO: 134 - protein, 16 aa, synthetic construct
  REGION 1..16: note = Description of Artificial Sequence: Synthetic peptide
AKSPWGQSTSFEYFEF

SEQ ID NO: 135 - protein, 16 aa, synthetic construct
  REGION 1..16: note = Description of Artificial Sequence: Synthetic peptide
AKSPWGQSSYFEYFEF

SEQ ID NO: 136 - protein, 16 aa, synthetic construct
  REGION 1..16: note = Description of Artificial Sequence: Synthetic peptide
ASVLWGLPQDDNSLDV

SEQ ID NO: 137 - protein, 16 aa, synthetic construct
  REGION 1..16: note = Description of Artificial Sequence: Synthetic peptide
ASVLWEVPQDDNSLDV

SEQ ID NO: 138 - protein, 16 aa, synthetic construct
  REGION 1..16: note = Description of Artificial Sequence: Synthetic peptide
ANVLWGLPQDDNSLDV

SEQ ID NO: 139 - protein, 11 aa, synthetic construct
  REGION 1..11: note = Description of Artificial Sequence: Synthetic peptide
ASLQRLGPIDY

SEQ ID NO: 140 - protein, 11 aa, synthetic construct
  REGION 1..11: note = Description of Artificial Sequence: Synthetic peptide
ASLQYFGPFEF

SEQ ID NO: 141 - protein, 11 aa, synthetic construct
  REGION 1..11: note = Description of Artificial Sequence: Synthetic peptide
ASLQYFGPFDF

SEQ ID NO: 142 - protein, 11 aa, synthetic construct
  REGION 1..11: note = Description of Artificial Sequence: Synthetic peptide
ARAERAGPFDY

SEQ ID NO: 143 - protein, 11 aa, synthetic construct
  REGION 1..11: note = Description of Artificial Sequence: Synthetic peptide
ARHPHLESFDY

SEQ ID NO: 144 - protein, 12 aa, synthetic construct
  REGION 1..12: note = Description of Artificial Sequence: Synthetic peptide
ARNYGNYGYFEF

SEQ ID NO: 145 - protein, 16 aa, synthetic construct
  REGION 1..16: note = Description of Artificial Sequence: Synthetic peptide
ATGPYWGDYYGRYFEL

SEQ ID NO: 146 - protein, 16 aa, synthetic construct
  REGION 1..16: note = Description of Artificial Sequence: Synthetic peptide
ATGPYWGDYYGRYFEF

SEQ ID NO: 147 - protein, 11 aa, synthetic construct
  REGION 1..11: note = Description of Artificial Sequence: Synthetic peptide
ATERRAGPVDY

SEQ ID NO: 148 - protein, 11 aa, synthetic construct
  REGION 1..11: note = Description of Artificial Sequence: Synthetic peptide
ATDRRAGPLDY

SEQ ID NO: 149 - protein, 12 aa, synthetic construct
  REGION 1..12: note = Description of Artificial Sequence: Synthetic peptide
AGTLAGTTSFDV

SEQ ID NO: 150 - protein, 12 aa, synthetic construct
  REGION 1..12: note = Description of Artificial Sequence: Synthetic peptide
AGGLGRTTSFDV

SEQ ID NO: 151 - protein, 15 aa, synthetic construct
  REGION 1..15: note = Description of Artificial Sequence: Synthetic peptide
ARVGSGWSTEGNFDY

SEQ ID NO: 152 - protein, 16 aa, synthetic construct
  REGION 1..16: note = Description of Artificial Sequence: Synthetic peptide
AKDWIQWLHLGSYFDF

SEQ ID NO: 153 - protein, 16 aa, synthetic construct
  REGION 1..16: note = Description of Artificial Sequence: Synthetic peptide
AKDWIQWVHLGSYFDY

SEQ ID NO: 154 - protein, 13 aa, synthetic construct
  REGION 1..13: note = Description of Artificial Sequence: Synthetic peptide
ARHSSTYVAPVDY

SEQ ID NO: 155 - protein, 12 aa, synthetic construct
  REGION 1..12: note = Description of Artificial Sequence: Synthetic peptide
ASAKGRLAPLDY

SEQ ID NO: 156 - protein, 9 aa, synthetic construct
  REGION 1..9: note = Description of Artificial Sequence: Synthetic peptide
ANWADYFDY

SEQ ID NO: 157 - protein, 16 aa, synthetic construct
  REGION 1..16: note = Description of Artificial Sequence: Synthetic peptide
ARDPVITITTRERFDV

SEQ ID NO: 158 - protein, 11 aa, synthetic construct
  REGION 1..11: note = Description of Artificial Sequence: Synthetic peptide
ARDQRTGPFDY

SEQ ID NO: 159 - protein, 11 aa, synthetic construct
  REGION 1..11: note = Description of Artificial Sequence: Synthetic peptide
ARQAFAGPTDS

SEQ ID NO: 160 - protein, 15 aa, synthetic construct
  REGION 1..15: note = Description of Artificial Sequence: Synthetic peptide
ARRGPVNWNGSSLDV

SEQ ID NO: 161 - protein, 16 aa, synthetic construct
  REGION 1..16: note = Description of Artificial Sequence: Synthetic peptide
TRDRADSWNFHDYFDY

SEQ ID NO: 162 - protein, 11 aa, synthetic construct
  REGION 1..11: note = Description of Artificial Sequence: Synthetic peptide
AKIAVAGPVDY

SEQ ID NO: 163 - protein, 14 aa, synthetic construct
  REGION 1..14: note = Description of Artificial Sequence: Synthetic peptide
ATTYSGSDYYRLDV

SEQ ID NO: 164 - protein, 13 aa, synthetic construct
  REGION 1..13: note = Description of Artificial Sequence: Synthetic peptide
ARPDSLWGAAFDY

SEQ ID NO: 165 - protein, 11 aa, synthetic construct
  REGION 1..11: note = Description of Artificial Sequence: Synthetic peptide
ARIGAAGPGDY

SEQ ID NO: 166 - protein, 15 aa, synthetic construct
  REGION 1..15: note = Description of Artificial Sequence: Synthetic peptide
AKYWGDYYGYSSLDV

SEQ ID NO: 167 - protein, 11 aa, synthetic construct
  REGION 1..11: note = Description of Artificial Sequence: Synthetic peptide
ARVEVVGPTGY

SEQ ID NO: 168 - protein, 13 aa, synthetic construct
  REGION 1..13: note = Description of Artificial Sequence: Synthetic peptide
ARRYSGSYSPFDC

SEQ ID NO: 169 - protein, 18 aa, synthetic construct
  REGION 1..18: note = Description of Artificial Sequence: Synthetic peptide
AREGMGCTGSGCSISFDY

SEQ ID NO: 170 - protein, 13 aa, synthetic construct
  REGION 1..13: note = Description of Artificial Sequence: Synthetic peptide
ARQGYSGYSLFDY

SEQ ID NO: 171 - protein, 11 aa, synthetic construct
  REGION 1..11: note = Description of Artificial Sequence: Synthetic peptide
ASEIAGGPVDY

SEQ ID NO: 172 - protein, 15 aa, synthetic construct
  REGION 1..15: note = Description of Artificial Sequence: Synthetic peptide
ARDSSGWPWDNRFDV

SEQ ID NO: 173 - protein, 12 aa, synthetic construct
  REGION 1..12: note = Description of Artificial Sequence: Synthetic peptide
ARVTGRIAPFDY

SEQ ID NO: 174 - protein, 16 aa, synthetic construct
  REGION 1..16: note = Description of Artificial Sequence: Synthetic peptide
ATNIWTGYSFYYGLDS

SEQ ID NO: 175 - protein, 11 aa, synthetic construct
  REGION 1..11: note = Description of Artificial Sequence: Synthetic peptide
AREGRIHPLDS

SEQ ID NO: 176 - protein, 12 aa, synthetic construct
  REGION 1..12: note = Description of Artificial Sequence: Synthetic peptide
AKDHDYGGGLDS

SEQ ID NO: 177 - protein, 12 aa, synthetic construct
  REGION 1..12: note = Description of Artificial Sequence: Synthetic peptide
AKKSSGSWEVDY

SEQ ID NO: 178 - protein, 19 aa, synthetic construct
  REGION 1..19: note = Description of Artificial Sequence: Synthetic peptide
ARHAYYNIWTGYSTNRFDV

SEQ ID NO: 179 - protein, 13 aa, synthetic construct
  REGION 1..13: note = Description of Artificial Sequence: Synthetic peptide
AEGSGSWNGRFGV

SEQ ID NO: 180 - protein, 17 aa, synthetic construct
  REGION 1..17: note = Description of Artificial Sequence: Synthetic peptide
ATGRYYGGSYYGDRFDV

SEQ ID NO: 181 - protein, 12 aa, synthetic construct
  REGION 1..12: note = Description of Artificial Sequence: Synthetic peptide
AKCSSSSTGLDY

SEQ ID NO: 182 - protein, 18 aa, synthetic construct
  REGION 1..18: note = Description of Artificial Sequence: Synthetic peptide
ARDRSVTPFSWVEYYFDY

SEQ ID NO: 183 - protein, 11 aa, synthetic construct
  REGION 1..11: note = Description of Artificial Sequence: Synthetic peptide
VRVVKYGPLDY

SEQ ID NO: 184 - protein, 20 aa, synthetic construct
  REGION 1..20: note = Description of Artificial Sequence: Synthetic peptide
ARNPPYYNLWTGYYTHSLDV

SEQ ID NO: 185 - protein, 17 aa, synthetic construct
  REGION 1..17: note = Description of Artificial Sequence: Synthetic peptide
AREGYCSYTYCSNLFEF

SEQ ID NO: 186 - protein, 11 aa, synthetic construct
  REGION 1..11: note = Description of Artificial Sequence: Synthetic peptide
ARARIAAPFDY

SEQ ID NO: 187 - protein, 11 aa, synthetic construct
  REGION 1..11: note = Description of Artificial Sequence: Synthetic peptide
ARAGRMAATDY

SEQ ID NO: 188 - protein, 11 aa, synthetic construct
  REGION 1..11: note = Description of Artificial Sequence: Synthetic peptide
VRDVTLGPIDN

SEQ ID NO: 189 - protein, 11 aa, synthetic construct
  REGION 1..11: note = Description of Artificial Sequence: Synthetic peptide
AREGRIQPLDS

SEQ ID NO: 190 - protein, 11 aa, synthetic construct
  REGION 1..11: note = Description of Artificial Sequence: Synthetic peptide
AKCRNWNDFAY

SEQ ID NO: 191 - protein, 11 aa, synthetic construct
  REGION 1..11: note = Description of Artificial Sequence: Synthetic peptide
ARVHRGGPFDY

SEQ ID NO: 192 - protein, 11 aa, synthetic construct
  REGION 1..11: note = Description of Artificial Sequence: Synthetic peptide
ARGGRVHPMDY

SEQ ID NO: 193 - protein, 11 aa, synthetic construct
  REGION 1..11: note = Description of Artificial Sequence: Synthetic peptide
ARGGPVSPFDY

SEQ ID NO: 194 - protein, 11 aa, synthetic construct
  REGION 1..11: note = Description of Artificial Sequence: Synthetic peptide
ARGQRVAPFDV

SEQ ID NO: 195 - protein, 21 aa, synthetic construct
  REGION 1..21: note = Description of Artificial Sequence: Synthetic peptide
AKETYEDDYGYYSLGYNRFDV

SEQ ID NO: 196 - protein, 12 aa, synthetic construct
  REGION 1..12: note = Description of Artificial Sequence: Synthetic peptide
ASAWREHLPIDY

SEQ ID NO: 197 - protein, 16 aa, synthetic construct
  REGION 1..16: note = Description of Artificial Sequence: Synthetic peptide
ARDLYPGVINPSGLDS

SEQ ID NO: 198 - protein, 16 aa, synthetic construct
  REGION 1..16: note = Description of Artificial Sequence: Synthetic peptide
ARDKGSSYYQPEYFEF

SEQ ID NO: 199 - protein, 16 aa, synthetic construct
  REGION 1..16: note = Description of Artificial Sequence: Synthetic peptide
VRDKGSSYYQPEYFEF

SEQ ID NO: 200 - protein, 11 aa, synthetic construct
  REGION 1..11: note = Description of Artificial Sequence: Synthetic peptide
ARTGKAAPVDY

SEQ ID NO: 201 - protein, 11 aa, synthetic construct
  REGION 1..11: note = Description of Artificial Sequence: Synthetic peptide
ARTGKAAPVDC

SEQ ID NO: 202 - protein, 16 aa, synthetic construct
  REGION 1..16: note = Description of Artificial Sequence: Synthetic peptide
AKGGDNYYDSGYYDDY

SEQ ID NO: 203 - protein, 13 aa, synthetic construct
  REGION 1..13: note = Description of Artificial Sequence: Synthetic peptide
ARNRGWGDLVFDY

SEQ ID NO: 204 - protein, 14 aa, synthetic construct
  REGION 1..14: note = Description of Artificial Sequence: Synthetic peptide
AKVLSGWFWDYFDY

SEQ ID NO: 205 - protein, 11 aa, synthetic construct
  REGION 1..11: note = Description of Artificial Sequence: Synthetic peptide
ARLAVAGPVDY

SEQ ID NO: 206 - protein, 14 aa, synthetic construct
  REGION 1..14: note = Description of Artificial Sequence: Synthetic peptide
ARGSSGWYGSGLDS

SEQ ID NO: 207 - protein, 16 aa, synthetic construct
  REGION 1..16: note = Description of Artificial Sequence: Synthetic peptide
ARDHIESWNKVNWFDV

SEQ ID NO: 208 - protein, 14 aa, synthetic construct
  REGION 1..14: note = Description of Artificial Sequence: Synthetic peptide
ATYSGSWYAEYFEF

SEQ ID NO: 209 - protein, 17 aa, synthetic construct
  REGION 1..17: note = Description of Artificial Sequence: Synthetic peptide
AKQEDYNFWSSYFLPDY

SEQ ID NO: 210 - protein, 13 aa, synthetic construct
  REGION 1..13: note = Description of Artificial Sequence: Synthetic peptide
ARDSSGWYEGFDY

SEQ ID NO: 211 - protein, 15 aa, synthetic construct
  REGION 1..15: note = Description of Artificial Sequence: Synthetic peptide
ATGRYYGPSWAIFDY

SEQ ID NO: 212 - protein, 11 aa, synthetic construct
  REGION 1..11: note = Description of Artificial Sequence: Synthetic peptide
ARDGNFGPIDY

SEQ ID NO: 213 - protein, 9 aa, synthetic construct
  REGION 1..9: note = Description of Artificial Sequence: Synthetic peptide
ASGPNWFDV

SEQ ID NO: 214 - protein, 15 aa, synthetic construct
  REGION 1..15: note = Description of Artificial Sequence: Synthetic peptide
AKSETDFWTSYYFNY

SEQ ID NO: 215 - protein, 20 aa, synthetic construct
  REGION 1..20: note = Description of Artificial Sequence: Synthetic peptide
ARDICSGSGCYWYRDNWFDV

SEQ ID NO: 216 - protein, 11 aa, synthetic construct
  REGION 1..11: note = Description of Artificial Sequence: Synthetic peptide
ASNRRIAPLDY

SEQ ID NO: 217 - protein, 9 aa, synthetic construct
  REGION 1..9: note = Description of Artificial Sequence: Synthetic peptide
ASGRYYFDY

SEQ ID NO: 218 - protein, 15 aa, synthetic construct
  REGION 1..15: note = Description of Artificial Sequence: Synthetic peptide
ARDRTVTPNRGYFEF

SEQ ID NO: 219 - protein, 15 aa, synthetic construct
  REGION 1..15: note = Description of Artificial Sequence: Synthetic peptide
ARDGPYSGGWSELDS

SEQ ID NO: 220 - protein, 13 aa, synthetic construct
  REGION 1..13: note = Description of Artificial Sequence: Synthetic peptide
ARWEYSGNWGLDY

SEQ ID NO: 221 - protein, 16 aa, synthetic construct
  REGION 1..16: note = Description of Artificial Sequence: Synthetic peptide
ARSTSSWPRTSDAFDF

SEQ ID NO: 222 - protein, 12 aa, synthetic construct
  REGION 1..12: note = Description of Artificial Sequence: Synthetic peptide
AKKRSSWSRIDY

SEQ ID NO: 223 - protein, 14 aa, synthetic construct
  REGION 1..14: note = Description of Artificial Sequence: Synthetic peptide
ARDGSGWRRVTFDY

SEQ ID NO: 224 - protein, 9 aa, synthetic construct
  REGION 1..9: note = Description of Artificial Sequence: Synthetic peptide
ATGRNYFDY

SEQ ID NO: 225 - protein, 12 aa, synthetic construct
  REGION 1..12: note = Description of Artificial Sequence: Synthetic peptide
AKTGAVTTGFDY

SEQ ID NO: 226 - protein, 14 aa, synthetic construct
  REGION 1..14: note = Description of Artificial Sequence: Synthetic peptide
ARLVGGSGYYYIGD

SEQ ID NO: 227 - protein, 13 aa, synthetic construct
  REGION 1..13: note = Description of Artificial Sequence: Synthetic peptide
AKVPYSSWSHFDY

SEQ ID NO: 228 - protein, 14 aa, synthetic construct
  REGION 1..14: note = Description of Artificial Sequence: Synthetic peptide
TSPRMRYSSGSFDY

SEQ ID NO: 229 - protein, 14 aa, synthetic construct
  REGION 1..14: note = Description of Artificial Sequence: Synthetic peptide
ARVRGYSGYSFFDY

SEQ ID NO: 230 - protein, 13 aa, synthetic construct
  REGION 1..13: note = Description of Artificial Sequence: Synthetic peptide
SRGSTWSGDWFDV

SEQ ID NO: 231 - protein, 15 aa, synthetic construct
  REGION 1..15: note = Description of Artificial Sequence: Synthetic peptide
TKRLAYSNPYNRFDV

SEQ ID NO: 232 - protein, 27 aa, synthetic construct
  REGION 1..27: note = Description of Artificial Sequence: Synthetic peptide
ARGGVGLDDVTYYYSGSYYYHRTSFDY

SEQ ID NO: 233 - protein, 18 aa, synthetic construct
  REGION 1..18: note = Description of Artificial Sequence: Synthetic peptide
AGDRGGYNYGFTDNWFDV

SEQ ID NO: 234 - protein, 20 aa, synthetic construct
  REGION 1..20: note = Description of Artificial Sequence: Synthetic peptide
TRGTAYYNFWSNSSPGYFDY

SEQ ID NO: 235 - protein, 16 aa, synthetic construct
  REGION 1..16: note = Description of Artificial Sequence: Synthetic peptide
ARDKGSSYYQPESFEF

SEQ ID NO: 236 - protein, 27 aa, synthetic construct
  REGION 1..27: note = Description of Artificial Sequence: Synthetic peptide
ARRYYEDDYGYYYPGPNIAGTTRGVEE

SEQ ID NO: 237 - protein, 18 aa, synthetic construct
  REGION 1..18: note = Description of Artificial Sequence: Synthetic peptide
ARGITRMITVTKTNWFDV

SEQ ID NO: 238 - protein, 11 aa, synthetic construct
  REGION 1..11: note = Description of Artificial Sequence: Synthetic peptide
ARLAVAGPFDY

SEQ ID NO: 239 - protein, 11 aa, synthetic construct
  REGION 1..11: note = Description of Artificial Sequence: Synthetic peptide
ARLGVAGPLDY

SEQ ID NO: 240 - protein, 8 aa, synthetic construct
  REGION 1..8: note = Description of Artificial Sequence: Synthetic peptide
ATYKTIDY

SEQ ID NO: 241 - protein, 8 aa, synthetic construct
  REGION 1..8: note = Description of Artificial Sequence: Synthetic peptide
ASYKNIDY

SEQ ID NO: 242 - protein, 11 aa, synthetic construct
  REGION 1..11: note = Description of Artificial Sequence: Synthetic peptide
ARDRHGIPFDY

SEQ ID NO: 243 - protein, 13 aa, synthetic construct
  REGION 1..13: note = Description of Artificial Sequence: Synthetic peptide
ARSRGYWGDLFDF

SEQ ID NO: 244 - protein, 13 aa, synthetic construct
  REGION 1..13: note = Description of Artificial Sequence: Synthetic peptide
ARLSGWGDFRIDY

SEQ ID NO: 245 - protein, 8 aa, synthetic construct
  REGION 1..8: note = Description of Artificial Sequence: Synthetic peptide
ATGIWFDV

SEQ ID NO: 246 - protein, 11 aa, synthetic construct
  REGION 1..11: note = Description of Artificial Sequence: Synthetic peptide
ARANNGGYFDY

SEQ ID NO: 247 - protein, 15 aa, synthetic construct
  REGION 1..15: note = Description of Artificial Sequence: Synthetic peptide
ARMTTVAAFGGYFDL

SEQ ID NO: 248 - protein, 9 aa, synthetic construct
  REGION 1..9: note = Description of Artificial Sequence: Synthetic peptide
ASGGNYADY

SEQ ID NO: 249 - protein, 11 aa, synthetic construct
  REGION 1..11: note = Description of Artificial Sequence: Synthetic peptide
ARRLSRRYFDY

SEQ ID NO: 250 - protein, 16 aa, synthetic construct
  REGION 1..16: note = Description of Artificial Sequence: Synthetic peptide
TREFCSGIYCYAPFDY

SEQ ID NO: 251 - protein, 8 aa, synthetic construct
  REGION 1..8: note = Description of Artificial Sequence: Synthetic peptide
ASFKTLDV

SEQ ID NO: 252 - protein, 14 aa, synthetic construct
  REGION 1..14: note = Description of Artificial Sequence: Synthetic peptide
AKGVGGFSYSYPHY

SEQ ID NO: 253 - protein, 14 aa, synthetic construct
  REGION 1..14: note = Description of Artificial Sequence: Synthetic peptide
ARDGHYNFWSPPGY

SEQ ID NO: 254 - protein, 14 aa, synthetic construct
  REGION 1..14: note = Description of Artificial Sequence: Synthetic peptide
ARAEDEDDYGSFDV

SEQ ID NO: 255 - protein, 16 aa, synthetic construct
  REGION 1..16: note = Description of Artificial Sequence: Synthetic peptide
ARLGSSGWYRDDAFDF

SEQ ID NO: 256 - protein, 11 aa, synthetic construct
  REGION 1..11: note = Description of Artificial Sequence: Synthetic peptide
AKPRGRWLEDY

SEQ ID NO: 257 - protein, 11 aa, synthetic construct
  REGION 1..11: note = Description of Artificial Sequence: Synthetic peptide
TRPRQYSTGDY

SEQ ID NO: 258 - protein, 17 aa, synthetic construct
  REGION 1..17: note = Description of Artificial Sequence: Synthetic peptide
AKMGGRGYSSYGPVFDY

SEQ ID NO: 259 - protein, 11 aa, synthetic construct
  REGION 1..11: note = Description of Artificial Sequence: Synthetic peptide
ARIVTRGPFDY

SEQ ID NO: 260 - protein, 17 aa, synthetic construct
  REGION 1..17: note = Description of Artificial Sequence: Synthetic peptide
ARDVTTRVVIIDHRFDV

SEQ ID NO: 261 - protein, 14 aa, synthetic construct
  REGION 1..14: note = Description of Artificial Sequence: Synthetic peptide
ARQLGGGQTDRFDV

SEQ ID NO: 262 - protein, 11 aa, synthetic construct
  REGION 1..11: note = Description of Artificial Sequence: Synthetic peptide
ARQAYSNYPDY

SEQ ID NO: 263 - protein, 12 aa, synthetic construct
  REGION 1..12: note = Description of Artificial Sequence: Synthetic peptide
VKLREKWETRGD

SEQ ID NO: 264 - protein, 13 aa, synthetic construct
  REGION 1..13: note = Description of Artificial Sequence: Synthetic peptide
AKSYGSMSNRFDV

SEQ ID NO: 265 - protein, 11 aa, synthetic construct
  REGION 1..11: note = Description of Artificial Sequence: Synthetic peptide
ARVIRLGPFDY

SEQ ID NO: 266 - protein, 22 aa, synthetic construct
  REGION 1..22: note = Description of Artificial Sequence: Synthetic peptide
ARETFEGDDYGYYYTPDNWFDV

SEQ ID NO: 267 - protein, 14 aa, synthetic construct
  REGION 1..14: note = Description of Artificial Sequence: Synthetic peptide
AKSGNSGSWNYFDY

SEQ ID NO: 268 - protein, 13 aa, synthetic construct
  REGION 1..13: note = Description of Artificial Sequence: Synthetic peptide
ARRRGWGDPYFDY

SEQ ID NO: 269 - protein, 14 aa, synthetic construct
  REGION 1..14: note = Description of Artificial Sequence: Synthetic peptide
ATGFSMITVALFDF

SEQ ID NO: 270 - protein, 18 aa, synthetic construct
  REGION 1..18: note = Description of Artificial Sequence: Synthetic peptide
ASQGYEDDYAYWAFKFDY

SEQ ID NO: 271 - protein, 12 aa, synthetic construct
  REGION 1..12: note = Description of Artificial Sequence: Synthetic peptide
ARSPGIVAPFDY

SEQ ID NO: 272 - protein, 15 aa, synthetic construct
  REGION 1..15: note = Description of Artificial Sequence: Synthetic peptide
ARSRSGSNSESRFDV

SEQ ID NO: 273 - protein, 17 aa, synthetic construct
  REGION 1..17: note = Description of Artificial Sequence: Synthetic peptide
ARPLYSGNWNVYWYFDL

SEQ ID NO: 274 - protein, 12 aa, synthetic construct
  REGION 1..12: note = Description of Artificial Sequence: Synthetic peptide
ARDGWGGWTIDY

SEQ ID NO: 275 - protein, 13 aa, synthetic construct
  REGION 1..13: note = Description of Artificial Sequence: Synthetic peptide
ARSGYGSGGTFDY

SEQ ID NO: 276 - protein, 16 aa, synthetic construct
  REGION 1..16: note = Description of Artificial Sequence: Synthetic peptide
ATTPGYCSSTYCRFDY

SEQ ID NO: 277 - protein, 19 aa, synthetic construct
  REGION 1..19: note = Description of Artificial Sequence: Synthetic peptide
ATKNYYDSGYHLSGEYFEF

SEQ ID NO: 278 - protein, 15 aa, synthetic construct
  REGION 1..15: note = Description of Artificial Sequence: Synthetic peptide
AQCPEYSWNMGWFDV

SEQ ID NO: 279 - protein, 16 aa, synthetic construct
  REGION 1..16: note = Description of Artificial Sequence: Synthetic peptide
ASPFYGSGYYTRRFDV

SEQ ID NO: 280 - protein, 18 aa, synthetic construct
  REGION 1..18: note = Description of Artificial Sequence: Synthetic peptide
ARDGYYSGDYYRHNWFAV

SEQ ID NO: 281 - protein, 10 aa, synthetic construct
  REGION 1..10: note = Description of Artificial Sequence: Synthetic peptide
ARDCVDAFDY

SEQ ID NO: 282 - protein, 12 aa, synthetic construct
  REGION 1..12: note = Description of Artificial Sequence: Synthetic peptide
ATGYNWNDPFDY

SEQ ID NO: 283 - protein, 16 aa, synthetic construct
  REGION 1..16: note = Description of Artificial Sequence: Synthetic peptide
TKVEGGYWGDYHRFDV

SEQ ID NO: 284 - protein, 11 aa, synthetic construct
  REGION 1..11: note = Description of Artificial Sequence: Synthetic peptide
QSYDSSLSGHL

SEQ ID NO: 285 - protein, 11 aa, synthetic construct
  REGION 1..11: note = Description of Artificial Sequence: Synthetic peptide
QSYDSSLSAGL

SEQ ID NO: 286 - protein, 11 aa, synthetic construct
  REGION 1..11: note = Description of Artificial Sequence: Synthetic peptide
QSYDSSLSAHV

SEQ ID NO: 287 - protein, 11 aa, synthetic construct
  REGION 1..11: note = Description of Artificial Sequence: Synthetic peptide
QSYDNSLSAWV

SEQ ID NO: 288 - protein, 11 aa, synthetic construct
  REGION 1..11: note = Description of Artificial Sequence: Synthetic peptide
QSYDSSLSVRV

SEQ ID NO: 289 - protein, 11 aa, synthetic construct
  REGION 1..11: note = Description of Artificial Sequence: Synthetic peptide
QSYDNSLSARV

SEQ ID NO: 290 - protein, 11 aa, synthetic construct
  REGION 1..11: note = Description of Artificial Sequence: Synthetic peptide
  MOD_RES 9: note = Any amino acid
QSYDNSLSXQV

SEQ ID NO: 291 - protein, 11 aa, synthetic construct
  REGION 1..11: note = Description of Artificial Sequence: Synthetic peptide
QSYDNSLSAHV

SEQ ID NO: 292 - protein, 11 aa, synthetic construct
  REGION 1..11: note = Description of Artificial Sequence: Synthetic peptide
QSYDSSLSADV

SEQ ID NO: 293 - protein, 11 aa, synthetic construct
  REGION 1..11: note = Description of Artificial Sequence: Synthetic peptide
LSYDSSLSAHI

SEQ ID NO: 294 - protein, 11 aa, synthetic construct
  REGION 1..11: note = Description of Artificial Sequence: Synthetic peptide
QVWDSSSDHPL

SEQ ID NO: 295 - protein, 11 aa, synthetic construct
  REGION 1..11: note = Description of Artificial Sequence: Synthetic peptide
GAWDSSLSAGL

SEQ ID NO: 296 - protein, 11 aa, synthetic construct
  REGION 1..11: note = Description of Artificial Sequence: Synthetic peptide
SAWDSSLSDVL

SEQ ID NO: 297 - protein, 11 aa, synthetic construct
  REGION 1..11: note = Description of Artificial Sequence: Synthetic peptide
AAWDDSLSGVL

SEQ ID NO: 298 - protein, 11 aa, synthetic construct
  REGION 1..11: note = Description of Artificial Sequence: Synthetic peptide
ETWDYSLNGPL

SEQ ID NO: 299 - protein, 9 aa, synthetic construct
  REGION 1..9: note = Description of Artificial Sequence: Synthetic peptide
YSGDDNNDV

SEQ ID NO: 300 - protein, 11 aa, synthetic construct
  REGION 1..11: note = Description of Artificial Sequence: Synthetic peptide
QSYDSSLSGHI

SEQ ID NO: 301 - protein, 7 aa, synthetic construct
  REGION 1..7: note = Description of Artificial Sequence: Synthetic peptide
QTWTTDV

SEQ ID NO: 302 - protein, 10 aa, synthetic construct
  REGION 1..10: note = Description of Artificial Sequence: Synthetic peptide
CSYTTSNTLL

SEQ ID NO: 303 - protein, 12 aa, synthetic construct
  REGION 1..12: note = Description of Artificial Sequence: Synthetic peptide
QSYDSSLSVHYI

SEQ ID NO: 304 - protein, 10 aa, synthetic construct
  REGION 1..10: note = Description of Artificial Sequence: Synthetic peptide
SSYASSSTWV

SEQ ID NO: 305 - protein, 11 aa, synthetic construct
  REGION 1..11: note = Description of Artificial Sequence: Synthetic peptide
QSYDSSLSAVL

SEQ ID NO: 306 - protein, 11 aa, synthetic construct
  REGION 1..11: note = Description of Artificial Sequence: Synthetic peptide
QSYDSSLSALL

SEQ ID NO: 307 - protein, 11 aa, synthetic construct
  REGION 1..11: note = Description of Artificial Sequence: Synthetic peptide
QSYDSSLSAVF

SEQ ID NO: 308 - protein, 11 aa, synthetic construct
  REGION 1..11: note = Description of Artificial Sequence: Synthetic peptide
QSYDSSLSARL

SEQ ID NO: 309 - protein, 11 aa, synthetic construct
  REGION 1..11: note = Description of Artificial Sequence: Synthetic peptide
QSYDSSLSNVL

SEQ ID NO: 310 - protein, 11 aa, synthetic construct
  REGION 1..11: note = Description of Artificial Sequence: Synthetic peptide
QSYDSSLSGVL

SEQ ID NO: 311 - protein, 11 aa, synthetic construct
  REGION 1..11: note = Description of Artificial Sequence: Synthetic peptide
QSYDNSLSAVL

SEQ ID NO: 312 - protein, 11 aa, synthetic construct
  REGION 1..11: note = Description of Artificial Sequence: Synthetic peptide
QSYDNNLSAVL

SEQ ID NO: 313 - protein, 11 aa, synthetic construct
  REGION 1..11: note = Description of Artificial Sequence: Synthetic peptide
QSYDSSLSAQV

SEQ ID NO: 314 - protein, 11 aa, synthetic construct
  REGION 1..11: note = Description of Artificial Sequence: Synthetic peptide
QSYDSSLSAHL

SEQ ID NO: 315 - protein, 11 aa, synthetic construct
  REGION 1..11: note = Description of Artificial Sequence: Synthetic peptide
QSYDSSLSAWV

SEQ ID NO: 316 - protein, 11 aa, synthetic construct
  REGION 1..11: note = Description of Artificial Sequence: Synthetic peptide
QSYDSYLSAGL

SEQ ID NO: 317 - protein, 11 aa, synthetic construct
  REGION 1..11: note = Description of Artificial Sequence: Synthetic peptide
QSYDNSLSDDV

SEQ ID NO: 318 - protein, 11 aa, synthetic construct
  REGION 1..11: note = Description of Artificial Sequence: Synthetic peptide
QSYDSSLSALV

SEQ ID NO: 319 - protein, 12 aa, synthetic construct
  REGION 1..12: note = Description of Artificial Sequence: Synthetic peptide
QSYDSNLSAHVL

SEQ ID NO: 320 - protein, 12 aa, synthetic construct
  REGION 1..12: note = Description of Artificial Sequence: Synthetic peptide
QSFDSNLSIHLL

SEQ ID NO: 321 - protein, 12 aa, synthetic construct
  REGION 1..12: note = Description of Artificial Sequence: Synthetic peptide
QSYDSSLSAHVL

SEQ ID NO: 322 - protein, 11 aa, synthetic construct
  REGION 1..11: note = Description of Artificial Sequence: Synthetic peptide
QSYDSSLSAYI

SEQ ID NO: 323 - protein, 11 aa, synthetic construct
  REGION 1..11: note = Description of Artificial Sequence: Synthetic peptide
DSWDSGGTHVL

SEQ ID NO: 324 - protein, 11 aa, synthetic construct
  REGION 1..11: note = Description of Artificial Sequence: Synthetic peptide
AAWDDSLSGPV

SEQ ID NO: 325 - protein, 11 aa, synthetic construct
  REGION 1..11: note = Description of Artificial Sequence: Synthetic peptide
QVWDSRSDHPL

SEQ ID NO: 326 - protein, 9 aa, synthetic construct
  REGION 1..9: note = Description of Artificial Sequence: Synthetic peptide
MIWHNNASI

SEQ ID NO: 327 - protein, 10 aa, synthetic construct
  REGION 1..10: note = Description of Artificial Sequence: Synthetic peptide
WLYYSGGHGL

SEQ ID NO: 328 - protein, 12 aa, synthetic construct
  REGION 1..12: note = Description of Artificial Sequence: Synthetic peptide
QVWDSSSDHHDV

SEQ ID NO: 329 - protein, 11 aa, synthetic construct
  REGION 1..11: note = Description of Artificial Sequence: Synthetic peptide
QSYDSSLRAQV

SEQ ID NO: 330 - protein, 11 aa, synthetic construct
  REGION 1..11: note = Description of Artificial Sequence: Synthetic peptide
QSHDSSLTAGL

SEQ ID NO: 331 - protein, 11 aa, synthetic construct
  REGION 1..11: note = Description of Artificial Sequence: Synthetic peptide
QSHDSSLSAGL

SEQ ID NO: 332 - protein, 11 aa, synthetic construct
  REGION 1..11: note = Description of Artificial Sequence: Synthetic peptide
AAWDDSLKGWV

SEQ ID NO: 333 - protein, 11 aa, synthetic construct
  REGION 1..11: note = Description of Artificial Sequence: Synthetic peptide
AAWDDSLSGWV

SEQ ID NO: 334 - protein, 11 aa, synthetic construct
  REGION 1..11: note = Description of Artificial Sequence: Synthetic peptide
AAWDDSLNGWV

SEQ ID NO: 335 - protein, 11 aa, synthetic construct
  REGION 1..11: note = Description of Artificial Sequence: Synthetic peptide
AAWDDSLSGPL

SEQ ID NO: 336 - protein, 9 aa, synthetic construct
  REGION 1..9: note = Description of Artificial Sequence: Synthetic peptide
MIWHNNVWA

SEQ ID NO: 337 - protein, 9 aa, synthetic construct
  REGION 1..9: note = Description of Artificial Sequence: Synthetic peptide
VIWHNNVWA

SEQ ID NO: 338 - protein, 9 aa, synthetic construct
  REGION 1..9: note = Description of Artificial Sequence: Synthetic peptide
MIWHNNAWI

SEQ ID NO: 339 - protein, 9 aa, synthetic construct
  REGION 1..9: note = Description of Artificial Sequence: Synthetic peptide
MIWHNNAWV

SEQ ID NO: 340 - protein, 11 aa, synthetic construct
  REGION 1..11: note = Description of Artificial Sequence: Synthetic peptide
AAWDDSLSGYI

SEQ ID NO: 341 - protein, 11 aa, synthetic construct
  REGION 1..11: note = Description of Artificial Sequence: Synthetic peptide
QSYDNSLSAYI

SEQ ID NO: 342 - protein, 11 aa, synthetic construct
  REGION 1..11: note = Description of Artificial Sequence: Synthetic peptide
QSYDSILSSYI

SEQ ID NO: 343 - protein, 11 aa, synthetic construct
  REGION 1..11: note = Description of Artificial Sequence: Synthetic peptide
QSYDSRLSADV

SEQ ID NO: 344 - protein, 12 aa, synthetic construct
  REGION 1..12: note = Description of Artificial Sequence: Synthetic peptide
QVWDGSTKYAGL

SEQ ID NO: 345 - protein, 12 aa, synthetic construct
  REGION 1..12: note = Description of Artificial Sequence: Synthetic peptide
QVWDDSTNYAGL

SEQ ID NO: 346 - protein, 11 aa, synthetic construct
  REGION 1..11: note = Description of Artificial Sequence: Synthetic peptide
GAWDSSLSALL

SEQ ID NO: 347 - protein, 11 aa, synthetic construct
  REGION 1..11: note = Description of Artificial Sequence: Synthetic peptide
QSYDSSLSDVL

SEQ ID NO: 348 - protein, 11 aa, synthetic construct
  REGION 1..11: note = Description of Artificial Sequence: Synthetic peptide
QSYDSSLSAQL

SEQ ID NO: 349 - protein, 10 aa, synthetic construct
  REGION 1..10: note = Description of Artificial Sequence: Synthetic peptide
MIWHEDDFVL

SEQ ID NO: 350 - protein, 12 aa, synthetic construct
  REGION 1..12: note = Description of Artificial Sequence: Synthetic peptide
GAWDSSLSAHWV

SEQ ID NO: 351 - protein, 11 aa, synthetic construct
  REGION 1..11: note = Description of Artificial Sequence: Synthetic peptide
DSWDSSGTHVL

SEQ ID NO: 352 - protein, 10 aa, synthetic construct
  REGION 1..10: note = Description of Artificial Sequence: Synthetic peptide
SSYVGSGTYI

SEQ ID NO: 353 - protein, 10 aa, synthetic construct
  REGION 1..10: note = Description of Artificial Sequence: Synthetic peptide
SSYAGSGTGL

SEQ ID NO: 354 - protein, 10 aa, synthetic construct
  REGION 1..10: note = Description of Artificial Sequence: Synthetic peptide
CSYTTSNTLI

SEQ ID NO: 355 - protein, 11 aa, synthetic construct
  REGION 1..11: note = Description of Artificial Sequence: Synthetic peptide
QVWDISSDHPV

SEQ ID NO: 356 - protein, 11 aa, synthetic construct
  REGION 1..11: note = Description of Artificial Sequence: Synthetic peptide
QVWDSSSAHPV

SEQ ID NO: 357 - protein, 12 aa, synthetic construct
  REGION 1..12: note = Description of Artificial Sequence: Synthetic peptide
QSYDSSLSAHYI

SEQ ID NO: 358 - protein, 11 aa, synthetic construct
  REGION 1..11: note = Description of Artificial Sequence: Synthetic peptide
QSADSSGNHWV

SEQ ID NO: 359 - protein, 9 aa, synthetic construct
  REGION 1..9: note = Description of Artificial Sequence: Synthetic peptide
QTWTTGIHV

SEQ ID NO: 360 - protein, 10 aa, synthetic construct
  REGION 1..10: note = Description of Artificial Sequence: Synthetic peptide
GSYRTGATFL

SEQ ID NO: 361 - protein, 10 aa, synthetic construct
  REGION 1..10: note = Description of Artificial Sequence: Synthetic peptide
SSYAGSNTFI

SEQ ID NO: 362 - protein, 11 aa, synthetic construct
  REGION 1..11: note = Description of Artificial Sequence: Synthetic peptide
QVWDSSSDHWV

SEQ ID NO: 363 - protein, 11 aa, synthetic construct
  REGION 1..11: note = Description of Artificial Sequence: Synthetic peptide
QSYDGSLSAQL

SEQ ID NO: 364 - protein, 9 aa, synthetic construct
  REGION 1..9: note = Description of Artificial Sequence: Synthetic peptide
QVWDSDHPL

SEQ ID NO: 365 - protein, 11 aa, synthetic construct
  REGION 1..11: note = Description of Artificial Sequence: Synthetic peptide
QSYDSTLSGGL

SEQ ID NO: 366 - protein, 11 aa, synthetic construct
  REGION 1..11: note = Description of Artificial Sequence: Synthetic peptide
QSYDSSLSGGL

SEQ ID NO: 367 - protein, 11 aa, synthetic construct
  REGION 1..11: note = Description of Artificial Sequence: Synthetic peptide
QSYDNTLSAGL

SEQ ID NO: 368 - protein, 11 aa, synthetic construct
  REGION 1..11: note = Description of Artificial Sequence: Synthetic peptide
QSYDSSLSVGL

SEQ ID NO: 369 - protein, 11 aa, synthetic construct
  REGION 1..11: note = Description of Artificial Sequence: Synthetic peptide
QSYDSSLTAGL

SEQ ID NO: 370 - protein, 11 aa, synthetic construct
  REGION 1..11: note = Description of Artificial Sequence: Synthetic peptide
QSYDNNLSAQV

SEQ ID NO: 371 - protein, 11 aa, synthetic construct
  REGION 1..11: note = Description of Artificial Sequence: Synthetic peptide
QSYDSSLSARV

SEQ ID NO: 372 - protein, 11 aa, synthetic construct
  REGION 1..11: note = Description of Artificial Sequence: Synthetic peptide
QSYDISLSAGL

SEQ ID NO: 373 - protein, 11 aa, synthetic construct
  REGION 1..11: note = Description of Artificial Sequence: Synthetic peptide
QSYDNILNAGL

SEQ ID NO: 374 - protein, 11 aa, synthetic construct
  REGION 1..11: note = Description of Artificial Sequence: Synthetic peptide
HSYDSSLSAQV

SEQ ID NO: 375 - protein, 11 aa, synthetic construct
  REGION 1..11: note = Description of Artificial Sequence: Synthetic peptide
QSYDNSLSAVI

SEQ ID NO: 376 - protein, 11 aa, synthetic construct
  REGION 1..11: note = Description of Artificial Sequence: Synthetic peptide
  MOD_RES 9: note = Any amino acid
QSYDSSLSXVL

SEQ ID NO: 377 - protein, 11 aa, synthetic construct
  REGION 1..11: note = Description of Artificial Sequence: Synthetic peptide
QSYDSRLSALL

SEQ ID NO: 378 - protein, 11 aa, synthetic construct
  REGION 1..11: note = Description of Artificial Sequence: Synthetic peptide
QSYDSSLSAVV

SEQ ID NO: 379 - protein, 11 aa, synthetic construct
  REGION 1..11: note = Description of Artificial Sequence: Synthetic peptide
QSYDNSLSALL

SEQ ID NO: 380 - protein, 11 aa, synthetic construct
  REGION 1..11: note = Description of Artificial Sequence: Synthetic peptide
  MOD_RES 7..9: note = Any amino acid
QSYDSSXXXHV

SEQ ID NO: 381 - protein, 11 aa, synthetic construct
  REGION 1..11: note = Description of Artificial Sequence: Synthetic peptide
QSYDSSLSTHV

SEQ ID NO: 382 - protein, 11 aa, synthetic construct
  REGION 1..11: note = Description of Artificial Sequence: Synthetic peptide
QSYDSSLTADV

SEQ ID NO: 383 - protein, 10 aa, synthetic construct
  REGION 1..10: note = Description of Artificial Sequence: Synthetic peptide
SSYAGSNTYI

SEQ ID NO: 384 - protein, 10 aa, synthetic construct
  REGION 1..10: note = Description of Artificial Sequence: Synthetic peptide
SSYAGSGTYI

SEQ ID NO: 385 - protein, 11 aa, synthetic construct
  REGION 1..11: note = Description of Artificial Sequence: Synthetic peptide
QSYDSRLSAHV

SEQ ID NO: 386 - protein, 11 aa, synthetic construct
  REGION 1..11: note = Description of Artificial Sequence: Synthetic peptide
QSYHSSLRAYI

SEQ ID NO: 387 - protein, 10 aa, synthetic construct
  REGION 1..10: note = Description of Artificial Sequence: Synthetic peptide
RSYRSGRTNI

SEQ ID NO: 388 - protein, 10 aa, synthetic construct
  REGION 1..10: note = Description of Artificial Sequence: Synthetic peptide
CSYRSGDTLI

SEQ ID NO: 389 - protein, 10 aa, synthetic construct
  REGION 1..10: note = Description of Artificial Sequence: Synthetic peptide
YSYRSGNTLV

SEQ ID NO: 390 - protein, 10 aa, synthetic construct
  REGION 1..10: note = Description of Artificial Sequence: Synthetic peptide
CSYRSGSTFL

SEQ ID NO: 391 - protein, 10 aa, synthetic construct
  REGION 1..10: note = Description of Artificial Sequence: Synthetic peptide
CSYTTSSTFI

SEQ ID NO: 392 - protein, 10 aa, synthetic construct
  REGION 1..10: note = Description of Artificial Sequence: Synthetic peptide
SSYAGINTLV

SEQ ID NO: 393 - protein, 10 aa, synthetic construct
  REGION 1..10: note = Description of Artificial Sequence: Synthetic peptide
SSYAGSNTFL

SEQ ID NO: 394 - protein, 11 aa, synthetic construct
  REGION 1..11: note = Description of Artificial Sequence: Synthetic peptide
QVWDSSSDHPV

SEQ ID NO: 395 - protein, 11 aa, synthetic construct
  REGION 1..11: note = Description of Artificial Sequence: Synthetic peptide
QVWDSSNDHYI

SEQ ID NO: 396 - protein, 11 aa, synthetic construct
  REGION 1..11: note = Description of Artificial Sequence: Synthetic peptide
AAWDDRLSGWV

SEQ ID NO: 397 - protein, 10 aa, synthetic construct
  REGION 1..10: note = Description of Artificial Sequence: Synthetic peptide
CSYTSGSTWV

SEQ ID NO: 398 - protein, 10 aa, synthetic construct
  REGION 1..10: note = Description of Artificial Sequence: Synthetic peptide
DSWDSSGTLV

SEQ ID NO: 399 - protein, 11 aa, synthetic construct
  REGION 1..11: note = Description of Artificial Sequence: Synthetic peptide
LSYDSSLSAGL

SEQ ID NO: 400 - protein, 11 aa, synthetic construct
  REGION 1..11: note = Description of Artificial Sequence: Synthetic peptide
QVWDSSSDHVL

SEQ ID NO: 401 - protein, 11 aa, synthetic construct
  REGION 1..11: note = Description of Artificial Sequence: Synthetic peptide
QVWDNSSDHYI

SEQ ID NO: 402 - protein, 8 aa, synthetic construct
  REGION 1..8: note = Description of Artificial Sequence: Synthetic peptide
QVWDSSCK

SEQ ID NO: 403 - protein, 11 aa, synthetic construct
  REGION 1..11: note = Description of Artificial Sequence: Synthetic peptide
SAWDSSLSAYI

SEQ ID NO: 404 - protein, 12 aa, synthetic construct
  REGION 1..12: note = Description of Artificial Sequence: Synthetic peptide
QSYDSRLRVNWV

SEQ ID NO: 405 - protein, 11 aa, synthetic construct
  REGION 1..11: note = Description of Artificial Sequence: Synthetic peptide
EAWDRSLSAWV

SEQ ID NO: 406 - protein, 11 aa, synthetic construct
  REGION 1..11: note = Description of Artificial Sequence: Synthetic peptide
DTWDNSLNGYI

SEQ ID NO: 407 - protein, 16 aa, synthetic construct
  REGION 1..16: note = Description of Artificial Sequence: Synthetic peptide
ARLGEYSWNSIGYFDY

SEQ ID NO: 408 - protein, 14 aa, synthetic construct
  REGION 1..14: note = Description of Artificial Sequence: Synthetic peptide
ARGGYYSGRVFDDY

SEQ ID NO: 409 - protein, 13 aa, synthetic construct
  REGION 1..13: note = Description of Artificial Sequence: Synthetic peptide
ARHSGWGDPYLDV

SEQ ID NO: 410 - protein, 13 aa, synthetic construct
  REGION 1..13: note = Description of Artificial Sequence: Synthetic peptide
AINSGSWNYYFDY

SEQ ID NO: 411 - protein, 15 aa, synthetic construct
  REGION 1..15: note = Description of Artificial Sequence: Synthetic peptide
TSDPATYSWNEYFEF

SEQ ID NO: 412 - protein, 14 aa, synthetic construct
  REGION 1..14: note = Description of Artificial Sequence: Synthetic peptide
AKEDGGWSNNRVDV

SEQ ID NO: 413 - protein, 11 aa, synthetic construct
  REGION 1..11: note = Description of Artificial Sequence: Synthetic peptide
AKGRGYNRFDV

SEQ ID NO: 414 - protein, 14 aa, synthetic construct
  REGION 1..14: note = Description of Artificial Sequence: Synthetic peptide
VRQGYSSWYNSLDV

SEQ ID NO: 415 - protein, 19 aa, synthetic construct
  REGION 1..19: note = Description of Artificial Sequence: Synthetic peptide
ARDMRDIAAGGYTYGYFDY

SEQ ID NO: 416 - protein, 17 aa, synthetic construct
  REGION 1..17: note = Description of Artificial Sequence: Synthetic peptide
VRDPSITPGPSYNRFDV

SEQ ID NO: 417 - protein, 13 aa, synthetic construct
  REGION 1..13: note = Description of Artificial Sequence: Synthetic peptide
AKGVYGSTNRFDV

SEQ ID NO: 418 - protein, 13 aa, synthetic construct
  REGION 1..13: note = Description of Artificial Sequence: Synthetic peptide
AKGVYGLTNRFDV

SEQ ID NO: 419 - protein, 20 aa, synthetic construct
  REGION 1..20: note = Description of Artificial Sequence: Synthetic peptide
TKEGGPEYYNIWTGWNRFDV

SEQ ID NO: 420 - protein, 16 aa, synthetic construct
  REGION 1..16: note = Description of Artificial Sequence: Synthetic peptide
AGGYLLFPLGYNSLDV

SEQ ID NO: 421 - protein, 15 aa, synthetic construct
  REGION 1..15: note = Description of Artificial Sequence: Synthetic peptide
AKGGGPPSWNDPFDF

SEQ ID NO: 422 - protein, 13 aa, synthetic construct
  REGION 1..13: note = Description of Artificial Sequence: Synthetic peptide
AKNGPPYWGMGDY

SEQ ID NO: 423 - protein, 16 aa, synthetic construct
  REGION 1..16: note = Description of Artificial Sequence: Synthetic peptide
AKDRGRGGSWSLGNDY

SEQ ID NO: 424 - protein, 17 aa, synthetic construct
  REGION 1..17: note = Description of Artificial Sequence: Synthetic peptide
AKGGEDDYIYYYTGADY

SEQ ID NO: 425 - protein, 19 aa, synthetic construct
  REGION 1..19: note = Description of Artificial Sequence: Synthetic peptide
ARGLFNFWSGYWGHNSLDV

SEQ ID NO: 426 - protein, 15 aa, synthetic construct
  REGION 1..15: note = Description of Artificial Sequence: Synthetic peptide
ARDYSSWPTYNSLDV

SEQ ID NO: 427 - protein, 12 aa, synthetic construct
  REGION 1..12: note = Description of Artificial Sequence: Synthetic peptide
AKSTLLRRSLDY

SEQ ID NO: 428 - protein, 18 aa, synthetic construct
  REGION 1..18: note = Description of Artificial Sequence: Synthetic peptide
AKDGGPSGSYYYGGRFDV

SEQ ID NO: 429 - protein, 18 aa, synthetic construct
  REGION 1..18: note = Description of Artificial Sequence: Synthetic peptide
AKDGGPSGSYYYRGRFDV

SEQ ID NO: 430 - protein, 11 aa, synthetic construct
  REGION 1..11: note = Description of Artificial Sequence: Synthetic peptide
ARGGGHSSFDF

SEQ ID NO: 431 - protein, 13 aa, synthetic construct
  REGION 1..13: note = Description of Artificial Sequence: Synthetic peptide
ARDSYKDSPAFDF

SEQ ID NO: 432 - protein, 17 aa, synthetic construct
  REGION 1..17: note = Description of Artificial Sequence: Synthetic peptide
AKDQTDLDWLLYGGFDY

SEQ ID NO: 433 - protein, 11 aa, synthetic construct
  REGION 1..11: note = Description of Artificial Sequence: Synthetic peptide
QSYDNSLSAQV

SEQ ID NO: 434 - protein, 12 aa, synthetic construct
  REGION 1..12: note = Description of Artificial Sequence: Synthetic peptide
HIWDSRVPTKWV

SEQ ID NO: 435 - protein, 12 aa, synthetic construct
  REGION 1..12: note = Description of Artificial Sequence: Synthetic peptide
HIWDSRRPTNWV

SEQ ID NO: 436 - protein, 12 aa, synthetic construct
  REGION 1..12: note = Description of Artificial Sequence: Synthetic peptide
HIYDARGGTNWV

SEQ ID NO: 437 - protein, 12 aa, synthetic construct
  REGION 1..12: note = Description of Artificial Sequence: Synthetic peptide
HMWDSRSGFSWS

SEQ ID NO: 438 - protein, 11 aa, synthetic construct
  REGION 1..11: note = Description of Artificial Sequence: Synthetic peptide
MWDSRSGFSWS

SEQ ID NO: 439 - protein, 11 aa, synthetic construct
  REGION 1..11: note = Description of Artificial Sequence: Synthetic peptide
QSSDTSDSYKM

SEQ ID NO: 440 - protein, 10 aa, synthetic construct
  REGION 1..10: note = Description of Artificial Sequence: Synthetic peptide
GSLVGNWDVI

SEQ ID NO: 441 - protein, 10 aa, synthetic construct
  REGION 1..10: note = Description of Artificial Sequence: Synthetic peptide
SSLVGNWDVI

SEQ ID NO: 442 - protein, 10 aa, synthetic construct
  REGION 1..10: note = Description of Artificial Sequence: Synthetic peptide
SSLFGRWDVV

SEQ ID NO: 443 - protein, 10 aa, synthetic construct
  REGION 1..10: note = Description of Artificial Sequence: Synthetic peptide
SSLSGRWDIV

SEQ ID NO: 444 - protein, 9 aa, synthetic construct
  REGION 1..9: note = Description of Artificial Sequence: Synthetic peptide
SFGGSATVV

SEQ ID NO: 445 - protein, 12 aa, synthetic construct
  REGION 1..12: note = Description of Artificial Sequence: Synthetic peptide
QVWDSSSDHPWV

SEQ ID NO: 446 - protein, 10 aa, synthetic construct
  REGION 1..10: note = Description of Artificial Sequence: Synthetic peptide
  MOD_RES 8..10: note = Any amino acid
SSYAGSNXXX

SEQ ID NO: 447 - protein, 9 aa, synthetic construct
  REGION 1..9: note = Description of Artificial Sequence: Synthetic peptide
SYAGSSTVI

SEQ ID NO: 448 - DNA, 24 bp, synthetic construct
  misc_feature 1..24: note = Description of Artificial Sequence: Synthetic primer
  source 1..24: mol_type = other DNA
acaggtgcccactcccaggtgcag

SEQ ID NO: 449 - DNA, 23 bp, synthetic construct
  misc_feature 1..23: note = Description of Artificial Sequence: Synthetic primer
  source 1..23: mol_type = other DNA
aaggtgtccagtgtgargtgcag

SEQ ID NO: 450 - DNA, 27 bp, synthetic construct
  misc_feature 1..27: note = Description of Artificial Sequence: Synthetic primer
  source 1..27: mol_type = other DNA
cccagatgggtcctgtcccaggtgcag

SEQ ID NO: 451 - DNA, 24 bp, synthetic construct
  misc_feature 1..24: note = Description of Artificial Sequence: Synthetic primer
  source 1..24: mol_type = other DNA
caaggagtctgttccgaggtgcag

SEQ ID NO: 452 - DNA, 17 bp, synthetic construct
  misc_feature 1..17: note = Description of Artificial Sequence: Synthetic primer
  source 1..17: mol_type = other DNA
ttctccaaggagtctgt

SEQ ID NO: 453 - DNA, 17 bp, synthetic construct
  misc_feature 1..17: note = Description of Artificial Sequence: Synthetic primer
  source 1..17: mol_type = other DNA
taaaaggtgtccagtgt

SEQ ID NO: 454 - DNA, 17 bp, synthetic construct
  misc_feature 1..17: note = Description of Artificial Sequence: Synthetic primer
  source 1..17: mol_type = other DNA
taagaggtgtccagtgt

SEQ ID NO: 455 - DNA, 17 bp, synthetic construct
  misc_feature 1..17: note = Description of Artificial Sequence: Synthetic primer
  source 1..17: mol_type = other DNA
tagaaggtgtccagtgt

SEQ ID NO: 456 - DNA, 20 bp, synthetic construct
  misc_feature 1..20: note = Description of Artificial Sequence: Synthetic primer
  source 1..20: mol_type = other DNA
atgaaacatctgtggttctt

SEQ ID NO: 457 - DNA, 17 bp, synthetic construct
  misc_feature 1..17: note = Description of Artificial Sequence: Synthetic primer
  source 1..17: mol_type = other DNA
tacaaggtgtccagtgt

SEQ ID NO: 458 - DNA, 17 bp, synthetic construct
  misc_feature 1..17: note = Description of Artificial Sequence: Synthetic primer
  source 1..17: mol_type = other DNA
ttaaagctgtccagtgt

SEQ ID NO: 459 - DNA, 20 bp, synthetic construct
  misc_feature 1..20: note = Description of Artificial Sequence: Synthetic primer
  source 1..20: mol_type = other DNA
gctgaggagacggtgaccag

SEQ ID NO: 460 - DNA, 21 bp, synthetic construct
  misc_feature 1..21: note = Description of Artificial Sequence: Synthetic primer
  source 1..21: mol_type = other DNA
gctgaggagatggtgattggg

SEQ ID NO: 461 - DNA, 21 bp, synthetic construct
  misc_feature 1..21: note = Description of Artificial Sequence: Synthetic primer
  source 1..21: mol_type = other DNA
gctgaagagacggtgaccctg

SEQ ID NO: 462 - DNA, 21 bp, synthetic construct
  misc_feature 1..21: note = Description of Artificial Sequence: Synthetic primer
  source 1..21: mol_type = other DNA
gctgaggagacggtgacgacg

SEQ ID NO: 463 - DNA, 48 bp, synthetic construct
  misc_feature 1..48: note = Description of Artificial Sequence: Synthetic primer
  source 1..48: mol_type = other DNA
ctagtagcaactgcaaccggtgtacattcccaggtgcagctggtgcag

SEQ ID NO: 464 - DNA, 48 bp, synthetic construct
  misc_feature 1..48: note = Description of Artificial Sequence: Synthetic primer
  source 1..48: mol_type = other DNA
ctagtagcaactgcaaccggtgtacattccgaggtgcagctggtgcag

SEQ ID NO: 465 - DNA, 48 bp, synthetic construct
  misc_feature 1..48: note = Description of Artificial Sequence: Synthetic primer
  source 1..48: mol_type = other DNA
ctagtagcaactgcaaccggtgtacattcccaggttcagctggtgcag

SEQ ID NO: 466 - DNA, 48 bp, synthetic construct
  misc_feature 1..48: note = Description of Artificial Sequence: Synthetic primer
  source 1..48: mol_type = other DNA
ctagtagcaactgcaaccggtgtacattcccaggtccagctggtacag

SEQ ID NO: 467 - DNA, 48 bp, synthetic construct
  misc_feature 1..48: note = Description of Artificial Sequence: Synthetic primer
  source 1..48: mol_type = other DNA
ctagtagcaactgcaaccggtgtacattctgaggtgcagctggtggag

SEQ ID NO: 468 - DNA, 48 bp, synthetic construct
  misc_feature 1..48: note = Description of Artificial Sequence: Synthetic primer
  source 1..48: mol_type = other DNA
ctagtagcaactgcaaccggtgtacattctcaggtgcagctggtggag

SEQ ID NO: 469 - DNA, 48 bp, synthetic construct
  misc_feature 1..48: note = Description of Artificial Sequence: Synthetic primer
  source 1..48: mol_type = other DNA
ctagtagcaactgcaaccggtgtacattctgaggtgcagctgttggag

SEQ ID NO: 470 - DNA, 48 bp, synthetic construct
  misc_feature 1..48: note = Description of Artificial Sequence: Synthetic primer
  source 1..48: mol_type = other DNA
ctagtagcaactgcaaccggtgtacattctgaagtgcagctggtggag

SEQ ID NO: 471 - DNA, 48 bp, synthetic construct
  misc_feature 1..48: note = Description of Artificial Sequence: Synthetic primer
  source 1..48: mol_type = other DNA
ctagtagcaactgcaaccggtgtacattcccaggtgcagctgcaggag

SEQ ID NO: 472 - DNA, 50 bp, synthetic construct
  misc_feature 1..50: note = Description of Artificial Sequence: Synthetic primer
  source 1..50: mol_type = other DNA
ctagtagcaactgcaaccggtgtacattcccaggtgcagctacagcagtg

SEQ ID NO: 473 - DNA, 48 bp, synthetic construct
  misc_feature 1..48: note = Description of Artificial Sequence: Synthetic primer
  source 1..48: mol_type = other DNA
ctagtagcaactgcaaccggtgtacattcccagctgcagctgcaggag

SEQ ID NO: 474 - DNA, 48 bp, synthetic construct
  misc_feature 1..48: note = Description of Artificial Sequence: Synthetic primer
  source 1..48: mol_type = other DNA
ctagtagcaactgcaaccggtgtacattcccaggtacagctgcagcag

SEQ ID NO: 475 - DNA, 40 bp, synthetic construct
  misc_feature 1..40: note = Description of Artificial Sequence: Synthetic primer
  source 1..40: mol_type = other DNA
ccgatgggcccttggtcgacgctgaggagacggtgaccag

SEQ ID NO: 476 - DNA, 42 bp, synthetic construct
  misc_feature 1..42: note = Description of Artificial Sequence: Synthetic primer
  source 1..42: mol_type = other DNA
ccgatgggcccttggtcgacgctgaagagacggtgaccattg

SEQ ID NO: 477 - DNA, 41 bp, synthetic construct
  misc_feature 1..41: note = Description of Artificial Sequence: Synthetic primer
  source 1..41: mol_type = other DNA
ccgatgggcccttggtcgacgctgaggagacggtgaccgtg

SEQ ID NO: 478 - DNA, 20 bp, synthetic construct
  misc_feature 1..20: note = Description of Artificial Sequence: Synthetic primer
  source 1..20: mol_type = other DNA
gtagcaactgcaaccggtgt

SEQ ID NO: 479 - DNA, 21 bp, synthetic construct
  misc_feature 1..21: note = Description of Artificial Sequence: Synthetic primer
  source 1..21: mol_type = other DNA
gcttcgttagaacgcggctac

SEQ ID NO: 480 - DNA, 23 bp, synthetic construct
  misc_feature 1..23: note = Description of Artificial Sequence: Synthetic primer
  source 1..23: mol_type = other DNA
ggaaggtgtgcacgccgctggtc

SEQ ID NO: 481 - DNA, 23 bp, synthetic construct
  misc_feature 1..23: note = Description of Artificial Sequence: Synthetic primer
  source 1..23: mol_type = other DNA
ggtcctgggcccagtctgtgctg

SEQ ID NO: 482 - DNA, 23 bp, synthetic construct
  misc_feature 1..23: note = Description of Artificial Sequence: Synthetic primer
  source 1..23: mol_type = other DNA
ggtcctgggcccagtctgccctg

SEQ ID NO: 483 - DNA, 23 bp, synthetic construct
  misc_feature 1..23: note = Description of Artificial Sequence: Synthetic primer
  source 1..23: mol_type = other DNA
gctctgtgacctcctatgagctg

SEQ ID NO: 484 - DNA, 23 bp, synthetic construct
  misc_feature 1..23: note = Description of Artificial Sequence: Synthetic primer
  source 1..23: mol_type = other DNA
ggtctctctcscagcytgtgctg

SEQ ID NO: 485 - DNA, 23 bp, synthetic construct
  misc_feature 1..23: note = Description of Artificial Sequence: Synthetic primer
  source 1..23: mol_type = other DNA
gttcttgggccaattttatgctg

SEQ ID NO: 486 - DNA, 23 bp, synthetic construct
  misc_feature 1..23: note = Description of Artificial Sequence: Synthetic primer
  source 1..23: mol_type = other DNA
ggtccaattcycaggctgtggtg

SEQ ID NO: 487 - DNA, 23 bp, synthetic construct
  misc_feature 1..23: note = Description of Artificial Sequence: Synthetic primer
  source 1..23: mol_type = other DNA
gagtggattctcagactgtggtg

SEQ ID NO: 488 - DNA, 24 bp, synthetic construct
  misc_feature 1..24: note = Description of Artificial Sequence: Synthetic primer
  source 1..24: mol_type = other DNA
caccagtgtggccttgttggcttg

SEQ ID NO: 489 - DNA, 48 bp, synthetic construct
  misc_feature 1..48: note = Description of Artificial Sequence: Synthetic primer
  source 1..48: mol_type = other DNA
ctagtagcaactgcaaccggttcctgggcccagtctgtgctgackcag

SEQ ID NO: 490 - DNA, 48 bp, synthetic construct
  misc_feature 1..48: note = Description of Artificial Sequence: Synthetic primer
  source 1..48: mol_type = other DNA
ctagtagcaactgcaaccggttcctgggcccagtctgccctgactcag

SEQ ID NO: 491 - DNA, 48 bp, synthetic construct
  misc_feature 1..48: note = Description of Artificial Sequence: Synthetic primer
  source 1..48: mol_type = other DNA
ctagtagcaactgcaaccggttctgtgacctcctatgagctgacwcag

SEQ ID NO: 492 - DNA, 47 bp, synthetic construct
  misc_feature 1..47: note = Description of Artificial Sequence: Synthetic primer
  source 1..47: mol_type = other DNA
ctagtagcaactgcaaccggttctctctcscagcytgtgctgactca

SEQ ID NO: 493 - DNA, 48 bp, synthetic construct
  misc_feature 1..48: note = Description of Artificial Sequence: Synthetic primer
  source 1..48: mol_type = other DNA
ctagtagcaactgcaaccggttcttgggccaattttatgctgactcag

SEQ ID NO: 494 - DNA, 48 bp, synthetic construct
  misc_feature 1..48: note = Description of Artificial Sequence: Synthetic primer
  source 1..48: mol_type = other DNA
ctagtagcaactgcaaccggttccaattcycagrctgtggtgacycag

SEQ ID NO: 495 - DNA, 37 bp, synthetic construct
  misc_feature 1..37: note = Description of Artificial Sequence: Synthetic primer
  source 1..37: mol_type = other DNA
ggcttgaagctcctcactcgagggygggaacagagtg

(19) United States
(12) Patent Application Publication
(10) Pub. No.: US 2025/0144197 A1
(43) Pub. Date: May 8, 2025

(54) HIV VACCINE IMMUNOGENS

(71) Applicants: The Rockefeller University, New York, NY (US); California Institute of Technology, Pasadena, CA (US)

(72) Inventors: Michel Nussenzweig, New York, NY (US); Pamela J. Bjorkman, Altadena, CA (US); Amelia Escolano, New York, NY (US); Harry Gristick, Pasadena, CA (US)

(21) Appl. No.: 19/014,555

(22) Filed: Jan. 9, 2025

Related U.S. Application Data
(62) Division of application No. 17/299,852, filed on Jun. 4, 2021, now Pat. No. 12,239,698.
(60) Provisional application No. 62/775,192, filed on Dec. 4, 2018.

(51) Int. Cl. A61K 39/12; A61K 39/00; A61P 31/18; C07K 14/005

(52) U.S. Cl. CPC A61K 39/12; A61P 31/18; C07K 14/005; A61K 2039/55577; C12N 2740/16023; C12N 2740/16134

(57) ABSTRACT

This disclosure provides HIV immunogens and use thereof for generating an immune response in a subject. Also disclosed is a method of isolating anti-HIV antibodies and use thereof. This disclosure further provides a method for treating or preventing a human immunodeficiency type 1 (HIV-1) infection in a subject using the disclosed HIV immunogens and/or antibodies.

### CROSS-REFERENCE TO RELATED APPLICATIONS

This application is a divisional application of U.S. patent application Ser. No. 17/299,852, filed on Jun. 4, 2021, which is a National Stage Application of PCT/US19/63619, filed on Nov. 27, 2019, which claims the benefit of U.S. Provisional Application No. 62/775, 192 filed on Dec. 4, 2018. The entire contents of these applications are incorporated herein by reference in their entirety.

### STATEMENT REGARDING FEDERALLY SPONSORED RESEARCH

This invention was made with government support under Grant No. AI100148 awarded by the National Institutes of Health. The government has certain rights in the invention.

### REFERENCE TO AN ELECTRONIC SEQUENCE LISTING

The contents of the electronic sequence listing (070413.20832SeqList.xml; Size: 654,540 bytes; and Date of Creation: Dec. 5, 2024) is herein incorporated by reference in its entirety.

### FIELD OF THE INVENTION

This disclosure relates to immunogenic polypeptides, and specifically to immunogenic polypeptides capable of stimulating an immune response to human immunodeficiency virus (HIV).

### BACKGROUND OF THE INVENTION

Single-cell antibody cloning from HIV-1-infected human donors revealed that broadly neutralizing anti-HIV-1 antibodies (bNAbs) are unusual in that they are highly somatically mutated. Moreover, the high degree of somatic mutations is essential for binding to native HIV-1 Env and for bNAb neutralizing activity. The accumulation of large numbers of mutations suggests that bNAbs evolve in response to iterative rounds of somatic hypermutation and selection in germinal centers (GCs). As revealed by prospective studies in humans, bNAbs do so in response to viral escape variants arising from antibody pressure. Together, these observations suggest that vaccination to elicit bNAbs requires a series of sequential immunogens starting with an immunogen that induces the expansion of B lymphocytes that carry germline precursors of bNAbs.

The idea that sequential immunization can shepherd bNAb development was confirmed by experiments in genetically-modified mice that carry the inferred germline (iGL) precursors of human bNAbs. However, the priming immunogens used to initiate the response failed to activate and expand B-cells expressing inferred precursors of bNAbs in animals with polyclonal antibody repertoires. Indeed, the iGLs of nearly all bNAbs fail to bind to native-like HIV-1 immunogens or neutralize HIV-1 strains. Thus, a critical goal of HIV-1 vaccine development has been to design immunogens that recruit B-cells expressing bNAb precursors into GC reactions in animals with polyclonal repertoires including primates.

To this end, the germline targeting approach to immunogen design has focused on producing immunogens that bind to specific bNAb precursors with high affinity, the rationale being that B-cell recruitment to GCs is in part dependent on receptor affinity for the antigen. However, this methodology effectively limits the repertoire of recruited B-cells qualitatively and quantitatively. Moreover, it fails to account for the finding that each GC can accommodate multiple different founder B-cells with a wide range of affinities and that GC entry is limited by competition and not absolute affinity. An alternative is to design immunogens that enhance the availability of the targeted epitope while masking off-target sites. This approach differs from germline targeting in that it is agnostic to the affinity of a specific germline antibody for the antigen. Instead, it aims to recruit and expand a diverse group of precursors specific to the target site. Both approaches aim to produce expanded clones of B-cells that can then be boosted by sequential immunogens to shepherd bNAb production. To date, neither of these methods has been shown to expand B-cell clones specific for a desired HIV-1 target in wild-type animals.

Accordingly, there remains a pressing need for immunogens capable of stimulating an immune response to human immunodeficiency virus (HIV), for example, by way of expanding B-cell clones specific for a desired HIV-1 target.

### SUMMARY OF THE INVENTION

Various embodiments described in this document address the above-mentioned unmet needs and/or other needs by providing HIV immunogens and uses thereof.

In one aspect, the disclosure relates to an immunogen for stimulating an immune response (e.g., HIV immune response) of a subject in need thereof. The immunogen comprises a polypeptide having a sequence that is at least 75% identical to a sequence selected from the group consisting of SEQ ID NOs: 2, 4, 6, 8, 11, and 13. The polypeptide includes substitutions at the positions corresponding to N133, N137, and N156 of SEQ ID NO: 1. In one example, the polypeptide includes an N156Q substitution or a conservative substitution of N156. In another example, the polypeptide includes V134Y, T135A, I138L, T139L, D140S, D141N, T320F, Q328M, T415V substitutions or conservative substitutions thereof.

The immunogen mentioned above binds to a broadly neutralizing antibody with an affinity (e.g., K_{D} of about 50 μM or less). Examples of broadly neutralizing antibodies may include 10-1074 and PGT121 broadly neutralizing antibodies.

Also within the scope of this invention are an isolated nucleic acid encoding the polypeptide described above, a vector comprising the nucleic acid, and a host cell comprising the nucleic acid. The host cell can be used in a method of producing the polypeptide. The method includes culturing the host cell in a medium under conditions permitting expression of a polypeptide encoded by the nucleic acid, and purifying the polypeptide from the cultured cell or the medium of the cell.

In another aspect, this disclosure provides a protein complex comprising at least one above-described polypeptide and a virus particle comprising at least one above-described polypeptide.

In another aspect, this disclosure provides an immunogenic composition for stimulating an immune response in a subject in need thereof. The immunogenic composition includes (i) the polypeptide, the nucleic acid, the host cell, the protein complex, or the virus particle described above; and (ii) a pharmaceutically acceptable carrier. The method may further include administering the composition two or more times. The administration of the composition may result in increased numbers of broadly-neutralizing antibodies in the serum capable of recognizing a V3-glycan epitope.

In another aspect, this disclosure provides a method of stimulating an immune response in a subject in need thereof. The method includes administrating to the subject an effective amount of a composition comprising the polypeptide, the nucleic acid, the host cell, the protein complex, or the virus-like particle (VLP) described above, or a combination thereof.

In another aspect, this disclosure provides a method of treating or preventing HIV infection in a subject in need thereof. The method includes administering to the subject a therapeutically effective amount of the polypeptide, the nucleic acid, the host cell, the protein complex, or the virus particle described above, or a combination thereof. In some embodiments, the method may also include administering to the subject a therapeutically effective amount of an anti-viral agent.

In another aspect, this disclosure provides use of the polypeptide, the nucleic acid, the host cell, the protein complex, or the virus particle described above, or a combination thereof in the preparation of a medicament to treat or prevent HIV infection in a subject.

In another aspect, this disclosure provides a method for detecting or isolating an HIV-1 binding antibody in a subject infected with HIV-1. The method includes: (i) providing the polypeptide, the nucleic acid, the host cell, the protein complex, or the virus particle described above, or a combination thereof; (ii) contacting the immunogenic composition with an amount of bodily fluid from the subject; and (iii) detecting binding of the HIV-1 binding antibody to the polypeptide, thereby detecting or isolating the HIV-1 binding antibody in a subject.

In yet another aspect, this disclosure provides a kit for stimulating an immune response in a subject. The kit includes (i) one or more unit dosages of the polypeptide, the nucleic acid, the host cell, the protein complex, or the virus particle described above; (ii) instructions for administrating the polypeptide, the nucleic acid, the host cell, the protein complex, or the virus particle; and (iii) optionally an adjuvant.

This disclosure also provides an isolated anti-HIV antibody, or antigen-binding portion thereof, comprising a complementarity-determining region having a sequence that is at least 75% identical to a polypeptide sequence listed in Tables 4, 5, 6, 7, 9, 10, and 11.

Also within the scope of this disclosure is a pharmaceutical composition comprising the isolated anti-HIV antibody, or antigen-binding portion thereof as described, and a pharmaceutically acceptable carrier or excipient.

In another aspect, this disclosure provides a method of preventing or treating an HIV infection or an HIV-related disease comprising the steps of: (i) identifying a patient in need of such prevention or treatment, and (ii) administering to said patient a first therapeutic agent comprising a therapeutically effective amount of at least one anti-HIV antibody describe above, or antigen-binding portion thereof. This disclosure further provides a kit comprising a pharmaceutically acceptable dose unit of a pharmaceutically effective amount of at least one above-described isolated anti-HIV antibody, or antigen-binding portion thereof.

The details of one or more embodiments of the invention are set forth in the description below. Other features, objectives, and advantages of the invention will be apparent from the description and from the claims.

### BRIEF DESCRIPTION OF THE DRAWINGS

FIGS. 1a, 1b, and 1c are diagrams showing the characterization of the RC1 immunogen. FIG. 1a shows positions of N-glycans (colored spheres) and GDIR motif (SEQ ID NO: 15) (red surfaces) in V3-glycan patches of wtBG505, 11MUTB, and RC1 Env trimers. Coordinates for glycans are mapped onto a surface representation of the wtBG505 Env trimer structure (PDB 5T3Z) (N137 glycan from PDB 5FYL) seen in the top-down orientation. FIG. 1b shows a comparison of the structures of wtBG505 (PDB 5T3Z) (left) and RC1 (right) (4.0 Å cryo-EM structure) complexed with 10-1074 Fab. Env trimer-Fab complexes are shown from the side as surface representations with glycan atoms as colored spheres. The middle panel shows a close-up superimposition of the boxed regions of the 10-1074 complexes with wtBG505 and RC1. Protein regions are shown in cartoon representations (10-1074 V_{H} and V_{L} in dark and light purple, Env GDIR regions in red (SEQ ID NO: 15), other portions of RC1 in wheat, wtBG505 in grey, and the N156 glycan coordinates from the wtBG505 structure shown as orange spheres. The locations of regions of V1 that show the largest displacement between the structures (gp120 residues 139-140) are indicated by dots with an arrow showing the displacement. V1 residues 149-151 are ordered in the RC1 structure, but not in the wtBG505 structure. FIG. 1c shows SPR binding data (R_{eq}, equilibrium binding response, versus the log of the concentration of injected protein) shown for experiments in which the Fab for the common iGL of PGT121 and 10-1074 was injected over the indicated immobilized Env trimers. N.B.=no binding above background.

FIGS. 2a, 2b, 2c, 2d, 2e, 2f, 2g, 2h, 2i, 2j, 2k, and 2l are diagrams showing wild-type mouse immunization with RC1 elicits anti-glycan patch antibodies. FIG. 2a is representative ELISA results showing the binding of serum from knock-in mice that carry the PGT121/10-1074 iGL antibody to 11MUTB after immunization with 11MUTB (left) and to RC1 after immunization with RC1 (right). Controls include naïve serum, purified PGT121 and iGL-PGT121. FIG. 2b shows area under the curve (AUC) for ELISAs as in FIG. 2a, but combined results from 2 independent experiments using 3 mice each. Each dot represents the scrum of one mouse. FIG. 2d shows representative ELISA results for binding of serum from wild-type mice immunized with 11MUTB (left) and RC1 (right) to 11MUTB and RC1 respectively. FIG. 2d shows AUC for ELISAs as in c, but combined results from 2 experiments using 3 mice each. Each dot represents the serum of one mouse. FIG. 2e shows binding of serum from one representative wild-type mouse immunized with RC1 to RC1 and RC1-glycanKO in ELISA. FIG. 2f shows the ratio of the AUC for RC1 vs. RC1-glycan KO ELISAs as in FIG. 2e. The graph shows the combined results from 7 experiments with 2-3 mice immunized with RC1. Each dot represents one mouse. FIG. 2g shows representative ELISA results showing the binding of serum from wild-type mice immunized with 11MUTBΔ301 to 11MUTBΔ301. FIG. 2h shows ratio of the AUC for RC1 vs. RC1-glycan KO ELISAs for wild-type mice immunized with RC1 or RC1-4fill. FIG. 2i is pie charts showing clonal expansion of RC1 binding germinal center B cells as determined by IgV_{H} gene sequencing. Colored pie slices are proportional to the number of clonal relatives. White indicates single IgV_{H} sequences. The number in the center indicates the number of heavy chains analyzed. FIG. 2j shows IgH nucleotide mutations from naïve and RC1 immunized mice in FIG. 2i. FIG. 2k shows binding of monoclonal antibodies obtained from RC1 immunized mice to RC1 and RC1-glycanKO in ELISA. FIG. 2l shows characterization of the binding pattern of Ab275_{MUR} and Ab276_{MUR} isolated from RC1 and RC1-4fill immunized wild-type mice by ELISA on the indicated Env proteins. FIG. 2l discloses “GAIA” as SEQ ID NO: 16.

FIGS. 3a, 3b, 3c, 3d, 3e, 3f, 3g, 3h, 3i, and 3j are a set of diagrams showing macaque immunization with RC1-4fill VLPs elicits anti-V3-glycan patch antibodies that resemble iGL bNAbs. FIG. 3a shows a representation of RC1-4fill VLPs showing RC1, spytag, spycatcher, and VLP. FIG. 3b shows electron micrographs of VLPs (top) and RC1-4fill-VLPs (bottom). FIG. 3c shows binding of the serum from 4 rabbits immunized with RC1-4fill VLP, a naïve control, and the monoclonal antibodies PGT121 and 3BNC117 to RC1 (black) and RC1-glycanKO (grey) shown as area under the ELISA curve (AUC). FIG. 3d shows binding of the serum from 8 Rhesus macaques immunized with RC1-4fill VLP, a naïve control and the monoclonal antibodies PGT121 and 3BNC117 to RC1 (black) and RC1-glycanKO (grey) shown as area under the ELISA curve (AUC). FIG. 3e is representative flow cytometry dot plots showing macaque germinal center B cell binding to RC1-PE (Y-axis) and RC1-AF647 or RC1-glycan KO (X-axis) for naïve (left) and immunized macaques (right). FIG. 3f shows percent of all B cells in the germinal centers from lymph node samples from 4 naïve or 4immunized macaques that bind to RC1 but not to RC1-glycanKO by flow cytometry. FIG. 3g is pie charts showing clonal expansion of RC1 binding germinal center B cells as determined by IgH gene sequencing. The number in the center indicates the number of IgV_{H} sequences analyzed. FIG. 3h shows IgV_{H} mutations for all sequences in FIG. 3g, each dot represents one IgV_{H}. FIG. 3i shows iGL sequence of CDRL3 (SEQ ID NO: 446) for PGT121/10-1074 and logo plots for all IgL chains cloned from RC1 binding GC B cells from immunized macaques. FIG. 3j shows fraction of IgL CDR3 sequences cloned from GC B cells from 4 naïve and 4 RC1 immunized macaques that show a DSS-like motif.

FIGS. 4a, 4b, 4c, 4d, and 4e show monoclonal antibodies from macaques immunized with RC1-4fill VLPs bind to the V3-glycan patch. FIG. 4a shows ELISA results for binding of monoclonal macaque antibodies to RC1 and RC1-glycanKO-GAIA (“GAIA” disclosed as SEQ ID NO: 16). Controls are 10-1074 and 3BNC117. FIG. 4b shows IgH CDR3 length of V3-glycan patch specific macaque antibodies. FIG. 4c shows a number of nucleotide mutations in IgV_{H} and IgV_{L} regions of V3-glycan patch specific macaque antibodies. FIG. 4d shows area under the curve (AUC) for ELISAs on the indicated proteins for antibodies Ab876_{NHP}, Ab897_{NHP}, Ab933_{NHP}, Ab936_{NHP}, Ab1170_{NHP}, 3BNC117 and 10-1074. FIG. 4e discloses “GAIA” as SEQ ID NO: 16.

FIGS. 5a, 5b, and 5c show a comparison of the structures of 10-1074 and elicited antibodies bound to the RC1 immunogen. FIG. 5a shows top-down views of the binding orientation of 10-1074 Fab compared with other V3-glycan patch bNAb Fabs (PGT128 and PGT135; PDB 5ACO and 4JM2) (left), Ab275_{MUR} (second from left), Ab874_{NHP} (third from left), and Ab897_{NHP} (right). Env and Fab structures are shown in cartoon representations. FIG. 5b (top panel) shows V_{H}-V_{L} domains of 10-1074 (left) and elicited antibody Fabs (three right panels) bound to the V3-glycan patch on one protomer of RC1 trimer (from cryo-EM structures of complexes of 10-1074 (left), Ab275_{MUR} (second from left), Ab874_{NHP} (third from left), and Ab897_{NHP} (right) bound to RC1 Env trimer). GDIR residues (SEQ ID NO: 15) on gp120 are highlighted in red, and glycan coordinates are shown as colored spheres. FIG. 5b (bottom panel) shows 90° rotation of complexes in top panels to show top-down views of antibody combining sites with CDRs highlighted as loops and gp120 glycans (colored spheres) and GDIR (SEQ ID NO: 15) (red) regions from RC1 mapped onto antibody combining sites. FIG. 5c shows comparisons of interactions of GDIR motif (SEQ ID NO: 15) with 10-1074 and with elicited antibodies.

FIGS. 6a and 6b show the characterization of RC1 by evaluating its interactions with bNAbs by ELISA. FIG. 6a shows that a V1-V2-specific bNAb that interacts with the N156 glycan32 showed reduced binding to RC1 as compared to BG505, and the absence of the N156 PNGS enhances neutralization by PGT121 and 10-1074. FIG. 6b shows that bNAbs targeting the V3-glycan epitope, the CD4 binding site, or the gp120-gp41 interface bound similarly to RC1 and BG505.

FIGS. 7a, 7b, 7c, and 7d show the single-particle cryo-EM study of RC1 respectively complexed with the antigen-binding fragment (Fab) of 10-1074 (FIG. 7a), Ab874_{NHP} (FIG. 7b), Ab275_{MUR} (FIG. 7c), and Ab897_{NHP} (FIG. 7d).

FIG. 8 shows that the serum from the RC1-immunized mice cross-reacted with 11MUTB but not to the more native 10MUT Env or to BG505.

FIGS. 9a, 9b, 9c, 9d, and 9e show the characterization of RC1 and RC1-4fill and their response to the off-target sites.

FIG. 10 shows the characterization of the humoral responses elicited by RC1 and RC1-4fill in wild-type mice. The antibody genes from single GC B-cells that bound to RC1 but not to RC1-glycanKO were sequenced.

FIGS. 11a, 11b, 11c, and 11d show that RC1 and RC1-4fill expanded V3-glycan patch specific B-cells in wild-type mice. Both antibodies isolated from mice immunized with RC1 (Ab275_{MUR}) or RC1-4fill (Ab276_{MUR}) bound 11MUTB, but not BG505 or a peptide that covers the crown of the V3 loop (FIG. 11a). Ab275_{MUR} bound RC1 with a K_{D}˜30 nM (FIG. 11b). Importantly, Ab275_{MUR} retained binding to 11MUTB (K_{D}˜230 nM), demonstrating that it could accommodate the N156 glycan (FIG. 11c). The acquired mutations were essential for binding because reversion to the iGL sequence led to the loss of binding to RC1 (FIG. 11d).

FIG. 12 shows that VLP-RC1-4fill elicits V3-glycan patch antibodies in rabbits and Rhesus macaques. The serum from the macaques primed with RC1-4fill VLPs showed sequentially reduced binding to the more native-like immunogens 11MUTB and 10MUT.

FIGS. 13a, 13b, 13c, 13d, 13e, 13f, 13g, 13h, 13i, and 13j (collectively “FIG. 13”) are a set of diagrams showing characterization of the immunogens including RC1-3fill. FIG. 13a is a diagram showing size-exclusion chromatography (SEC) traces for the RC1, RC1-3fill, and RC1-4fill immunogens. FIG. 13b provides the representative yields from a IL expression in HEK 293T 6E cells for each immunogen. FIGS. 13c, 13d, 13e, and 13f are a set of diagrams showing SEC traces and electron micrographs for the RC1 and RC1-3fill immunogens. FIG. 13g shows representative SEC traces for the purification of the AP205-RC1-VLPs and AP205-RC1-3fill-VLPs. FIG. 13d shows electron micrographs of the AP205-RC1-VLPs (left) and AP205-RC1-3fill-VLPs (right). FIG. 13e shows representative SEC traces for the purification of the mi3-RC1-NPs and mi3-RC1-3fill-NPs. FIG. 13f shows electron micrographs of the mi3-RC1-NPs (left) and mi3-RC1-3fill-NPs (right). FIGS. 13f and 13h show the SEC profiles for both the initial purification of the AP205-RC1-VLPs (FIG. 13f) and the mi3-RC1-NPs (FIG. 13h), and reinjection of the sample at 28 days (AP205) and 11 days (mi3). FIGS. 13i and 13j show binding of the serum from 6 WT mice immunized with cither mi3-RC1-NPs (FIG. 13i) or mi3-RC1-3fill-NPs (FIG. 13j), a naïve control and the monoclonal antibodies 10-1074 and 3BNC117 to RC1 and RC1-glycanKO shown as area under the ELISA curve (AUC).

### DETAILED DESCRIPTION OF THE INVENTION

The disclosed immunogens for stimulating an immune response in a subject are based on an unexpected discovery that a novel immunogen, RC1, and its variants, activate B-cells expressing precursors of bNAbs within polyclonal repertoires.

Broadly neutralizing antibodies (bNAbs) protect against HIV-1 infection, suggesting that a vaccine that elicits them would be effective. However, one of the major hurdles is that vaccination does not elicit bNAbs, in part, because B-cells expressing germline bNAb precursors do not respond to native-like HIV-1 envelope (Env) antigens. Accordingly, this disclosure provides immunogens that facilitate recognition of the V3-glycan patch on HIV-1 Env while concealing non-conserved immunodominant regions, for example, by addition of glycans and/or multimerization on virus-like particles. This disclosure demonstrates that mouse, rabbit, and Rhesus macaque immunizations with the disclosed immunogens (e.g., RC1, RC1-4fill, RC1-3fill) elicited serologic responses targeting the V3-glycan patch. Further, antibody cloning and cryo-EM structures of antibody-Env complexes confirmed that RC1 immunization expands clones of B-cells carrying anti-V3-glycan patch antibodies that resemble predicted precursors of human bNAbs. Thus, the disclosed immunogens, such as RC1, are a suitable priming immunogen for sequential vaccination strategies to stimulate an immune response (e.g., HIV immune response) in a subject.

### I. IMMUNOGENS AND IMMUNOGENIC COMPOSITIONS

### A. Polypeptide

This disclosure provides an immunogen and its variants for stimulating an immune response (e.g., HIV immune response) of a subject in need thereof. In some embodiments, the immunogen includes a portion of the HIV envelope protein, i.e., gp120, which is located on the surface of the HIV. gp120 is the N-terminal segment of the HIV envelope protein gp160, anchored in the membrane bilayer at its carboxyl-terminal region. gp120 protrudes into the aqueous environment surrounding the virion, whereas its C-terminal counterpart, gp41, spans the membrane. The gp120 molecule consists of a polypeptide core of 60,000 daltons, which is extensively modified by N-linked glycosylation to increase the apparent molecular weight of the molecule to 120,000 daltons. The amino acid sequence of gp120 contains five relatively conserved domains interspersed with five hypervariable domains. The positions of the 18 cysteine residues in the gp 120 primary sequence and the positions of 13 of the approximately 24 N-linked glycosylation sites in the gp 120 sequence are common to all gp 120 sequences.

In some embodiments, the immunogen may include the Env V3 region of gp120. The Env V3 region of gp 120 encompasses the V3-glycan patch epitope, which includes a group of high-mannose and complex-type N-glycans surrounding the Env V3 region. In the V3-glycan patch epitope, glycosylation generally occurs at gp120 residues N133, N137, N156, N295, N301, N332, N339, N385, and N392. bNAbs, such as PGT121, 10-1074, and BG18, target the V3-glycan patch epitope. They reach through the glycans using elongated CDRH3 loops and portions of CDRL1 and CDRL3 to contact the highly-conserved GDIR motif (G324-D325-1326-R327) (SEQ ID NO: 15) at the base of the V3 loop.

In some embodiments, the immunogen may include one or more modifications in the Env V3 region of gp120. The immunogen comprises a polypeptide having a sequence that is at least 75% identical to a sequence selected from the group consisting of SEQ ID NOs: 2, 4, 6, 8, 11, and 13 listed in Table 1. The polypeptide may include substitutions at one or more glycosylation sites (e.g., N133, N137, N156, N295, N301, N332, N339, N385, and N392) in the Env region. For example, the polypeptide may include a substitution at the positions corresponding to N133, N137, and N156 of SEQ ID NO: 1. In one example, the polypeptide includes an N156Q substitution or a conservative substitution of N156. In another example, the polypeptide, such as RC1 (SEQ ID NO: 2; Table 1), includes deletions at N133, N137, and N156 and additional substitutions including V134Y, T135A, 1138L, T139L, D140S, D141N, T320F, Q328M, and T415V. As will be illustrated in the examples, the disclosed immunogens, such as RC1, activate and expand a diverse group of B-cells expressing antibodies that resemble human V3-glycan bNAb precursors in mice, rabbits, and Rhesus macaques.

TABLE 1
Sequences of HIV Immunogens.

| SEQ ID | | Other |
|---|---|---|
| NO. | Sequence | information |
| SEQ ID | MDAMKRGLCCVLLLCGAVEVSPSQEIHARFRRGARAENLWVTV | 11MUTB |
| NO: 1 | YYGVPVWKDAETTLFCASDAKAYETEKHNVWATHACVPTDPNPQE | (SOSIP.664) |
| | IHLENVTEEFNMWKNNMVEQMHTDIISLWDQSLKPCVKLTPLCVTL | derived |
| | QCTNVTNNITDDMRGELKNCSFNMTTELRDKKQKVYSLFYRLDVV | from |
| | QINENQGNRSNNSNKEYRLINCNTSAITQACPKVSFEPIPIHYCAPAGF | wtBG505 |
| | AILKCKDKKFNGTGPCPSVSTVQCTHGIKPWSTQLLLNGSLAEEEVM | (changes |
| | IRSENITNNAKNILVQFNTPVQINCTRPNNNTRKSIRIGPGQAFYATGD | made to |
| | IIGDIRQAHCNVSKATWNETLGKWKQLRKHFGNNTIIRFANSSGGDL | wtBG505 are |
| | EVTTHSFNCGGEFFYCNTSGLFNSTWISNTSVQGSNSTGSNDSITLPC | underlined |
| | RIKQIINMWQRIGQAMYAPPIQGVIRCVSNITGLILTRDGGSTNSTTET | and in bold) |
| | FRPGGGDMRDNWRSELYKYKWKIEPLGVAPTRCKRRVVGRRRRR | |
| | RAVGIGAVFLGFLGAAGSTMGAASMTLTVQARNLLSGIVQQQSNLL | |
| | RAPEAQQHLLKLTVWGIKQLQARVLAVERYLRDQQLLGIWGCSGK | |
| | LICCTNVPWNSSWSNRNLSEIWDNMTWLQWDKEISNYTQIIYGLLEE | |
| | SQNQQEKNEQDLLALD | |
| SEQ ID | MDAMKRGLCCVLLLCGAVFVSPAGAGSNLWVTVYYGVPVWKDAE | RC1 |
| NO: 2 | TTLFCASDAKAYETEKHNVWATHACVPTDPNPQEIHLENVTEEFNM | |
| | WKNNMVEQMHEDIISLWDQSLKPCVKLTPLCVTLQCTNYAPNLLSN | |
| | MRGELKQCSFNMTTELRDKKQKVYSLFYRLDVVQINENQGNRSNNS | |
| | NKEYRLINCNTSAITQACPKVSFEPIPIHYCAPAGFAILKCKDKKFNGT | |
| | GPCPSVSTVQCTHGIKPVVSTQLLLNGSLAEEEVIIRSENITNNAKNIL | |
| | VQLNTPVQINCTRPNNNTVKSIRIGPGQAFYYFGDIIGDIRMAHCNVS | |
| | KATWNETLGKVVKQLRKHFGNNTIIRFAQSSGGDLEVTTHSFNCGG | |
| | EFFYCNTSGLFNSTWISNTSVQGSNSTGSNDSIVLPCRIKQIINMWQRI | |
| | GQAMYAPPIQGVIRCVSNITGLILTRDGGSTNSTTETFRPGGGDMRD | |
| | NWRSELYKYKVVKIEPLGVAPTRCKRRVVGRRRRRRAVGIGAVSLG | |
| | FLGAAGSTMGAASMTLTVQARNLLSGIVQQQSNLLRAPEPQQHLLK | |
| | DTHWGIKQLQARVLAVEHYLRDQQLLGIWGCSGKLICCTNVPWNSS | |
| | WSNRNLSEIWDNMTWLQWDKEISNYTQIIYGLLEESQNQQEKNEQD | |
| | LLALD | |
| SEQ ID | ATGGACGCCATGAAGAGGGGACTTTGCTGTGTTCTTCTGCTGTGT | RC1 |
| NO: 3 | GGCGCCGTGTTTGTTAGCCCCGCTGGGGCCGGATCCAACCTGTGG | |
| | GTCACTGTGTATTATGGTGTGCCAGTGTGGAAGGATGCAGAGACA | |
| | ACACTCTTTTGCGCCTCCGACGCTAAAGCATACGAAACGGAGAAG | |
| | CACAACGTGTGGGCGACCCATGCCTGTGTCCCTACAGACCCTAAC | |
| | CCTCAGGAAATTCATCTTGAAAATGTCACAGAAGAGTTTAACATG | |
| | TGGAAAAACAACATGGTGGAACAGATGCACGAGGATATCATTTC | |
| | CCTGTGGGACCAGAGTCTGAAACCATGTGTCAAACTTACTCCTCT | |
| | GTGCGTGACTCTCCAGTGTACAAACTACGCACCCAACCTTTTGAG | |
| | TAATATGCGGGGCGAGCTCAAGCAGTGCAGTTTCAATATGACAAC | |
| | CGAATTGAGAGACAAAAAACAGAAAGTATACTCCCTCTTCTACCG | |
| | GCTGGACGTGGTGCAGATCAATGAGAACCAAGGAAATAGAAGCA | |
| | ACAACAGTAACAAGGAATACCGGCTCATAAATTGCAATACCAGC | |
| | GCTATTACGCAGGCTTGCCCTAAGGTGAGCTTTGAGCCAATCCCG | |
| | ATACATTATTGTGCCCCGGCAGGCTTCGCTATACTGAAATGCAAG | |
| | GATAAGAAGTTTAATGGGACAGGCCCTTGCCCTAGCGTTTCAACG | |
| | GTCCAATGTACCCACGGGATCAAGCCCGTAGTGTCTACACAGCTC | |
| | CTGCTGAACGGCAGCCTGGCCGAAGAGGAGGTCATAATTAGGAG | |
| | CGAGAACATAACTAACAACGCTAAAAACATTCTCGTCCAGCTCAA | |
| | TACACCTGTGCAGATCAACTGCACCCGGCCCAACAACAACACCGT | |
| | GAAGTCCATTAGAATTGGTCCGGGACAGGCATTTTACTACTTCGG | |
| | AGATATAATAGGCGATATCAGAATGGCGCACTGTAACGTGAGCA | |
| | AGGCCACCTGGAACGAGACCCTGGGCAAGGTGGTCAAACAGTTG | |
| | CGCAAGCACTTTGGGAACAACACCATTATTCGGTTTGCCCAGTCT | |
| | TCCGGCGGCGACCTTGAAGTGACCACTCATAGCTTCAACTGTGGA | |
| | GGGGAGTTTTTCTATTGCAATACATCAGGCCTGTTCAACTCTACAT | |
| | GGATCTCAAATACCAGTGTCCAGGGGTCAAATTCCACCGGTAGCA | |
| | ACGACAGCATCGTCTTGCCTTGTCGAATCAAGCAGATCATTAATA | |
| | TGTGGCAGAGGATTGGTCAGGCCATGTACGCACCTCCAATACAGG | |
| | GAGTCATTCGGTGCGTCAGCAATATTACTGGATTGATCCTCACCA | |
| | GAGATGGCGGGAGTACCAATAGCACTACCGAAACTTTCCGCCCA | |
| | GGAGGAGGCGACATGCGGGATAATTGGAGATCAGAGCTGTATAA | |
| | GTATAAGGTGGTGAAAATTGAACCCCTGGGAGTGGCGCCAACTA | |
| | GATGTAAACGGCGAGTGGTTGGCCGGAGACGGCGGCGGAGAGCA | |
| | GTGGGGATTGGCGCTGTCTCACTCGGTTTCCTGGGTGCTGCCGGC | |
| | AGTACAATGGGCGCCGCCAGCATGACGCTCACAGTGCAGGCCCG | |
| | GAATCTTCTTAGCGGAATTGTGCAACAACAAAGCAATCTGTTGAG | |
| | AGCCCCGGAACCGCAGCAACATCTGTTGAAGGACACACATTGGG | |
| | GCATCAAGCAGCTGCAAGCTCGGGTTCTGGCTGTTGAGCATTACC | |
| | TGAGAGACCAACAGCTGCTGGGCATATGGGGATGCTCAGGAAAA | |
| | CTGATCTGCTGCACCAATGTCCCATGGAACAGCTCATGGTCAAAC | |
| | AGGAACCTGAGCGAGATCTGGGATAACATGACCTGGTTGCAGTG | |
| | GGACAAAGAAATTAGCAATTACACACAGATCATCTACGGCCTCCT | |
| | GGAGGAAAGCCAGAATCAGCAGGAGAAAAATGAGCAGGATCTG | |
| | CTTGCCCTTGACTGA | |
| SEQ ID | MDAMKRGLCCVLLLCGAVFVSPAGAGSNLWVTVYYGVPVWKDAE | RC1 spytag |
| NO: 4 | TTLFCASDAKAYETEKHNVWATHACVPTDPNPQEIHLENVTEEFNM | |
| | WKNNMVEQMHEDIISLWDQSLKPCVKLTPLCVTLQCTNYAPNLLSN | |
| | MRGELKQCSFNMTTELRDKKQKVYSLFYRLDVVQINENQGNRSNNS | |
| | NKEYRLINCNTSAITQACPKVSFEPIPIHYCAPAGFAILKCKDKKFNGT | |
| | GPCPSVSTVQCTHGIKPVVSTQLLLNGSLAEEEVIIRSENITNNAKNIL | |
| | VQLNTPVQINCTRPNNNTVKSIRIGPGQAFYYFGDIIGDIRMAHCNVS | |
| | KATWNETLGKVVKQLRKHFGNNTIIRFAQSSGGDLEVTTHSFNCGG | |
| | EFFYCNTSGLFNSTWISNTSVQGSNSTGSNDSIVLPCRIKQIINMWQRI | |
| | GQAMYAPPIQGVIRCVSNITGLILTRDGGSTNSTTETFRPGGGDMRD | |
| | NWRSELYKYKVVKIEPLGVAPTRCKRRVVGRRRRRRAVGIGAVSLG | |
| | FLGAAGSTMGAASMTLTVQARNLLSGIVQQQSNLLRAPEPQQHLLK | |
| | DTHWGIKQLQARVLAVEHYLRDQQLLGIWGCSGKLICCTNVPWNSS | |
| | WSNRNLSEIWDNMTWLQWDKEISNYTQIIYGLLEESQNQQEKNEQD | |
| | LLALDGGGGSGGGSGGGSGSGAHIVMVDAYKPTK | |
| SEQ ID | ATGGACGCCATGAAGAGGGGACTTTGCTGTGTTCTTCTGCTGTGT | RC1 spytag |
| NO: 5 | GGCGCCGTGTTTGTTAGCCCCGCTGGGGCCGGATCCAACCTGTGG | |
| | GTCACTGTGTATTATGGTGTGCCAGTGTGGAAGGATGCAGAGACA | |
| | ACACTCTTTTGCGCCTCCGACGCTAAAGCATACGAAACGGAGAAG | |
| | CACAACGTGTGGGCGACCCATGCCTGTGTCCCTACAGACCCTAAC | |
| | CCTCAGGAAATTCATCTTGAAAATGTCACAGAAGAGTTTAACATG | |
| | TGGAAAAACAACATGGTGGAACAGATGCACGAGGATATCATTTC | |
| | CCTGTGGGACCAGAGTCTGAAACCATGTGTCAAACTTACTCCTCT | |
| | GTGCGTGACTCTCCAGTGTACAAACTACGCACCCAACCTTTTGAG | |
| | TAATATGCGGGGCGAGCTCAAGCAGTGCAGTTTCAATATGACAAC | |
| | CGAATTGAGAGACAAAAAACAGAAAGTATACTCCCTCTTCTACCG | |
| | GCTGGACGTGGTGCAGATCAATGAGAACCAAGGAAATAGAAGCA | |
| | ACAACAGTAACAAGGAATACCGGCTCATAAATTGCAATACCAGC | |
| | GCTATTACGCAGGCTTGCCCTAAGGTGAGCTTTGAGCCAATCCCG | |
| | ATACATTATTGTGCCCCGGCAGGCTTCGCTATACTGAAATGCAAG | |
| | GATAAGAAGTTTAATGGGACAGGCCCTTGCCCTAGCGTTTCAACG | |
| | GTCCAATGTACCCACGGGATCAAGCCCGTAGTGTCTACACAGCTC | |
| | CTGCTGAACGGCAGCCTGGCCGAAGAGGAGGTCATAATTAGGAG | |
| | CGAGAACATAACTAACAACGCTAAAAACATTCTCGTCCAGCTCAA | |
| | TACACCTGTGCAGATCAACTGCACCCGGCCCAACAACAACACCGT | |
| | GAAGTCCATTAGAATTGGTCCGGGACAGGCATTTTACTACTTCGG | |
| | AGATATAATAGGCGATATCAGAATGGCGCACTGTAACGTGAGCA | |
| | AGGCCACCTGGAACGAGACCCTGGGCAAGGTGGTCAAACAGTTG | |
| | CGCAAGCACTTTGGGAACAACACCATTATTCGGTTTGCCCAGTCT | |
| | TCCGGCGGCGACCTTGAAGTGACCACTCATAGCTTCAACTGTGGA | |
| | GGGGAGTTTTTCTATTGCAATACATCAGGCCTGTTCAACTCTACAT | |
| | GGATCTCAAATACCAGTGTCCAGGGGTCAAATTCCACCGGTAGCA | |
| | ACGACAGCATCGTCTTGCCTTGTCGAATCAAGCAGATCATTAATA | |
| | TGTGGCAGAGGATTGGTCAGGCCATGTACGCACCTCCAATACAGG | |
| | GAGTCATTCGGTGCGTCAGCAATATTACTGGATTGATCCTCACCA | |
| | GAGATGGCGGGAGTACCAATAGCACTACCGAAACTTTCCGCCCA | |
| | GGAGGAGGCGACATGCGGGATAATTGGAGATCAGAGCTGTATAA | |
| | GTATAAGGTGGTGAAAATTGAACCCCTGGGAGTGGCGCCAACTA | |
| | GATGTAAACGGCGAGTGGTTGGCCGGAGACGGCGGCGGAGAGCA | |
| | GTGGGGATTGGCGCTGTCTCACTCGGTTTCCTGGGTGCTGCCGGC | |
| | AGTACAATGGGCGCCGCCAGCATGACGCTCACAGTGCAGGCCCG | |
| | GAATCTTCTTAGCGGAATTGTGCAACAACAAAGCAATCTGTTGAG | |
| | AGCCCCGGAACCGCAGCAACATCTGTTGAAGGACACACATTGGG | |
| | GCATCAAGCAGCTGCAAGCTCGGGTTCTGGCTGTTGAGCATTACC | |
| | TGAGAGACCAACAGCTGCTGGGCATATGGGGATGCTCAGGAAAA | |
| | CTGATCTGCTGCACCAATGTCCCATGGAACAGCTCATGGTCAAAC | |
| | AGGAACCTGAGCGAGATCTGGGATAACATGACCTGGTTGCAGTG | |
| | GGACAAAGAAATTAGCAATTACACACAGATCATCTACGGCCTCCT | |
| | GGAGGAAAGCCAGAATCAGCAGGAGAAAAATGAGCAGGATCTG | |
| | CTTGCCCTTGACGGTGGAGGCGGTTCAGGCGGCGGATCTGGCGGT | |
| | GGGAGCGGTTCGGGAGCCCATATAGTGATGGTTGATGCCTATAAA | |
| | CCGACCAAGTGA | |
| SEQ ID | MDAMKRGLCCVLLLCGAVFVSPAGAGSNLWVTVYYGVPVWKDAE | RC1-4fill |
| NO: 6 | TTLFCASDAKAYETEKHNVWATHACVPTDPNPQEIHLENVTEEFNM | |
| | WKNNMVEQMHEDIISLWDQSLKPCVKLTPLCVTLQCTNYAPNLLSN | |
| | MRGELKQCSFNMTTELRDKKQKVYSLFYRLDVVQINENQGNRSNNS | |
| | NKEYRLINCNTSAITQACPKVSFEPIPIHYCAPAGFAILKCKNKTFNGT | |
| | GPCPNVSTVQCTHGIKPVVSTQLLLNGSLAEEEVIIRSENITNNAKNIL | |
| | VQLNTSVQINCTRPNNNTVKSIRIGPGQAFYYFGDIIGDIRMAHCNVS | |
| | KATWNETLGNVSKQLRKHFGNNTIIRFAQSSGGDLEVTTHSFNCGGE | |
| | FFYCNTSGLFNSTWISNTSVQGSNSTGSNDSIVLPCRIKQIINMWQRIG | |
| | QAMYAPPIQGVIRCVSNITGLILTRDGGSTNSTTETFRPGGGDMRDN | |
| | WRSELYKYKVVKIEPLGVAPTRCKRRVVGRRRRRRAVGIGAVSLGF | |
| | LGAAGSTMGAASMTLTVQARNLLSGIVQQQSNLLRAPEPQQHLLKD | |
| | THWGIKQLQARVLAVEHYLRDQQLLGIWGCSGKLICCTNVPWNSS | |
| | WSNRNLSEIWDNMTWLQWDKEISNYTQIIYGLLEESQNQQEKNEQD | |
| | LLALD | |
| SEQ ID | ATGGACGCCATGAAGAGGGGACTTTGCTGTGTTCTTCTGCTGTGT | RC1-4fill |
| NO: 7 | GGCGCCGTGTTTGTTAGCCCCGCTGGGGCCGGATCCAACCTGTGG | |
| | GTCACTGTGTATTATGGTGTGCCAGTGTGGAAGGATGCAGAGACA | |
| | ACACTCTTTTGCGCCTCCGACGCTAAAGCATACGAAACGGAGAAG | |
| | CACAACGTGTGGGCGACCCATGCCTGTGTCCCTACAGACCCTAAC | |
| | CCTCAGGAAATTCATCTTGAAAATGTCACAGAAGAGTTTAACATG | |
| | TGGAAAAACAACATGGTGGAACAGATGCACGAGGATATCATTTC | |
| | CCTGTGGGACCAGAGTCTGAAACCATGTGTCAAACTTACTCCTCT | |
| | GTGCGTGACTCTCCAGTGTACAAACTACGCACCCAACCTTTTGAG | |
| | TAATATGCGGGGCGAGCTCAAGCAGTGCAGTTTCAATATGACAAC | |
| | CGAATTGAGAGACAAAAAACAGAAAGTATACTCCCTCTTCTACCG | |
| | GCTGGACGTGGTGCAGATCAATGAGAACCAAGGAAATAGAAGCA | |
| | ACAACAGTAACAAGGAATACCGGCTCATAAATTGCAATACCAGC | |
| | GCTATTACGCAGGCTTGCCCTAAGGTGAGCTTTGAGCCAATCCCG | |
| | ATACATTATTGTGCCCCGGCAGGCTTCGCTATACTGAAATGCAAG | |
| | AATAAGACGTTTAATGGGACAGGCCCTTGCCCTAACGTTTCAACG | |
| | GTCCAATGTACCCACGGGATCAAGCCCGTAGTGTCTACACAGCTC | |
| | CTGCTGAACGGCAGCCTGGCCGAAGAGGAGGTCATAATTAGGAG | |
| | CGAGAACATAACTAACAACGCTAAAAACATTCTCGTCCAGCTCAA | |
| | TACAAGTGTGCAGATCAACTGCACCCGGCCCAACAACAACACCG | |
| | TGAAGTCCATTAGAATTGGTCCGGGACAGGCATTTTACTACTTCG | |
| | GAGATATAATAGGCGATATCAGAATGGCGCACTGTAACGTGAGC | |
| | AAGGCCACCTGGAACGAGACCCTGGGCAATGTGAGCAAACAGTT | |
| | GCGCAAGCACTTTGGGAACAACACCATTATTCGGTTTGCCCAGTC | |
| | TTCCGGCGGCGACCTTGAAGTGACCACTCATAGCTTCAACTGTGG | |
| | AGGGGAGTTTTTCTATTGCAATACATCAGGCCTGTTCAACTCTAC | |
| | ATGGATCTCAAATACCAGTGTCCAGGGGTCAAATTCCACCGGTAG | |
| | CAACGACAGCATCGTCTTGCCTTGTCGAATCAAGCAGATCATTAA | |
| | TATGTGGCAGAGGATTGGTCAGGCCATGTACGCACCTCCAATACA | |
| | GGGAGTCATTCGGTGCGTCAGCAATATTACTGGATTGATCCTCAC | |
| | CAGAGATGGCGGGAGTACCAATAGCACTACCGAAACTTTCCGCC | |
| | CAGGAGGAGGCGACATGCGGGATAATTGGAGATCAGAGCTGTAT | |
| | AAGTATAAGGTGGTGAAAATTGAACCCCTGGGAGTGGCGCCAAC | |
| | TAGATGTAAACGGCGAGTGGTTGGCCGGAGACGGCGGCGGAGAG | |
| | CAGTGGGGATTGGCGCTGTCTCACTCGGTTTCCTGGGTGCTGCCG | |
| | GCAGTACAATGGGCGCCGCCAGCATGACGCTCACAGTGCAGGCC | |
| | CGGAATCTTCTTAGCGGAATTGTGCAACAACAAAGCAATCTGTTG | |
| | AGAGCCCCGGAACCGCAGCAACATCTGTTGAAGGACACACATTG | |
| | GGGCATCAAGCAGCTGCAAGCTCGGGTTCTGGCTGTTGAGCATTA | |
| | CCTGAGAGACCAACAGCTGCTGGGCATATGGGGATGCTCAGGAA | |
| | AACTGATCTGCTGCACCAATGTCCCATGGAACAGCTCATGGTCAA | |
| | ACAGGAACCTGAGCGAGATCTGGGATAACATGACCTGGTTGCAG | |
| | TGGGACAAAGAAATTAGCAATTACACACAGATCATCTACGGCCTC | |
| | CTGGAGGAAAGCCAGAATCAGCAGGAGAAAAATGAGCAGGATCT | |
| | GCTTGCCCTTGACTGA | |
| SEQ ID | MDAMKRGLCCVLLLCGAVFVSPAGAGSNLWVTVYYGVPVWKDAE | RC1-4fill |
| NO: 8 | TTLFCASDAKAYETEKHNVWATHACVPTDPNPQEIHLENVTEEFNM | spytag |
| | WKNNMVEQMHEDIISLWDQSLKPCVKLTPLCVTLQCTNYAPNLLSN | |
| | MRGELKQCSFNMTTELRDKKQKVYSLFYRLDVVQINENQGNRSNNS | |
| | NKEYRLINCNTSAITQACPKVSFEPIPIHYCAPAGFAILKCKNKTFNGT | |
| | GPCPNVSTVQCTHGIKPVVSTQLLLNGSLAEEEVIIRSENITNNAKNIL | |
| | VQLNTSVQINCTRPNNNTVKSIRIGPGQAFYYFGDIIGDIRMAHCNVS | |
| | KATWNETLGNVSKQLRKHFGNNTIIRFAQSSGGDLEVTTHSFNCGGE | |
| | FFYCNTSGLFNSTWISNTSVQGSNSTGSNDSIVLPCRIKQIINMWQRIG | |
| | QAMYAPPIQGVIRCVSNITGLILTRDGGSTNSTTETFRPGGGDMRDN | |
| | WRSELYKYKVVKIEPLGVAPTRCKRRVVGRRRRRRAVGIGAVSLGF | |
| | LGAAGSTMGAASMTLTVQARNLLSGIVQQQSNLLRAPEPQQHLLKD | |
| | THWGIKQLQARVLAVEHYLRDQQLLGIWGCSGKLICCTNVPWNSS | |
| | WSNRNLSEIWDNMTWLQWDKEISNYTQIIYGLLEESQNQQEKNEQD | |
| | LLALDGGGGSGGGSGGGSGSGAHIVMVDAYKPTK | |
| SEQ ID | ATGGACGCCATGAAGAGGGGACTTTGCTGTGTTCTTCTGCTGTGT | RC1-4fill |
| NO: 9 | GGCGCCGTGTTTGTTAGCCCCGCTGGGGCCGGATCCAACCTGTGG | spytag |
| | GTCACTGTGTATTATGGTGTGCCAGTGTGGAAGGATGCAGAGACA | |
| | ACACTCTTTTGCGCCTCCGACGCTAAAGCATACGAAACGGAGAAG | |
| | CACAACGTGTGGGCGACCCATGCCTGTGTCCCTACAGACCCTAAC | |
| | CCTCAGGAAATTCATCTTGAAAATGTCACAGAAGAGTTTAACATG | |
| | TGGAAAAACAACATGGTGGAACAGATGCACGAGGATATCATTTC | |
| | CCTGTGGGACCAGAGTCTGAAACCATGTGTCAAACTTACTCCTCT | |
| | GTGCGTGACTCTCCAGTGTACAAACTACGCACCCAACCTTTTGAG | |
| | TAATATGCGGGGCGAGCTCAAGCAGTGCAGTTTCAATATGACAAC | |
| | CGAATTGAGAGACAAAAAACAGAAAGTATACTCCCTCTTCTACCG | |
| | GCTGGACGTGGTGCAGATCAATGAGAACCAAGGAAATAGAAGCA | |
| | ACAACAGTAACAAGGAATACCGGCTCATAAATTGCAATACCAGC | |
| | GCTATTACGCAGGCTTGCCCTAAGGTGAGCTTTGAGCCAATCCCG | |
| | ATACATTATTGTGCCCCGGCAGGCTTCGCTATACTGAAATGCAAG | |
| | AATAAGACGTTTAATGGGACAGGCCCTTGCCCTAACGTTTCAACG | |
| | GTCCAATGTACCCACGGGATCAAGCCCGTAGTGTCTACACAGCTC | |
| | CTGCTGAACGGCAGCCTGGCCGAAGAGGAGGTCATAATTAGGAG | |
| | CGAGAACATAACTAACAACGCTAAAAACATTCTCGTCCAGCTCAA | |
| | TACAAGTGTGCAGATCAACTGCACCCGGCCCAACAACAACACCG | |
| | TGAAGTCCATTAGAATTGGTCCGGGACAGGCATTTTACTACTTCG | |
| | GAGATATAATAGGCGATATCAGAATGGCGCACTGTAACGTGAGC | |
| | AAGGCCACCTGGAACGAGACCCTGGGCAATGTGAGCAAACAGTT | |
| | GCGCAAGCACTTTGGGAACAACACCATTATTCGGTTTGCCCAGTC | |
| | TTCCGGCGGCGACCTTGAAGTGACCACTCATAGCTTCAACTGTGG | |
| | AGGGGAGTTTTTCTATTGCAATACATCAGGCCTGTTCAACTCTAC | |
| | ATGGATCTCAAATACCAGTGTCCAGGGGTCAAATTCCACCGGTAG | |
| | CAACGACAGCATCGTCTTGCCTTGTCGAATCAAGCAGATCATTAA | |
| | TATGTGGCAGAGGATTGGTCAGGCCATGTACGCACCTCCAATACA | |
| | GGGAGTCATTCGGTGCGTCAGCAATATTACTGGATTGATCCTCAC | |
| | CAGAGATGGCGGGAGTACCAATAGCACTACCGAAACTTTCCGCC | |
| | CAGGAGGAGGCGACATGCGGGATAATTGGAGATCAGAGCTGTAT | |
| | AAGTATAAGGTGGTGAAAATTGAACCCCTGGGAGTGGCGCCAAC | |
| | TAGATGTAAACGGCGAGTGGTTGGCCGGAGACGGCGGCGGAGAG | |
| | CAGTGGGGATTGGCGCTGTCTCACTCGGTTTCCTGGGTGCTGCCG | |
| | GCAGTACAATGGGCGCCGCCAGCATGACGCTCACAGTGCAGGCC | |
| | CGGAATCTTCTTAGCGGAATTGTGCAACAACAAAGCAATCTGTTG | |
| | AGAGCCCCGGAACCGCAGCAACATCTGTTGAAGGACACACATTG | |
| | GGGCATCAAGCAGCTGCAAGCTCGGGTTCTGGCTGTTGAGCATTA | |
| | CCTGAGAGACCAACAGCTGCTGGGCATATGGGGATGCTCAGGAA | |
| | AACTGATCTGCTGCACCAATGTCCCATGGAACAGCTCATGGTCAA | |
| | ACAGGAACCTGAGCGAGATCTGGGATAACATGACCTGGTTGCAG | |
| | TGGGACAAAGAAATTAGCAATTACACACAGATCATCTACGGCCTC | |
| | CTGGAGGAAAGCCAGAATCAGCAGGAGAAAAATGAGCAGGATCT | |
| | GCTTGCCCTTGACGGTGGAGGCGGTTCAGGCGGCGGATCTGGCGG | |
| | TGGGAGCGGTTCGGGAGCCCATATAGTGATGGTTGATGCCTATAA | |
| | ACCGACCAAGTGA | |
| SEQ ID | KGKGKGKGKGCTRPNNNTRKSIRIGPGQTFYATGDIIGDIRQAHC | V3 loop- |
| NO: 10 | | Consensus |
| | | C peptide |
| SEQ ID | MDAMKRGLCCVLLLCGAVFVSPAGAGSNLWVTVYYGVPVW | RC1-3fill |
| NO: 11 | KDAETTLFCASDAKAYETEKHNVWATHACVPTDPNPQEIHLE | |
| | NVTEEFNMWKNNMVEQMHEDIISLWDQSLKPCVKLTPLCVTL | |
| | QCTNYAPNLLSNMRGELKQCSFNMTTELRDKKQKVYSLFYRL | |
| | DVVQINENQGNRSNNSNKEYRLINCNTSAITQACPKVSFEPIPIH | |
| | YCAPAGFAILKCKNKTFNGTGPCPNVSTVQCTHGIKPVVSTQL | |
| | LLNGSLAEEEVIIRSENITNNAKNILVQLNTPVQINCTRPNNNTV | |
| | KSIRIGPGQAFYYFGDIIGDIRMAHCNVSKATWNETLGNVSKQ | |
| | LRKHFGNNTIIRFAQSSGGDLEVTTHSFNCGGEFFYCNTSGLFN | |
| | STWISNTSVQGSNSTGSNDSIVLPCRIKQIINMWQRIGQAMYAP | |
| | PIQGVIRCVSNITGLILTRDGGSTNSTTETFRPGGGDMRDNWRS | |
| | ELYKYKVVKIEPLGVAPTRCKRRVVGRRRRRRAVGIGAVSLG | |
| | FLGAAGSTMGAASMTLTVQARNLLSGIVQQQSNLLRAPEPQQ | |
| | HLLKDTHWGIKQLQARVLAVEHYLRDQQLLGIWGCSGKLICC | |
| | TNVPWNSSWSNRNLSEIWDNMTWLQWDKEISNYTQIIYGLLE | |
| | ESQNQQEKNEQDLLALD | |
| SEQ ID | ATGGACGCCATGAAGAGGGGACTTTGCTGTGTTCTTCTGCT | RC1-3fill |
| NO: 12 | GTGTGGCGCCGTGTTTGTTAGCCCCGCTGGGGCCGGATCCA | |
| | ACCTGTGGGTCACTGTGTATTATGGTGTGCCAGTGTGGAAG | |
| | GATGCAGAGACAACACTCTTTTGCGCCTCCGACGCTAAAGC | |
| | ATACGAAACGGAGAAGCACAACGTGTGGGCGACCCATGCC | |
| | TGTGTCCCTACAGACCCTAACCCTCAGGAAATTCATCTTGA | |
| | AAATGTCACAGAAGAGTTTAACATGTGGAAAAACAACATG | |
| | GTGGAACAGATGCACGAGGATATCATTTCCCTGTGGGACCA | |
| | GAGTCTGAAACCATGTGTCAAACTTACTCCTCTGTGCGTGA | |
| | CTCTCCAGTGTACAAACTACGCACCCAACCTTTTGAGTAAT | |
| | ATGCGGGGCGAGCTCAAGCAGTGCAGTTTCAATATGACAAC | |
| | CGAATTGAGAGACAAAAAACAGAAAGTATACTCCCTCTTCT | |
| | ACCGGCTGGACGTGGTGCAGATCAATGAGAACCAAGGAAA | |
| | TAGAAGCAACAACAGTAACAAGGAATACCGGCTCATAAAT | |
| | TGCAATACCAGCGCTATTACGCAGGCTTGCCCTAAGGTGAG | |
| | CTTTGAGCCAATCCCGATACATTATTGTGCCCCGGCAGGCTT | |
| | CGCTATACTGAAATGCAAGAATAAGACGTTTAATGGGACAG | |
| | GCCCTTGCCCTAACGTTTCAACGGTCCAATGTACCCACGGG | |
| | ATCAAGCCCGTAGTGTCTACACAGCTCCTGCTGAACGGCAG | |
| | CCTGGCCGAAGAGGAGGTCATAATTAGGAGCGAGAACATA | |
| | ACTAACAACGCTAAAAACATTCTCGTCCAGCTCAATACACC | |
| | TGTGCAGATCAACTGCACCCGGCCCAACAACAACACCGTGA | |
| | AGTCCATTAGAATTGGTCCGGGACAGGCATTTTACTACTTC | |
| | GGAGATATAATAGGCGATATCAGAATGGCGCACTGTAACGT | |
| | GAGCAAGGCCACCTGGAACGAGACCCTGGGCAATGTGAGC | |
| | AAACAGTTGCGCAAGCACTTTGGGAACAACACCATTATTCG | |
| | GTTTGCCCAGTCTTCCGGCGGCGACCTTGAAGTGACCACTC | |
| | ATAGCTTCAACTGTGGAGGGGAGTTTTTCTATTGCAATACAT | |
| | CAGGCCTGTTCAACTCTACATGGATCTCAAATACCAGTGTC | |
| | CAGGGGTCAAATTCCACCGGTAGCAACGACAGCATCGTCTT | |
| | GCCTTGTCGAATCAAGCAGATCATTAATATGTGGCAGAGGA | |
| | TTGGTCAGGCCATGTACGCACCTCCAATACAGGGAGTCATT | |
| | CGGTGCGTCAGCAATATTACTGGATTGATCCTCACCAGAGA | |
| | TGGCGGGAGTACCAATAGCACTACCGAAACTTTCCGCCCAG | |
| | GAGGAGGCGACATGCGGGATAATTGGAGATCAGAGCTGTA | |
| | TAAGTATAAGGTGGTGAAAATTGAACCCCTGGGAGTGGCGC | |
| | CAACTAGATGTAAACGGCGAGTGGTTGGCCGGAGACGGCG | |
| | GCGGAGAGCAGTGGGGATTGGCGCTGTCTCACTCGGTTTCC | |
| | TGGGTGCTGCCGGCAGTACAATGGGCGCCGCCAGCATGACG | |
| | CTCACAGTGCAGGCCCGGAATCTTCTTAGCGGAATTGTGCA | |
| | ACAACAAAGCAATCTGTTGAGAGCCCCGGAACCGCAGCAA | |
| | CATCTGTTGAAGGACACACATTGGGGCATCAAGCAGCTGCA | |
| | AGCTCGGGTTCTGGCTGTTGAGCATTACCTGAGAGACCAAC | |
| | AGCTGCTGGGCATATGGGGATGCTCAGGAAAACTGATCTGC | |
| | TGCACCAATGTCCCATGGAACAGCTCATGGTCAAACAGGAA | |
| | CCTGAGCGAGATCTGGGATAACATGACCTGGTTGCAGTGGG | |
| | ACAAAGAAATTAGCAATTACACACAGATCATCTACGGCCTC | |
| | CTGGAGGAAAGCCAGAATCAGCAGGAGAAAAATGAGCAGG | |
| | ATCTGCTTGCCCTTGACTGA | |
| SEQ ID | MDAMKRGLCCVLLLCGAVFVSPAGAGSNLWVTVYYGVPVW | RC1-3fill- |
| NO: 13 | KDAETTLFCASDAKAYETEKHNVWATHACVPTDPNPQEIHLE | Spytag |
| | NVTEEFNMWKNNMVEQMHEDIISLWDQSLKPCVKLTPLCVTL | |
| | QCTNYAPNLLSNMRGELKQCSFNMTTELRDKKQKVYSLFYRL | |
| | DVVQINENQGNRSNNSNKEYRLINCNTSAITQACPKVSFEPIPIH | |
| | YCAPAGFAILKCKNKTFNGTGPCPNVSTVQCTHGIKPVVSTQL | |
| | LLNGSLAEEEVIIRSENITNNAKNILVQLNTPVQINCTRPNNNTV | |
| | KSIRIGPGQAFYYFGDIIGDIRMAHCNVSKATWNETLGNVSKQ | |
| | LRKHFGNNTIIRFAQSSGGDLEVTTHSFNCGGEFFYCNTSGLFN | |
| | STWISNTSVQGSNSTGSNDSIVLPCRIKQIINMWQRIGQAMYAP | |
| | PIQGVIRCVSNITGLILTRDGGSTNSTTETFRPGGGDMRDNWRS | |
| | ELYKYKVVKIEPLGVAPTRCKRRVVGRRRRRRAVGIGAVSLG | |
| | FLGAAGSTMGAASMTLTVQARNLLSGIVQQQSNLLRAPEPQQ | |
| | HLLKDTHWGIKQLQARVLAVEHYLRDQQLLGIWGCSGKLICC | |
| | TNVPWNSSWSNRNLSEIWDNMTWLQWDKEISNYTQIIYGLLE | |
| | ESQNQQEKNEQDLLALDGGGGSGGGSGGGSGSGAHIVMVDA | |
| | YKPTK | |
| SEQ ID | ATGGACGCCATGAAGAGGGGACTTTGCTGTGTTCTTCTGCTGTGT | RC1-3fill- |
| NO: 14 | GGCGCCGTGTTTGTTAGCCCCGCTGGGGCCGGATCCAACCTGTGG | Spytag |
| | GTCACTGTGTATTATGGTGTGCCAGTGTGGAAGGATGCAGAGACA | |
| | ACACTCTTTTGCGCCTCCGACGCTAAAGCATACGAAACGGAGAAG | |
| | CACAACGTGTGGGCGACCCATGCCTGTGTCCCTACAGACCCTAAC | |
| | CCTCAGGAAATTCATCTTGAAAATGTCACAGAAGAGTTTAACATG | |
| | TGGAAAAACAACATGGTGGAACAGATGCACGAGGATATCATTTC | |
| | CCTGTGGGACCAGAGTCTGAAACCATGTGTCAAACTTACTCCTCT | |
| | GTGCGTGACTCTCCAGTGTACAAACTACGCACCCAACCTTTTGAG | |
| | TAATATGCGGGGCGAGCTCAAGCAGTGCAGTTTCAATATGACAAC | |
| | CGAATTGAGAGACAAAAAACAGAAAGTATACTCCCTCTTCTACCG | |
| | GCTGGACGTGGTGCAGATCAATGAGAACCAAGGAAATAGAAGCA | |
| | ACAACAGTAACAAGGAATACCGGCTCATAAATTGCAATACCAGC | |
| | GCTATTACGCAGGCTTGCCCTAAGGTGAGCTTTGAGCCAATCCCG | |
| | ATACATTATTGTGCCCCGGCAGGCTTCGCTATACTGAAATGCAAG | |
| | AATAAGACGTTTAATGGGACAGGCCCTTGCCCTAACGTTTCAACG | |
| | GTCCAATGTACCCACGGGATCAAGCCCGTAGTGTCTACACAGCTC | |
| | CTGCTGAACGGCAGCCTGGCCGAAGAGGAGGTCATAATTAGGAG | |
| | CGAGAACATAACTAACAACGCTAAAAACATTCTCGTCCAGCTCAA | |
| | TACACCTGTGCAGATCAACTGCACCCGGCCCAACAACAACACCGT | |
| | GAAGTCCATTAGAATTGGTCCGGGACAGGCATTTTACTACTTCGG | |
| | AGATATAATAGGCGATATCAGAATGGCGCACTGTAACGTGAGCA | |
| | AGGCCACCTGGAACGAGACCCTGGGCAATGTGAGCAAACAGTTG | |
| | CGCAAGCACTTTGGGAACAACACCATTATTCGGTTTGCCCAGTCT | |
| | TCCGGCGGCGACCTTGAAGTGACCACTCATAGCTTCAACTGTGGA | |
| | GGGGAGTTTTTCTATTGCAATACATCAGGCCTGTTCAACTCTACAT | |
| | GGATCTCAAATACCAGTGTCCAGGGGTCAAATTCCACCGGTAGCA | |
| | ACGACAGCATCGTCTTGCCTTGTCGAATCAAGCAGATCATTAATA | |
| | TGTGGCAGAGGATTGGTCAGGCCATGTACGCACCTCCAATACAGG | |
| | GAGTCATTCGGTGCGTCAGCAATATTACTGGATTGATCCTCACCA | |
| | GAGATGGCGGGAGTACCAATAGCACTACCGAAACTTTCCGCCCA | |
| | GGAGGAGGCGACATGCGGGATAATTGGAGATCAGAGCTGTATAA | |
| | GTATAAGGTGGTGAAAATTGAACCCCTGGGAGTGGCGCCAACTA | |
| | GATGTAAACGGCGAGTGGTTGGCCGGAGACGGCGGCGGAGAGCA | |
| | GTGGGGATTGGCGCTGTCTCACTCGGTTTCCTGGGTGCTGCCGGC | |
| | AGTACAATGGGCGCCGCCAGCATGACGCTCACAGTGCAGGCCCG | |
| | GAATCTTCTTAGCGGAATTGTGCAACAACAAAGCAATCTGTTGAG | |
| | AGCCCCGGAACCGCAGCAACATCTGTTGAAGGACACACATTGGG | |
| | GCATCAAGCAGCTGCAAGCTCGGGTTCTGGCTGTTGAGCATTACC | |
| | TGAGAGACCAACAGCTGCTGGGCATATGGGGATGCTCAGGAAAA | |
| | CTGATCTGCTGCACCAATGTCCCATGGAACAGCTCATGGTCAAAC | |
| | AGGAACCTGAGCGAGATCTGGGATAACATGACCTGGTTGCAGTG | |
| | GGACAAAGAAATTAGCAATTACACACAGATCATCTACGGCCTCCT | |
| | GGAGGAAAGCCAGAATCAGCAGGAGAAAAATGAGCAGGATCTG | |
| | CTTGCCCTTGACGGTGGAGGCGGTTCAGGCGGCGGATCTGGCGGT | |
| | GGGAGCGGTTCGGGAGCCCATATAGTGATGGTTGATGCCTATAAA | |
| | CCGACCAAGTGA | |

The above amino acid or nucleic acid sequences of HIV Immunogens include the secretion leader sequence at the N-terminal end (for amino acid sequences) or the 5′ end (for nucleic acid sequences). The secretion leader sequence is a general secretion signal and is not part of the final/mature expressed protein. As will be understood by persons having ordinary skill in the art that other secretion leader sequences can also be used to generate the same final/mature HIV Immunogens.

TABLE 2
Immunogen Variants and Specific Modifications.

| | PNGS | | | |
|---|---|---|---|---|
| Protein | Deleted | Added | Other modifications | Purpose |
| RC1 | 133, 137, | — | V134Y, T135A, | Immunization/ |
| | 156 | | I138L, T139L, D140S, | ELISA |
| | | | D141N, T320F, | |
| | | | Q328M, T415V, | |
| | | | MD39* | |
| RC1-3fill | 133, 137, | N230, | V134Y, T135A, | Immunization/ |
| | 156 | N241, | I138L, T139L, D140S, | ELISA |
| | | N344 | D141N, T320F, | |
| | | | Q328M, T415V, | |
| | | | MD39* | |
| RC1-4fill | 133, 137, | N230, | V134Y, T135A, | Immunization/ |
| | 156 | N241, | I138L, T139L, D140S, | ELISA |
| | | N289, | D141N, T320F, | |
| | | N344 | Q328M, T415V, | |
| | | | MD39* | |
| RC1-glycanKO | 133, 137, | — | V134Y, T135A, | ELISA |
| | 156, 301, | | I138L, T139L, D140S, | |
| | 332 | | D141N, T320F, | |
| | | | Q328M, T415V, | |
| | | | H330A, MD39* | |
| RC1-glycanKO-GAIA | 133, 137, | — | V134Y, T135A, | ELISA |
| (“GAIA” disclosed as | 156, 301, | | I138L, T139L, D140S, | |
| SEQ ID NO: 16) | 332 | | D141N, T320F, | |
| | | | Q328M, T415V, | |
| | | | GDIR (SEQ ID NO: | |
| | | | 15)/GAIA (SEQ ID | |
| | | | NO: 16), H330A, | |
| | | | MD39* | |
| RC1-GAIA | 133, 137, | — | V134Y, T135A, | ELISA |
| (“GAIA” disclosed as | 156 | | I138L, T139L, D140S, | |
| SEQ ID NO: 16) | | | D141N, T320F, | |
| | | | Q328M, T415V, | |
| | | | GDIR (SEQ ID NO: | |
| | | | 15)/GAIA (SEQ ID | |
| | | | NO: 16), MD39* | |
| 11MUTBD301 | 133, 137, | — | V134Y, T135A, | Immunization/ |
| | 301 | | I138L, T139L, D140S, | ELISA |
| | | | D141N, T320F, | |
| | | | Q328M, T415V, | |
| | | | MD39* | |
| RC1D301 | 133, 137, | — | V134Y, T135A, | ELISA |
| | 156, 301 | | I138L, T139L, D140S, | |
| | | | D141N, T320F, | |
| | | | Q328M, T415V, | |
| | | | MD39* | |
| RC1D332 | 133, 137, | | V134Y, T135A, | ELISA |
| | 156, 332 | | I138L, T139L, D140S, | |
| | | | D141N, T320F, | |
| | | | Q328M, T415V, | |
| | | | MD39* | |
| 11MUTB | 133, 137 | — | V134Y, T135A, | Immunization/ |
| | | | I138L, T139L, D140S, | ELISA |
| | | | D141N, T320F, | |
| | | | Q328M, T415V, | |
| | | | MD39* | |
| 10MUT | 133, 137 | — | V134Y, T135A, | ELISA |
| | | | N136P, N137F, I138L, | |
| | | | T139I, D140N, T320F, | |
| | | | Q328M, MD39* | |
| 7MUT | 133, 137 | — | V134Y, T135A, | ELISA |
| | | | N136P, N137F, I138L, | |
| | | | T139I, D140N, | |
| | | | MD39* | |
| 5MUT | — | — | V134Y, N136P, | ELISA |
| | | | I138L, D140N, | |
| | | | MD39* | |
| BG505 | — | — | MD39* | ELISA |
| RC1-4fill VLP | 133, 137, | N230, | V134Y, T135A, | Immunization |
| | 156 | N241, | I138L, T139L, D140S, | |
| | | N289, | D141N, T320F, | |
| | | N344 | Q328M, T415V, | |
| | | | Spytag, MD39* | |
| RC1-Avitag | 133, 137, | — | V134Y, T135A, | Sort |
| | 156 | | I138L, T139L, D140S, | |
| | | | D141N, T320F, | |
| | | | Q328M, T415V, | |
| | | | Avitag, MD39* | |
| RC1-glycanKO-Avitag | 133, 137, | — | V134Y, T135A, | Sort |
| | 156, 301, | | I138L, T139L, D140S, | |
| | 332 | | D141N, T320F, | |
| | | | Q328M, T415V, | |
| | | | H330A, Avitag, | |
| | | | MD39* | |

“Polypeptide” is used in its conventional meaning, i.e., as a sequence of amino acids. The polypeptides are not limited to a specific length of the product. Peptides, polypeptides, and proteins are included within the definition of polypeptide, and such terms can be used interchangeably herein unless specifically indicated otherwise. This term also includes post-expression modifications of the polypeptide, for example, glycosylations, acetylations, phosphorylations and the like, as well as other modifications known in the art, both naturally occurring and non-naturally occurring. A polypeptide can be an entire protein or a subsequence thereof. A polypeptide “variant,” as the term is used herein, is a polypeptide that typically differs from a polypeptide specifically disclosed herein in one or more substitutions, deletions, additions and/or insertions. Such variants can be naturally occurring or can be synthetically generated, for example, by modifying one or more of the above polypeptide sequences of the disclosure and evaluating one or more biological activities of the polypeptide as described herein and/or using any of some techniques well known in the art.

For example, certain amino acids can be substituted for other amino acids in a protein structure without appreciable loss of its ability to bind other polypeptides (for example, antigens) or cells. Since it is the binding capacity and nature of a protein that defines that protein's biological functional activity, certain amino acid sequence substitutions can be made in a protein sequence, and, accordingly, its underlying DNA coding sequence, whereby a protein with like properties is obtained. It is thus contemplated that various changes can be made in the peptide sequences of the disclosed compositions, or corresponding DNA sequences that encode said peptides without appreciable loss of their biological utility or activity.

Variant sequences include those wherein conservative substitutions have been introduced by modification of polynucleotides encoding polypeptides of this disclosure. Amino acids can be classified according to physical properties and contribution to secondary and tertiary protein structure. Such conservative modifications include amino acid substitutions, additions, and deletions. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

“Sequence identity” or “homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least about 70%, at least about 75%, at least about 80%, at least about 90%, at least about 95%, at least about 98%, or at least about 99% polynucleotide or polypeptide homology with a polynucleotide or polypeptide described herein.

Polypeptide variant sequences may share 70% or more (i.e. 80%, 85%, 90%, 95%, 97%, 98%, 99% or more) sequence identity with the sequences recited in this disclosure. Polypeptide variants may also include polypeptide fragments comprising various lengths of contiguous stretches of amino acid sequences disclosed herein. Polypeptide variant sequences include at least about 5, 10, 15, 20, 30, 40, 50, 75, 100, 150, or more contiguous peptides of one or more of the sequences disclosed herein as well as all intermediate lengths therebetween.

The above-described immunogens may bind specifically to bNAbs. bNAbs are neutralizing antibodies that neutralize multiple HIV-1 viral strains. bNAbs are unique in that they target conserved epitopes of the virus. Examples of broadly neutralizing antibodies may include, without limitation, VRC26.25, PCT64-24E, VRC38.01, PG9, PGDM1400, CH01, BG18, DH270.1, DH270.6, PGDM12, VRC41.01, PGDM21, PCDN-33A, BF520.1, VRC29.03, PGT121, 10-1074, N49-P7, N6, NC-Cow1, IOMA, CH235, CH235.12, b12, VRC01, 3BNC117, CH103, VRC-PG05, VRC34.01, ACS202, PGT151, 35O22, 8ANC195, DH511.11P. Among these bNAbs, BG18, DH270.1, DH270.6, PGDM12, VRC41.01, PGDM21, PCDN-33A, BF520.1, VRC29.03, PGT121, 10-1074 broadly neutralizing antibodies bind specifically to V3 glycans. In some embodiments, the disclosed immunogens bind to the PGT121 or 10-1074 broadly neutralizing antibody with an affinity having dissociation constant (K_{D}) about 50 μM or less.

The terms “specific binding,” “selective binding,” “selectively binds,” and “specifically binds,” refer to antibody binding to an epitope on a predetermined antigen but not to other antigens. Typically, the antibody binds with an equilibrium dissociation constant (K_{D}) of approximately less than 10⁻⁶ M, such as approximately less than 10⁻⁷ M, 10⁻⁸ M, 10⁻⁹ M or 10⁻¹⁰ M or even lower when determined by, e.g., ELISA, equilibrium dialysis or surface plasmon resonance (SPR) technology in a BIACORE® 2000 surface plasmon resonance instrument using the predetermined antigen, e.g., an epitope on the viral envelope of HIV-1, e.g., gp120, as the analyte and the antibody as the ligand, or Scatchard analysis of binding of the antibody to antigen-positive cells, and (ii) binds to the predetermined antigen with an affinity that is at least two-fold greater than its affinity for binding to a non-specific antigen (e.g., BSA, casein) other than the predetermined antigen or a closely-related antigen.

In another aspect, this disclosure provides immunogen polypeptides that are multimerized on a virus-like particle (VLP) (e.g., retrovirus-like particle, HIV-like particle). Virus-like particles, or retrovirus-like particles, in the context of the present disclosure, arc membrane-surrounded structures comprising viral envelope proteins embedded within the membrane of the host cell in which they are produced, and preferably, additional viral core proteins in the VLPs. These VLPs do not contain intact viral nucleic acid, and they are non-infectious. Desirably, there is sufficient envelope protein on the surface of the VLP so that when a VLP preparation is formulated into an immunogenic composition and administered to an animal or human, an immune response (cell-mediated or humoral) is raised. Desirably, the Env protein is truncated from the carboxy terminus as compared with the naturally occurring virus envelope protein. In the context of the present invention, a “truncated” envelope protein is one which contains less than a full-length cytoplasmic domain, which but retains surface antigenic determinants against which an immune response is generated, preferably a protective immune response, and it retains sufficient envelope sequence for proper precursor processing and membrane insertion. The skilled artisan can produce truncated virus envelope proteins using recombinant DNA technology and virus coding sequences, which are readily available to the public. For example, the coding sequence of a virus envelope protein can be engineered for expression in a baculovirus expression vector, for example, using a commercially available baculovirus vector, under the regulatory control of a virus promoter, with appropriate modifications of the sequence to allow functional linkage of the coding sequence to the regulatory sequence, and truncation (deletion) of the portion of the coding sequence which encodes the cytoplasmic domain of the envelope protein, again with appropriate translation stop signals and sequences which allow operable splicing of the truncated envelope and associated sequences into the vector. A specifically exemplified truncated SIV envelope protein lacks the 89 amino acids at the carboxy terminus of the naturally occurring SIV envelope protein.

In another aspect, this disclosure provides a protein complex comprising at least one above-described immunogen polypeptide multimerized via covalent or non-covalent bonding/interaction (e.g., van der Waals interactions). For example, two or more immunogen polypeptides may be cross-linked by one or more cross-linkers. Crosslinkers are reagents having reactive ends to specific functional groups (e.g., primary amines or sulfhydryls) on proteins or other molecules. Crosslinkers are capable of joining two or more molecules by a covalent bond. Crosslinkers include but are not limited to amine-to-amine crosslinkers (e.g., disuccinimidyl suberate(DSS)), amine-to-sulfhydryl crosslinkers (e.g., N-γ-maleimidobutyryl-oxysuccinimide ester (GMBS)), carboxyl-to-amine crosslinkers (e.g., dicyclohexylcarbodiimide (DCC)), sulfhydryl-to-carbohydrate crosslinkers (e.g., N-β-malcimidopropionic acid hydrazide (BMPH)), sulfhydryl-to-sulfhydryl crosslinkers (e.g., 1,4-bismaleimidobutane (BMB)), photorcactive crosslinkers (e.g., N-5-azido-2-nitrobenzoyloxysuccinimide (ANB-NOS)), chemoselective ligation crosslinkers (e.g., NHS-PEG4-Azide).

### B. Nucleic Acids

Another aspect of this disclosure features an isolated nucleic acid comprising a sequence that encodes the polypeptide or protein described above. A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

An “isolated nucleic acid” refers to a nucleic acid the structure of which is not identical to that of any naturally occurring nucleic acid or to that of any fragment of a naturally occurring genomic nucleic acid. The term, therefore, covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the polypeptide, fusion protein, or antibody of this invention. For this purpose, one can operatively link the nucleic acid to suitable regulatory sequences to generate an expression vector.

The nucleic acid and amino acid sequences disclosed herein are shown using standard letter abbreviations for nucleotide bases, and one letter code for amino acids. Only one strand of each nucleic acid sequence is shown, but the complementary strand is understood as included by any reference to the displayed strand.

This disclosure also includes vectors containing a coding sequence for the disclosed immunogen, host cells containing the vectors, and methods of making substantially pure immunogen comprising the steps of introducing the coding sequence for the immunogen into a host cell, and cultivating the host cell under appropriate conditions such that the immunogen is produced and secreted. The immunogen so produced may be harvested in conventional ways. Therefore, the present invention also relates to methods of expressing the immunogen and biological equivalents disclosed herein, assays employing these gene products, and recombinant host cells which comprise DNA constructs which express these receptor proteins.

The disclosed immunogens may be recombinantly expressed by molecular cloning the nucleic acid encoding the immunogens into an expression vector (such as pcDNA3.neo, pcDNA3.1, pCR2.1, pBlueBacHis2 or pLITMUS28) containing a suitable promoter and other appropriate transcription regulatory elements, and transferred into prokaryotic or eukaryotic host cells to produce the immunogens. Techniques for such manipulations can be found described in Sambrook et al., 1989, Molecular Cloning: A Laboratory Manual; Cold Spring Harbor Laboratory, Cold Spring Harbor, New York, are well known and readily available to the artisan of ordinary skill in the art. Therefore, another aspect of the present invention includes host cells that have been engineered to contain and/or express DNA sequences encoding the immunogens. Such recombinant host cells can be cultured under suitable conditions to produce the disclosed immunogens or a biologically equivalent form. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines.

For instance, one insect expression system utilizes Spodoptera frugiperda (Sf21) insect cells (Invitrogen) in tandem with a baculovirus expression vector (pAcG2T, Pharmingen). Also, mammalian species which may be suitable and which are commercially available, include but are not limited to, L cells L-M (TK˜) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

A variety of mammalian expression vectors may be used to express recombinant immunogens in mammalian cells. Expression vectors are defined herein as DNA sequences that are required for the transcription of cloned DNA and the translation of their mRNAs in an appropriate host. Such vectors can be used to express eukaryotic DNA in a variety of hosts such as bacteria, blue-green algae, plant cells, insect cells, and animal cells. Specifically designed vectors allow the shuttling of DNA between hosts such as bacteria-yeast or bacteπa-animal cells. An appropriately constructed expression vector should contain: an oπgm of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency.

Expression vectors may include, but are not limited to, cloning vectors, modified cloning vectors, specifically designed plasmids or viruses. Commercially available mammalian expression vectors which may be suitable for immunogen expression, include but are not limited to, pIRES-hyg (Clontech), PIRES-puro (Clontech), pcDNA3.neo (Invitrogen), pcDNA3.1 (Invitrogen), pCI-neo (Promega), pLITMUS28, pLITMUS29, pLITMUS38 and pLITMUS39 (New England Bioloabs), pcDNAI, pcDNAIamp (Invitrogen), pcDNA3 (Invitrogen), pMC1neo (Stratagene), pXT1 (Stratagene), pSG5 (Stratagene), EBO-pSV2-neo (ATCC 37593) pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

Also, a variety of bacterial expression vectors may be used to express the disclosed immunogens in bacterial cells. Commercially available bacteπal expression vectors that may be suitable for immunogen expression include, but are not limited to pCR2.1 (Invitrogen), pET1 1a (Novagen), lambda gt1 1 (Invitrogen), and pKK223-3 (Pharmacia).

In addition, a variety of fungal cell expression vectors may be used to express the immunogens in fungal cells. Commercially available fungal cell expression vectors which may be suitable for recombinant immunogen expression include but are not limited to pYES2 (Invitrogen) and Pichia expression vector (Invitrogen).

Also, a variety of insect cell expression vectors may be used to express a recombinant receptor in insect cells. Commercially available insect cell expression vectors which may be suitable for recombinant expression of the immunogens include but are not limited to pBlueBacIII and pBlueBacHιs2 (Invitrogen), and pAcG2T (Pharmingen).

The expression vector may be introduced into host cells via any one of a number of techniques including but not limited to transformation, transfection, protoplast fusion, and electroporation. Transformation is meant to encompass a genetic change to the target cell resulting from incorporation of DNA. Transfection is meant to include any method known in the art for introducing the immunogens into the test cells. For example, transfection includes calcium phosphate or calcium chloride mediated transfection, lipofection, electroporation, as well as infection with, for example, a viral vector such as a recombinant retroviral vector containing the nucleotide sequence which encodes the immunogens, and combinations thereof. The expression vector-containing cells are individually analyzed to determine whether they produce the immunogens. Identification of immunogen expressing cells may be done by several means, including but not limited to immunological reactivity with specific bNAbs, labeled ligand binding and the presence of host cell-associated activity with respect to the immunogens.

Also within the scope of this invention is a host cell that contains the above-described nucleic acid. Examples include bacterial cells (e.g., E. coli cells, insect cells (e.g., using baculovirus expression vectors), yeast cells, or mammalian cells. See, e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, e.g., using T7 promoter regulatory sequences and T7 polymerase.

### C. Compositions

In another aspect, this disclosure provides an immunogenic composition for stimulating an immune response in a subject in need thereof. The immunogenic composition includes (i) the immunogen, the nucleic acid, the host cell, the protein complex, or the virus particle described above; and (ii) a pharmaceutically acceptable carrier. The method may further include administering the composition two or more times. The administration of the composition may result in increased numbers of broadly-neutralizing antibodies in the serum capable of recognizing a V3-glycan epitope.

An immunogenic composition is a composition comprising an immunogenic peptide that induces a measurable CTL response against a virus expressing the immunogenic peptide, or induces a measurable B cell response (such as production of antibodies) against the immunogenic peptide. In one example, an “immunogenic composition” is composition includes a disclosed immunogen derived from a gp120 or an antigenic fragment thereof. It further refers to isolated nucleic acids encoding an immunogen, such as a nucleic acid that can be used to express the immunogen (and thus be used to elicit an immune response against this polypeptide).

For in vitro use, an immunogenic composition may consist of the isolated protein, peptide epitope, or nucleic acid encoding the protein or peptide epitope. For in vivo use, the immunogenic composition will typically include the protein, immunogenic peptide or nucleic acid in pharmaceutically acceptable carriers and/or other agents. Any particular peptide, such as a disclosed immunogen or a nucleic acid encoding the immunogen, can be readily tested for its ability to induce a CTL or B cell response by art-recognized assays. Immunogenic compositions can include adjuvants, which are well known to one of skill in the art.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically-acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, TWEENS or SPANS or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally comprise one or more excipients or diluents. In some preferred embodiments, the topical formulations include a material that would enhance absorption or penetration of the active agent(s) through the skin or other affected areas. The topical composition is useful for treating inflammatory disorders in the skin, including, but not limited to, eczema, acne, rosacea, psoriasis, contact dermatitis, and reactions to poison ivy.

A topical composition contains a safe and effective amount of a dermatologically acceptable carrier suitable for application to the skin. A “cosmetically acceptable” or “dermatologically-acceptable” composition or component refers to a composition or component that is suitable for use in contact with human skin without undue toxicity, incompatibility, instability, allergic response, and the like. The carrier enables an active agent and an optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular, one that has a sufficient thickness or yield point to prevent the active materials from sedimenting. The carrier can be inert or possess dermatological benefits. It also should be physically and chemically compatible with the active components described herein, and should not unduly impair stability, efficacy, or other use benefits associated with the composition. The topical composition may be a cosmetic or dermatologic product in the form known in the art for topical or transdermal applications, including solutions, aerosols, creams, gels, patches, ointment, lotion, or foam.

Pharmaceutically acceptable carrier includes any and all solvents, dispersion media, coatings, antibacterial and antifungal agents, isotonic and absorption delaying agents, and the like that are physiologically compatible. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see, e.g., Berge, S. M., et al. (1977) J. Pharm. Sci. 66:1-19).

The host cells provided in the immunogenic compositions may be inactivated or chemically/genetically attenuated bacterial vaccine that does not elicit the cytotoxic T-lymphocyte (CTL) immune response necessary for the lysis of tumor cells and cells infected with intracellular pathogens.

### II. METHODS FOR STIMULATING IMMUNE RESPONSE USING THE DISCLOSED IMMUNOGENS

The immunogens, as disclosed herein, a nucleic acid molecule encoding the disclosed immunogen, the host cell, the protein complex, or the virus particle can be administered to a subject in order to generate an immune response to a pathogen, such as HIV. In another aspect, this disclosure provides a method of treating or preventing HIV infection in a subject in need thereof. The method includes administering to the subject a therapeutically effective amount of the immunogen, the nucleic acid, the host cell, the protein complex, or the virus particle described above, or a combination thereof. This disclosure also provides use of the immunogen, the nucleic acid, the host cell, the protein complex, or the virus particle described above, or a combination thereof in the preparation of a medicament to treat or prevent HIV infection in a subject.

In exemplary applications, compositions are administered to a subject suffering from HIV infection or at risk of becoming infected from HIV. In other applications, the immunogens disclosed herein can be administered prophylactically, for example, as part of an immunization regimen.

The immunogen is administered in an amount sufficient to raise an immune response against the HIV virus. Administration induces a sufficient immune response to treat the pathogenic infection, for example, to inhibit the infection and/or reduce the signs and/or symptoms of the infection. Amounts effective for this use will depend upon the severity of the disease, the general state of the subject's health, and the robustness of the subject's immune system. A therapeutically effective amount of the immunogen is that which provides either subjective relief of a symptom(s) or an objectively identifiable improvement as noted by the clinician or other qualified observers.

Therapeutically effective amount or effective amount refers to the amount of agents, such as nucleic acid vaccine or other therapeutic agents, that is sufficient to prevent, treat (including prophylaxis), reduce and/or ameliorate the symptoms and/or underlying causes of any of a disorder or disease, for example to prevent, inhibit, and/or treat HIV. In some embodiments, an “effective amount” is sufficient to reduce or eliminate a symptom of a disease, such as AIDS. For instance, this can be the amount necessary to inhibit viral replication or to measurably alter outward symptoms of the viral infection, such as an increase of T cell counts in the case of HIV-1 infection. In general, this amount will be sufficient to measurably inhibit virus (for example, HIV) replication or infectivity. When administered to a subject, a dosage will generally be used that will achieve target tissue concentrations (for example, in lymphocytes) that have been shown to achieve in vitro inhibition of viral replication.

An immunogen can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, PA, 1995) either locally or systemically, such as by intramuscular, subcutaneous, or intravenous injection, but even oral, nasal, or anal administration is contemplated. In one embodiment, the administration is by subcutaneous or intramuscular injection. To extend the time during which the disclosed immunogen is available to stimulate a response, the immunogen can be provided as an implant, an oily injection, or as a particulate system. The particulate system can be a microparticle, a microcapsule, a microsphere, a nanocapsule, or similar particle, (see, e.g., Banga, supra). A particulate carrier based on a synthetic polymer has been shown to act as an adjuvant to enhance the immune response, in addition to providing a controlled release. Aluminum salts can also be used as adjuvants to produce an immune response.

Optionally, one or more cytokines, such as interleukin (IL)-2, IL-6, IL-12, IL-15, RANTES, granulocyto-macrophage colony-stimulating factor (GM-CSF), tumor necrosis factor (TNF)-a, interferon (IFN)-a or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2): 122-38; Lotze et al., 2000, Cancer J Sci. Am. 6(Suppl 1):S61-6; Cao et al., 1998, Stem Cells 16(Suppl 1 J.-251-60; Kuiper et al., 2000, Adv. Exp. Med. Biol. 465:381-90). These molecules can be administered systemically (or locally) to the host. In several examples, IL-2, RANTES, GM-CSF, TNF-a, IFN-γ, G-CSF, LFA-3, CD72, B7-1, B7-2, B7-1 B.7-2, OX-40L, 41 BBL, and ICAM-1 are administered.

A pharmaceutical composition including an isolated immunogen is provided. In some embodiments, the immunogen is mixed with an adjuvant containing two or more of a stabilizing detergent, a micelle-forming agent, and an oil. Suitable stabilizing detergents, micelle-forming agents, and oils are detailed in U.S. Pat. Nos. 5,585,103; 5,709,860; 5,270,202; and 5,695,770. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, DE), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54: 110, 1977, and Hunter et al., J. Immunol 129: 1244, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, PA, 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, NY, pp. 219-342, 1994; Tice & Tabibi, Treatise on Controlled Drug Delivery, A. Kydonieus, ed., Marcel Dekker, Inc. New York, NY, pp. 315-339, 1992).

Polymers can be used for ion-controlled release. Various degradable and nondegradable polymeric matrices for use in controlled drug delivery are known in the art (Langer, Accounts Chem. Res. 26:53′, 1993). For example, the block copolymer, poloxamer 407 exists as a viscous yet mobile liquid at low temperatures but forms a semisolid gel at body temperature. It has shown to be an effective vehicle for formulation and sustained delivery of recombinant interleukin-2 and urease (Johnston et ah, Pharm. Res. 9:425, 1992; and Pec,/. Parent. Sci. Tech. 44(2):58, 1990). Alternatively, hydroxyapatite has been used as a microcarrier for controlled release of proteins (Ijntema et ah, Int. J. Pharm. 112:215, 1994). In yet another aspect, liposomes are used for controlled release as well as drug targeting of the lipid-capsulated drug (Betageri et ah, Liposome Drug Delivery Systems, Technomic Publishing Co., Inc., Lancaster, PA, 1993). Numerous additional systems for controlled delivery of therapeutic proteins are known (e.g., U.S. Pat. Nos. 5,055,303; 5,188,837; 4,235,871; 4,501,728; 4,837,028; 4,957,735; and 5,019,369; 5,055,303; 5,514,670; 5,413,797; 5,268,164; 5,004,697; 4,902,505; 5,506,206; 5,271,961; 5,254,342; and 5,534,496).

In another embodiment, a pharmaceutical composition includes a nucleic acid encoding a disclosed immunogen. A therapeutically effective amount of the nucleic acid can be administered to a subject in order to generate an immune response. In one specific, non-limiting example, a therapeutically effective amount of a nucleic acid encoding a disclosed gp120immunogen or immunogenic fragment thereof is administered to a subject to treat or prevent or inhibit HIV infection.

Optionally, one or more cytokines, such as IL-2, IL-6, IL-12, RANTES, GM-CSF, TNF-a, or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2): 122-38; Lotze et al., 2000, Cancer J Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically to the host. It should be noted that these molecules can be co-administered via insertion of a nucleic acid encoding the molecules into a vector, for example, a recombinant pox vector (see, for example, U.S. Pat. No. 6,045,802). In various embodiments, the nucleic acid encoding the biological adjuvant can be cloned into the same vector as the disclosed immunogen coding sequence, or the nucleic acid can be cloned into one or more separate vectors for co-administration. In addition, nonspecific immunomodulating factors such as Bacillus Cahnette-Guerin (BCG) and levamisole can be co-administered. One approach to administration of nucleic acids is direct immunization with plasmid DNA, such as with a mammalian expression plasmid. As described above, the nucleotide sequence encoding the disclosed immunogen can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired immunogen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachic, Immunol. Today 12:383, 1991). Doses of antigen as low as 1 μg encapsulated in ISCOMS™ have been found to produce Class I mediated CTL responses (Takahashi et ah, Nature 344:873, 1990).

In another approach to using nucleic acids for immunization, a disclosed immunogen can also be expressed by attenuated viral hosts or vectors or bacterial vectors. Recombinant vaccinia virus, adeno-associated virus (AAV), herpes virus, retrovirus, cytomegalovirus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed immunogen is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 g/kg to about 50 mg/kg, and typically are about 0.005 mg/kg to about 5 mg/kg (see, e.g., U.S. Pat. No. 5,589,466).

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

It may be advantageous to administer the immunogenic compositions disclosed herein with other agents such as proteins, peptides, antibodies, and other antiviral agents, such as anti-HIV agents. Examples of such anti-HIV therapeutic agents include nucleoside reverse transcriptase inhibitors, such as abacavir, AZT, didanosine, emtricitabine, lamivudine, stavudine, tenofovir, zalcitabine, zidovudine, and the like, non-nucleoside reverse transcriptase inhibitors, such as delavirdine, efavirenz, nevirapine, protease inhibitors such as amprenavir, atazanavir, indinavir, lopinavir, nelfinavir, fosamprenavir, ritonavir, saquinavir, tipranavir, and the like, and fusion protein inhibitors such as enfuvirtide and the like. In certain embodiments, immunogenic compositions are administered concurrently with other anti-HIV therapeutic agents. In some examples, the disclosed immunogens are administered with T-helper cells, such as exogenous T-helper cells. Exemplary methods for producing and administering T-helper cells can be found in International Patent Publication WO 03/020904, which is incorporated herein by reference. In certain embodiments, the immunogenic compositions are administered sequentially with other anti-HIV therapeutic agents, such as before or after the other agent. One of ordinary skill in the art would know that sequential administration can mean immediately following or after an appropriate period of time, such as hours, days, weeks, months, or even years later.

The disclosed gp120 immunogen or immunogenic fragments thereof and nucleic acids encoding these immunogens can be used in a multistep immunization regime. In some examples, the regime includes administering to a subject a therapeutically effective amount of a first immunogen or immunogenic fragments thereof as disclosed herein (the prime) and boosting the immunogenic response with one or more additional immunogens or immunogenic fragments thereof after an appropriate period of time. The method of eliciting such an immune reaction is what is known as “prime-boost.” In this method, the antibody response to the selected immunogenic surface is focused by giving the subject's immune system a chance to “see” the antigenic surface in multiple contexts. In other words, the use of multiple immunogens or immunogenic fragments thereof with an antigenic surface in common selects for antibodies that bind the immunogen's surface in common.

In some examples, the immunogens or immunogenic fragments thereof and nucleic acids encoding these immunogens can are administered in “prime-boost” immunization regimes. For example, the immunogens or immunogenic fragments thereof and nucleic acids encoding these immunogens can are administered to a subject, before, during, after a stabilized gp140 trimer (see for example Yang et al. J Virol. 76(9):4634-42, 2002) is administered.

One can also use cocktails containing the disclosed immunogenic agents, for example, the immunogen, the nucleic acid encoding the immunogen, the host cell, the protein complex, or the virus particle described above, or a combination thereof to prime and then boost with trimers from a variety of different HIV strains or with trimers that are a mixture of multiple HIV strains. The prime can be administered as a single dose or multiple doses, for example, two doses, three doses, four doses, five doses, six doses or more can be administered to a subject over days, weeks or months. The boost can be administered as a single dose or multiple doses, for example, two to six doses or more can be administered to a subject over a day, a week or months. Multiple boosts can also be given, such as one to five, or more. Different dosages can be used in a series of sequential inoculations. For example, a relatively large dose in a primary inoculation and then a boost with relatively smaller doses. The immune response against the selected antigenic surface can be generated by one or more inoculations of a subject with an immunogenic composition disclosed herein.

### III. IMMUNODIAGNOSTIC REAGENTS AND KITS

This disclosure provides a method for detecting or isolating an HIV-1 binding antibody in a subject infected with HIV-1. The method includes contacting a sample from a subject, such as, but not limited to a blood, serum, plasma, urine or sputum sample from the subject with one or more of the disclosed immunogenic agents, for example, the immunogen, the nucleic acid encoding the immunogen, the host cell, the protein complex, or the virus particle described above, or a combination thereof. The method may also include detecting binding of antibodies in the sample to the disclosed immunogenic agents. The binding can be detected by any means known to one of skill in the art, including the use of labeled secondary antibodies that specifically bind the antibodies from the sample. Labels include radiolabels, enzymatic labels, and fluorescent labels. In some embodiments, the method may further include isolating the HIV-1 binding antibody in a subject.

The disclosed immunogenic agents can be as components of a kit. Such a kit may also include additional components including packaging, instructions and various other reagents, such as buffers, substrates, antibodies or ligands, such as control antibodies or ligands, and detection reagents. The kit may optionally include an adjuvant.

An adjuvant is a vehicle used to enhance antigenicity. Adjuvants include a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants (for example see U.S. Pat. Nos. 6,194,388; 6,207,646; 6,214,806; 6,218,371; 6,239,116; 6,339,068; 6,406,705; and 6,429,199). Adjuvants include biological molecules (a “biological adjuvant”), such as costimulatory molecules. Exemplary adjuvants include IL-2, RANTES, GM-CSF, TNF-a, IFN-γ, G-CSF, LFA-3, CD72, B7-1, B7-2, OX-40L, and 41 BBL. Adjuvants can be used in combination with the disclosed immunogens.

### IV. DEFINITIONS

As used in this document, the singular forms “a,” “an,” and “the” include plural references unless the context clearly dictates otherwise. Unless defined otherwise, all technical and scientific terms used here.

The term “and/or” means any one of the items, any combination of the items, or all of the items with which this term is associated.

The compositions of the present invention can comprise, consist essentially of, or consist of the claimed ingredients. The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

The term “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental, non-human animal or animal suitable as a disease model.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example, “about 1” may also mean from 0.5 to 1.4.

gp120 is an envelope protein from human immunodeficiency virus (HIV). The mature gp120 wild-type polypeptides have about 500 amino acids in the primary sequence. The gp120 is heavily N-glycosylated giving rise to an apparent molecular weight of 120 kD. The polypeptide is comprised of five conserved regions (C1-C5) and five regions of high variability (V1-V5). Exemplary sequences of wild-type gp160 polypeptides are shown on GENBANK®, for example, Accession Nos. AAB05604 and AAD12142, which are incorporated herein by reference in their entirety as available on Jun. 29, 2010. Exemplary sequences of gp120 polypeptides from HIV-1 DU156 are shown on GENBANK®, for example, Accession Nos. ABD83635, AAO50350, and AAT91997, which are incorporated herein by reference in their entirety as available on Sep. 27, 2010. Exemplary sequences of gp120 polypeptides from HIV-1 ZA012 are shown on GENBANK®, for example, Accession No. ACF75939, which is incorporated herein by reference in its entirety as available on Sep. 27, 2010.

“Glycosylation site” refers to an amino acid sequence on the surface of a polypeptide, such as a protein, which accommodates the attachment of a glycan. An N-linked glycosylation site is triplet sequence of NXS/T in which N is asparagine, X is any residues except proline, S/T means serine or threonine. A glycan is a polysaccharide or oligosaccharide. Glycan may also be used to refer to the carbohydrate portion of a glycoconjugate, such as a glycoprotein, glycolipid, or a proteoglycan.

“Immunogenic polypeptide” refers to a protein or a portion thereof that is capable of inducing an immune response in a mammal, such as a mammal infected or at risk of infection with a pathogen. Administration of an immunogenic polypeptide derived from a pathogen of interest that inducing an immune response. Administration of an immunogenic polypeptide can lead to protective immunity against a pathogen of interest. In some examples, an immunogenic polypeptide is an antigen that is resurfaced to focus immunogenicity to a target epitope. An “immunogenic gp120 polypeptide” is gp120 molecule, a resurfaced gp120 molecule, or a portion thereof capable of inducing an immune response in a mammal, such as a mammal with or without an HIV infection. Administration of an immunogenic gp120 polypeptide that induces an immune response can lead to protective immunity against HIV.

“Immune response” refers to a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response and results in the production of specific antibodies.

“Isolated” refers to an “isolated” biological component (such as a protein, for example, a disclosed antigen or nucleic acid encoding such an antigen) has been substantially separated or purified away from other biological components in which the component naturally occurs, such as other chromosomal and extrachromosomal DNA, RNA, and proteins. Proteins, peptides, and nucleic acids that have been “isolated” include proteins purified by standard purification methods. The term also embraces proteins or peptides prepared by recombinant expression in a host cell as well as chemically synthesized proteins, peptides, and nucleic acid molecules. Isolated (or purified) does not require absolute purity, and can include protein, peptide, or nucleic acid molecules that are at least 50% isolated, such as at least 75%, 80%, 90%, 95%, 98%, 99%, or even 99.9% isolated.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In some examples, a nucleic acid encodes a disclosed antigen. “Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

Unless defined otherwise, all technical and scientific terms used herein have the same meaning as commonly understood by one of ordinary skill in the art to which this invention belongs. Although any methods and materials similar or necessarily to those described herein can also be used in the practice or testing of the present invention, the preferred methods and materials are now described. All publications mentioned herein are incorporated herein by reference in their entireties.

### V. EXAMPLES

### Example 1

This example describes the materials and methods used in Examples 2-6 bellow.

### Envelope Proteins

Env trimers were expressed as soluble native-like soluble gp 140 trimers that included the SOSIP substitutions: ‘SOS’ substitutions (A501C_{gp120}, T605C_{gp41}), ‘IP’ (I559P_{gp41}), addition of the N-linked glycan sequence at residue 332_{gp120} (T332N_{gp120}), an enhanced gp120-gp41 cleavage site (REKR (SEQ ID NO: 17) to RRRRRR (SEQ ID NO: 18)), and a stop codon after residue 664_{gp41} (Env numbering according to HX nomenclature). The newly-engineered Env trimers RC1, RC1-4fill, RC1-Avitag, RC1-Spytag, RC1-glycanKO, RC1-glycanKO-Avitag, RC1-glycanKO-GAIA (“GAIA” disclosed as SEQ ID NO: 16) and RC1-GAIA (“GAIA” disclosed as SEQ ID NO: 16), wtBG505, and the previously-reported BG505 variants 11MUTB, 10MUT, 7MUT, 5MUT were cloned in the pPPPI4 expression vector using synthetic gene fragments (Integrated DNA Technologies (IDT)). The glycan variants RC1Δ301, RC1Δ332, and 11MUTBΔ301 were produced by site-directed mutagenesis (QuikChange Lightning Multi-site directed mutagenesis kit, Catalog #210515, Agilent Technologies).

Non-tagged versions of Env proteins were used in ELISAs (see ELISA section) and for immunizations in wild-type mice (see Animals section). The Spytagged version of RC1-4fill was conjugated to virus-like particles (VLPs) and used for immunizations in rabbits and macaques (see VLP production and conjugation and Animals sections). The Avitagged versions of RC1 and RC1-glycanKO were biotinylated and used as baits in FACS (See Flow cytometry and single B-cell sorting section).

Soluble Env trimers were expressed by transient transfection in HEK293-6E cells (National Research Council of Canada) or Expi293 cells (Life Technologies) and purified from cell supernatants by 2G12 or NIH45-46 immunoaffinity chromatography and size exclusion chromatography (SEC) as previously described (Wang, H. et al. Elife 6 (2017). Proteins were stored at 4° C. in 20 mM Tris pH 8.0 and 150 mM sodium chloride (TBS buffer). SpyTagged immunogens were buffer exchanged into 20 mM sodium phosphate pH 7.5, 150 mM NaCl.

### VLP Production and Conjugation

For attachment to VLPs, a C-terminal SpyTag sequence (13 residues) was added to RC1-4fill to form an irreversible isopeptide bond to SpyCatcher protein (Zakeri, B. et al. Proc Natl Acad Sci USA 109, E690-697 (2012)). The gene encoding bacteriophage AP205 coat protein to which the SpyCatcher protein was attached was the kind gift of Dr. Mark Howarth, Oxford University). SpyCatcher-AP205 VLPs was purified as described (Brune, K. D. et al. Sci Rep 6, 19234 (2016)), incubated with 3-fold molar excess SpyTagged RC1-4fill Env trimers, and separated conjugated VLPs from free Env trimers by SEC on a Superdex 200 column equilibrated with 20 mM sodium phosphate pH 7.5, 150 mM NaCl. Conjugation of Env trimers was verified by negative-stain EM and/or SDS-PAGE, and immunogen concentrations were estimated by comparing to known amounts of free immunogen run on the same SDS-PAGE gel.

### Animals

Mice carrying the Ig V(D)J genes encoding the iGL IgH and IgL corresponding to the human PGT121 and 10-1074 broadly neutralizing antibodies (GL_{HL}121 knock-in mice) were previously described (Escolano, A. et al. Cell 166, 1445-1458 e1412, (2016)). 6-8 week old C57BL6 male mice from The Jackson Laboratory were used for immunizations. All animal procedures were performed in accordance with protocols approved by the Rockefeller University IACUC. Male and female GL_{HL}121 knock-in mice or male C57BL6 wild-type mice were equally distributed in groups and immunized intraperitoneally with 10 μg of soluble SOSIP Envelope trimer in Ribi adjuvant (Sigma) (1:1).

Six-month-old New Zealand White rabbits (Covance) were used for immunizations. Rabbits were immunized subcutaneously with ˜22 μg of RC1-4fill SOSIP Env trimer conjugated to VLP (RC1-4fill VLP) in an ISCOMs-like saponin adjuvant (see Adjuvant synthesis section). Serum samples were collected from mice and rabbits on weeks 0 and 2 after immunization.

Eight rhesus macaques (Macaca mulatta) of Indian genetic origin, 2 to 4 years of age, were housed and cared for in accordance with Guide for Care and Use of Laboratory Animals Report no. NIH 82-53 (Department of Health and Human Services, Bethesda, Maryland, 1985) in a biosafety level 2 NIH facility. All animal procedures and experiments were performed according to protocols approved by the Institutional Animal Care and Use Committee of NIAID, NIH.

Animals were immunized subcutaneously (s.c) with approximately 200 μg of RC1-4fill SOSIP Env trimer conjugated to VLP (RC1-4fill VLP) adjuvanted in IscoMPLA into the medial inner forelegs and hind legs (total of 4 sites/animal). Blood was drawn regularly to monitor serum neutralizing activity and characterize serum antibody binding by ELISA. Lymph node biopsies were obtained from naïve macaques and from the immunized macaques 3 weeks after immunization.

### Adjuvant Synthesis

ISCOM-like saponin adjuvant was prepared as previously described (K. Lövgren-Bengtsson, et al, in Methods in Molecular Medicine, Vaccine Adjuvants: Preparation Methods and Research Protocols, D. O'Hagan, Ed. (Humana Press, Totowa, NJ, 2000), vol. 42, pp. 239-258). Briefly, 20 mg/ml solutions of cholesterol (Avanti Polar Lipids 700000) and 1,2-dipalmitoyl-sn-glycero-3-phosphocholine (DPPC) (Avanti Polar Lipids 850355) were prepared in 20% MEGA-10 (Sigma D6277) detergent. Quil-A saponin (InvivoGen vac-quil) was dissolved in Milli-Q water at a final concentration of 100 mg/ml. All components were mixed at a ratio of 1:1:5 (chol:DPPC: Quil-A) followed by dilution with 1×PBS for a final concentration of 1 mg/ml cholesterol. For ISCOM-MPLA saponin adjuvant, a 5 mg/ml solution of MPLA (Avanti 699800) was prepared in 20% MEGA-10, and the components were mixed at a ratio of 2:1:1:10 (chol:DPPC:MPLA: Quil-A). The solutions were allowed to equilibrate overnight at RT, followed by dialysis against 1×PBS using a 10 k MWCO membrane (ThermoFisher 66456). The adjuvant solution was then sterile filtered, concentrated using 50 k MWCO Centricon spin filters (Millipore Sigma UFC905024), and further purified by Fast Protein Liquid Chromatography (FPLC) using a Sephacryl S-500 HR size exclusion column (GE Life Sciences 28-9356-06). The final adjuvant concentration was determined by cholesterol quantification (Sigma MAK043).

### ELISA

ELISAs with SOSIP Env trimers 11MUTB, RC1, 11MUTBΔ301, RC1Δ301, RC1-GAIA (“GAIA” disclosed as SEQ ID NO: 16), RC1-glycan-knock-out (RC1-glycanKO), RC1-glycanKO-GAIA (“GAIA” disclosed as SEQ ID NO: 16), RC1Δ332, BG505), 10MUT, 7MUT, 5MUT or the V3 loop-Consensus C peptide (SEQ ID NO: 10: KGKGKGKGKGCTRPNNNTRKSIRIGPGQTFYATGDIIGDIRQAHC) were performed by direct coating of high binding 96-well plates (Corning #9018) with 50 μl per well of protein solution at 2 μg/ml in 1×PBS overnight at 4° C. Plates were washed 3 times with washing buffer (1×PBS with 0.05% Tween 20 (Sigma-Aldrich)) and incubated in blocking buffer (1×PBS with 2% Milk) for 1 hour (h) at room temperature (RT). Immediately after blocking, monoclonal antibodies or serum samples were added in blocking buffer and incubated for 2 h at RT. Serum samples were assayed at a 1:100 or 1:30 starting dilution and seven additional 3-fold serial dilutions. Mouse and human monoclonal antibodies (IgGs) or human Fabs were evaluated at the concentrations specified in the Results section. Plates were washed 3 times with washing buffer and then incubated with anti-mouse IgG (Jackson ImmunoResearch #115-035-071), anti-human IgG heavy chain (Jackson ImmunoResearch #109-035-098) or anti-human Ig heavy and light chain (Jackson ImmunoResearch #109-036-088) conjugated to horseradish peroxidase (HRP) in washing buffer at a 1:5000 dilution. Plates were developed by addition of the HRP substrate, ABTS Single Solution (Life Technologies #00-2024), and absorbance was measured at 405 nm with an ELISA microplate reader (FluoStar Omega, BMG Labtech).

In other ELISAs, high binding 96-well plates were directly coated with 50 μl of a solution of Fab at 20 μg/ml in 1×PBS overnight at 4° C. Plates were washed 3 times with washing buffer and incubated in blocking buffer for 1 hour at RT. Immediately after blocking, plates were incubated in 50 μl of a solution of RC1 or RC1-glycanKO-GAIA (“GAIA” disclosed as SEQ ID NO: 16) at 2 μg/ml in blocking buffer for 1 h at RT. Plates were washed 3 times with washing buffer and incubated for 1 h at RT with 50 μl of a chimeric version (human Fabs and mouse Fc) of the CD4-binding site bNAb 3BNC60 in blocking buffer at 3-fold serial dilutions starting at 5 μg/ml. Plates were washed 3 times with washing buffer and incubated for 1 h at RT with anti-mouse IgG secondary antibody conjugated to HRP (Jackson ImmunoResearch #115-035-071). Plates were washed and developed as above.

### Flow Cytometry and Single B-Cell Sorting

Single-cell suspensions were obtained from the draining lymph nodes and spleens of immunized mice, and mature B-cells were isolated by negative selection using anti-CD43 magnetic beads (MACS) following the manufacturer's instructions.

Frozen PBMCs or cells from lymph node biopsies obtained from the naïve and immunized macaques were thawed and washed in RPMI medium 1640 (1×) (Gibco #11875-093). Mouse or macaque cells were incubated with 100 μl of a solution of FACS buffer (PBS 1× with 2% fetal bovine serum and 1 mM Ethylenediaminetetraacetic acid (EDTA)) with mouse (BD Biosciences #553142) or human (BD Biosciences #564219) Fc blocker respectively at a 1:500 dilution for 30 min on ice.

RC1 and RC1-glycanKO (RC1⁺/RC1 glycanKO⁻) tetramers were prepared by incubating 5 μg of Avitagged and biotinylated RC1 (RC1-AviBio) or Avitagged and biotinylated RC1-glycanKO (RC1-glycanKO AviBio) with fluorophored streptavidin at a 1:200 dilution in 1×PBS for 30 min on ice.

RC1⁺/RC1-glycanKO⁻ mouse B-cells were isolated using RC1-AviBio conjugated to streptavidin BV711 (BD Biosciences, #563262) and RC1-glycanKO AviBio conjugated to streptavidin-PE (BD Biosciences, #554061) as baits. RC1⁺/RC1-glycan KO⁻ macaque B-cells were isolated using three baits: RC1-AviBio conjugated with streptavidin-PE and streptavidin AF647 and RC1-glycanKO AviBio conjugated with streptavidin BV605 (BD Biosciences, #563260). Tetramers were mixed with the human or mouse antibody cocktails indicated below to a final concentration of 5 μg/ml for each of them.

Mouse cells were stained with the following fluorophored antibodies against mouse cell surface markers: anti CD4 APC-eFluor780 (Invitrogen, #47-0042-82), anti CD8 APC-eFluor780 (Invitrogen, #47-0081-82), anti F4/80 APC-eFluor780 (Invitrogen, #47-4801-82), anti NK1.1 APC-eFluor780 (Invitrogen, #47-5941-82), anti CD11b APC-eFluor780 (eBioscience #47-0112-82), anti CD11c APC-eFluor780 (eBioscience #47-0114-82),anti Gr-1 APC-eFluor780 (Invitrogen, #47-5931-82), anti B220 APC (Biolegend, #103212), anti GL7 FITC (BD Biosciences #553666) and anti CD95 BV421 (BD Biosciences #562633) at 1:200 dilution and the live/dead marker Zombie NIR (Biolegend, #77184) at a 1:400 dilution in FACS buffer. Macaque cells were stained with the following anti human antibodies: anti-CD16 APC-eFluor780 (Invitrogen, #47-0168-41), anti-CD8a APC-eFluor780 (Invitrogen, #47-0086-42), anti-CD3 APC-eFluor780 (Invitrogen, #47-0037-41), anti-CD14 APC-eFluor780 (eBiosciences, #47-0149-41), anti-CD20 PeCy7 (BD, #335793), anti CD38 FITC (Stem Cell technologies, #60131FI), anti-IgG BV421 (BD Biosciences, #562581), anti-IgM PerCP-Cy5.5 (BD Biosciences, #561285) at a 1:200 dilution and the live/dead marker Zombie NIR at a 1:400 dilution in FACS buffer. Mouse or macaque cells were incubated with the corresponding antibody cocktail containing the RC1 and RC1-glycanKO baits for 30 minutes on ice, washed with FACS buffer and resuspended in 1 ml of FACS buffer. Before sorting or analysis, the cell suspensions were filtered through a 40 μM cell strainer.

Zombie NIR⁻/CD4⁻/CD8⁻/F4/80⁻/NK1.1⁻/CD11b⁻/CD11c⁻/B220⁺/GL7⁺/CD95⁺RC1⁺/RC1-glycanKO⁻ single cells were isolated from the mouse cell homogenates and Zombie NIR⁻/CD16⁻/CD8a⁻/CD3⁻/CD14⁻/CD20⁺/CD38⁺/IgG^{+/-}/double RC1⁺/RC1-glycanKO⁻ single cells were isolated from the macaque cell homogenates using a FACS Aria III (Becton Dickinson).

Single cells were sorted into individual wells of a 96-well plate containing 5 μl of lysis buffer (TCL buffer (Qiagen #1031576) with 1% of 2-β-mercaptoethanol). Plates were immediately frozen on dry ice and stored at −80° C.

### Antibody Sequencing and Cloning

96-well plates containing single-cell lysates were thawed on ice. Single-cell RNA was purified in a designated clean area using magnetic beads (RNAClean XP, #A63987 Beckman Coulter) following the manufacturer instructions. In the final step of the purification protocol, RNA was eluted from the magnetic beads with 11 μl of a solution containing (14.5 ng/μl of random primers (Invitrogen, #48190-011), 0.5% of tergitol, (Type NP-40, 70% in H₂O, Sigma-Aldrich, #NP40S-100ML), 0.6 U/μl of RNase inhibitor (Promega #N2615) in nuclease-free water (Qiagen), and incubated at 65° C. for 3 min. cDNA was subsequently synthesized by reverse transcription (SuperScript® III Reverse Transcriptase, Invitrogen, #18080-044,10,000 U) as previously described (von Bochmer, L. et al. Nat Protoc 11, 1908-1923 (2016)). cDNA was stored at −80° C. or used for antibody gene amplification by nested Polymerase chain reaction (PCR). To amplify the antibody genes from single B-cells, 10 μl of nuclease-free water was added to the solution containing cDNA.

Mouse and macaque antibody genes were amplified by nested PCR as previously described (von Bochmer, L. et al. Nat Protoc 11, 1908-1923 (2016)). PCR protocols: (annealing (° C.)/elongation (sec)/number of cycles): 1^{st} PCR (IgG IgH and Igλ): 46/55/50; 2^{nd} PCR (IgG IgH and Igλ): 50/55/50. Amplified heavy chain and light chain cDNAs were individually cloned into expression vectors containing the complete mouse or human IgG antibody constant regions or the human heavy chain constant region 1 (Fragment antigen-binding (Fab) vector) by using the sequence and ligation-independent cloning (SLIC) methodology (Li, M. Z. & Elledge, S. J. Nat Methods 4, 251-256 (2007)).

### Antibody Production and Purification

Igs were purified from 200 μl of mouse or macaque serum using Ab Spin Trap Protein G Sepharose columns (GE Healthcare, #28-4083-47) following the manufacturer's instructions. Igs were eluted in 4 fractions of 200 μl. The Ig-containing fractions were buffer exchanged with PBS by overnight dialysis at 4° C. (dialysis cassettes 20000 MWCO Thermo Scientific, #66005).

For structural studies, mouse IgGs and macaque His-tagged Fabs (“His₆” disclosed as SEQ ID NO: 19) were expressed by transient transfection in HEK293-6E or Expi293 cells and purified from cell supernatants using protein A or G (GE Healthcare) (for IgGs) or Ni-NTA (GE Healthcare) or Ni Sepharose 6 Fast Flow (GE Healthcare) (for Fabs) chromatography and SEC as described (Scharf, L. et al. Cell 162, 1379-1390 (2015)). Mouse Fab was obtained by digesting IgG at 1-5 mg ml⁻¹ with ficin (Sigma) using a protocol modified from Thermo Scientific. Fab was purified by protein G (GE Healthcare) and SEC chromatography as described, followed by Mono Q 5/50 (GE Healthcare) ion-exchange chromatography (Diskin, R. et al., Nat Struct Mol Biol 17, 608-613). The common iGL of the PGT121 and 10-1074 bNAbs was expressed as a His-tagged Fab (“His₆” disclosed as SEQ ID NO: 19) as described above.

### In Vitro Neutralization Assay

TZM-bl assays were performed as described (Montefiori, D. C. Curr Protoc Immunol Chapter 12, Unit 12 11 (2005).). In brief, neutralization activity was calculated as a function of the reduction in Tat-induced luciferase expression in the TZM-bl reporter cell line after a single round of virus infection.

### SPR

SPR experiments were performed using a Biacore T200 (Biacore). For measuring the affinity for PGT121/10-1074 iGL Fab, Protein A was immobilized on a CM5 chip (Biacore) by primary amine chemistry (Biacore manual) and 200 nM 8ANC195G52K5 anti-Env IgG was injected over experimental flow cells as described (Scharf, L. et al. Cell 162, 1379-1390 (2015)). A reference flow cell was made by injecting 200 nM mG053 IgG, which does not bind HIV Envs. Human Fc was injected at 1 μM to block the remaining protein A sites. After capturing 10 μM SOSIP protein (RC1, 11MUTB, or 10MUT), a concentration series of PGT121/10-1074 iGL Fab (4-fold dilutions from a top concentration of 160 μM for 10MUT, and 2-fold dilutions from a top concentration of 150 μM for 11MUTB and RC1) was injected, and the binding reactions were allowed to reach equilibrium. Flow cells were regenerated with 10 mM glycine pH 2.0 and 1 M guanidine HCl at a flow rate of 90 μl/min as described (Scharf, L. et al. Cell 162, 1379-1390 (2015)). K_{D}s were derived by nonlinear regression analysis of plots of R_{eq} (the equilibrium binding response) versus the log of the injected protein concentration, and the data were fit to a 1:1 binding model as described (Vaughn, et al. Biochemistry 36, 9374-9380 (1997)).

For measuring the relative binding of antibodies isolated from mice and monkeys, SOSIP Env trimers were immobilized on a CM5 chip by primary amine chemistry, and selected Fabs were injected at 200 nM. Flow cells were regenerated with 10 mM glycine pH 2.0.

### Cryo-EM Sample Preparation

RC1 complexed with 10-1074 was prepared by incubating purified RC1 with 10-1074 Fab and a CD4-binding site (CD4bs) Fab at a 1:3:3 molar ratio (gp 140 protomer: 10-1074 Fab: CD4bs Fab) overnight at room temperature. The RC1-Fab complex was isolated by SEC in TBS (20 mM Tris pH 8.0, 100 mM NaCl) using a Superdex-200 Increase 10/300 column (GE Healthcare). RC1 complexes with mouse and macaque Fabs were prepared by incubating purified RC1 with a mouse or macaque Fab and with 8ANC195 Fab at a 1:1.3:1.3 molar ratio (gp140 protomer: mouse or macaque Fab: 8ANC195 Fab) overnight at room temperature and used without SEC purification. RC1-Fab complexes were diluted to 0.75-1.4 mg/mL in TBS, added to glow-discharged 300 Mesh Quantifoil R1.2/1.3 copper grids, and vitrified in liquid ethane using a Mark IV Vitrobot (FEI).

### Cryo-EM Data Collection

RC1-Fab complexes were imaged on a Talos Arctica cryo-electron microscope operating at 200 kV and equipped with a Falcon 3EC direct electron detector using EPU automated image acquisition software (Tan, et al. Microscopy (Oxf) 65, 43-56 (2016)). The RC1-10-1074 data were collected on two separate days and combined during processing. Each micrograph was collected at a magnification of 73,000, which results in a pixel size of 1.436 Å.

### Cryo-EM Data Processing

Movie micrographs were motion-corrected in RELION-3 and dose weighted using MotionCor2, CTFs were estimated using Getf, and particles were picked from micrographs using Gaussian blob auto-picking (Zivanov, J. et al. Elife 7 (2018); Zheng, S. Q. et al. Nat Methods 14, 331-332 (2017); Zhang, K. Getf. J Struct Biol 193, 1-12 (2016)). Extracted particles were imported into cryoSPARC v2 and classified into 2D class averages (Punjani, A., et al. Nat Methods 14, 290-296 (2017)). Selected particles were sorted into two ab initio models, and the selected model was used as a reference in the homogenous refinement of those selected particles. Resolutions were estimated using the Gold Standard Fourier shell correlation of independently-refined half-maps (where FSC=0.143), and maps were auto-sharpened in cryoSPARC (Punjani, A., et al. Nat Methods 14, 290-296 (2017); Scheres, S. H. & Chen, S. Nat Methods 9, 853-854 (2012);). For interpreting N-linked glycans, a series of maps were generated with overall B-factors ranging from-150 to-400 Å² to improve local features and map connectivity at PNGSs (Terwilliger, T. C., et al. Acta Crystallogr D Struct Biol 74, 545-559 (2018).).

### Model Building

Coordinates for the individual components of each complex were docked into the maps using UCSF Chimera. For the RC1-10-1074 complex, BG505 (PDB 5T3Z), 10-1074 Fab (PDB 5T3Z), and 8ANC131 Fab (PDB 4RWY) were docked into the density (Goddard, T. D., et al. J Struct Biol 157, 281-287 (2007)). For the mouse or macaque Fab complexes with RC1, BG505 Env (PDB 5CEZ), PGT121/10-1074 iGL Fab (PDB 4FQQ), and 8ANC195 Fab (PDB 5CJX) coordinates were docked into density maps. After replacing sequences for the Fabs in the complexes and for RC1, the models were built following iterative rounds of refinement in Coot and Phenix (Adams, P. D. et al. Acta Crystallogr D Biol Crystallogr 66, 213-221 (2010); Emsley, P., et al. Acta Crystallogr D Biol Crystallogr 66, 486-501 (2010)). Coordinates for glycans were added as Man₉ and then trimmed to fit the maps at σ=5. Model validation was performed using MolProbity and Privateer (Chen, V. B. et al. Acta Crystallogr D Biol Crystallogr 66, 12-21 (2010); Agirre, J. et al. Nat Struct Mol Biol 22, 833-834 (2015)).

The CD4-binding site Fab in the RC1-10-1074 complex and the 8ANC195 Fab in the RC1 complexes with mouse and macaque Fabs were not shown in structure figures, and their coordinates were not included in the RC1-Fab complex structures deposited in the EMDB and PDB.

### Analysis Software

Geneious X and MacVector 15.5.3 were used for sequence analysis and graphs were created using R language. Flow cytometry data were processed using FlowJo 10.5.0. GraphPad Prism 7 was used for data analysis.

### Quantification and Statistical Analysis

Statistical information including n, mean and statistical significance values are indicated in the text or the figure legends. GraphPad Prism 7 was used for statistical analysis by unpaired T-Test. Data were considered statistically significant at *p≤0.05, **p≤0.01, ***p≤0.001 and ****p≤0.0001.

### Example 2

### RC1 Facilitates Antibody Binding to the V3-Glycan Epitope

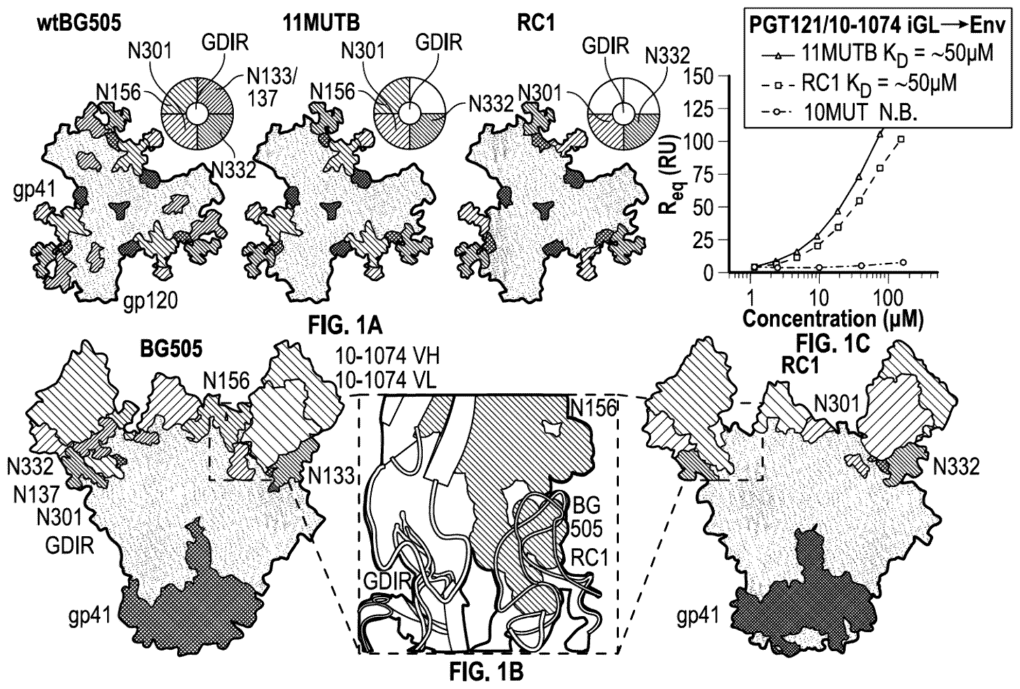

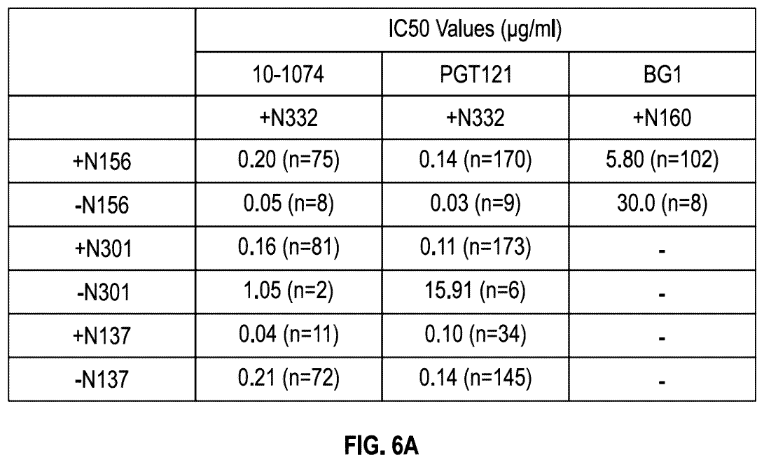

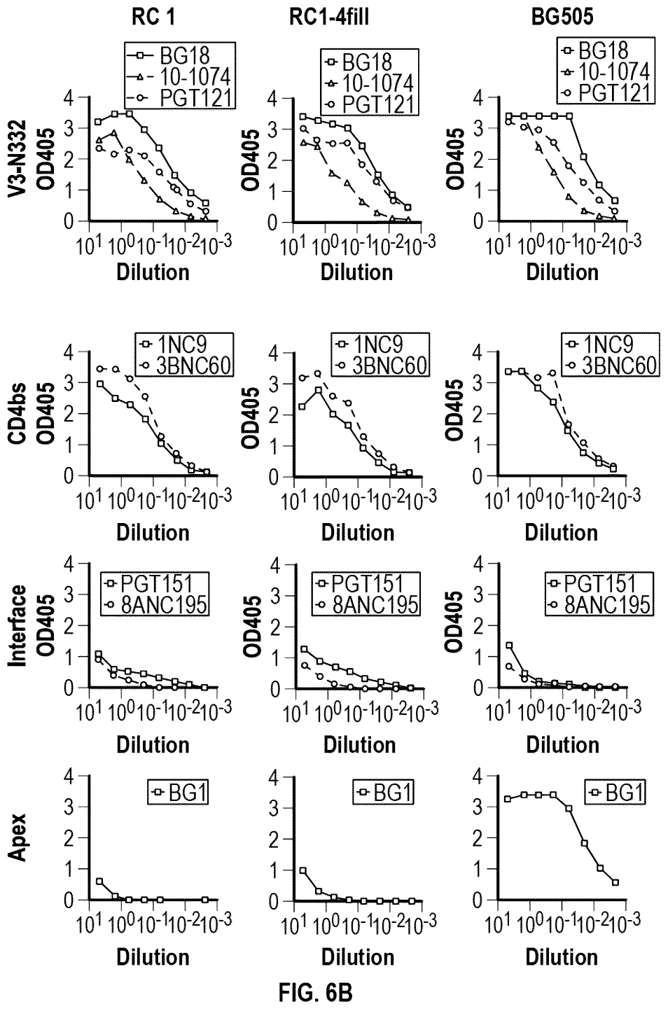

RC1 was designed using 11MUTB, a modified native-like soluble Env trimer (SOSIP.664) derived from the clade A/E BG505 Env, as a template. Compared to BG505, 11MUTB includes multiple substitutions in V1 and lacks potential N-linked glycosylation sites (PNGS) at positions N133 and N137 (FIG. 1a) (Steichen, J. M. et al. Immunity 45, 483-496(2016); Sanders, R. W. et al. PLoS Pathog 9, e1003618 (2013)). It was hypothesized that additional removal of the PNGS at position 156 (N156Q) would facilitate recognition of the V3-glycan patch by increasing accessibility of the parts of VI that interact with V3-glycan patch bNAbs. Consistent with this idea, absence of the N156 PNGS enhances neutralization by PGT121 and 10-1074 (FIG. 6a). In addition, it was further hypothesized that the removal of the N156 glycan, which includes negatively-charged terminal sialic acids, would produce a more electrostatically-neutral Env surface that could facilitate the binding of the largely neutral antibody precursor of the V3-glycan bNAbs PGT121 and 10-1074 (iGL PGT121/10-1074).

RC1 was initially characterized by evaluating its interactions with bNAbs by ELISA. As expected, a V1-V2-specific bNAb that interacts with the N156 glycan showed reduced binding to RC1 as compared to BG505 (FIGS. 6a and 6b). In contrast, bNAbs targeting the V3-glycan epitope, the CD4 binding site, or the gp 120-gp41 interface bound similarly to RC1 and BG505 (FIG. 6b). Thus, RC1 retained the overall antigenic properties of BG505.

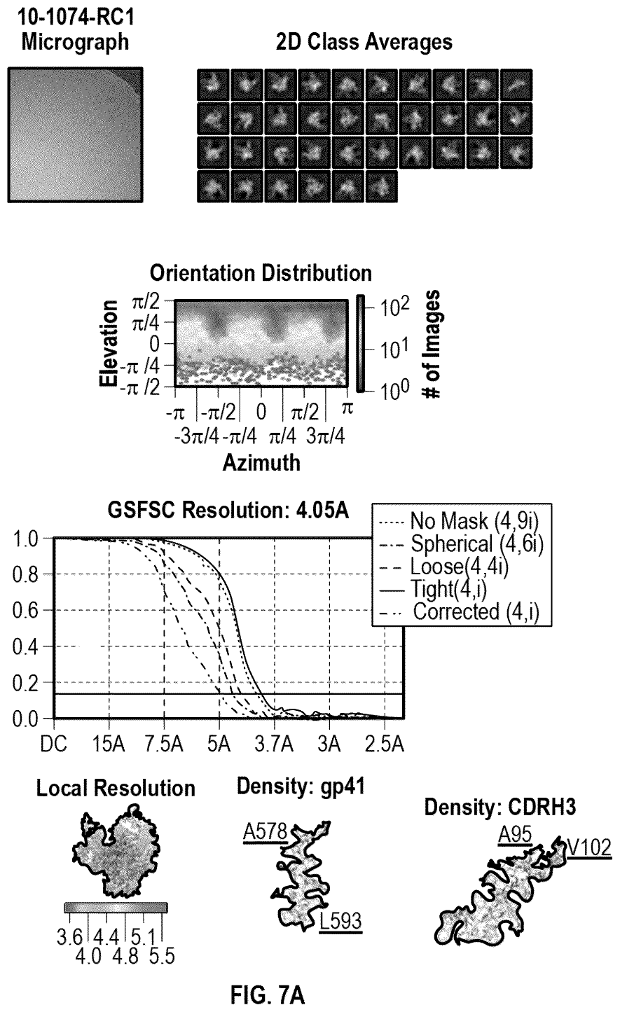

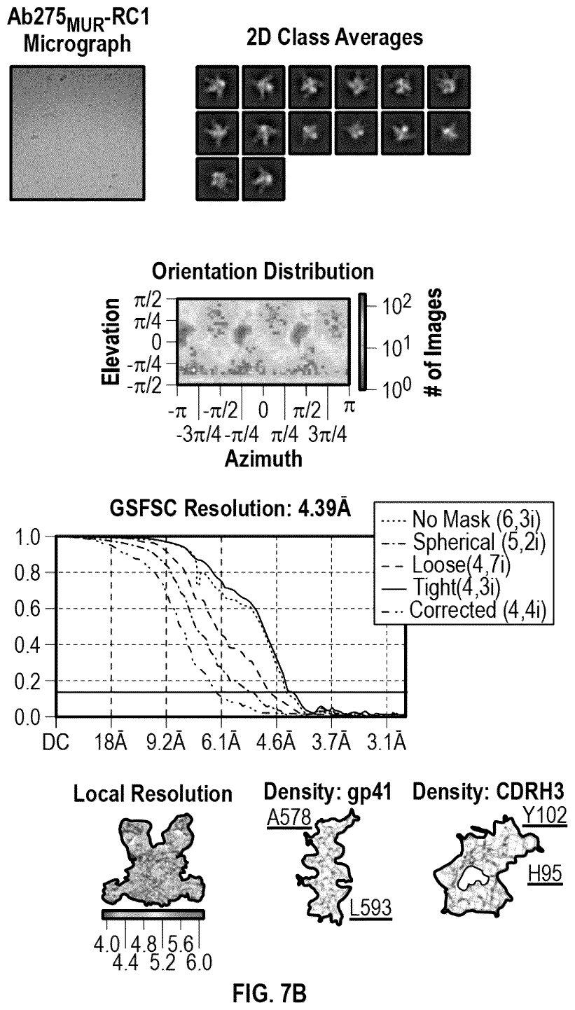

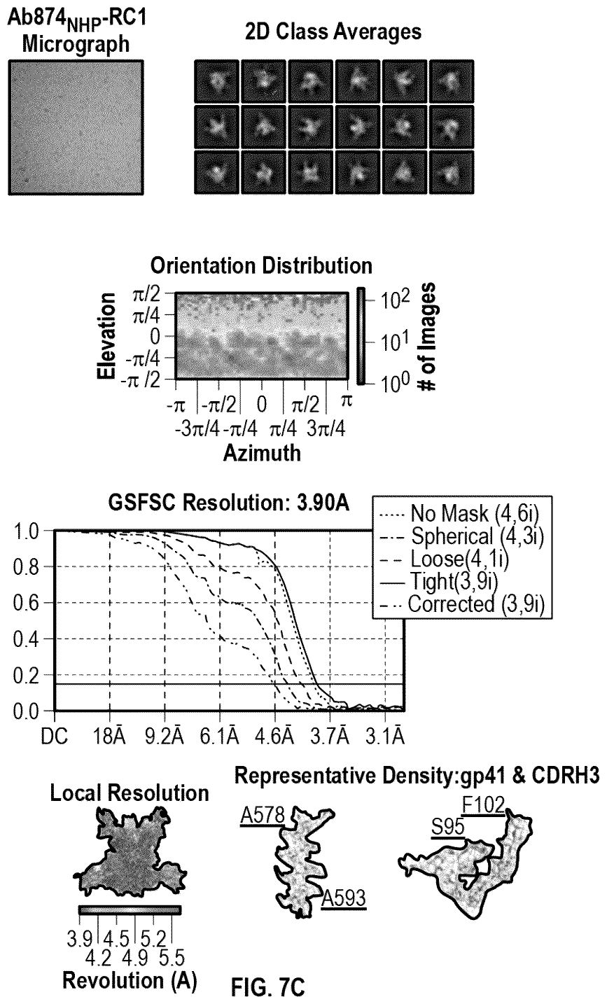

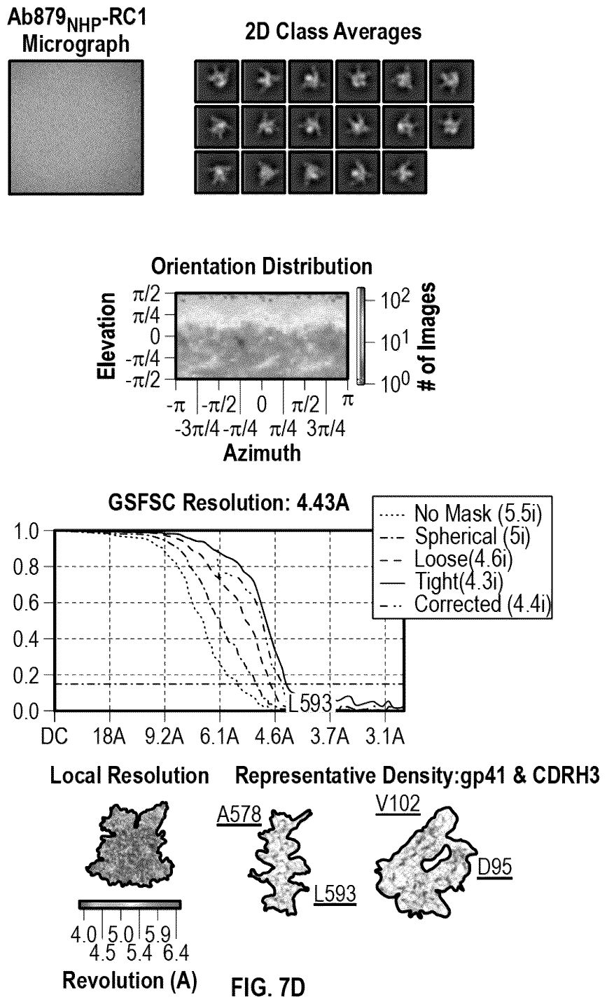

To further characterize RC1, a 4.0 Å single-particle cryo-EM structure of RC1 complexed with the antigen-binding fragment (Fab) of 10-1074 was solved and compared it to a structure of the same bNAb bound to BG505 (FIG. 1b; FIG. 7; Table 3). The RC1 structure was similar to BG505, with both showing the closed conformation of Env and containing three 10-1074 Fabs binding to the three V3-glycan patch epitopes (FIG. 1b). Compared with BG505, the V1 loop in RC1 included more ordered residues and was shifted towards the CDRH3 of 10-1074, allowing for increased interactions between the RC1 and 10-1074 (FIG. 1b).

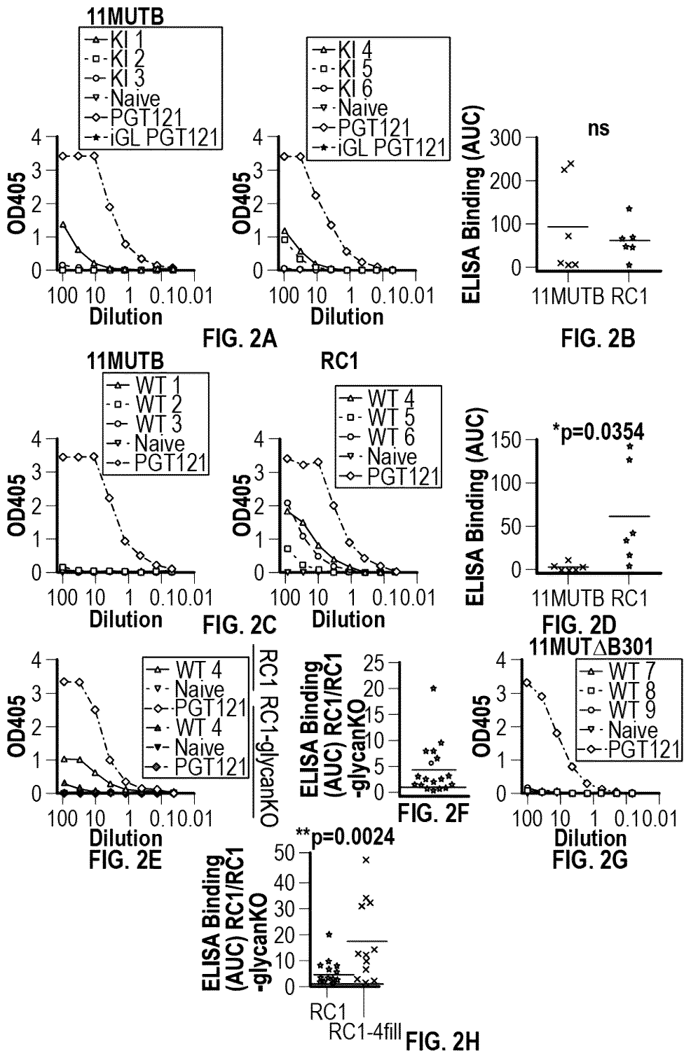

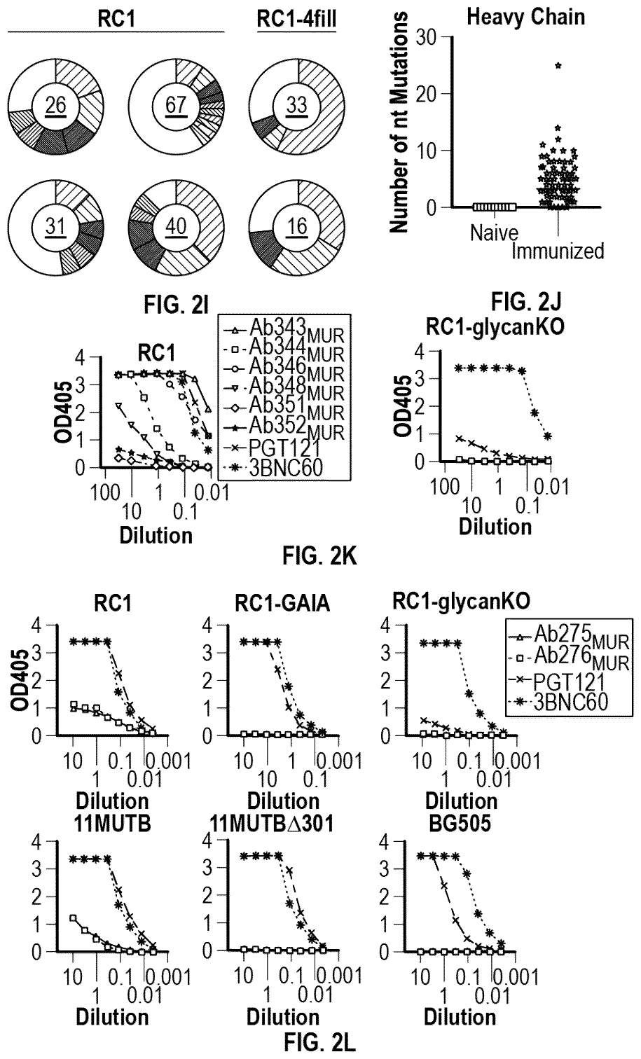

Despite structural changes in V1 resulting from deletion of the N156 glycan (FIG. 1b), the common iGL precursor of PGT121 and 10-1074 bound RC1 and 11MUTB with similar affinities (K_{D}˜50 μM) (FIG. 1c). Consistent with these observations, RC1 and 11MUTB elicited comparable V3-glycan epitope-specific serologic responses in knock-in (KI) mice carrying genes encoding the iGL PGT121/10-1074 (FIGS. 2a and 2b). In conclusion, RC1 exhibited structural changes from BG505, but these did not affect its affinity for the iGL PGT121/10-1074 precursor antibody.

### Example 3

### RC1 Elicits V3-Glycan Patch Antibodies in Wild-Type Mice

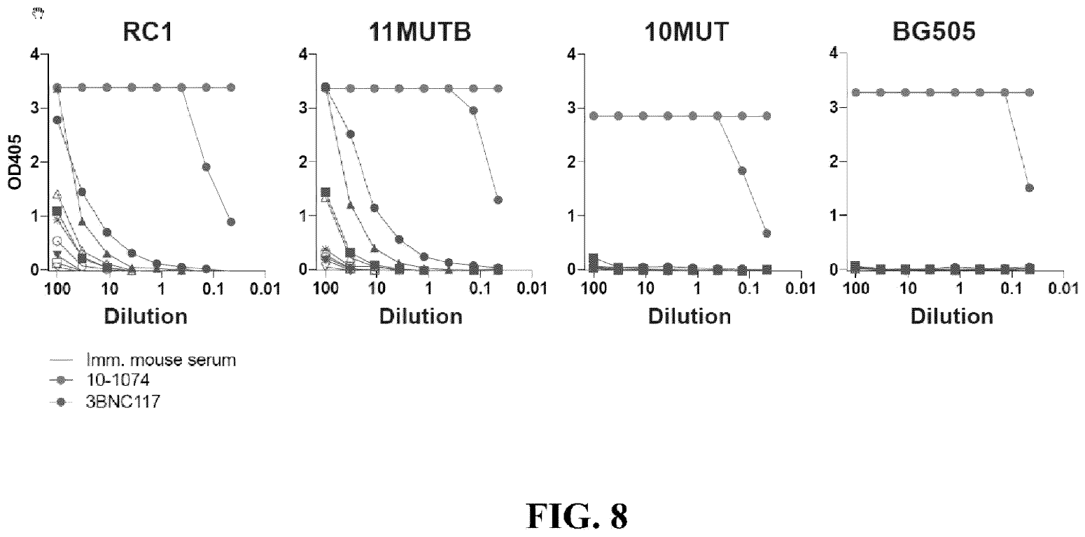

To determine whether RC1 can activate B-cells that carry V3-glycan patch-specific antibodies in wild-type mice, C57B1/6 mice were immunized with RC1 or 11MUTB. 11MUTB failed to produce a measurable serologic response (FIG. 2c). In contrast, RC1-immunized mice showed reproducible anti-V3-glycan patch responses as determined by ELISA comparing the binding to RC1 and to a mutant RC1 that lacks two additional V3 PNGSs at positions 301 and 332 (RC1-glycanKO) (FIGS. 2c, 2d, 2e, and 2f; Table 2). Moreover, the serum from the RC1-immunized mice cross-reacted with 11MUTB but not to the more native 10MUT Env or to BG505 (FIG. 8). The improved immunogenicity of the V3-glycan patch epitope of RC1 is the result of the specific removal of the N156 glycan from 11MUTB because removal of the N301 glycan from 11MUTB (11MUTBΔ301) (see Table 2) failed to induce detectable serologic responses in mice (FIG. 2g). It was concluded that, unlike 11MUTB and 11MUTBΔ301, RC1 elicits V3-glycan-specific serologic responses in wild-type mice.

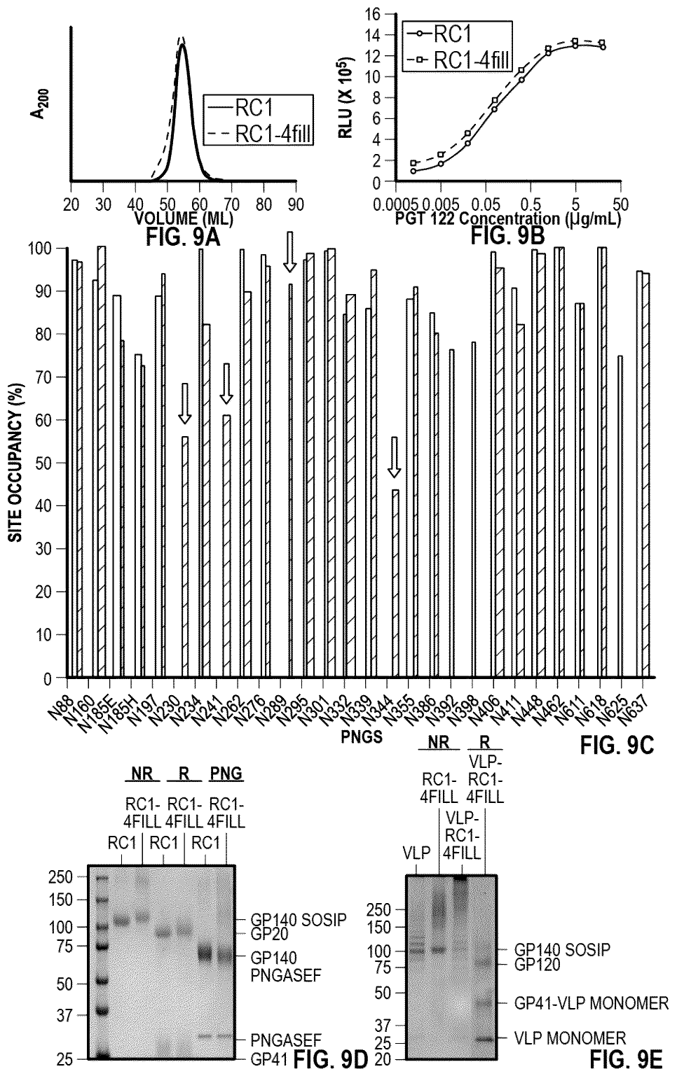

To reduce the antibody responses to off-target epitopes and further focus the response on the V3-glycan patch, an RC1 variant, RC1-4fill, was produced by adding PNGSs to cover potential off-target sites with glycans at gp 120 positions 230, 241, 289 and 344 (FIG. 9). RC1-4fill elicited serologic responses that were more specific to the V3-glycan patch in wild-type mice than those elicited by RC1, as determined by ELISAs against RC1 and RC1-glycanKO (FIG. 2h). It was concluded that RC1-4fill focuses the antibody responses to the V3-glycan patch epitope.

### Example 4

### Clonal Expansion of V3-Glycan Patch Specific B-Cells in Wild-Type Mice

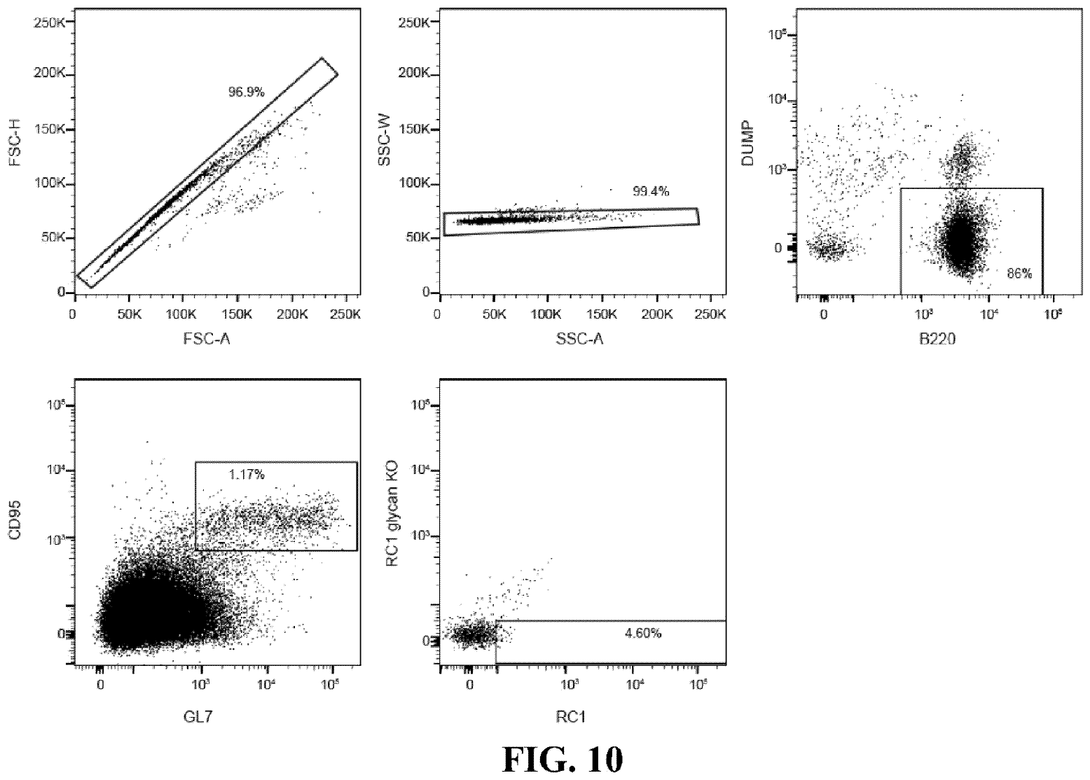

To further characterize the humoral responses elicited by RC1 and RC1-4fill in wild-type mice, the antibody genes from single GC B-cells that bound to RC1 but not to RC1-glycanKO was sequenced (FIG. 10). All RC1- and RC1-4fill-immunized mice analyzed showed expansion of GC B-cell clones (FIG. 2i). The expanded clones predominantly expressed heavy chain V gene segments VH5-6, VH9-3 and VH2-9, and light chain segments VK3-4 and VK14-111 (FIG. 2i; Tables 4, 5, and 6). The CDRH3 sequences in expanded clones showed similarities to human V3-glycan patch bNAbs such as Tyr-rich or RxY motifs (Tables 4 and 6) and longer-than-average CDRH3s but none had insertions or deletions. The V_{H} genes of the expanded clones had an average of 3.2 nucleotide mutations (FIG. 2j; Table 4).

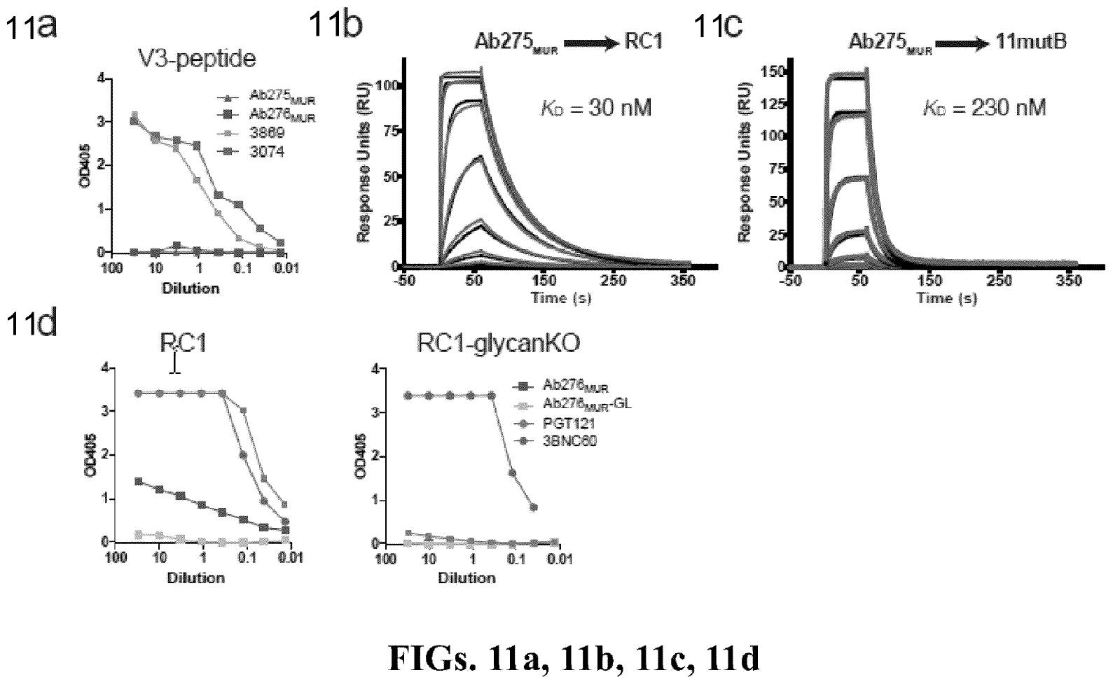

To determine the target site of the antibodies produced by the expanded B-cell clones, selected antibodies were cloned and produced, and ELISAs were performed against RC1 and RC1 mutant proteins. A diverse group of monoclonal antibodies (mAbs) showed V3-glycan patch-specific binding in ELISA (FIG. 2k). Further characterization of the Env-binding properties of two mAbs isolated from mice immunized with RC1 (Ab275_{MUR}) or RC1-4fill (Ab276_{MUR}) showed that these antibodies bind the V3-glycan patch epitope in a GDIR (SEQ ID NO: 15)- and N301-glycan-dependent manner (FIG. 2l; Table 2). Both antibodies bound 11MUTB, but not BG505 or a peptide that covers the crown of the V3 loop (FIG. 2l; FIG. 11a). Ab275_{MUR} bound RC1 with a K_{D}˜30 nM (FIG. 11b). Importantly, Ab275_{MUR} retained 30 binding to 11MUTB (K_{D}˜230 nM), demonstrating that it could accommodate the N156 glycan (FIG. 11c). The acquired mutations were essential for binding because reversion to the iGL sequence led to the loss of binding to RC1 (FIG. 11d). As expected, neither Ab275_{MUR} nor Ab276_{MUR} showed detectable neutralizing activity against a small panel of tier 1B and tier 2 HIV-1 isolates in TZMb1 assays (data not shown). Thus, it was concluded that RC1 and RC1-4fill expand mouse B-cell clones expressing antibodies that target the V3-glycan patch.

### Example 5

VLP-RC1-4Fill Elicits V3-Glycan Patch Antibodies in Rabbits and Rhesus macaques

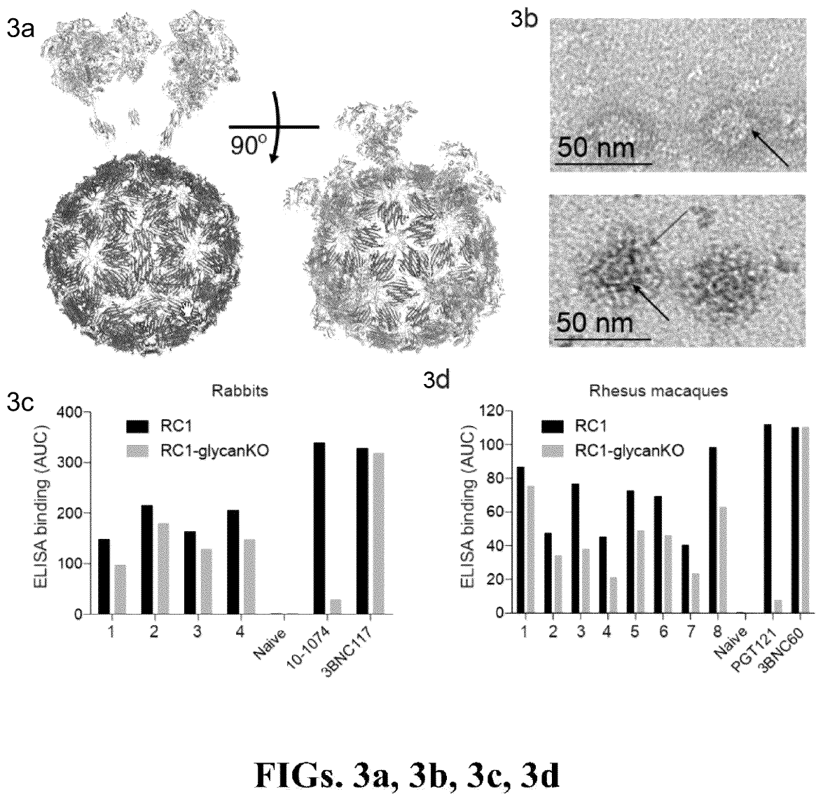

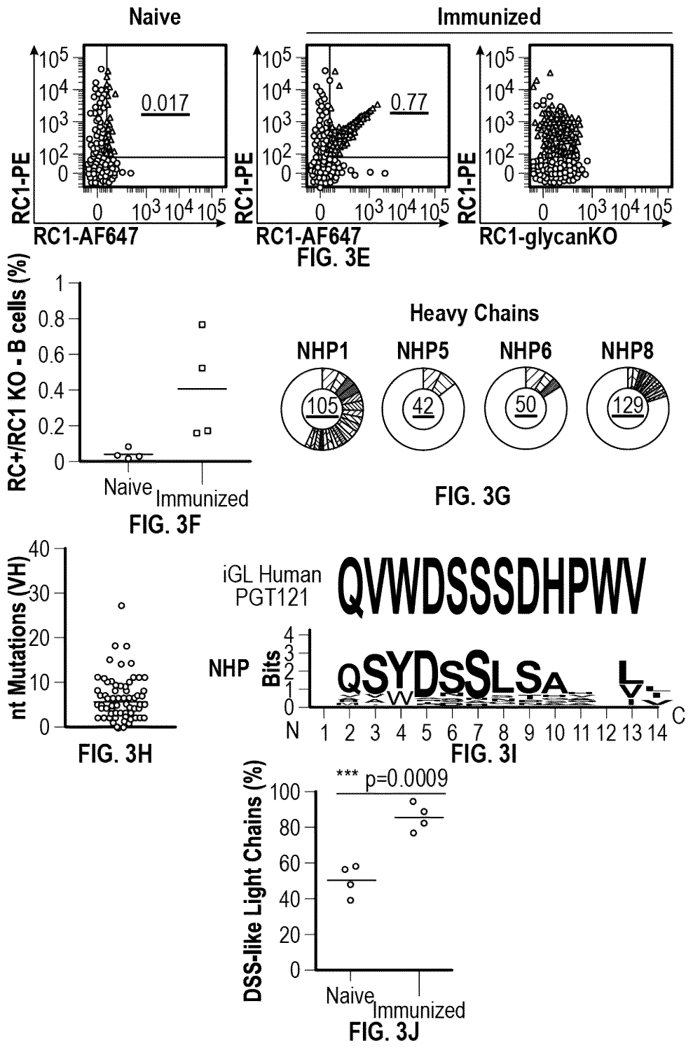

To enhance potential avidity effects and limit exposure of additional off-target epitopes at the base of the Env trimer, RC1-4fill was multimerized on virus-like particles (VLPs) using the Spytag-SpyCatcher system (FIGS. 3a and 3b). Rabbits and Rhesus macaques are thought to be better models than mice for HIV-1 vaccine studies because their antibodies have longer CDRH3s than mouse (average of 11 residues in mice, 13 in rabbits, and 15 in both Rhesus macaques and humans).

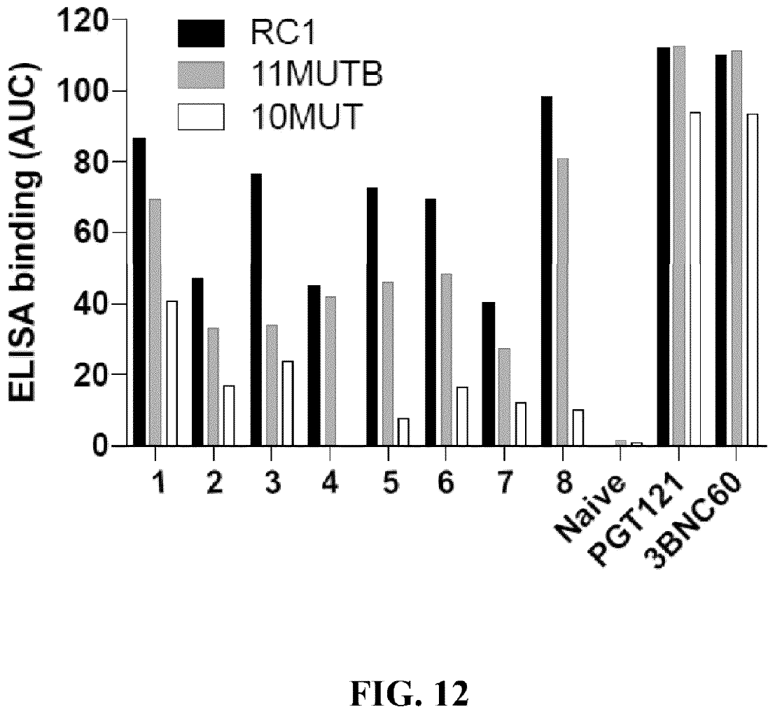

Immunization of 4 rabbits and 8 Rhesus macaques with RC1-4fill VLPs elicited serologic responses that were in part specific for the V3-glycan patch in all animals, as determined by ELISAs against RC1 and the RC1-glycanKO (FIGS. 3c and 3d). The serum from the macaques primed with RC1-4fill VLPs showed sequentially reduced binding to the more native-like immunogens 11MUTB and 10MUT (FIG. 12). Thus, RC1-4fill VLPs elicited robust serologic responses that mapped to the V3-glycan patch in rabbits and Rhesus macaques.

To further characterize responses elicited by RC1-4fill VLPs in macaques, draining lymph node GC B-cells that bound RC1 but not RC1-glycanKO was purified by flow cytometry (RC1⁺/RC1-glycanKO⁻). Whereas RC1+ cells were absent from the GCs of naïve macaques, RC1⁺/RC1-glycanKO⁻ GC B-cells were found at an average frequency of 0.4% of all GC B cells in the lymph nodes in the 4 macaques analyzed (FIGS. 3e and 3f).

Antibody cloning from 4 immunized macaques revealed that all showed expanded B-cell clones that used a variety of V_{H} genes with an average of 5.6 nucleotide somatic mutations (FIGS. 3g and 3h; Table 7). Most characterized human V3-glycan patch bNAbs contain a lambda light chain. Analysis of lambda gene usage revealed that macaque RC1 binding cells preferentially used genes VL132, which is 90.6% identical to VL2-8 in PGT125-128 and PGT130-131, and VL124, which is 93.8% identical to VL3-21 in PGT121-123/10-1074 (FIG. 3i; Table 8). Moreover, 86% of the lambda light chains had CDRL3s that included a DSS motif present in the iGLs of PGT121-123, 10-1074 and PGT124 (FIG. 3j; Table 9). This motif mutates to DSR in the mature bNAbs, and this substitution is critical for the neutralization activity of PGT121. It was concluded that macaque immunization with RC1-4fill VLPs expands B-cell clones whose antibody sequences resemble human V3-glycan patch bNAb precursors.

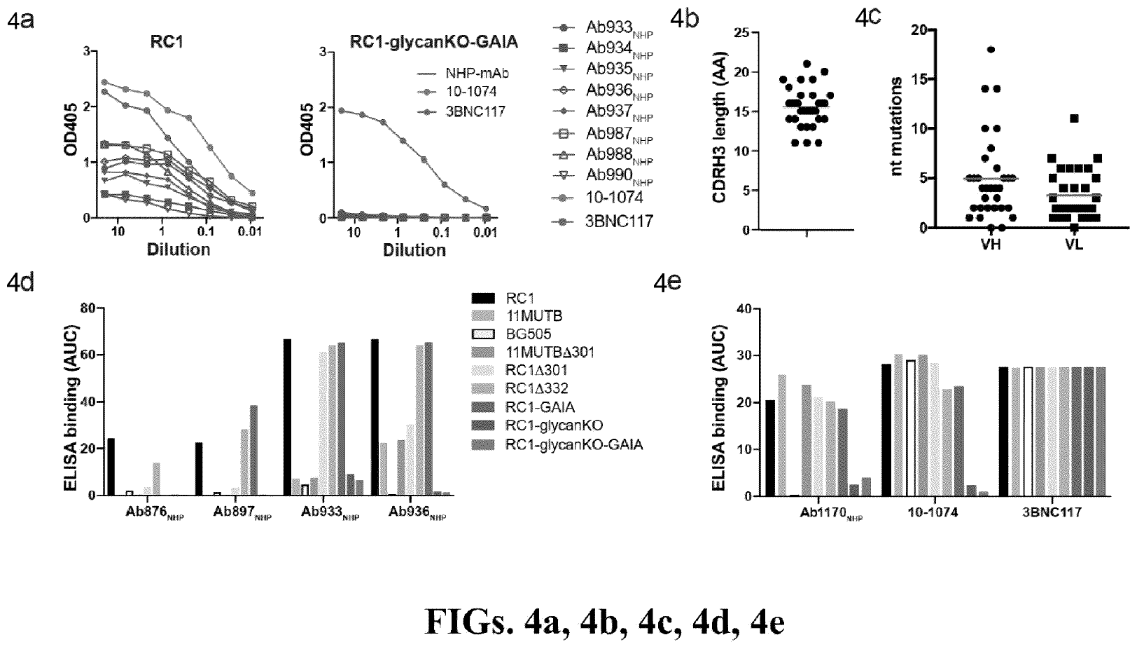

38 macaque GC antibodies were expressed with CDRL3s that resembled the CDRL3s of iGL V3-glycan patch bNAbs (Table 10). The CDRL3s of 33 of these antibodies contained a DSS motif and a Q at position 89 (QxxDSS motif (SEQ ID NO: 20)), also found in the CDRL3s of the PGT121-3, 10-1074, PGT124 and BG18 IGLs (Table 11). The other five antibodies contained an SYAG motif (SEQ ID NO: 21), which is present in the CDRL3s of the PGT125-7, PGT128, PGT130, and PGT131 iGLs (Table 11). Thirty of the 33 QxxDSS motif-containing antibodies (“QxxDSS” disclosed as SEQ ID NO: 20) and 2 of the 5 SYAG motif-containing antibodies (“SYAG” disclosed as SEQ ID NO: 21) bound to the V3-glycan patch epitope, as determined by ELISA using RC1 and RC1-glycanKO⁻ GAIA (“GAIA” disclosed as SEQ ID NO: 16) (FIG. 4a; Table 10). The CDRH3 length of these 38 V3-glycan patch antibodies ranged from 11 to 21 residues (average=15.5 residues) (FIG. 4b). Longer CDRH3s included a high content of Tyr and/or Phe residues, similar to the long CDRH3s of human V3-glycan patch bNAbs (Table 10). The V_{H} and VL genes of these antibodies had an average of 4.9 and 3.3 nucleotide mutations, respectively (FIG. 4c).

To further characterize antibody recognition of RC1, ELISAs were performed against additional mutants RC1-glycanKO, RC1-GAIA (“GAIA” disclosed as SEQ ID NO: 16), RC1-glycanKO-GAIA (“GAIA” disclosed as SEQ ID NO: 16), 11MUTBΔ301, RC1Δ301, RC1Δ332, 11MUTB and BG505 (FIGS. 4d and 4e; Table 2). The ELISAs suggested four different binding patterns to RC1 among the antibodies that contained a QxxDSS motif (SEQ ID NO: 20) in the CDRL3 (FIG. 4d) and an additional pattern among the antibodies containing an SYAG motif (SEQ ID NO: 21) (FIG. 4e). Whereas all of the antibodies were glycan-dependent as determined by the absence of binding to RC1-glycanKO, they differed in their binding to 11MUTB or 10MUT, dependence on GDIR motif (SEQ ID NO: 15) and on the N301, N332, and N156 glycans (FIGS. 4a, 4d, and 4e). None of the antibodies tested bound to BG505 or had neutralizing activity against a small panel of tier 1B and tier 2 HIV-1 isolates in TZMb1 assays (FIGS. 4d and 4e; data not shown). It was concluded that macaque immunization with RC1-4fill VLPs elicits V3-glycan patch-specific antibodies that resemble the precursors of human bNAbs that target this site.

### Example 6

### Cryo-EM Structures of Mouse and Macaque Antibodies in Complex With RC1

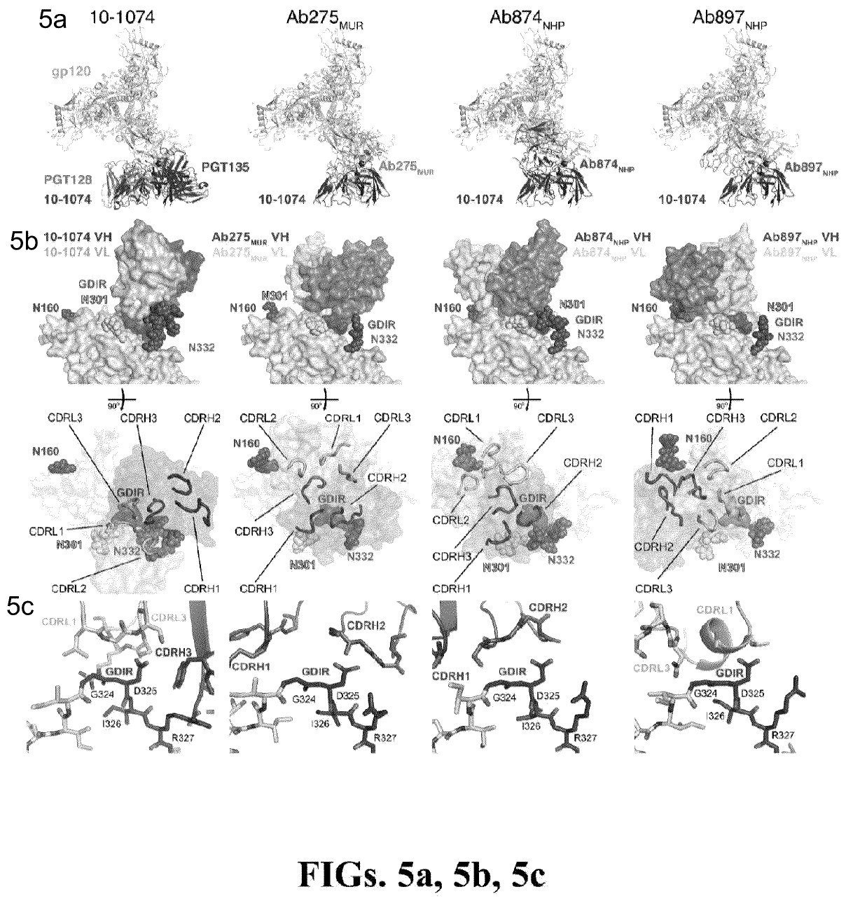

To define the molecular mechanism of binding and compare modes of V3-glycan patch recognition, structures of one mouse and two macaque Fabs complexed with RC1 were determined using single-particle cryo-electron microscopy. All three antibodies bound to the V3-glycan patch epitope with footprints overlapping the 10-1074 footprint, but bound with different angles of approach compared to 10-1074 (FIGS. 5a and 5b). Ab275_{MUR} (4.4 Å resolution) and Ab874_{NHP} (3.9 Å) (derived from the same clone as Ab876_{NHP}) bound similarly to each other, consistent with their 69% sequence identity, whereas Ab897_{NHP} (4.4 Å) (related by 48% and 54% sequence identity to Ab275_{MUR} and Ab874_{NHP}, respectively) adopted a distinct angle of approach (FIG. 5b).

All three Fabs in the RC1 complexes contacted the GDIR motif (SEQ ID NO: 15), but with different footprints compared with each other and with 10-1074. Whereas 10-1074 contacted the conserved GDIR motif (SEQ ID NO: 15) using CDRH3, CDRL1, and CDRL3 (FIGS. 1c and 5b), Ab874_{MUR}, and Ab275_{NHP} mainly made GDIR (SEQ ID NO: 15) contacts using their CDRH2s, and Ab897_{NHP} utilized CDRL1 and CDRL3 (FIGS. 5b and 5c). In addition to GDIR (SEQ ID NO: 15) contacts, Ab275_{MUR} and Ab874_{NHP} interacted with the N332 glycan (FIGS. 5a and 5b). However, unlike 10-1074, which interacts extensively with the N332 glycan via its CDRL1, FRWL3, CDRH2, and CDRH3, Ab275_{MUR} made minimal contacts using only its CDRH2, and Ab874_{NHP} engaged the N332 glycan with its CDRH2 and FRWH3. Interactions with the N332 glycan were not observed in the Ab897_{NHP}-RC1 structure. Despite the reduced binding of Ab275_{MUR}, Ab876_{NHP} (same clone as Ab874_{NHP}) and Ab897_{NHP} to RC1Δ301 (FIG. 2l), none of the Fabs in the RC1 complexes showed interactions with the N301 glycan, suggesting either glycan heterogeneity that would obscure this interaction and/or a conformational change in a V3-glycan patch lacking this glycan that would diminish binding. It was concluded that RC1 elicits V3-glycan patch-targeting antibodies with distinct binding modes in animals with polyclonal antibody repertoires including primates.

HIV-1 bNAbs develop in infected humans by sequential rounds of somatic mutation in response to a rapidly-evolving pathogen. Vaccination with a series of related antigens can reproduce this progression of events in genetically-engineered mice that carry super-physiologic numbers of B lymphocytes expressing the iGL precursors of bNAbs. An important goal of HIV-1 vaccine design is to design immunogens that initiate this response in organisms with polyclonal immune systems with the goal of reproducing these responses in humans.

HIV-1 vaccine immunogen design has focused upon increasing the affinity of candidate immunogens for specific iGL bNAb precursors with the objective of recruiting a specific group of rare precursors into the GC. This approach typically fails to account for increases in apparent affinity produced by interactions between multimerized antigen and polyvalent antigen receptors on the surface of a B-cell. Moreover, GC entry is primarily limited by competition. Thus, the importance of affinity is relative, as evidenced by the observation that B-cells bearing low-affinity receptors are frequently found in GCs under physiologic conditions.

The principles employed to produce RC1 did not take affinity into account. Instead, RC1 was designed to increase the number of bNAb progenitors that can compete for GC entry. This was done by making the antigenic target site more available while facilitating binding to electrostatically-neutral iGL precursors. In addition, the RC1-4fill VLP incorporates the idea that masking competing for off-target epitopes minimizes competition for GC entry.

RC1 differs from other HIV-1 vaccine candidates in that it induces B-cells expressing antibodies against a targeted epitope to undergo clonal expansion in GCs in animals with a fully polyclonal B-cell repertoire. In macaques, these B-cells express antibodies that show sequence and structural similarities to iGL precursors of bNAbs targeting the V3-glycan patch. Thus, RC1-4fill VLPs are a suitable candidate immunogen for sequential vaccination strategies that aim to elicit V3-glycan bNAbs.

### Example 7

### RC1-3Fill VLPs and NPs Behave Similarly to RC1 VLPs and NPs

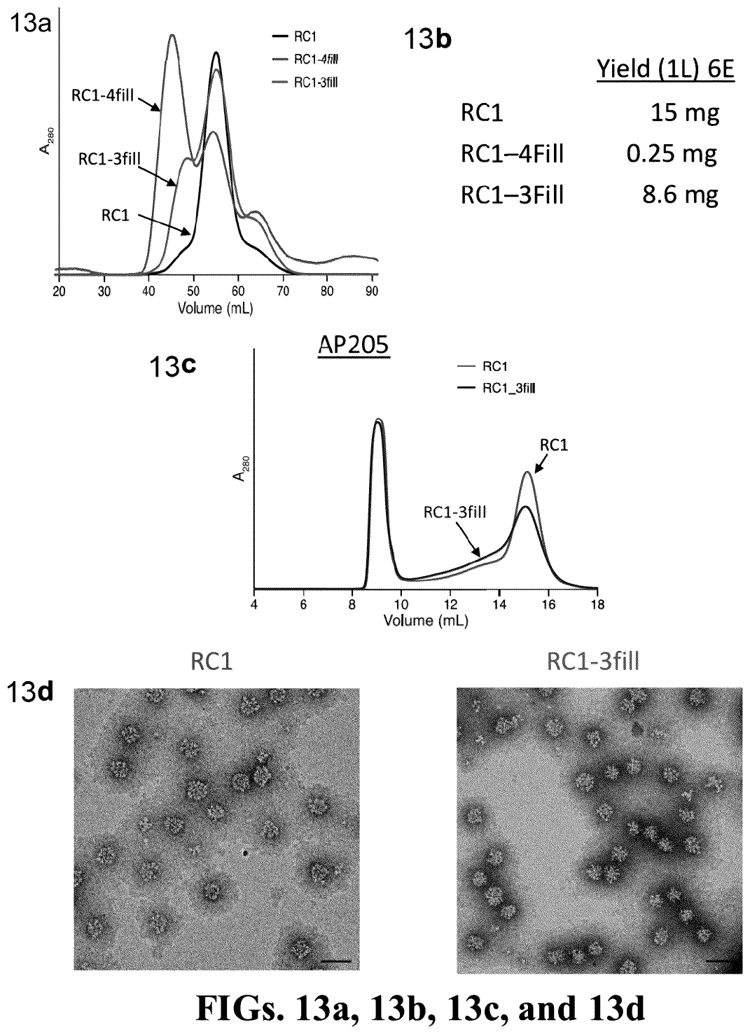

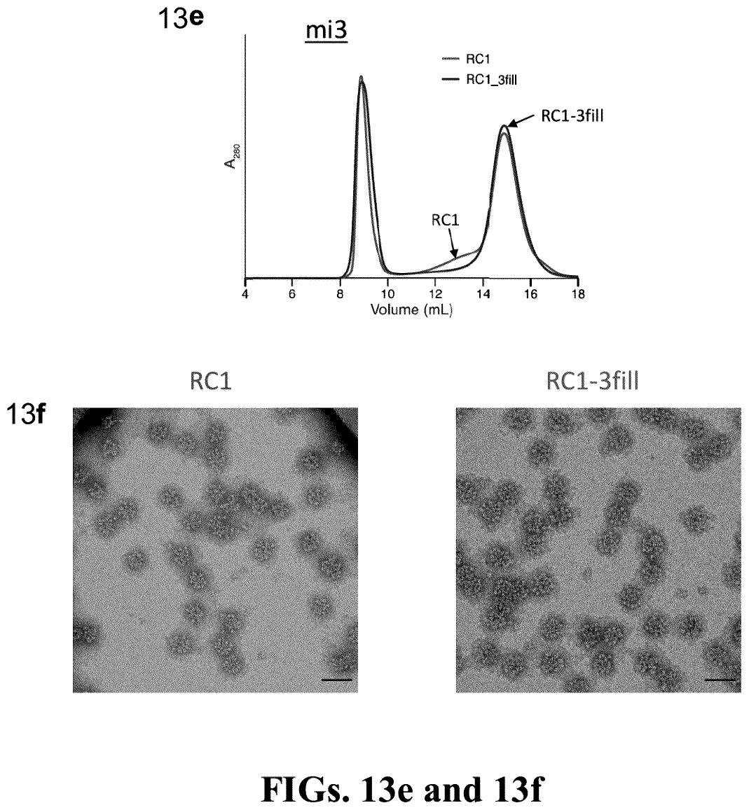

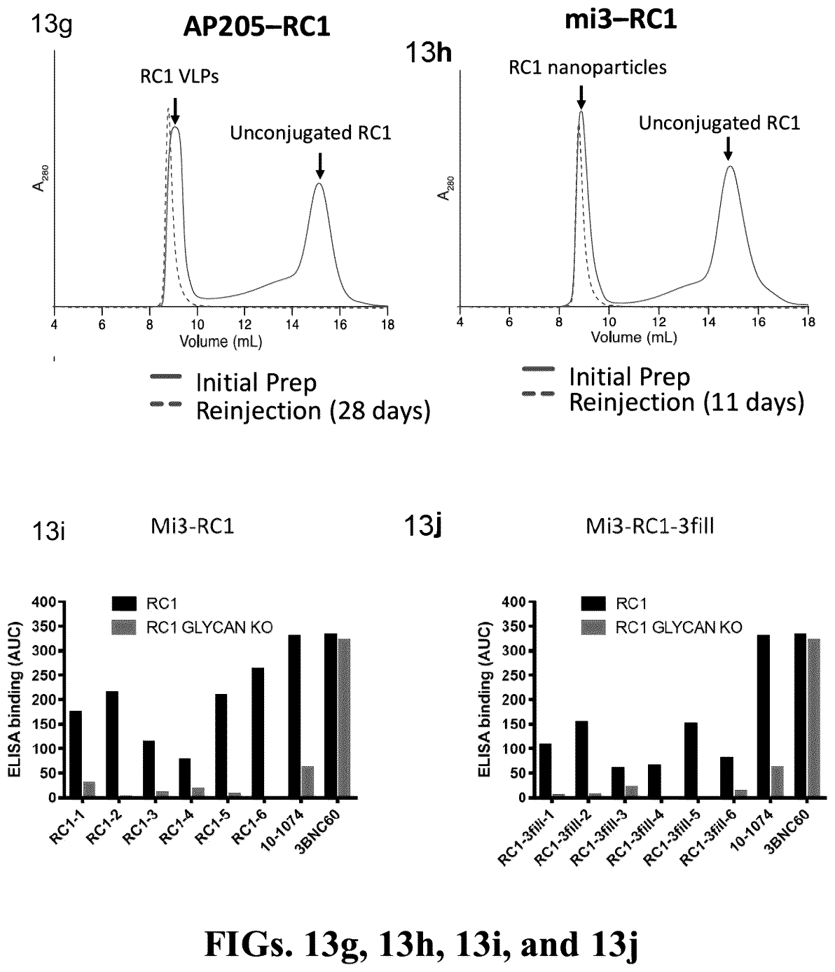

Size-exclusion chromatography (SEC) traces for the RC1, RC1-3fill, and RC1-4fill immunogens (FIG. 13a) show that a smaller fraction of the RC1 and RC1-3fill immunogens elute in the void volume compared to RC1-4fill, demonstrating that RC1 and RC1-3fill are more stable and less-prone to aggregate than RC1-4fill. Representative yields from a 1 L expression in HEK 293T 6E cells for each immunogen (FIG. 13b) suggest that RC1-3fill was expressed at a higher level than RC1-4fill and at a similar level to RC1.

FIG. 13c shows representative SEC traces for the purification of the AP205-RC1-VLPs (dark gray) and AP205-RC1-3fill-VLPs (black). FIG. 13d shows electron micrographs of the AP205-RC1-VLPs (left) and AP205-RC1-3fill-VLPs (right), showing the AP205-RC1-3fill-VLPs look similar to AP205-RC1-VLPs and have a similar number of conjugated trimers per particle. The micrographs also show that the purification strategy was sufficient and no free trimer was present in either sample. Representative SEC traces for the purification of the mi3-RC1-NPs (dark gray) and mi3-RC1-3fill-NPs (black) (FIG. 13e) showing that RC1 and RC1-3fill can be conjugated to mi3 NPs. Electron micrographs of the mi3-RC1-NPs (left) and mi3-RC1-3fill-NPs (right) (FIG. 13f) show the mi3-RC1-3fill-NPs look similar to mi3-RC1-NPs and have a similar number of conjugated trimers per particle. The micrographs also show that the purification strategy was sufficient and no free trimer was present in either sample. SEC profiles for both the initial purification of the AP205-RC1-VLPs (FIG. 13g) and the mi3-RC1-NPs (FIG. 13h), and a reinjection of the sample at 28 days (AP205) and 11 days (mi3) show that the conjugated particles were stable over time and no unconjugated RC1 or degradation products were seen after storage for 28 or 11 days.

Serum from six WT mice immunized with either mi3-RC1-NPs (FIG. 13i) or mi3-RC1-3fill-NPs (FIG. 13j) was tested for binding to RC1 (black) and RC1 glycan KO (gray). Serum from all six mice immunized with either Mi3-RC1 or Mi3-RC1-3fill bound to RC1 in an ELISA and had reduced binding to RC1 glycan KO, suggesting a serum response specific to the V3/N332 glycan patch. Monoclonal antibodies 10-1074 and 3BNC117 were included as positive and negative controls. ELISA data are shown as area under the ELISA curve (AUC).

TABLE 3
Cryo-EM data collection and processing statistics.

| Env | RC1 | RC1 | RC1 | RC1 |
|---|---|---|---|---|
| Fabs | 10-1074; | Ab275_{MUR}; | Ab874_{NHP}; | Ab897_{NHP}; |
| | CD4bs | 8ANC195 | 8ANC195 | 8ANC195 |
| Concentration | 0.75 | 1.25 | 1.25 | 1.4 |
| (mg/mL) | | | | |
| Blot time (s) | 3.5 | 3.0 | 2.0 | 3.5 |
| Microscope | FEI Talos | FEI Talos | FEI Talos | FEI Talos |
| | Arctica | Arctica | Arctica | Arctica |
| Voltage (kV) | 200 | 200 | 200 | 200 |
| Detector | Falcon 3EC | Falcon 3EC | Falcon 3EC | Falcon 3EC |
| Recording mode | counting | counting | counting | counting |
| Magnification | 73k | 73k | 73k | 73k |
| Pixel size (Å) | 1.436 | 1.436 | 1.436 | 1.436 |
| Dose rate (e-/px/s) | 0.73, 0.77 | 1.28 | 1.3 | 1.3 |
| frames per micrograph | 39 | 40 | 39 | 39 |
| Total dose (e-/Å²) | 39.1 | 40 | 40 | 40 |
| Defocus range (μm) | 1-3.4 | 0.8-2.5 | 0.8-2.5 | 0.8-2.5 |
| number of micrographs | 684 | 328 | 465 | 510 |
| number of particles | 122,013 | 49,308 | 86,564 | 158,954 |
| symmetry | C3 | C3 | C3 | C3 |
| resolution | 4.05 | 4.39 | 3.90 | 4.43 |
| (FSC 0.143) (Å) | | | | |
| B-factor (Å²) | −281.9 | −252.4 | −230.0 | −322.1 |

TABLE 4
Sequences of Antibodies Generated from RC1- and RC1-4fill-immunized Mice.

| | | | | | | |
|---|---|---|---|---|---|---|
| | | | | SEQ | LENGTH | Nt |
| VH | DH | JH | CDRH3 | ID NO: | (AA) | mut. |

Mouse 5

| | | | | | | |
|---|---|---|---|---|---|---|
| IGHV1-84*01 | IGHD4-1*01 | IGHJ3*01 | ASGDELAWFAY | 22 | 11 | 5 |
| IGHV1-84*01 | IGHD4-1*01 | IGHJ3*01 | ASGDELAWFAY | 22 | 11 | 5 |
| IGHV1-84*01 | IGHD4-1*01 | IGHJ3*01 | ASGDELAWFAY | 22 | 11 | 4 |
| IGHV1-84*01 | IGHD4-1*01 | IGHJ3*01 | ASGDELAWFAY | 22 | 11 | 3 |
| IGHV1-84*01 | IGHD4-1*01 | IGHJ3*01 | ASGDELAWFAY | 22 | 11 | 6 |
| IGHV1-84*01 | IGHD4-1*01 | IGHJ3*01 | ASGDELAWFAY | 22 | 11 | 2 |
| IGHV1-84*01 | IGHD4-1*01 | IGHJ3*01 | ASGDELACFAY | 23 | 11 | 3 |
| IGHV1-84*01 | IGHD4-1*01 | IGHJ3*01 | ASGDELAWFAY | 22 | 11 | 6 |
| IGHV1-84*01 | | IGHJ3*01 | ASGDELAWFAY | 22 | 11 | 4 |
| IGHV1-84*01 | IGHD4-1*01 | IGHJ3*01 | ASGDELAWFAY | 22 | 11 | 2 |
| IGHV1-84*01 | | IGHJ3*01 | ANGDALAWFAY | 24 | 11 | 5 |
| IGHV1-84*01 | IGHD4-1*01 | IGHJ3*01 | ASGDELAWFAY | 22 | 11 | 2 |
| IGHV1-84*01 | IGHD4-1*01 | IGHJ3*01 | ASGDELAWFAY | 22 | 11 | 5 |
| IGHV1-84*01 | IGHD4-1*01 | IGHJ3*01 | ASGDELAWFAY | 22 | 11 | 6 |
| IGHV1-84*01 | IGHD4-1*01 | IGHJ3*01 | ASGDELAWFAY | 22 | 11 | 5 |
| IGHV1-84*01 | IGHD4-1*01 | IGHJ3*01 | ASGDELAWFAY | 22 | 11 | 4 |
| IGHV1-84*01 | IGHD4-1*01 | IGHJ3*01 | ACGDELAWFAY | 25 | 11 | 3 |
| IGHV1-84*01 | IGHD4-1*01 | IGHJ3*01 | ASGDELAWFAY | 22 | 11 | 2 |
| IGHV1-84*01 | IGHD4-1*01 | IGHJ3*01 | AGGDELAWFAY | 26 | 11 | 7 |
| IGHV1-72*01 | IGHD2-4*01 | IGHJ3*01 | VRGEVYYDYDGFAY | 27 | 14 | 8 |
| IGHV1-72*01 | IGHD2-4*01 | IGHJ3*01 | ARGEVYYDYDGFAY | 28 | 14 | 1 |
| IGHV1-9*01 | IGHD2-4*01 | IGHJ1*03 | ARIRSDYDVGWWYFDV | 29 | 16 | 4 |
| IGHV1-9*01 | IGHD2-4*01 | IGHJ1*03 | ARIRSDYDVGWWYFDV | 29 | 16 | 4 |
| IGHV1-72*01 | IGHD1-1*01 | IGHJ2*01 | ARYYYGHYFDY | 30 | 11 | 5 |
| IGHV1-9*01 | IGHD2-14*01 | IGHJ2*01 | VRSGIYYFDY | 31 | 10 | 5 |
| IGHV1-72*01 | IGHD1-1*01 | IGHJ2*01 | ARYLLLRPFDY | 32 | 11 | 4 |
| IGHV1-22*01 | IGHD1-1*01 | IGHJ4*01 | ARAGTTGYVMDY | 33 | 12 | 3 |
| IGHV1-74*01 | IGHD6-1*01 | IGHJ4*01 | AIASYYYTLDY | 34 | 11 | 5 |
| IGHV1-19*01 | IGHD3-2*02 | IGHJ3*01 | ARRGAAQAPFAY | 35 | 12 | 3 |
| IGHV1-82*01 | IGHD4-1*01 | IGHJ3*01 | VRSELGPAFAY | 36 | 11 | 7 |
| IGHV1-22*01 | IGHD2-2*01 | IGHJ4*01 | ARRGYGYGAMDY | 37 | 12 | 1 |
| IGHV1-61*01 | IGHD2-5*01 | IGHJ3*01 | ARAYSNYVPWFAY | 38 | 13 | 0 |
| IGHV1-69*01 | IGHD2-10*02 | IGHJ2*01 | ARREYGFFDY | 39 | 10 | 6 |

Mouse 6

| | | | | | | |
|---|---|---|---|---|---|---|
| IGHV5-6*01 | IGHD4-1*01 | IGHJ4*01 | ARHGRLTGTGAMDY | 40 | 14 | 3 |
| IGHV5-6*01 | IGHD4-1*01 | IGHJ4*01 | ARHGRLTGTGAMDY | 40 | 14 | 6 |
| IGHV5-6*01 | IGHD4-1*01 | IGHJ4*01 | ARHGRLTGTGAMDY | 40 | 14 | 0 |
| IGHV5-6*01 | IGHD4-1*01 | IGHJ4*01 | ARHGRLTGTGAMDY | 40 | 14 | 2 |
| IGHV5-6*01 | IGHD4-1*01 | IGHJ4*01 | ARHGRLTGTGAMDY | 40 | 14 | 2 |
| IGHV5-6*01 | IGHD3-3*01 | IGHJ4*01 | ARHGAGNALDY | 41 | 11 | 2 |
| IGHV5-6*01 | | IGHJ4*01 | ARHGAGNAMDY | 42 | 11 | 3 |
| IGHV5-6*01 | | IGHJ4*01 | ARHGAGNAMDY | 42 | 11 | 6 |
| IGHV5-6*01 | | IGHJ4*01 | ARHGAGNAMDY | 42 | 11 | 2 |
| IGHV9-3*01 | IGHD2-1*01 | IGHJ2*01 | QVEVTMWTT | 43 | 9 | 0 |
| IGHV9-3*01 | | IGHJ2*01 | ASGRNYVDY | 44 | 9 | 3 |
| IGHV9-3*01 | | IGHJ2*01 | ASGPNYFDY | 45 | 9 | 3 |
| IGHV5-6*01 | IGHD1-1*01 | IGHJ4*01 | ARHGHYYGSSYGMDY | 46 | 15 | 2 |
| IGHV1-75*01 | IGHD1-1*02 | IGHJ1*01 | ARDDGGYWYFDV | 47 | 12 | 1 |
| IGHV2-9*01 | IGHD1-3*01 | IGHJ4*01 | ANIPKDRLCYGP | 48 | 12 | 2 |
| IGHV1-62-2* | IGHD2-3*01 | IGHJ3*01 | ARHEEDGYWFAY | 49 | 12 | 11 |
| 01 | | | | | | |

TABLE 5
Sequences of Antibodies Generated from RC1- and RC1-4fill-immunized Mice.
LIGHT CHAINS

| | | | | SEQ ID | LENGTH | Nt |
|---|---|---|---|---|---|---|
| MOUSE | VH | JH | CDRL3 | NO: | (AA) | mut. |
| 4 | IGKV14-111*01 | IGKJ2*01 | LQYDEFPYT | 50 | 9 | 2 |
| 4 | IGKV14-111*01 | IGKJ2*01 | LHYDDFPYT | 51 | 9 | 3 |
| 4 | IGKV14-111*01 | IGKJ2*01 | LHYDDFPYT | 51 | 9 | 4 |
| 4 | IGKV14-111*01 | IGKJ2*01 | LQYDEFPFT | 52 | 9 | 2 |
| 4 | IGKV14-111*01 | IGKJ2*01 | LQYDEFPYT | 50 | 9 | 10 |
| 4 | IGKV14-111*01 | IGKJ2*01 | LRYDDFPYT | 53 | 9 | 5 |
| 4 | IGKV14-111*01 | IGKJ2*01 | LQYDEFPYT | 50 | 9 | 5 |
| 4 | IGKV14-111*01 | IGKJ2*01 | LQYDEFPYT | 50 | 9 | 4 |
| 4 | IGKV14-111*01 | IGKJ2*01 | LQYDEFPYT | 50 | 9 | 3 |
| 4 | IGKV14-111*01 | IGKJ2*01 | LHYDDFPYT | 51 | 9 | 8 |
| 4 | IGKV14-111*01 | IGKJ2*01 | LQYDEFPYT | 50 | 9 | 0 |
| 4 | IGKV14-111*01 | IGKJ2*01 | LQYDEFPYT | 50 | 9 | 4 |
| 4 | IGKV14-111*01 | IGKJ2*01 | IQYDEFPYT | 54 | 9 | 4 |
| 4 | IGKV14-111*01 | IGKJ2*01 | LQYDEFPFT | 52 | 9 | 2 |
| 4 | IGKV14-111*01 | IGKJ2*01 | LHYDDFPYT | 51 | 9 | 5 |
| 4 | IGKV14-111*01 | IGKJ2*01 | LQYDEFPYT | 50 | 9 | 2 |
| 4 | IGKV14-111*01 | IGKJ2*01 | LQYDEFPYT | 50 | 9 | 6 |
| 4 | IGKV14-111*01 | IGKJ2*01 | LQYDEFPYT | 50 | 9 | 2 |
| 4 | IGKV14-111*01 | IGKJ2*01 | LHYDEFPYT | 55 | 9 | 2 |
| 4 | IGKV14-111*01 | IGKJ2*01 | LHYDDLPYT | 56 | 9 | 6 |
| 4 | IGKV14-111*01 | IGKJ2*01 | LQYDEFPFT | 52 | 9 | 1 |
| 4 | IGKV14-111*01 | IGKJ2*01 | LQYDEFPYT | 50 | 9 | 1 |
| 4 | IGKV14-111*01 | IGKJ2*01 | LHYDDLPYT | 56 | 9 | 5 |
| 4 | IGKV14-111*01 | IGKJ2*01 | LQYDEFPYT | 50 | 9 | 3 |
| 4 | IGKV14-111*01 | IGKJ2*01 | LQYDEFPYT | 50 | 9 | 4 |
| 4 | IGKV14-111*01 | IGKJ2*01 | LQYDEFPYT | 50 | 9 | 8 |
| 4 | IGKV14-111*01 | IGKJ2*01 | LQYDEFPYT | 50 | 9 | 5 |
| 4 | IGKV14-111*01 | IGKJ2*01 | LQYDEFPHT | 57 | 9 | 4 |
| 4 | IGKV14-111*01 | IGKJ2*01 | LQYDEFPYT | 50 | 9 | 6 |
| 4 | IGKV14-111*01 | IGKJ2*01 | LHYDDFPYT | 51 | 9 | 3 |
| 4 | IGKV14-111*01 | IGKJ2*01 | LQYDEFPYT | 50 | 9 | 0 |
| 4 | IGKV14-111*01 | IGKJ2*01 | LQYDESPYT | 58 | 9 | 9 |
| 4 | IGKV14-111*01 | IGKJ2*01 | LQYDEFPYT | 50 | 9 | 5 |
| 6 | IGKV14-111*01 | IGKJ2*01 | LQYDEFPYT | 50 | 9 | 3 |
| 6 | IGKV14-111*01 | IGKJ2*01 | LQYDEFPYT | 50 | 9 | 3 |
| 6 | IGKV14-111*01 | IGKJ2*01 | LQYDEFPCT | 59 | 9 | 1 |
| 1 | IGKV14-111*01 | IGKJ2*01 | LQYDEFPYT | 50 | 9 | 0 |
| 1 | IGKV14-111*01 | IGKJ2*01 | LQYDEFPYT | 50 | 9 | 5 |
| 1 | IGKV14-111*01 | IGKJ2*01 | LQYDEFPYT | 50 | 9 | 2 |
| 4 | IGKV14-111*01 | IGKJ2*01 | LQYDEFPYT | 50 | 9 | 3 |
| 4 | IGKV14-111*01 | IGKJ2*01 | LQYDEFPHT | 57 | 9 | 3 |
| 4 | IGKV14-111*01 | IGKJ2*01 | LQYDDFPHT | 60 | 9 | 5 |
| 4 | IGKV14-111*01 | IGKJ2*01 | LQYDEFPYT | 50 | 9 | 2 |
| 4 | IGKV14-111*01 | IGKJ2*01 | LQYDEFPYT | 50 | 9 | 3 |
| 4 | IGKV3-4*01 | IGKJ2*01 | QQSNEDPYT | 61 | 9 | 1 |
| 4 | IGKV3-4*01 | IGKJ2*01 | QQSNVDPYT | 62 | 9 | 2 |
| 4 | IGKV3-4*01 | IGKJ2*01 | QQSHEDPYT | 63 | 9 | 11 |
| 4 | IGKV3-4*01 | IGKJ2*01 | QQSNEDPYT | 61 | 9 | 8 |
| 4 | IGKV3-4*01 | IGKJ2*01 | QQSNEDPYT | 61 | 9 | 2 |
| 4 | IGKV3-4*01 | IGKJ2*01 | QQSNEDPYT | 61 | 9 | 1 |
| 4 | IGKV3-4*01 | IGKJ2*01 | QQSNEDPYT | 61 | 9 | 8 |
| 4 | IGKV3-4*01 | IGKJ2*01 | QQSNVDPYT | 62 | 9 | 27 |
| 4 | IGKV3-4*01 | IGKJ2*01 | QQSNEDPYT | 61 | 9 | 2 |
| 4 | IGKV3-4*01 | IGKJ2*01 | QQSNEDPYT | 61 | 9 | 7 |
| 4 | IGKV3-4*01 | IGKJ2*01 | QQSNEDPYT | 61 | 9 | 12 |
| 6 | IGKV3-4*01 | IGKJ2*01 | QHSNEDPYT | 64 | 9 | 2 |
| 6 | IGKV3-4*01 | IGKJ2*01 | QQSNEDPYT | 61 | 9 | 1 |
| 6 | IGKV3-4*01 | IGKJ2*01 | QQSNEDPYT | 61 | 9 | 1 |
| 6 | IGKV3-4*01 | IGKJ2*01 | QQSNEDPYT | 61 | 9 | 3 |
| 1 | IGKV3-4*01 | IGKJ2*01 | QQSNEDPYT | 61 | 9 | 0 |
| 1 | IGKV3-4*01 | IGKJ2*01 | QQSNEDPYT | 61 | 9 | 1 |
| 1 | IGKV3-4*01 | IGKJ2*01 | QQSNEDPYT | 61 | 9 | 2 |
| 1 | IGKV3-4*01 | IGKJ2*01 | QQSNEDPYT | 61 | 9 | 2 |
| 3 | IGKV3-4*01 | IGKJ2*01 | QQSNEDPYT | 61 | 9 | 0 |
| 4 | IGKV3-4*01 | IGKJ2*01 | QQSNEDPYT | 61 | 9 | 2 |
| 4 | IGKV3-4*01 | IGKJ2*01 | QQSNEDPYT | 61 | 9 | 0 |
| 6 | IGKV3-4*01 | IGKJ1*01 | QQSNEDPPWT | 65 | 10 | 1 |
| 6 | IGKV3-4*01 | IGKJ1*01 | QQSNEDPPWT | 65 | 10 | 2 |
| 6 | IGKV3-4*01 | IGKJ1*01 | QQGNEDPPWT | 66 | 10 | 1 |
| 6 | IGKV3-4*01 | IGKJ1*01 | QQSNEDPPWT | 65 | 10 | 2 |
| 6 | IGKV3-4*01 | IGKJ1*01 | QQSNEDPPWT | 65 | 10 | 2 |
| 6 | IGKV3-4*01 | IGKJ1*01 | HQSNEDPPWT | 67 | 10 | 2 |
| 6 | IGKV3-4*01 | IGKJ1*01 | QQINEDPPWT | 68 | 10 | 3 |
| 6 | IGKV3-4*01 | IGKJ1*01 | QQSNEDPPWT | 65 | 10 | 7 |
| 6 | IGKV3-4*01 | IGKJ1*01 | QQSNEDPPWT | 65 | 10 | 10 |
| 1 | IGKV3-4*01 | IGKJ1*01 | QQSNEDPPWT | 65 | 10 | 0 |
| 1 | IGKV3-4*01 | IGKJ1*01 | QQSNEDPPWT | 65 | 10 | 11 |
| 1 | IGKV3-4*01 | IGKJ1*01 | QQSYEDPPWT | 69 | 10 | 1 |
| 1 | IGKV3-4*01 | IGKJ1*01 | QQSNEDPPWT | 65 | 10 | 10 |
| 1 | IGKV3-4*01 | IGKJ1*01 | QQSYEDPPWT | 69 | 10 | 11 |
| 1 | IGKV3-4*01 | IGKJ1*01 | QQSNEDPPWT | 65 | 10 | 13 |
| 1 | IGKV3-4*01 | IGKJ1*01 | QQSNEDPPWT | 65 | 10 | 7 |
| 6 | IGKV3-4*01 | IGKJ1*01 | QQSNEDPWT | 70 | 9 | 1 |
| 6 | IGKV3-4*01 | IGKJ1*01 | QQSNEDPWT | 70 | 9 | 3 |
| 6 | IGKV3-4*01 | IGKJ1*01 | QQNNEDPWT | 71 | 9 | 3 |
| 6 | IGKV3-4*01 | IGKJ1*01 | QQSNEDPWT | 70 | 9 | 6 |
| 1 | IGKV3-4*01 | IGKJ1*01 | QQSNEDPWT | 70 | 9 | 3 |
| 1 | IGKV3-4*01 | IGKJ1*01 | QQSNEDPWT | 70 | 9 | 0 |
| 1 | IGKV3-4*01 | IGKJ1*01 | QQSNEDPWT | 70 | 9 | 8 |
| 1 | IGKV3-4*01 | IGKJ1*01 | QQSNEDPWT | 70 | 9 | 1 |
| 1 | IGKV3-4*01 | IGKJ1*01 | QQSNEDPWT | 70 | 9 | 15 |
| 1 | IGKV3-4*01 | IGKJ1*01 | QQSNEDPWT | 70 | 9 | 0 |
| 1 | IGKV3-4*01 | IGKJ1*01 | QQSNEDPWT | 70 | 9 | 4 |
| 1 | IGKV3-4*01 | IGKJ1*01 | QQSNEDPWT | 70 | 9 | 16 |
| 2 | IGKV3-4*01 | IGKJ1*01 | QQSNEDPWT | 70 | 9 | 1 |
| 2 | IGKV3-4*01 | IGKJ1*01 | QQSNEDPWT | 70 | 9 | 2 |
| 2 | IGKV3-4*01 | IGKJ1*01 | QQSNEDPWT | 70 | 9 | 5 |
| 6 | IGKV14-111*01 | IGKJ4*01 | LQYDEFPFT | 52 | 9 | 4 |
| 6 | IGKV14-111*01 | IGKJ4*01 | LQYDEFPFT | 52 | 9 | 2 |
| 6 | IGKV14-111*01 | IGKJ4*01 | LQYDEFTFT | 72 | 9 | 2 |
| 6 | IGKV14-111*01 | IGKJ4*01 | LQYDEFPFT | 52 | 9 | 8 |
| 1 | IGKV14-111*01 | IGKJ4*01 | LQYDEFPFT | 52 | 9 | 3 |
| 1 | IGKV14-111*01 | IGKJ4*01 | LQYDEFPFT | 52 | 9 | 0 |
| 1 | IGKV14-111*01 | IGKJ4*01 | LQYDEFPFT | 52 | 9 | 0 |
| 1 | IGKV14-111*01 | IGKJ4*01 | LQYDEFPFT | 52 | 9 | 0 |
| 1 | IGKV14-111*01 | IGKJ4*01 | LQYDEFPFT | 52 | 9 | 4 |
| 1 | IGKV14-111*01 | IGKJ4*01 | LQYDEFPFT | 52 | 9 | 3 |
| 6 | IGKV6-15*01 | IGKJ2*01 | QQYDSYPYT | 73 | 9 | 7 |
| 6 | IGKV6-15*01 | IGKJ2*01 | QQYNNYPYT | 74 | 9 | 2 |
| 6 | IGKV6-15*01 | IGKJ2*01 | QQYNSYPYT | 75 | 9 | 8 |
| 6 | IGKV6-15*01 | IGKJ2*01 | QQYNTYPYT | 76 | 9 | 10 |
| 6 | IGKV6-15*01 | IGKJ2*01 | QQYNSYPYT | 75 | 9 | 2 |
| 6 | IGKV6-15*01 | IGKJ2*01 | QQYNSYPYT | 75 | 9 | 8 |
| 6 | IGKV6-15*01 | IGKJ2*01 | QQYNIYPYT | 77 | 9 | 5 |
| 6 | IGKV6-15*01 | IGKJ2*01 | QQYNSYPYT | 75 | 9 | 6 |
| 6 | IGKV6-15*01 | IGKJ2*01 | QQYNSYPYT | 75 | 9 | 4 |
| 6 | IGKV6-15*01 | IGKJ2*01 | QQYNSYPYT | 75 | 9 | 2 |
| 1 | IGKV3-4*01 | IGKJ4*01 | QQSNEDPFT | 78 | 9 | 5 |
| 1 | IGKV3-4*01 | IGKJ4*01 | QQSNEDPFT | 78 | 9 | 2 |
| 1 | IGKV3-4*01 | IGKJ4*01 | QQSNEDPFT | 78 | 9 | 2 |
| 1 | IGKV3-4*01 | IGKJ4*01 | QQSNEDPFT | 78 | 9 | 5 |
| 2 | IGKV3-4*01 | IGKJ4*01 | QQSNEDPFT | 78 | 9 | 1 |
| 2 | IGKV3-4*01 | IGKJ4*01 | QQSNEDPFT | 78 | 9 | 1 |
| 2 | IGKV3-4*01 | IGKJ4*01 | QQSNEDPFT | 78 | 9 | 5 |
| 1 | IGKV14-111*01 | IGKJ2*01 | LQYDEYMYT | 79 | 9 | 2 |
| 1 | IGKV14-111*01 | IGKJ2*01 | LQYDEYMYT | 79 | 9 | 0 |
| 1 | IGKV14-111*01 | IGKJ2*01 | LQYDEYMYT | 79 | 9 | 0 |
| 1 | IGKV14-111*01 | IGKJ2*01 | LQYDEYMYT | 79 | 9 | 2 |
| 1 | IGKV10-96*01 | IGKJ1*01 | QQGNTLPWT | 80 | 9 | 1 |
| 1 | IGKV10-96*01 | IGKJ1*01 | QQGNTIPWT | 81 | 9 | 4 |
| 2 | IGKV10-96*01 | IGKJ1*01 | QQGNTLPRT | 82 | 9 | 1 |
| 2 | IGKV10-96*01 | IGKJ1*01 | QQGNTLPRT | 82 | 9 | 5 |
| 6 | IGKV1-110*01 | IGKJ1*01 | SQSTHVPT | 83 | 8 | 3 |
| 6 | IGKV1-110*01 | IGKJ1*01 | SQSTHVPT | 83 | 8 | 0 |
| 6 | IGKV1-110*01 | IGKJ1*01 | SQSTHVPT | 83 | 8 | 2 |
| 2 | IGKV14-100*01 | IGKJ5*01 | VQYVQFPLT | 84 | 9 | 2 |
| 2 | IGKV14-100*01 | IGKJ5*01 | VQYAQFPLT | 85 | 9 | 2 |
| 2 | IGKV14-100*01 | IGKJ5*01 | VQYAQFPLT | 85 | 9 | 1 |
| 3 | IGKV3-4*01 | IGKJ2*01 | QQSNEDPYT | 61 | 9 | 2 |
| 3 | IGKV3-4*01 | IGKJ2*01 | QQSNEDPYT | 61 | 9 | 1 |
| 1 | IGKV1-117*01 | IGKJ1*01 | FQGSHVPWT | 86 | 9 | 2 |
| 1 | IGKV1-117*01 | IGKJ1*01 | FQGSHVPWT | 86 | 9 | 1 |
| 3 | IGKV14-111*01 | IGKJ2*01 | LQYDEFPYT | 50 | 9 | 3 |
| 3 | IGKV14-111*01 | IGKJ2*01 | LQYDEFPYT | 50 | 9 | 2 |
| 1 | IGKV14-100*01 | IGKJ4*01 | VQYAQFPFT | 87 | 9 | 4 |
| 1 | IGKV14-126*01 | IGKJ2*01 | LQHGESPYT | 88 | 9 | 0 |
| 6 | IGKV4-50*01 | IGKJ2*01 | QQFTSSPYT | 89 | 9 | 2 |
| 2 | IGKV10-96*01 | IGKJ2*01 | QQGNTLPYT | 90 | 9 | 3 |
| 1 | IGKV14-111*01 | IGKJ2*01 | LQYDEFRTT | 91 | 9 | 5 |
| 4 | IGKV6-15*01 | IGKJ5*01 | QQYNSYPFT | 92 | 9 | 1 |
| 2 | IGKV4-62*01 | IGKJ5*01 | QQCSGYPLT | 93 | 9 | 3 |
| 6 | IGKV10-94*01 | IGKJ1*01 | QQYSKLPWT | 94 | 9 | 1 |
| 2 | IGKV14-111*01 | IGKJ2*01 | LQYDEFPYT | 50 | 9 | 4 |
| 2 | IGKV3-4*01 | IGKJ4*01 | QQSNEDPFT | 78 | 9 | 3 |
| 3 | IGKV3-4*01 | IGKJ2*01 | QQSNEDPYT | 61 | 9 | 3 |
| 2 | IGKV14-111*01 | IGKJ2*01 | LQYDEFPPFT | 95 | 10 | 2 |
| 2 | IGKV14-111*01 | IGKJ4*01 | LQYDEFPFT | 52 | 9 | 5 |

TABLE 6
Sequences of Antibodies Generated from RC1- and RC1-4fill-immunized Mice.

| | | | | | SEQ | | | | SEQ | | |
|---|---|---|---|---|---|---|---|---|---|---|---|
| ANTI- | | | | | ID | LENGTH | | | ID | LENGTH | RC1 |
| BODY | MOUSE | IMM. | VH | CDRH3 | NO: | (AA). | VK | CDRL3 | NO: | (AA). | BINDING |
| 271 | 1 | RC1 | IGHV5-6*01 | ARHSRTGTGAMDY | 96 | 13 | IGKV3-4*01 | QQSNEDPPWT | 65 | 10 | YES |
| 340 | 2 | RC1 | IGHV1-81*01 | ARPYYYGSSPYFDY | 97 | 14 | IGKV4-57*01 | QQRSSYPPT | 109 | 9 | NO |
| 341 | 2 | RC1 | IGHV5-17*01 | ARSIVPDY | 98 | 8 | IGKV14-100*01 | VQYVQFPLT | 84 | 9 | YES* |
| 343 | 2 | RC1 | IGHV5-6*01 | ASLYGNAFDY | 99 | 10 | IGKV3-4*01 | QQSNEDPFT | 78 | 9 | YES |
| 344 | 2 | RC1 | IGHV9-3*01 | ASGGNYFDY | 100 | 9 | IGKV14-111*01 | LQYDEFPPFT | 95 | 10 | YES |
| 346 | 2 | RC1 | IGHV5-6*01 | ARHVGDHAMDY | 101 | 11 | IGKV3-4*01 | QQSNEDPFT | 78 | 9 | YES |
| 347 | 2 | RC1 | IGHV1-81*01 | ARPYYYGSSPNFDY | 102 | 14 | IGKV3-4*01 | QQSNEDPWT | 70 | 9 | NO |
| 351 | 3 | RC1 | IGHV9-3*01 | GTGKNYFDH | 103 | 9 | IGKV14-111*01 | LQYDEFPYT | 50 | 9 | YES |
| 352 | 3 | RC1 | IGHV5-6*01 | ATNYGAWFPY | 104 | 10 | IGKV3-4*01 | QQSNEDPYT | 61 | 9 | YES |
| 274 | 4 | RC1 | IGHV5-6*01 | ARHGITTVGVAMDY | 105 | 14 | IGKV3-4*01 | QQSNEDPWT | 70 | 9 | YES |
| 275 | 4 | RC1 | IGHV5-6*01 | ARHGITTVGVAMDY | 105 | 14 | IGKV3-4*01 | QQSNEDPYT | 61 | 9 | YES |
| 276 | 6 | RC1-4 | IGHV5-6*01 | ARHGRLTGTGAMDY | 40 | 14 | IGKV3-4*01 | QQSNEDPPWT | 65 | 10 | YES |
| 278 | 6 | RC1-4 | IGHV5-6*01 | ARHGRLTGTGAMDY | 40 | 14 | IGKV3-4*01 | HQSNEDPPWT | 67 | 10 | YES |
| 280 | 6 | RC1-4 | IGHV5-6*01 | ARHGHYYGSSYGMDY | 46 | 15 | IGKV3-4*01 | QQSNEDPPWT | 65 | 10 | YES |
| 294 | 6 | RC1-4 | IGHV2-9*01 | ANIPKDRLCYG | 106 | 11 | IGKV3-4*01 | QQSNEDPWT | 70 | 9 | YES |
| 348 | NS | RC1 | IGHV1-62-2*01 | ARHEGNYLYAMDY | 107 | 13 | IGKV4-62*01 | QQCSGYPLT | 93 | 9 | YES |
| 349 | NS | RC1 | IGHV1-7*01 | ARPPFITVVANYFDY | 108 | 15 | IGKV10-94*01 | QQYSKLPWT | 94 | 9 | YES |

TABLE 7
Sequences of Antibodies Generated from RC1-4fill VLPs-immunized macaques.
NHP 1

| | | | | SEQ | LENGTH | Nt |
|---|---|---|---|---|---|---|
| VH | DH | JH | CDRH3 | ID NO: | (AA) | mut. |
| IGHV4_11* | IGHD4- | IGHJ4* | ARVVNYGPLDY | 110 | 11 | 3 |
| S4129 | 1*01 | 01 | | | | |
| IGHV4_11* | IGHD3- | IGHJ4* | ARVVKNGPLDY | 111 | 11 | 6 |
| S4129 | 2*01 | 01 | | | | |
| IGHV4_11* | IGHD3- | IGHJ4* | ARVVKYGPLDY | 112 | 11 | 3 |
| S4129 | 1*01 | 01 | | | | |
| IGHV4_11* | IGHD3- | IGHJ4* | ARLVRYGPLDY | 113 | 11 | 7 |
| S4129 | 1*01 | 01 | | | | |
| IGHV4_11* | IGHD3- | IGHJ4* | ARIVKYGPLDF | 114 | 11 | 6 |
| S4129 | 1*01 | 01 | | | | |
| IGHV4_11* | IGHD3- | IGHJ4* | ARVVKYGPLDY | 112 | 11 | 4 |
| S4129 | 1*010 | 01 | | | | |
| IGHV4_11* | IGHD3- | IGHJ4* | ARVVKYGPLDY | 112 | 11 | 2 |
| S4129 | 1*01 | 01 | | | | |
| IGHV4_11* | IGHD1- | IGHJ4* | ARGSRIAPFDY | 115 | 11 | 7 |
| S0762 | 2*01 | 01 | | | | |
| IGHV4_11* | IGHD1- | IGHJ4* | ARGSRIAPFDY | 115 | 11 | 5 |
| S0762 | 2*01 | 01 | | | | |
| IGHV4_11* | IGHD1- | IGHJ4* | ARGSRIAPFDH | 116 | 11 | 7 |
| S0762 | 2*01 | 01 | | | | |
| IGHV4_11* | IGHD1- | IGHJ4* | ARGSRIAPFDY | 115 | 11 | 9 |
| S0762 | 2*01 | 01 | | | | |
| IGHV4_11* | IGHD3- | IGHJ4* | SRYQARGPIDS | 117 | 11 | 3 |
| S4129 | 3*01 | 01 | | | | |
| IGHV4_11* | IGHD3- | IGHJ4* | ARDQARGPIDY | 118 | 11 | 4 |
| S4129 | 3*01 | 01 | | | | |
| IGHV4_11* | IGHD3- | IGHJ4* | ARNQARGPIDY | 119 | 11 | 27 |
| S4129 | 3*01 | 01 | | | | |
| IGHV4_11* | IGHD3- | IGHJ4* | ARDQARGPIDY | 118 | 11 | 9 |
| S4129 | 3*01 | 01 | | | | |
| IGHV4_2C* | IGHD1- | IGHJ4* | ARDNRIGPFDY | 120 | 11 | 6 |
| F124 | 3*01 | 01 | | | | |
| IGHV4_2C* | IGHD1- | IGHJ4* | ARDNRIGPFDY | 120 | 11 | 6 |
| F124 | 3*01 | 01 | | | | |
| IGHV4_2C* | IGHD1- | IGHJ4* | ARDNRIGPFDY | 120 | 11 | 4 |
| F124 | 3*01 | 01 | | | | |
| IGHV4_2C* | IGHD2- | IGHJ4* | ARDKRIGPFDY | 121 | 11 | 6 |
| F124 | 1*01 | 01 | | | | |
| IGHV3_4I* | IGHD6- | IGHJ4* | AKKRRQLENDY | 122 | 11 | 4 |
| F130 | 3*01 | 01 | | | | |
| IGHV3_4I* | IGHD6- | IGHJ4* | VKKRRQLENDY | 123 | 11 | 4 |
| F130 | 3*01 | 01 | | | | |
| IGHV3_4I* | IGHD6- | IGHJ4* | AKKRRQLENDY | 122 | 11 | 6 |
| F130 | 3*01 | 01 | | | | |
| IGHV3_4I* | IGHD6- | IGHJ4* | VKKRRQLENDY | 123 | 11 | 4 |
| F130 | 3*01 | 01 | | | | |
| IGHV4_11* | IGHD6- | IGHJ4* | ASRIAGGPFDY | 124 | 11 | 4 |
| S4664 | 2*01 | 01 | | | | |
| IGHV4_11* | IGHD6- | IGHJ4* | ASRIAGGPFDF | 125 | 11 | 8 |
| S4664 | 2*01 | 01 | | | | |
| IGHV4_11* | IGHD6- | IGHJ4* | ASLIAAGPFDY | 126 | 11 | 8 |
| S4664 | 2*01 | 01 | | | | |
| IGHV4_11* | IGHD1- | IGHJ4* | ASRIRGGPFDY | 127 | 11 | 0 |
| S4664 | 3*01 | 01 | | | | |
| IGHV4_3M* | IGHD4- | IGHJ4* | ARDIVVGPIDY | 128 | 11 | 7 |
| F133 | 2*01 | 01 | | | | |
| IGHV4_3M* | IGHD2- | IGHJ4* | ARDIVIGPIDY | 129 | 11 | 11 |
| F133 | 5*01 | 01 | | | | |
| IGHV4_3M* | IGHD2- | IGHJ4* | ARDIVIGPIDY | 129 | 11 | 6 |
| F133 | 5*01 | 01 | | | | |
| IGHV4_11* | IGHD6- | IGHJ4* | ATVGRLAPFDY | 130 | 11 | 5 |
| S4129 | 1*01 | 01 | | | | |
| IGHV4_11* | IGHD2- | IGHJ4* | ARVGRVVPFDY | 131 | 11 | 5 |
| S4129 | 2*01 | 01 | | | | |
| IGHV4_11* | IGHD6- | IGHJ4* | ARVGRVAPFDY | 132 | 11 | 6 |
| S4129 | 5*01 | 01 | | | | |
| IGHV3_2N* | IGHD6- | IGHJ1* | AKSPWGQSSSFEYFE | 133 | 16 | 4 |
| F134 | 6*01 | 01 | F | | | |
| IGHV3_2N* | IGHD6- | IGHJ1* | AKSPWGQSTSFEYFE | 134 | 16 | 5 |
| F134 | 6*01 | 01 | F | | | |
| IGHV3_2N* | IGHD4- | IGHJ1* | AKSPWGQSSYFEYF | 135 | 16 | 3 |
| F134 | 1*01 | 01 | EF | | | |
| IGHV3_45* | IGHD1- | IGHJ5- | ASVLWGLPQDDNSL | 136 | 16 | 6 |
| S5348 | 8*01 | 2*02 | DV | | | |
| IGHV3_45* | IGHD1- | IGHJ5- | ASVLWEVPQDDNSL | 137 | 16 | 3 |
| S5348 | 8*01 | 2*02 | DV | | | |
| IGHV3_45* | IGHD1- | IGHJ5- | ANVLWGLPQDDNSL | 138 | 16 | 2 |
| S5348 | 8*01 | 2*02 | DV | | | |
| IGHV4_2C* | IGHD6- | IGHJ4* | ASLQRLGPIDY | 139 | 11 | 6 |
| F124 | 1*01 | 01 | | | | |
| IGHV4_2C* | IGHD6- | IGHJ4* | ASLQRLGPIDY | 139 | 11 | 4 |
| F124 | 1*01 | 01 | | | | |
| IGHV4_2C* | IGHD6- | IGHJ4* | ASLQRLGPIDY | 139 | 11 | 2 |
| F124 | 1*01 | 01 | | | | |
| IGHV4_11* | IGHD3- | IGHJ1* | ASLQYFGPFEF | 140 | 11 | 0 |
| S4129 | 4*01 | 01 | | | | |
| IGHV4_11* | IGHD3- | IGHJ1* | ASLQYFGPFDF | 141 | 11 | 5 |
| S4129 | 4*01 | 01 | | | | |
| IGHV4_11* | IGHD6- | IGHJ4* | ARAERAGPFDY | 142 | 11 | 10 |
| S4129 | 1*01 | 01 | | | | |
| IGHV4_11* | IGHD6- | IGHJ4* | ARAERAGPFDY | 142 | 11 | 5 |
| S4129 | 1*01 | 01 | | | | |
| IGHV3_45* | IGHD1- | IGHJ4* | ARHPHLESFDY | 143 | 11 | 4 |
| S5348 | 1*01 | 01 | | | | |
| IGHV3_45* | IGHD1- | IGHJ4* | ARHPHLESFDY | 143 | 11 | 2 |
| S5348 | 1*01 | 01 | | | | |
| IGHV4_11* | IGHD4- | IGHJ1* | ARNYGNYGYFEF | 144 | 12 | 5 |
| S4129 | 3*01 | 01 | | | | |
| IGHV4_11* | IGHD4- | IGHJ1* | ARNYGNYGYFEF | 144 | 12 | 2 |
| S4129 | 3*01 | 01 | | | | |
| IGHV1_53* | IGHD3- | IGHJ1* | ATGPYWGDYYGRY | 145 | 16 | 2 |
| S2078 | 3*01 | 01 | FEL | | | |
| IGHV1_53* | IGHD3- | IGHJ1* | ATGPYWGDYYGRY | 146 | 16 | 2 |
| S2078 | 3*01 | 01 | FEF | | | |
| IGHV4_11* | IGHD6- | IGHJ4* | ATERRAGPVDY | 147 | 11 | 4 |
| S4129 | 3*01 | 01 | | | | |
| IGHV4_11* | IGHD2- | IGHJ4* | ATDRRAGPLDY | 148 | 11 | 2 |
| S4129 | 5*01 | 01 | | | | |
| IGHV3_1E* | IGHD1- | IGHJ5- | AGTLAGTTSFDV | 149 | 12 | 11 |
| F130 | 2*01 | 1*01 | | | | |
| IGHV3_1E* | IGHD1- | IGHJ5- | AGGLGRTTSFDV | 150 | 12 | 14 |
| F130 | 7*01 | 1*01 | | | | |
| IGHV4_11* | IGHD6- | IGHJ4* | ARVGSGWSTEGNFD | 151 | 15 | 4 |
| S3777 | 1*01 | 01 | Y | | | |
| IGHV4_11* | IGHD6- | IGHJ4* | ARVGSGWSTEGNFD | 151 | 15 | 2 |
| S3777 | 1*01 | 01 | Y | | | |
| IGHV3_2N* | IGHD4- | IGHJ4* | AKDWIQWLHLGSYF | 152 | 16 | 6 |
| F134 | 2*01 | 01 | DF | | | |
| IGHV3_2N* | IGHD4- | IGHJ4* | AKDWIQWVHLGSYF | 153 | 16 | 3 |
| F134 | 2*01 | 01 | DY | | | |
| IGHV4_11* | IGHD4- | IGHJ4* | ARHSSTYVAPVDY | 154 | 13 | 7 |
| S4664 | 1*01 | 01 | | | | |
| IGHV4_2C* | IGHD3- | IGHJ4* | ASAKGRLAPLDY | 155 | 12 | 8 |
| F124 | 1*01 | 01 | | | | |
| IGHV4_11* | IGHD3- | IGHJ4* | ANWADYFDY | 156 | 9 | 1 |
| S5305 | 3*01 | 01 | | | | |
| IGHV4_2C* | IGHD3- | IGHJ5- | ARDPVITITTRERFD | 157 | 16 | 10 |
| F124 | 4*01 | 1*01 | V | | | |
| IGHV4_11* | IGHD6- | IGHJ4* | ARDQRTGPFDY | 158 | 11 | 1 |
| S4129 | 1*01 | 01 | | | | |
| IGHV4_11* | IGHD1- | IGHJ6* | ARQAFAGPTDS | 159 | 11 | 6 |
| S4129 | 1*01 | 01 | | | | |
| IGHV4_11* | IGHD1- | IGHJ5- | ARRGPVNWNGSSLD | 160 | 15 | 4 |
| S0762 | 3*01 | 2*02 | V | | | |
| IGHV3_1W* | IGHD1- | IGHJ4* | TRDRADSWNFHDYF | 161 | 16 | 3 |
| F134 | 1*01 | 01 | DY | | | |
| IGHV4_11* | IGHD6- | IGHJ4* | AKIAVAGPVDY | 162 | 11 | 4 |
| S5305 | 5*01 | 01 | | | | |
| IGHV4_2C* | IGHD2- | IGHJ5- | ATTYSGSDYYRLDV | 163 | 14 | 6 |
| F124 | 3*01 | 2*02 | | | | |
| IGHV1_2B* | IGHD3- | IGHJ4* | ARPDSLWGAAFDY | 164 | 13 | 4 |
| F134 | 3*01 | 01 | | | | |
| IGHV4_11* | IGHD6- | IGHJ4* | ARIGAAGPGDY | 165 | 11 | 11 |
| S4664 | 2*01 | 01 | | | | |
| IGHV4_11* | IGHD3- | IGHJ5- | AKYWGDYYGYSSL | 166 | 15 | 6 |
| S9724 | 3*01 | 2*02 | DV | | | |
| IGHV4_11* | IGHD1- | IGHJ4* | ARVEVVGPTGY | 167 | 11 | 9 |
| S4664 | 8*01 | 01 | | | | |
| IGHV4_3M* | IGHD2- | IGHJ4* | ARRYSGSYSPFDC | 168 | 13 | 3 |
| F133 | 4*01 | 01 | | | | |
| IGHV4_11* | IGHD2- | IGHJ4* | AREGMGCTGSGCSIS | 169 | 18 | 0 |
| S6427 | 5*01 | 01 | FDY | | | |
| IGHV4_11* | IGHD5- | IGHJ4* | ARQGYSGYSLFDY | 170 | 13 | 7 |
| S9724 | 3*01 | 01 | | | | |
| IGHV4_11* | IGHD6- | IGHJ4* | ASEIAGGPVDY | 171 | 11 | 3 |
| S4664 | 2*01 | 01 | | | | |
| IGHV4_11* | IGHD6- | IGHJ5- | ARDSSGWPWDNRFD | 172 | 15 | 4 |
| S5305 | 1*01 | 1*01 | V | | | |
| IGHV4_11* | IGHD2- | IGHJ4* | ARVTGRIAPFDY | 173 | 12 | 4 |
| S4129 | 3*01 | 01 | | | | |
| IGHV5_1A* | IGHD3- | IGHJ6* | ATNIWTGYSFYYGL | 174 | 16 | 18 |
| F124 | 2*01 | 01 | DS | | | |
| IGHV4_11* | IGHD3- | IGHJ6* | AREGRIHPLDS | 175 | 11 | 31 |
| S4129 | 1*01 | 01 | | | | |
| IGHV3_4I* | IGHD4- | IGHJ6* | AKDHDYGGGLDS | 176 | 12 | 3 |
| F130 | 1*01 | 01 | | | | |
| IGHV3_4I* | IGHD6- | IGHJ4* | AKKSSGSWEVDY | 177 | 12 | 5 |
| F130 | 3*01 | 01 | | | | |
| IGHV4_11* | IGHD3- | IGHJ5- | ARHAYYNIWTGYST | 178 | 19 | 0 |
| S5891 | 4*01 | 1*01 | NRFDV | | | |
| IGHV5_1A* | IGHD6- | IGHJ5- | AEGSGSWNGRFGV | 179 | 13 | 3 |
| F124 | 3*01 | 1*01 | | | | |
| IGHV1_53* | IGHD3- | IGHJ5- | ATGRYYGGSYYGDR | 180 | 17 | 7 |
| S2078 | 2*01 | 1*01 | FDV | | | |
| IGHV3_4I* | IGHD6- | IGHJ4* | AKCSSSSTGLDY | 181 | 12 | 3 |
| F130 | 6*01 | 01 | | | | |
| IGHV1_2B* | IGHD1- | IGHJ4* | ARDRSVTPFSWVEY | 182 | 18 | 6 |
| F134 | 7*01 | 01 | YFDY | | | |
| IGHV4_5L* | IGHD6- | IGHJ4* | VRVVKYGPLDY | 183 | 11 | 2 |
| F134 | 2*01 | 01 | | | | |
| IGHV4_11* | IGHD3- | IGHJ5- | ARNPPYYNLWTGYY | 184 | 20 | 2 |
| S3915 | 2*01 | 2*02 | THSLDV | | | |
| IGHV4_1F* | IGHD3- | IGHJ4* | ARVVKYGPLDY | 112 | 11 | 1 |
| F130 | 1*01 | 01 | | | | |
| IGHV4_11* | IGHD2- | IGHJ4* | AREGYCSYTYCSNL | 185 | 17 | 4 |
| S3915 | 3*01 | 01 | FEF | | | |
| IGHV4_2C* | IGHD6- | IGHJ4* | ARARIAAPFDY | 186 | 11 | 6 |
| F124 | 1*01 | 01 | | | | |
| IGHV4_11* | IGHD1- | IGHJ4* | ARAGRMAATDY | 187 | 11 | 5 |
| S4664 | 8*01 | 01 | | | | |
| IGHV4_11* | IGHD1- | IGHJ4* | VRDVTLGPIDN | 188 | 11 | 3 |
| S4129 | 8*01 | 01 | | | | |
| IGHV4_11* | IGHD4- | IGHJ6* | AREGRIQPLDS | 189 | 11 | 4 |
| S4129 | 4*01 | 01 | | | | |
| IGHV3_4I* | IGHD1- | IGHJ4* | AKCRNWNDFAY | 190 | 11 | 4 |
| F130 | 3*01 | 01 | | | | |
| IGHV4_2C* | IGHD6- | IGHJ4* | ARVHRGGPFDY | 191 | 11 | 9 |
| F124 | 1*01 | 01 | | | | |
| IGHV4_11* | IGHD1- | IGHJ4* | ARGGRVHPMDY | 192 | 11 | 6 |
| S4129 | 1*01 | 01 | | | | |
| IGHV4_11* | IGHD3- | IGHJ4* | ARGGPVSPFDY | 193 | 11 | 9 |
| S4129 | 3*01 | 01 | | | | |
| IGHV4_11* | IGHD4- | IGHJ5- | ARGQRVAPFDV | 194 | 11 | 8 |
| S4664 | 2*01 | 1*01 | | | | |
| IGHV5_1A* | IGHD3- | IGHJ5- | AKETYEDDYGYYSL | 195 | 21 | 2 |
| F124 | 1*01 | 1*01 | GYNRFDV | | | |
| IGHV5_1F* | IGHD1- | IGHJ4* | ASAWREHLPIDY | 196 | 12 | 7 |
| F134 | 7*01 | 01 | | | | |
| IGHV3_1Z* | IGHD3- | IGHJ6* | ARDLYPGVINPSGLD | 197 | 16 | 4 |
| F134 | 3*01 | 01 | S | | | |

Table 7 (Continued)
Sequences of Antibodies Generated from RC1-4fill VLPs-immunized macaques.

| | | | | | | |
|---|---|---|---|---|---|---|
| | | | | SEQ ID | LENGTH | Nt |
| VH | DH | JH | CDRH3 | NO: | (AA) | mut. |

NHP 5

| | | | | | | |
|---|---|---|---|---|---|---|
| IGHV3_3F* | IGHD3- | IGHJ1* | ARDKGSSYYQPEYFEF | 198 | 16 | 9 |
| F132 | 2*01 | 01 | | | | |
| IGHV3_3F* | IGHD3- | IGHJ1* | ARDKGSSYYQPEYFEF | 198 | 16 | 10 |
| F132 | 2*01 | 01 | | | | |
| IGHV3_3F* | IGHD3- | IGHJ1* | VRDKGSSYYQPEYFEF | 199 | 16 | 7 |
| F132 | 2*01 | 01 | | | | |
| IGHV4_1M* | IGHD6- | IGHJ4* | ARTGKAAPVDY | 200 | 11 | 11 |
| F130 | 3*01 | 01 | | | | |
| IGHV4_1M* | IGHD6- | IGHJ4* | ARTGKAAPVDY | 200 | 11 | 11 |
| F130 | 3*01 | 01 | | | | |
| IGHV4_1M* | IGHD6- | IGHJ4* | ARTGKAAPVDC | 201 | 11 | 7 |
| F130 | 3*01 | 01 | | | | |
| IGHV5_1C* | IGHD3- | IGHJ4* | AKGGDNYYDSGYYDDY | 202 | 16 | 0 |
| F130 | 2*01 | 01 | | | | |
| IGHV4_3N* | IGHD3- | IGHJ4* | ARNRGWGDLVFDY | 203 | 13 | 3 |
| F133 | 3*01 | 01 | | | | |
| IGHV5_1H* | IGHD6- | IGHJ4* | AKVLSGWFWDYFDY | 204 | 14 | 8 |
| F132 | 1*01 | 01 | | | | |
| IGHV4_11* | IGHD6- | IGHJ4* | ARLAVAGPVDY | 205 | 11 | 5 |
| S4664 | 5*01 | 01 | | | | |
| IGHV3_3F* | IGHD6- | IGHJ6* | ARGSSGWYGSGLDS | 206 | 14 | 7 |
| F132 | 1*01 | 01 | | | | |
| IGHV4_1U* | IGHD1- | IGHJ5- | ARDHIESWNKVNWFDV | 207 | 16 | 7 |
| F130 | 1*01 | 1*01 | | | | |
| IGHV1_1G* | IGHD6- | IGHJ1* | ATYSGSWYAEYFEF | 208 | 14 | 1 |
| F133 | 3*01 | 01 | | | | |
| IGHV5_1F* | IGHD2- | IGHJ4* | AKQEDYNFWSSYFLPDY | 209 | 17 | 1 |
| F134 | 3*01 | 01 | | | | |
| IGHV1_1G* | IGHD6- | IGHJ4* | ARDSSGWYEGFDY | 210 | 13 | 1 |
| F133 | 1*01 | 01 | | | | |
| IGHV1_53* | IGHD3- | IGHJ4* | ATGRYYGPSWAIFDY | 211 | 15 | 3 |
| S2078 | 1*01 | 01 | | | | |
| IGHV4_11* | IGHD1- | IGHJ4* | ARDGNFGPIDY | 212 | 11 | 4 |
| S4290 | 8*01 | 01 | | | | |
| IGHV7_1A* | | IGHJ5- | ASGPNWFDV | 213 | 9 | 7 |
| F124 | | 1*01 | | | | |
| IGHV5_1C* | IGHD2- | IGHJ4* | AKSETDFWTSYYFNY | 214 | 15 | 8 |
| F130 | 5*01 | 01 | | | | |
| IGHV4_2M* | IGHD2- | IGHJ5- | ARDICSGSGCYWYRDN | 215 | 20 | 1 |
| F130 | 5*01 | 1*01 | WFDV | | | |
| IGHV4_1T* | IGHD2- | IGHJ4* | ASNRRIAPLDY | 216 | 11 | 6 |
| F130 | 1*01 | 01 | | | | |
| IGHV7_1A* | IGHD3- | IGHJ4* | ASGRYYFDY | 217 | 9 | 5 |
| F124 | 1*01 | 01 | | | | |
| IGHV3_3F* | IGHD4- | IGHJ1* | ARDRTVTPNRGYFEF | 218 | 15 | 8 |
| F132 | 3*01 | 01 | | | | |
| IGHV1_2B* | IGHD6- | IGHJ6* | ARDGPYSGGWSELDS | 219 | 15 | 1 |
| F134 | 5*01 | 01 | | | | |
| IGHV4_5F* | IGHD6- | IGHJ4* | ARWEYSGNWGLDY | 220 | 13 | 22 |
| F132 | 3*01 | 01 | | | | |
| IGHV4_11* | IGHD6- | IGHJ3* | ARSTSSWPRTSDAFDF | 221 | 16 | 1 |
| S5305 | 2*01 | 01 | | | | |
| IGHV3_4I* | IGHD6- | IGHJ4* | AKKRSSWSRIDY | 222 | 12 | 1 |
| F130 | 2*01 | 01 | | | | |
| IGHV3_3F* | IGHD6- | IGHJ4* | ARDGSGWRRVTFDY | 223 | 14 | 10 |
| F132 | 1*01 | 01 | | | | |
| IGHV7_1A* | IGHD6- | IGHJ4* | ATGRNYFDY | 224 | 9 | 4 |
| F124 | 1*01 | 01 | | | | |
| IGHV3_4I* | IGHD4- | IGHJ4* | AKTGAVTTGFDY | 225 | 12 | 4 |
| F130 | 4*01 | 01 | | | | |
| IGHV4_11* | IGHD3- | IGHJ4* | ARLVGGSGYYYIGD | 226 | 14 | 0 |
| S0762 | 2*01 | 01 | | | | |
| IGHV3_1B* | IGHD6- | IGHJ4* | AKVPYSSWSHFDY | 227 | 13 | 6 |
| F124 | 2*01 | 01 | | | | |
| IGHV3_2M* | IGHD6- | IGHJ4* | TSPRMRYSSGSFDY | 228 | 14 | 3 |
| F132 | 1*01 | 01 | | | | |
| IGHV1_2B* | IGHD4- | IGHJ4* | ARVRGYSGYSFFDY | 229 | 14 | 0 |
| F134 | 2*01 | 01 | | | | |
| IGHV3_4S* | IGHD6- | IGHJ5- | SRGSTWSGDWFDV | 230 | 13 | 7 |
| F133 | 2*01 | 1*01 | | | | |
| IGHV3_3O* | IGHD4- | IGHJ5- | TKRLAYSNPYNRFDV | 231 | 15 | 2 |
| F130 | 4*01 | 1*01 | | | | |
| IGHV3_2W* | IGHD3- | IGHJ4* | ARGGVGLDDVTYYYSGS | 232 | 27 | 1 |
| F134 | 2*01 | 01 | YYYHRTSFDY | | | |
| IGHV4_2M* | IGHD5- | IGHJ5- | AGDRGGYNYGFTDNWF | 233 | 18 | 5 |
| F130 | 2*01 | 1*01 | DV | | | |
| IGHV3_2C* | IGHD3- | IGHJ4* | TRGTAYYNFWSNSSPGY | 234 | 20 | 3 |
| F133 | 4*01 | 01 | FDY | | | |
| IGHV3_4V* | IGHD3- | IGHJ1* | ARDKGSSYYQPESFEF | 235 | 16 | 8 |
| F133 | 2*01 | 01 | | | | |
| IGHV4_1N* | IGHD3- | IGHJ4* | ARRYYEDDYGYYYPGP | 236 | 27 | 6 |
| F130 | 1*01 | 01 | NIAGTTRGVEE | | | |
| IGHV4_1I* | IGHD6- | IGHJ3* | ARSTSSWPRTSDAFDF | 221 | 16 | 1 |
| F130 | 2*01 | 01 | | | | |

NHP 6

| | | | | | | |
|---|---|---|---|---|---|---|
| IGHV3_45* | IGHD3- | IGHJ5- | ARGITRMITVTKTNWFD | 237 | 18 | 4 |
| S5257 | 1*01 | 1*01 | V | | | |
| IGHV3_45* | IGHD3- | IGHJ5- | ARGITRMITVTKTNWFD | 237 | 18 | 1 |
| S5257 | 1*01 | 1*01 | V | | | |
| IGHV3_45* | IGHD3- | IGHJ5- | ARGITRMITVTKTNWFD | 237 | 18 | 1 |
| S5257 | 1*01 | 1*01 | V | | | |
| IGHV3_45* | IGHD3- | IGHJ5- | ARGITRMITVTKTNWFD | 237 | 18 | 6 |
| S5257 | 1*01 | 1*01 | V | | | |
| IGHV4_11* | IGHD6- | IGHJ4* | ARLAVAGPFDY | 238 | 11 | 5 |
| S5305 | 5*01 | 01 | | | | |
| IGHV4_11* | IGHD6- | IGHJ4* | ARLGVAGPLDY | 239 | 11 | 2 |
| S5305 | 5*01 | 01 | | | | |
| IGHV1_2B* | IGHD1- | IGHJ4* | ATYKTIDY | 240 | 8 | 3 |
| F134 | 8*01 | 01 | | | | |
| IGHV1_2B* | IGHD2- | IGHJ4* | ASYKNIDY | 241 | 8 | 1 |
| F134 | 3*01 | 01 | | | | |
| IGHV4_11* | IGHD4- | IGHJ4* | ARDRHGIPFDY | 242 | 11 | 2 |
| S9280 | 1*01 | 01 | | | | |
| IGHV1_2B* | IGHD3- | IGHJ4* | ARSRGYWGDLFDF | 243 | 13 | 0 |
| F134 | 3*01 | 01 | | | | |
| IGHV3_45* | IGHD3- | IGHJ4* | ARLSGWGDFRIDY | 244 | 13 | 2 |
| S5348 | 3*01 | 01 | | | | |
| IGHV1_53* | IGHD2- | IGHJ5- | ATGIWFDV | 245 | 8 | 4 |
| S2078 | 1*01 | 1*01 | | | | |
| IGHV3_45* | IGHD2- | IGHJ4* | ARANNGGYFDY | 246 | 11 | 6 |
| S5257 | 3*01 | 01 | | | | |
| IGHV1_2B* | IGHD4- | IGHJ2* | ARMTTVAAFGGYFDL | 247 | 15 | 5 |
| F134 | 1*01 | 01 | | | | |
| IGHV7_1A* | IGHD1- | IGHJ4* | ASGGNYADY | 248 | 9 | 1 |
| F124 | 8*01 | 01 | | | | |
| IGHV4_11* | IGHD6- | IGHJ4* | ARRLSRRYFDY | 249 | 11 | 0 |
| S4664 | 3*01 | 01 | | | | |
| IGHV3_2L* | IGHD2- | IGHJ4* | TREFCSGIYCYAPFDY | 250 | 16 | 0 |
| F132 | 4*01 | 01 | | | | |
| IGHV1_2B* | IGHD3- | IGHJ5- | ASFKTLDV | 251 | 8 | 3 |
| F134 | 4*01 | 2*02 | | | | |
| IGHV5_1F* | IGHD5- | IGHJ4* | AKGVGGFSYSYPHY | 252 | 14 | 8 |
| F134 | 1*01 | 01 | | | | |
| IGHV3_45* | IGHD3- | IGHJ4* | ARDGHYNFWSPPGY | 253 | 14 | 5 |
| S5257 | 4*01 | 01 | | | | |
| IGHV4_11* | IGHD3- | IGHJ5- | ARAEDEDDYGSFDV | 254 | 14 | 6 |
| S9724 | 1*01 | 1*01 | | | | |
| IGHV4_11* | IGHD6- | IGHJ3* | ARLGSSGWYRDDAFDF | 255 | 16 | 3 |
| S5305 | 1*01 | 01 | | | | |
| IGHV3_1C* | IGHD6- | IGHJ4* | AKPRGRWLEDY | 256 | 11 | 7 |
| F124 | 5*01 | 01 | | | | |
| IGHV3_1V* | IGHD4- | IGHJ4* | TRPRQYSTGDY | 257 | 11 | 0 |
| F124 | 4*01 | 01 | | | | |
| IGHV3_1C* | IGHD4- | IGHJ4* | AKMGGRGYSSYGPVFD | 258 | 17 | 2 |
| F124 | 4*01 | 01 | Y | | | |
| IGHV4_11* | IGHD1- | IGHJ4* | ARIVTRGPFDY | 259 | 11 | 3 |
| S4129 | 8*01 | 01 | | | | |
| IGHV3_45* | IGHD3- | IGHJ5- | ARDVTTRVVIIDHRFDV | 260 | 17 | 1 |
| S5257 | 3*01 | 1*01 | | | | |
| IGHV7_1A* | IGHD6- | IGHJ5- | ARQLGGGQTDRFDV | 261 | 14 | 3 |
| F124 | 1*01 | 1*01 | | | | |
| IGHV7_1A* | IGHD4- | IGHJ4* | ARQAYSNYPDY | 262 | 11 | 3 |
| F124 | 4*01 | 01 | | | | |
| IGHV3_4I* | IGHD1- | IGHJ4* | VKLREKWETRGD | 263 | 12 | 4 |
| F130 | 8*01 | 01 | | | | |
| IGHV5_1F* | IGHD4- | IGHJ5- | AKSYGSMSNRFDV | 264 | 13 | 3 |
| F134 | 1*01 | 1*01 | | | | |
| IGHV4_11* | IGHD3- | IGHJ4* | ARVIRLGPFDY | 265 | 11 | 2 |
| S4664 | 2*01 | 01 | | | | |
| IGHV4_11* | IGHD3- | IGHJ5- | ARETFEGDDYGYYYTPD | 266 | 22 | 3 |
| S4129 | 1*01 | 1*01 | NWFDV | | | |
| IGHV3_2P* | IGHD6- | IGHJ4* | AKSGNSGSWNYFDY | 267 | 14 | 9 |
| F133 | 3*01 | 01 | | | | |
| IGHV3_45* | IGHD3- | IGHJ4* | ARRRGWGDPYFDY | 268 | 13 | 1 |
| S5348 | 3*01 | 01 | | | | |
| IGHV1_53* | IGHD3- | IGHJ1* | ATGFSMITVALFDF | 269 | 14 | 1 |
| S2078 | 1*01 | 01 | | | | |
| IGHV4_11* | IGHD3- | IGHJ4* | ASQGYEDDYAYWAFKF | 270 | 18 | 1 |
| S4359 | 1*01 | 01 | DY | | | |
| IGHV4_11* | IGHD6- | IGHJ4* | ARSPGIVAPFDY | 271 | 12 | 10 |
| S4664 | 1*01 | 01 | | | | |
| IGHV7_A* | IGHD4- | IGHJ5- | ARSRSGSNSESRFDV | 272 | 15 | 5 |
| F132 | 1*01 | 1*01 | | | | |
| IGHV7_1A* | IGHD6- | IGHJ2* | ARPLYSGNWNVYWYFD | 273 | 17 | 4 |
| F124 | 3*01 | 01 | L | | | |
| IGHV4_11* | IGHD6- | IGHJ4* | ARDGWGGWTIDY | 274 | 12 | 3 |
| S4290 | 1*01 | 01 | | | | |
| IGHV4_11* | IGHD4- | IGHJ4* | ARSGYGSGGTFDY | 275 | 13 | 4 |
| S9724 | 1*01 | 01 | | | | |
| IGHV1_53* | IGHD2- | IGHJ4* | ATTPGYCSSTYCRFDY | 276 | 16 | 0 |
| S2078 | 3*01 | 01 | | | | |
| IGHV1_53* | IGHD3- | IGHJ1* | ATKNYYDSGYHLSGEYF | 277 | 19 | 4 |
| S2078 | 2*01 | 01 | EF | | | |
| IGHV3_4I* | IGHD1- | IGHJ5- | AQCPEYSWNMGWFDV | 278 | 15 | 2 |
| F130 | 2*01 | 1*01 | | | | |
| IGHV4_11* | IGHD3- | IGHJ5- | ASPFYGSGYYTRRFDV | 279 | 16 | 9 |
| S3915 | 2*01 | 1*01 | | | | |
| IGHV4_5B* | IGHD3- | IGHJ5- | ARDGYYSGDYYRHNWF | 280 | 18 | 5 |
| F133 | 3*01 | 1*01 | AV | | | |
| IGHV4_11* | IGHD2- | IGHJ4* | ARDCVDAFDY | 281 | 10 | 0 |
| S4290 | 1*01 | 01 | | | | |
| IGHV1_53* | IGHD1- | IGHJ4* | ATGYNWNDPFDY | 282 | 12 | 2 |
| S2078 | 3*01 | 01 | | | | |
| IGHV5_1E* | IGHD3- | IGHJ5- | TKVEGGYWGDYHRFDV | 283 | 16 | 5 |
| F133 | 3*01 | 1*01 | | | | |

TABLE 8
Usage of Lambda Gene.

| | IGLV124*4 | IGLV3-21*01\|Homo |
|---|---|---|
| IGLV124*4 | | 93.8 |
| IGLV3-21*01\|Homo | 93.8 | |
| | IGLV132*15 | IGLV2-8*01\|Homo |
| IGLV132*15 | | 90.6 |
| IGLV2-8*01\|Homo | 90.6 | |

TABLE 9
DSS-motif-containing Sequences of Antibodies Generated from RC1-4fill
VLPs-immunized macaques.
NHP 1

| | | | SEQ ID | LENGTH | Nt |
|---|---|---|---|---|---|
| VH | JH | CDRL3 | NO: | (AA) | mut. |
| IGLV132*12 | IGLJ2*01 | QSYDSSLSGHL | 284 | 11 | 1 |
| IGLV132*12 | IGLJ2*01 | QSYDSSLSAGL | 285 | 11 | 1 |
| IGLV132*12 | IGLJ2*01 | QSYDSSLSAHV | 286 | 11 | 1 |
| IGLV132*12 | IGLJ2*01 | QSYDNSLSAWV | 287 | 11 | 2 |
| IGLV132*12 | IGLJ2*01 | QSYDSSLSVRV | 288 | 11 | 1 |
| IGLV132*12 | IGLJ2*01 | QSYDNSLSARV | 289 | 11 | 4 |
| IGLV132*12 | IGLJ2*01 | QSYDNSLSXQV | 290 | 11 | 6 |
| IGLV132*12 | IGLJ2*01 | QSYDSSLSAGL | 285 | 11 | 5 |
| IGLV132*12 | IGLJ2*01 | QSYDSSLSAGL | 285 | 11 | 7 |
| IGLV132*12 | IGLJ6*01 | QSYDSSLSAHV | 286 | 11 | 1 |
| IGLV132*12 | IGLJ6*01 | QSYDNSLSAHV | 291 | 11 | 3 |
| IGLV132*12 | IGLJ6*01 | QSYDSSLSADV | 292 | 11 | 1 |
| IGLV124*30 | IGLJ1*01 | LSYDSSLSAHI | 293 | 11 | 10 |
| IGLV124*30 | IGLJ1*01 | LSYDSSLSAHI | 293 | 11 | 12 |
| IGLV124*43 | IGLJ3*01 | QVWDSSSDHPL | 294 | 11 | 2 |
| IGLV124*43 | IGLJ3*01 | QVWDSSSDHPL | 294 | 11 | 1 |
| IGLV124*30 | IGLJ2*01 | GAWDSSLSAGL | 295 | 11 | 27 |
| IGLV124*41 | IGLJ3*01 | SAWDSSLSDVL | 296 | 11 | 2 |
| IGLV132*20 | IGLJ3*01 | AAWDDSLSGVL | 297 | 11 | 4 |
| IGLV124*30 | IGLJ2*01 | ETWDYSLNGPL | 298 | 11 | 21 |
| IGLV124*24 | IGLJ6*01 | YSGDDNNDV | 299 | 9 | 2 |
| IGLV132*12 | IGLJ1*01 | QSYDSSLSGHI | 300 | 11 | 2 |
| IGLV130*2 | IGLJ6*01 | QTWTTDV | 301 | 7 | 82 |
| IGLV130*2 | IGLJ3*01 | CSYTTSNTLL | 302 | 10 | 2 |
| IGLV124*30 | IGLJ3*01 | ETWDYSLNGPL | 298 | 11 | 22 |
| IGLV132*12 | IGLJ1*01 | QSYDSSLSVHYI | 303 | 12 | 2 |
| IGLV130*31 | IGLJ2*01 | SSYASSSTWV | 304 | 10 | 1 |

NHP 5

| | | | SEQ ID | LENGTH | Nt |
|---|---|---|---|---|---|
| VH | JH | CDRL3 | NO: | (AA) | mut. |
| IGLV132*12 | IGLJ3*01 | QSYDSSLSAVL | 305 | 11 | 3 |
| IGLV132*12 | IGLJ3*01 | QSYDSSLSAVL | 305 | 11 | 3 |
| IGLV132*12 | IGLJ3*01 | QSYDSSLSALL | 306 | 11 | 3 |
| IGLV132*12 | IGLJ3*01 | QSYDSSLSAVF | 307 | 11 | 3 |
| IGLV132*12 | IGLJ3*01 | QSYDSSLSAVL | 305 | 11 | 4 |
| IGLV132*12 | IGLJ3*01 | QSYDSSLSARL | 308 | 11 | 5 |
| IGLV132*12 | IGLJ3*01 | QSYDSSLSNVL | 309 | 11 | 1 |
| IGLV132*12 | IGLJ3*01 | QSYDSSLSGVL | 310 | 11 | 4 |
| IGLV132*12 | IGLJ3*01 | QSYDNSLSAVL | 311 | 11 | 3 |
| IGLV132*12 | IGLJ3*01 | QSYDNNLSAVL | 312 | 11 | 7 |
| IGLV132*12 | IGLJ2*01 | QSYDSSLSAQV | 313 | 11 | 2 |
| IGLV132*12 | IGLJ2*01 | QSYDSSLSAHL | 314 | 11 | 3 |
| IGLV132*12 | IGLJ2*01 | QSYDSSLSAHL | 314 | 11 | 3 |
| IGLV132*12 | IGLJ2*01 | QSYDSSLSAHL | 314 | 11 | 6 |
| IGLV132*12 | IGLJ2*01 | QSYDSSLSAWV | 315 | 11 | 2 |
| IGLV132*12 | IGLJ2*01 | QSYDSSLSAGL | 285 | 11 | 1 |
| IGLV132*12 | IGLJ2*01 | QSYDSSLSGHL | 284 | 11 | 1 |
| IGLV132*12 | IGLJ2*01 | QSYDSYLSAGL | 316 | 11 | 9 |
| IGLV132*12 | IGLJ2*01 | QSYDSSLSAWV | 315 | 11 | 3 |
| IGLV132*9 | IGLJ6*01 | QSYDSSLSADV | 292 | 11 | 13 |
| IGLV132*12 | IGLJ6*01 | QSYDSSLSAHV | 286 | 11 | 2 |
| IGLV132*12 | IGLJ6*01 | QSYDNSLSDDV | 317 | 11 | 3 |
| IGLV132*12 | IGLJ6*01 | QSYDSSLSAHV | 286 | 11 | 5 |
| IGLV132*12 | IGLJ6*01 | QSYDSSLSALV | 318 | 11 | 1 |
| IGLV132*12 | IGLJ3*01 | QSYDSNLSAHVL | 319 | 12 | 3 |
| IGLV132*12 | IGLJ3*01 | QSFDSNLSIHLL | 320 | 12 | 4 |
| IGLV132*12 | IGLJ3*01 | QSYDSSLSAHVL | 321 | 12 | 1 |
| IGLV132*12 | IGLJ3*01 | QSYDSSLSAHVL | 321 | 12 | 3 |
| IGLV132*12 | IGLJ1*01 | QSYDSSLSAYI | 322 | 11 | 1 |
| IGLV132*12 | IGLJ1*01 | QSYDSSLSAYI | 322 | 11 | 7 |
| IGLV132*39 | IGLJ3*01 | DSWDSGGTHVL | 323 | 11 | 29 |
| IGLV132*20 | IGLJ2*01 | AAWDDSLSGPV | 324 | 11 | 1 |
| IGLV132*11 | IGLJ3*01 | QVWDSRSDHPL | 325 | 11 | 8 |
| IGLV124*38 | IGLJ1*01 | MIWHNNASI | 326 | 9 | 4 |
| IGLV132*43 | IGLJ2*01 | WLYYSGGHGL | 327 | 10 | 4 |
| IGLV130*33 | IGLJ1*01 | QSYDSSLSAYI | 322 | 11 | 8 |
| IGLV130*21 | IGLJ6*01 | QVWDSSSDHHDV | 328 | 12 | 4 |

NHP 6

| | | | SEQ ID | LENGTH | Nt |
|---|---|---|---|---|---|
| VH | JH | CDRL3 | NO: | (AA) | mut. |
| IGLV132*12 | IGLJ2*01 | QSYDNSLSAWV | 287 | 11 | 4 |
| IGLV132*12 | IGLJ2*01 | QSYDSSLSAGL | 285 | 11 | 1 |
| IGLV132*12 | IGLJ2*01 | QSYDSSLSAWV | 315 | 11 | 4 |
| IGLV132*12 | IGLJ2*01 | QSYDSSLRAQV | 329 | 11 | 7 |
| IGLV132*12 | IGLJ2*01 | QSYDSSLSAWV | 315 | 11 | 10 |
| IGLV132*12 | IGLJ2*01 | QSYDSSLSAQV | 313 | 11 | 0 |
| IGLV132*12 | IGLJ2*01 | QSHDSSLTAGL | 330 | 11 | 3 |
| IGLV132*12 | IGLJ2*01 | QSYDSSLSAGL | 285 | 11 | 5 |
| IGLV132*12 | IGLJ2*01 | QSHDSSLSAGL | 331 | 11 | 2 |
| IGLV132*20 | IGLJ2*01 | AAWDDSLKGWV | 332 | 11 | 10 |
| IGLV132*20 | IGLJ2*01 | AAWDDSLSGWV | 333 | 11 | 3 |
| IGLV132*20 | IGLJ2*01 | AAWDDSLNGWV | 334 | 11 | 4 |
| IGLV132*20 | IGLJ2*01 | AAWDDSLSGPL | 335 | 11 | 3 |
| IGLV132*21 | IGLJ2*01 | MIVVHNNVWA | 336 | 9 | 5 |
| IGLV132*21 | IGLJ2*01 | VIVVHNNVWA | 337 | 9 | 6 |
| IGLV132*21 | IGLJ2*01 | MIVVHNNAWI | 338 | 9 | 3 |
| IGLV132*21 | IGLJ2*01 | MIVVHNNAWV | 339 | 9 | 5 |
| IGLV132*20 | IGLJ1*01 | AAWDDSLSGYI | 340 | 11 | 4 |
| IGLV132*20 | IGLJ1*01 | AAWDDSLSGYI | 340 | 11 | 2 |
| IGLV132*20 | IGLJ1*01 | AAWDDSLSGYI | 340 | 11 | 3 |
| IGLV132*12 | IGLJ1*01 | QSYDNSLSAYI | 341 | 11 | 6 |
| IGLV132*12 | IGLJ1*01 | QSYDSILSSYI | 342 | 11 | 7 |
| IGLV132*12 | IGLJ1*01 | QSYDSSLSAYI | 322 | 11 | 4 |
| IGLV132*12 | IGLJ6*01 | QSYDSRLSADV | 343 | 11 | 13 |
| IGLV132*12 | IGLJ6*01 | QSYDSSLSAHV | 286 | 11 | 3 |
| IGLV132*33 | IGLJ2*01 | QVWDGSTKYAGL | 344 | 12 | 13 |
| IGLV132*33 | IGLJ2*01 | QVWDDSTNYAGL | 345 | 12 | 16 |
| IGLV124*30 | IGLJ3*01 | GAWDSSLSALL | 346 | 11 | 20 |
| IGLV124*30 | IGLJ3*01 | GAWDSSLSALL | 346 | 11 | 20 |
| IGLV132*12 | IGLJ3*01 | QSYDSSLSDVL | 347 | 11 | 4 |
| IGLV132*12 | IGLJ3*01 | QSYDSSLSAQL | 348 | 11 | 1 |
| IGLV132*21 | IGLJ3*01 | MIWHEDDFVL | 349 | 10 | 24 |
| IGLV130*33 | IGLJ2*01 | GAWDSSLSAHWV | 350 | 12 | 28 |
| IGLV132*39 | IGLJ3*01 | DSWDSSGTHVL | 351 | 11 | 24 |
| IGLV132*15 | IGLJ1*01 | SSYVGSGTYI | 352 | 10 | 6 |
| IGLV132*15 | IGLJ2*01 | SSYAGSGTGL | 353 | 10 | 2 |
| IGLV130*2 | IGLJ2*01 | CSYTTSNTLI | 354 | 10 | 4 |
| IGLV124*17 | IGLJ2*01 | QVWDISSDHPV | 355 | 11 | 2 |
| IGLV130*21 | IGLJ2*01 | QVWDSSSAHPV | 356 | 11 | 1 |
| IGLV124*17 | IGLJ3*01 | QVWDSSSDHPL | 294 | 11 | 6 |
| IGLV130*33 | IGLJ1*01 | QSYDSSLSAHYI | 357 | 12 | 9 |
| IGLV132*29 | IGLJ2*01 | QSADSSGNHWV | 358 | 11 | 23 |
| IGLV132*27 | IGLJ6*01 | QTWTTGIHV | 359 | 9 | 75 |
| IGLV124*3 | IGLJ2*01 | GSYRTGATFL | 360 | 10 | 17 |
| IGLV124*6 | IGLJ1*01 | SSYAGSNTFI | 361 | 10 | 2 |
| IGLV132*11 | IGLJ2*01 | QVWDSSSDHWV | 362 | 11 | 7 |
| IGLV130*33 | IGLJ3*01 | QSYDGSLSAQL | 363 | 11 | 11 |
| IGLV130*21 | IGLJ2*01 | QVWDSDHPL | 364 | 9 | 3 |

NHP 8

| | | | SEQ ID | LENGTH | Nt |
|---|---|---|---|---|---|
| VH | JH | CDRL3 | NO: | (AA) | mut. |
| IGLV132*12 | IGLJ2*01 | QSYDSTLSGGL | 365 | 11 | 2 |
| IGLV132*12 | IGLJ2*01 | QSYDSSLSAQV | 313 | 11 | 3 |
| IGLV132*12 | IGLJ2*01 | QSYDSSLSGGL | 366 | 11 | 1 |
| IGLV132*12 | IGLJ2*01 | QSYDNTLSAGL | 367 | 11 | 3 |
| IGLV132*12 | IGLJ2*01 | QSYDSSLSGHL | 284 | 11 | 1 |
| IGLV132*12 | IGLJ2*01 | QSYDSSLSVGL | 368 | 11 | 2 |
| IGLV132*12 | IGLJ2*01 | QSYDSSLSAGL | 285 | 11 | 1 |
| IGLV132*12 | IGLJ2*01 | QSYDSSLSAWV | 315 | 11 | 3 |
| IGLV132*12 | IGLJ2*01 | QSYDSSLSAGL | 285 | 11 | 2 |
| IGLV132*12 | IGLJ2*01 | QSYDSSLTAGL | 369 | 11 | 2 |
| IGLV132*12 | IGLJ2*01 | QSYDNNLSAQV | 370 | 11 | 6 |
| IGLV132*12 | IGLJ2*01 | QSHDSSLSAGL | 331 | 11 | 2 |
| IGLV132*12 | IGLJ2*01 | QSYDSSLSAWV | 315 | 11 | 3 |
| IGLV132*12 | IGLJ2*01 | QSYDSSLSARV | 371 | 11 | 5 |
| IGLV132*12 | IGLJ2*01 | QSYDSSLSAWV | 315 | 11 | 2 |
| IGLV132*12 | IGLJ2*01 | QSYDSSLSAWV | 315 | 11 | 4 |
| IGLV132*12 | IGLJ2*01 | QSYDSSLSGHL | 284 | 11 | 0 |
| IGLV132*12 | IGLJ2*01 | QSYDSSLSGHL | 284 | 11 | 1 |
| IGLV132*12 | IGLJ2*01 | QSYDSSLSAHL | 314 | 11 | 2 |
| IGLV132*12 | IGLJ2*01 | QSYDSSLSAHL | 314 | 11 | 2 |
| IGLV132*12 | IGLJ2*01 | QSYDSSLSAWV | 315 | 11 | 2 |
| IGLV132*12 | IGLJ2*01 | QSYDISLSAGL | 372 | 11 | 3 |
| IGLV132*12 | IGLJ2*01 | QSYDSSLSAGL | 285 | 11 | 2 |
| IGLV132*12 | IGLJ2*01 | QSYDNILNAGL | 373 | 11 | 4 |
| IGLV132*12 | IGLJ2*01 | HSYDSSLSAQV | 374 | 11 | 2 |
| IGLV132*12 | IGLJ2*01 | QSYDSSLSAWV | 315 | 11 | 1 |
| IGLV132*12 | IGLJ3*01 | QSYDNSLSAVL | 311 | 11 | 2 |
| IGLV132*12 | IGLJ3*01 | QSYDSSLSAVL | 305 | 11 | 4 |
| IGLV132*12 | IGLJ3*01 | QSYDSSLSALL | 306 | 11 | 5 |
| IGLV132*12 | IGLJ3*01 | QSYDNSLSAVI | 375 | 11 | 5 |
| IGLV132*12 | IGLJ3*01 | QSYDSSLSXVL | 376 | 11 | 3 |
| IGLV132*12 | IGLJ3*01 | QSYDSSLSAVL | 305 | 11 | 0 |
| IGLV132*12 | IGLJ3*01 | QSYDSSLSAQL | 348 | 11 | 1 |
| IGLV132*12 | IGLJ3*01 | QSYDSSLSGVL | 310 | 11 | 1 |
| IGLV132*12 | IGLJ3*01 | QSYDSRLSALL | 377 | 11 | 5 |
| IGLV132*12 | IGLJ3*01 | QSYDSSLSAVV | 378 | 11 | 5 |
| IGLV132*12 | IGLJ3*01 | QSYDNSLSAVL | 311 | 11 | 2 |
| IGLV132*12 | IGLJ3*01 | QSYDNSLSALL | 379 | 11 | 3 |
| IGLV132*12 | IGLJ6*01 | QSYDSSLSADV | 292 | 11 | 4 |
| IGLV132*12 | IGLJ6*01 | QSYDSSLSADV | 292 | 11 | 4 |
| IGLV132*12 | IGLJ6*01 | QSYDSSLSALV | 318 | 11 | 1 |
| IGLV132*12 | IGLJ6*01 | QSYDSSLSAHV | 286 | 11 | 4 |
| IGLV132*12 | IGLJ6*01 | QSYDSSLSADV | 292 | 11 | 5 |
| IGLV132*12 | IGLJ6*01 | QSYDSSLSAHV | 286 | 11 | 1 |
| IGLV132*12 | IGLJ6*01 | QSYDSWOCHV | 380 | 11 | 3 |
| IGLV132*12 | IGLJ6*01 | QSYDSSLSAHV | 286 | 11 | 2 |
| IGLV132*12 | IGLJ6*01 | QSYDSSLSTHV | 381 | 11 | 9 |
| IGLV132*12 | IGLJ6*01 | QSYDSSLTADV | 382 | 11 | 4 |
| IGLV132*1 | IGLJ1*01 | SSYAGSNTYI | 383 | 10 | 1 |
| IGLV132*15 | IGLJ1*01 | SSYAGSGTYI | 384 | 10 | 2 |
| IGLV132*15 | IGLJ1*01 | SSYAGSNTYI | 383 | 10 | 5 |
| IGLV132*15 | IGLJ1*01 | SSYAGSNTYI | 383 | 10 | 6 |
| IGLV132*12 | IGLJ1*01 | QSYDSRLSAHV | 385 | 11 | 6 |
| IGLV132*12 | IGLJ1*01 | QSYDSSLSAYI | 322 | 11 | 4 |
| IGLV132*12 | IGLJ1*01 | QSYHSSLRAYI | 386 | 11 | 5 |
| IGLV124*3 | IGLJ1*01 | RSYRSGRTNI | 387 | 10 | 4 |
| IGLV124*3 | IGLJ1*01 | CSYRSGDTLI | 388 | 10 | 4 |
| IGLV124*3 | IGLJ2*01 | YSYRSGNTLV | 389 | 10 | 3 |
| IGLV124*3 | IGLJ2*01 | CSYRSGSTFL | 390 | 10 | 2 |
| IGLV130*2 | IGLJ1*01 | CSYTTSSTFI | 391 | 10 | 2 |
| IGLV130*2 | IGLJ1*01 | CSYTTSSTFI | 391 | 10 | 2 |
| IGLV132*1 | IGLJ2*01 | SSYAGINTLV | 392 | 10 | 1 |
| IGLV132*15 | IGLJ2*01 | SSYAGSNTFL | 393 | 10 | 9 |
| IGLV132*17 | IGLJ3*01 | DSWDSSGTHVL | 351 | 11 | 15 |
| IGLV132*39 | IGLJ3*01 | DSWDSSGTHVL | 351 | 11 | 28 |
| IGLV124*4 | IGLJ2*01 | QVWDSSSDHWV | 362 | 11 | 1 |
| IGLV124*4 | IGLJ2*01 | QVWDISSDHPV | 355 | 11 | 5 |
| IGLV130*21 | IGLJ6*01 | QVWDSSSDHPV | 394 | 11 | 2 |
| IGLV130*21 | IGLJ2*01 | QVWDSSSDHWV | 362 | 11 | 0 |
| IGLV130*21 | IGLJ1*01 | QVWDSSNDHYI | 395 | 11 | 2 |
| IGLV132*37 | IGLJ2*01 | AAWDDRLSGWV | 396 | 11 | 1 |
| IGLV132*2 | IGLJ2*01 | CSYTSGSTWV | 397 | 10 | 4 |
| IGLV132*17 | IGLJ6*01 | DSWDSSGTLV | 398 | 10 | 29 |
| IGLV124*30 | IGLJ2*01 | LSYDSSLSAGL | 399 | 11 | 12 |
| IGLV132*33 | IGLJ3*01 | QVWDSSSDHVL | 400 | 11 | 3 |
| IGLV132*11 | IGLJ1*01 | QVWDNSSDHYI | 401 | 11 | 10 |
| IGLV130*21 | IGLJ2*01 | QVWDSSCK | 402 | 8 | 2 |
| IGLV132*29 | IGLJ2*01 | QSADSSGNHWV | 358 | 11 | 24 |
| IGLV124*30 | IGLJ1*01 | SAWDSSLSAYI | 403 | 11 | 33 |
| IGLV132*12 | IGLJ2*01 | QSYDSRLRVNWV | 404 | 12 | 2 |
| IGLV130*2 | IGLJ3*01 | CSYTTSNTLL | 302 | 10 | 3 |
| IGLV130*21 | IGLJ3*01 | QVWDSSSDHVL | 400 | 11 | 1 |
| IGLV124*30 | IGLJ2*01 | GAWDSSLSAGL | 295 | 11 | 19 |
| IGLV132*11 | IGLJ2*01 | QVWDSSSDHWV | 362 | 11 | 5 |
| IGLV130*35 | IGLJ2*01 | EAWDRSLSAWV | 405 | 11 | 34 |
| IGLV124*30 | IGLJ1*01 | DTWDNSLNGYI | 406 | 11 | 20 |

TABLE 10
Macaque GC Antibodies with CDRL3s resembling the CDRL3s of iGL V3-glycan Patch bNAbs.

| | | | | | | | | | SEQ | | | | | | SEQ | | | |
|---|---|---|---|---|---|---|---|---|---|---|---|---|---|---|---|---|---|---|
| ANTI- | | | | | | | nt | | ID | LENGTH | | | nt | | ID | LENGTH | V3-GL | |
| BODY | FORMAT | CELLS | NHP | VH | DH | JH | mut | CDRH3 | NO: | (AA) | VL | JL | mut | CDRL3 | NO: | (AA) | SPEC. | METHOD |
| 874 | Fab | LN | 1 | IgHV3_ | IgH | IgHJ1* | 5 | AKSPWG | 134 | 16 | IgLV | IgLJ | 1 | QV | 294 | 11 | YES | OCTET/ |
| | | | | 2N*F | D6- | 0 | | QSTSFEY | | | 124*4 | 3*0 | | WD | | | | SPR/ |
| | | | | 134 | 6*0 | 1 | | FEF | | | 3 | 1 | | SSS | | | | Cryo-EM |
| | | | | 1 | | | | | | | | | | DHPL | | | | |
| 876 | Ig | LN | 1 | IgHV3_ | IgH | IgHJ | 4 | AKSPWG | 133 | 16 | IgLV | IgLJ | 2 | QV | 294 | 11 | YES | ELISA |
| | | | | 2N*F | D6- | 1*0 | | QSSSFEY | | | 124*4 | 3*0 | | WD | | | | |
| | | | | 134 | 6*0 | 1 | | FEF | | | 3 | 1 | | SSS | | | | |
| | | | | | 1 | | | | | | | | | DHPL | | | | |
| 890 | Ig | LN | 1 | IgHV1_ | IgH | IgHJ | 6 | ARDRSVT | 182 | 18 | IgLV | IgLJ | 7 | QSY | 285 | 11 | YES | ELISA |
| | | | | 2B*F | D1- | 4*0 | | PFSWVEY | | | 132*1 | 2*0 | | DSS | | | | |
| | | | | 134 | 7*0 | 1 | | YFDY | | | 2 | 1 | | LSA | | | | |
| | | | | | 1 | | | | | | | | | GL | | | | |
| 890 | Fab | LN | 1 | IgHV1_ | IgH | IgHJ | 6 | ARDRSVT | 182 | 18 | IgLV | IgLJ | 7 | QSY | 285 | 11 | YES | OCTET |
| | | | | 2B*F | D1- | 4*0 | | PFSWVEY | | | 132*1 | 2*0 | | DSS | | | | |
| | | | | 134 | 7*0 | 1 | | YFDY | | | 2 | 1 | | LSA | | | | |
| | | | | | 1 | | | | | | | | | GL | | | | |
| 893 | Ig | LN | 1 | IgHV1_ | IgH | IgHJ | 2 | ATGPW | 145 | 16 | IgLV | IgLJ | 3 | QSY | 291 | 11 | YES | ELISA |
| | | | | 53*S2 | D3- | 1*0 | | GDYYGR | | | 132*1 | 6*0 | | DNS | | | | |
| | | | | 078 | 3*0 | 1 | | YFEL | | | 2 | 1 | | LSA | | | | |
| | | | | | 1 | | | | | | | | | HV | | | | |
| 893 | Fab | LN | 1 | IgHV1_ | IgH | IgHJ | 2 | ATGPW | 145 | 16 | IgLV | IgLJ | 3 | QSY | 291 | 11 | YES | OCLET |
| | | | | 53*S2 | D3- | 1*0 | | GDYYGR | | | 132*1 | 6*0 | | DNS | | | | |
| | | | | 078 | 3*0 | 1 | | YFEL | | | 2 | 1 | | LSA | | | | |
| | | | | | 1 | | | | | | | | | HV | | | | |
| 897 | Ig | LN | 1 | IgHV_4 | IgH | IgHJ | 4 | ARDSSG | 172 | 15 | IgLV | IgLJ | 6 | QSY | 433 | 11 | YES | ELISA |
| | | | | 11*S5 | D6- | 5- | | WPWDNR | | | 132*1 | 2*0 | | DNS | | | | |
| | | | | 305 | 1*0 | 1*0 | | FDV | | | 2 | 1 | | LSA | | | | |
| | | | | | 1 | 1 | | | | | | | | QV | | | | |
| 897 | Fab | LN | 1 | IgHV4_ | IgH | IgHJ | 4 | ARDSSG | 172 | 15 | IgLV | IgLJ | 6 | QSY | 433 | 11 | YES | OCTET/ |
| | | | | 11*S5 | D6- | 5- | | WPWDNR | | | 132*1 | 2*0 | | DNS | | | | SPR/ |
| | | | | 305 | 1*0 | 1*0 | | FDV | | | 2 | 1 | | LSA | | | | Cryo-EM |
| | | | | | 1 | 1 | | | | | | | | QV | | | | |
| 901 | Fab | LN | 1 | IgHV5_ | IgH | IgHJ | 2 | AKETYED | 195 | 21 | IgLV | IgLJ | 5 | QSY | 285 | 11 | YES | OCLET |
| | | | | 1A*F | D3- | 5- | | DYGYYS | | | 132*1 | 2*0 | | DSS | | | | |
| | | | | 124 | 1*0 | 1*0 | | LGYNRFD | | | 2 | 1 | | LSA | | | | |
| | | | | | 1 | 1 | | V | | | | | | GL | | | | |
| 933 | Ig | LN | 8 | IgHV7_ | IgH | IgHJ | 5 | ARLGEYS | 407 | 16 | IgLV | IgLJ | 2 | QSY | 314 | 11 | YES | ELISA |
| | | | | 1A*F | D1- | 4*0 | | WNSIGYF | | | 132*1 | 2*0 | | DSS | | | | |
| | | | | 124 | 2*0 | 1 | | DY | | | 2 | 1 | | LSA | | | | |
| | | | | | 1 | | | | | | | | | HL | | | | |
| 934 | Ig | LN | 8 | IgHV3_ | IgH | IgHJ | 10 | ARGGYY | 408 | 14 | IgLV | IgLJ | 0 | QSY | 284 | 11 | YES | ELISA |
| | | | | 3F*F1 | D1- | 4*0 | | SGRVFDD | | | 132*1 | 2*0 | | DSS | | | | |
| | | | | 32 | 8*0 | 1 | | Y | | | 2 | 1 | | LSG | | | | |
| | | | | | 1 | | | | | | | | | HL | | | | |
| 935 | Ig | LN | 8 | IgHV4_ | IgH | IgHJ | 2 | ARHSGW | 409 | 13 | IgLV | IgLJ | 6 | QV | 362 | 11 | YES | ELISA |
| | | | | 3I*F1 | D3- | 5- | | GDPYLD | | | 132*1 | 2*0 | | WD | | | | |
| | | | | 32 | 3*0 | 2*0 | | V | | | 1 | 1 | | SSS | | | | |
| | | | | | 1 | 2 | | | | | | | | DHWV | | | | |
| 936 | Ig | LN | 8 | IgHV4_ | IgH | IgHJ | 14 | ANSGSW | 410 | 13 | IgLV | IgLJ | 2 | QV | 394 | 11 | YES | ELISA |
| | | | | 6G*F | D6- | 4*0 | | NYYFDY | | | 130*2 | 6*0 | | WD | | | | |
| | | | | 124 | 3*0 | 1 | | | | | 1 | 1 | | SSS | | | | |
| | | | | | 1 | | | | | | | | | DHPV | | | | |
| 937 | Ig | LN | 8 | IgHV3_ | IgH | IgHJ | 1 | TSDPATY | 411 | 15 | IgLV | IgLJ | 2 | QSY | 311 | 11 | YES | ELISA |
| | | | | 1T*F | D1- | 1*0 | | SWNEYFE | | | 132*1 | 3*0 | | DNS | | | | |
| | | | | 132 | 2*0 | 1 | | F | | | 2 | 1 | | LSA | | | | |
| | | | | | 1 | | | | | | | | | VL | | | | |
| 938 | Ig | LN | 8 | IgHV5_ | IgH | IgHJ | 1 | AKEDGG | 412 | 14 | IgLV | IgLJ | 1 | QSY | 348 | 11 | YES | ELISA |
| | | | | 1H*F | D6- | 5- | | WSNNRV | | | 132*1 | 3*0 | | DSS | | | | |
| | | | | 132 | 5*0 | 1*0 | | DV | | | 2 | 1 | | LSA | | | | |
| | | | | | 1 | 1 | | | | | | | | QL | | | | |
| 986 | Fab | LN | 8 | IgHV5_ | | IgHJ | 3 | AKGRGY | 413 | 11 | IgLV | IgLJ | 3 | QV | 395 | 11 | YES | ELISA |
| | | | | 1F*F1 | | 5- | | NRFDV | | | 130*2 | 1*0 | | WD | | | | |
| | | | | 34 | | 1*0 | | DH | | | 1 | 1 | | SSN | | | | |
| | | | | | | 1 | | | | | | | | YI | | | | |
| 987 | Ig | LN | 8 | IgHV7_ | IgH | IgHJ | 8 | VRQGYSS | 414 | 14 | IgLV | IgLJ | 2 | QV | 400 | 11 | YES | ELISA |
| | | | | 1A*F | D6- | 5- | | WYNSLD | | | 130*2 | 3*0 | | WD | | | | |
| | | | | 124 | 2*0 | 2*0 | | V | | | 1 | 1 | | SSS | | | | |
| | | | | | 1 | 2 | | | | | | | | DH | | | | |
| | | | | | | | | | | | | | | VL | | | | |
| 987 | Fab | LN | 8 | IgHV7_ | IgH | IgHJ | 8 | VRQGYSS | 414 | 14 | IgLV | IgLJ | 2 | QV | 400 | 11 | YES | ELISA |
| | | | | 1A*F | D6- | 5- | | WYNSLD | | | 130*2 | 3*0 | | WD | | | | |
| | | | | 124 | 2*0 | 2*0 | | V | | | 1 | 1 | | SSS | | | | |
| | | | | | 1 | 2 | | | | | | | | DH | | | | |
| | | | | | | | | | | | | | | VL | | | | |
| 988 | Ig | LN | 8 | IgHV3_ | IgH | IgHJ | 18 | ARDMRDI | 415 | 19 | IgLV | IgLJ | 2 | QV | 402 | 8 | YES | ELISA |
| | | | | 4J*F1 | D5- | 4*0 | | AAGGYT | | | 130*2 | 2*0 | | WD | | | | |
| | | | | 32 | 2*0 | 1 | | YGYFDY | | | 1 | 1 | | SSC | | | | |
| | | | | | 1 | | | | | | | | | K | | | | |
| 988 | Fab | LN | 8 | IgHV3_ | IgH | IgHJ | 18 | ARDMRDI | 415 | 19 | IgLV | IgLJ | 2 | QV | 402 | 8 | YES | ELISA |
| | | | | 4J*F1 | D5- | 4*0 | | AAGGYT | | | 130*2 | 2*0 | | WD | | | | |
| | | | | 32 | 2*0 | 1 | | YGYFDY | | | 1 | 1 | | SSC | | | | |
| | | | | | 1 | | | | | | | | | K | | | | |
| 990 | Ig | LN | 8 | IgHV3_ | IgH | IgHJ | 10 | VRDPSITP | 416 | 17 | IgLV | IgLJ | 4 | QSY | 286 | 11 | YES | ELISA |
| | | | | 3F*F1 | D6- | 5- | | GPSYNRF | | | 132*1 | 6*0 | | DSS | | | | |
| | | | | 32 | 2*0 | 1*0 | | DV | | | 2 | 1 | | LSA | | | | |
| | | | | | 1 | 1 | | | | | | | | HV | | | | |
| 990 | Fab | LN | 8 | IgHV3_ | IgH | IgHJ | 10 | VRDPSITP | 416 | 17 | IgLV | IgLJ | 4 | QSY | 286 | 11 | YES | ELISA |
| | | | | 3F*F1 | D6- | 5- | | GPSYNRF | | | 132*1 | 6*0 | | DSS | | | | |
| | | | | 32 | 2*0 | 1*0 | | DV | | | 2 | 1 | | LSA | | | | |
| | | | | | 1 | 1 | | | | | | | | HV | | | | |
| 992 | Ig | LN | 8 | IgHV5_ | IgH | IgHJ | 2 | AKGVYG | 417 | 13 | IgLV | IgLJ | 1 | QSY | 284 | 11 | ND | ELISA |
| | | | | 1F*F1 | D4- | 5- | | STNRFDV | | | 132*1 | 2*0 | | DSS | | | | |
| | | | | 34 | 1*0 | 1*0 | | | | | 2 | 1 | | LSG | | | | |
| | | | | | 1 | 1 | | | | | | | | HL | | | | |
| 992 | Fab | LN | 8 | IgHV5_ | IgH | IgHJ | 2 | AKGVYG | 418 | 13 | IgLV | IgLJ | 1 | QSY | 284 | 11 | ND | ELISA |
| | | | | 1F*F1 | D4- | 5- | | LTNRFDV | | | 132*1 | 2*0 | | DSS | | | | |
| | | | | 34 | 1*0 | 1*0 | | | | | 2 | 1 | | LSG | | | | |
| | | | | | 1 | 1 | | | | | | | | HL | | | | |
| 996 | Ig | LN | 8 | IgHV3_ | IgH | IgHJ | 5 | TKEGGPE | 419 | 20 | IgLV | IgLJ | 1 | QSY | 318 | 11 | YES | ELISA |
| | | | | 3O*F | D3- | 5- | | YYNIWT | | | 132*1 | 6*0 | | DSS | | | | |
| | | | | 130 | 4*0 | 1*0 | | GWNRFD | | | 2 | 1 | | LSA | | | | |
| | | | | | 1 | 1 | | V | | | | | | LV | | | | |
| 997 | Ig | LN | 8 | IgHV4_ | IgH | IgHJ | 2 | AGGYLLF | 420 | 16 | IgLV | IgLJ | 1 | QSY | 286 | 11 | YES | ELISA |
| | | | | 3M*F | D2- | 5- | | PLGYNSL | | | 132*1 | 6*0 | | DSS | | | | |
| | | | | 133 | 3*0 | 2*0 | | DV | | | 2 | 1 | | LSA | | | | |
| | | | | | 1 | 2 | | | | | | | | HV | | | | |
| 997 | Fab | LN | 8 | IgHV4_ | IgH | IgHJ | 2 | AGGYLLF | 420 | 16 | IgLV | IgLJ | 1 | QSY | 286 | 11 | YES | OCLET |
| | | | | 3M*F | D2- | 5- | | PLGYNSL | | | 132*1 | 6*0 | | DSS | | | | |
| | | | | 133 | 3*01 | 2*02 | | DV | | | 2 | 1 | | LSAHV | | | | |
| 998 | Fab | LN | 8 | IgHV5_ | IgH | IgHJ | 1 | AKGGGPP | 421 | 15 | IgLV | IgLJ | 3 | QSY | 315 | 11 | ND | ELISA |
| | | | | 1F*F1 | D1- | 3*0 | | SWNDPF | | | 132*1 | 2*0 | | DSS | | | | |
| | | | | 34 | 2*01 | 1 | | DF | | | 2 | 1 | | LSAWV | | | | |
| 1000 | Fab | LN | 8 | IgHV5_ | IgH | IgHJ | 4 | AKNGPPY | 422 | 13 | IgLV | IgLJ | 2 | QSY | 285 | 11 | YES | ELISA |
| | | | | 1F*F1 | D3- | 4*0 | | WGMGDY | | | 132*1 | 2*0 | | DSS | | | | |
| | | | | 34 | 3*01 | 1 | | | | | 2 | 1 | | LSAGL | | | | |
| 1000 | Ig | LN | 8 | IgHV5_ | IgH | IgHJ | 4 | AKNGPPY | 422 | 13 | IgLV | IgLJ | 2 | QSY | 285 | 11 | YES | ELISA |
| | | | | 1F*F1 | D3- | 4*0 | | WGMGDY | | | 132*1 | 2*0 | | DSS | | | | |
| | | | | 34 | 3*01 | 1 | | | | | 2 | 1 | | LSAGL | | | | |
| 1002 | Fab | LN | 8 | IgHV5_ | IgH | IgHJ | 2 | AKDRGR | 423 | 16 | IgLV | IgLJ | 4 | QSY | 373 | 11 | YES | ELISA |
| | | | | 1F*F1 | D6- | 4*0 | | GGSWSL | | | 132*1 | 2*0 | | DNI | | | | |
| | | | | 34 | 3*01 | 1 | | GNDY | | | 2 | 1 | | LNAGL | | | | |
| 1003 | Ig | LN | 8 | IgHV3_ | IgH | IgHJ | 5 | AKGGED | 424 | 17 | IgLV | IgLJ | 1 | QSY | 315 | 11 | YES | ELISA |
| | | | | 1I*F1 | D3- | 4*0 | | DYIYYYT | | | 132*1 | 2*0 | | DSS | | | | |
| | | | | 30 | 1*01 | 1 | | GADY | | | 2 | 1 | | LSAWV | | | | |
| 1004 | Ig | LN | 8 | IgHV4_ | IgH | IgHJ | 3 | ARGLFNF | 425 | 19 | IgLV | IgLJ | 4 | QSY | 382 | 11 | YES | ELISA |
| | | | | 3M*F | D3- | 5- | | WSGWG | | | 132*1 | 6*0 | | DSS | | | | |
| | | | | 133 | 2*01 | 2*02 | | HNSLDV | | | 2 | 1 | | LTADV | | | | |
| 1005 | Ig | LN | 8 | IgHV4_ | IgH | IgHJ | 14 | ARDYSS | 426 | 15 | IgLV | IgLJ | 2 | QSY | 311 | 11 | YES | ELISA |
| | | | | 11*54 | D6- | 5- | | WPTYNSL | | | 132*1 | 3*0 | | DNS | | | | |
| | | | | 970 | 2*01 | 2*02 | | DV | | | 2 | 1 | | LSAVL | | | | |
| 1013 | Fab | LN | 8 | IgHV5_ | IgH | IgHJ | 1 | AKSTLLR | 427 | 12 | IgLV | IgLJ | 4 | QSY | 305 | 11 | ND | ELISA |
| | | | | 1F*F1 | D2- | 4*0 | | RSLDY | | | 132*1 | 3*0 | | DSS | | | | |
| | | | | 34 | 1*0 | 11 | | | | | 2 | 1 | | LSAVL | | | | |
| 1053 | Ig | LN | 5 | IgHV5_ | IgH | IgHJ | 1 | AKSETDF | 214 | 15 | IgLV | IgLJ | 2 | QSY | 313 | 11 | YES | ELISA |
| | | | | 1C*F | D2- | 4*0 | | WTSYYF | | | 132*1 | 2*0 | | DSS | | | | |
| | | | | 130 | 5*01 | 1 | | NY | | | 2 | 1 | | LSAQV | | | | |
| 1054 | Ig | LN | 5 | IgHV1_ | IgH | IgHJ | 2 | ARDGPYS | 219 | 15 | IgLV | IgLJ | 1 | QSY | 284 | 11 | YES | ELISA |
| | | | | 2B*F | D6- | 6*0 | | GGWSEL | | | 132*1 | 2*0 | | DSS | | | | |
| | | | | 134 | 5*01 | 1 | | DS | | | 2 | 1 | | LSGHL | | | | |
| 1061 | Ig | LN | 6 | IgHV1_ | IgH | IgHJ | 0 | ATTPGYC | 276 | 16 | IgLV | IgLJ | 7 | QSY | 342 | 11 | YES | ELISA |
| | | | | 53*52 | D2- | 4*0 | | SSTYCRF | | | 132*1 | 1*0 | | DSI | | | | |
| | | | | 078 | 3*01 | 1 | | DY | | | 2 | 1 | | LSSYI | | | | |
| 1062 | Ig | LN | 6 | IgHV5_ | IgH | IgHJ | 4 | AKGVGG | 252 | 14 | IgLV | IgLJ | 5 | QSY | 322 | 11 | YES | ELISA |
| | | | | 1F*F1 | D5- | 4*0 | | FSYSYPH | | | 132*12 | 1*01 | | DSS | | | | |
| | | | | 34 | 1*01 | 1 | | Y | | | | | | LSAYI | | | | |
| 1063 | Ig | LN | 6 | IgHV1_ | IgH | IgHJ | 5 | ARMTTV | 247 | 15 | IgLV | IgLJ | 6 | QSY | 347 | 11 | YES | ELISA |
| | | | | 2B*F | D4- | 2*0 | | AAFGGYF | | | 132*1 | 3*0 | | DSS | | | | |
| | | | | 134 | 1*01 | 1 | | DL | | | 2 | 1 | | LSDVL | | | | |
| 1064 | Ig | LN | 6 | IgHV3_ | IgH | IgHJ | 36 | TRPRQYS | 257 | 11 | IgLV | IgLJ | 2 | QVW | 355 | 11 | YES | ELISA |
| | | | | 1V*F | D4- | 4*0 | | TGDY | | | 124*1 | 2*0 | | DISS | | | | |
| | | | | 124 | 4*01 | 1 | | | | | 7 | 1 | | DHPV | | | | |
| 1068 | Ig | LN | 6 | IgHV1_ | IgH | IgHJ | 4 | ATKNYY | 277 | 19 | IgLV | IgLJ | 11 | QSY | 363 | 11 | YES | ELISA |
| | | | | 53*52 | D3- | 1*0 | | DSGYHLS | | | 130*3 | 3*0 | | DGS | | | | |
| | | | | 078 | 2*01 | 1 | | GEYFEF | | | 3 | 1 | | LSAQL | | | | |
| 1169 | Ig | LN | 8 | IgHV5_ | IgH | IgHJ | 2 | AKDGGPS | 428 | 18 | IgLV | IgLJ | 6 | SSY | 383 | 10 | ND | ELISA |
| | | | | 1F*F1 | D2- | 5- | | GSYYYG | | | 132*1 | 1*0 | | AGS | | | | |
| | | | | 34 | 4*01 | 1*01 | | GRFDV | | | 5 | 1 | | NTYI | | | | |
| 1169 | Fab | LN | 8 | IgHV5_ | IgH | IgHJ | 2 | AKDGGPS | 429 | 18 | IgLV | IgLJ | 6 | SSY | 383 | 10 | ND | OCLET |
| | | | | 1F*F1 | D2- | 5- | | GSYYYR | | | 132*1 | 1*0 | | AGS | | | | |
| | | | | 34 | 4*01 | 1*01 | | GRFDV | | | 5 | 1 | | NTYI | | | | |
| 1170 | Ig | LN | 8 | IgHV1_ | IgH | IgHJ | 7 | ARGGGH | 430 | 11 | IgLV | IgLJ | 2 | SSY | 392 | 10 | YES | ELISA |
| | | | | 2G*F | D2- | 4*0 | | SSFDF | | | 132*1 | 2*0 | | AGI | | | | |
| | | | | 130 | 2*01 | 1 | | | | | | 1 | | NTLV | | | | |
| 1170 | Fab | LN | 8 | IgHV1_ | IgH | IgHJ | 7 | ARGGGH | 430 | 11 | IgLV | IgLJ | 2 | SSY | 392 | 10 | YES | ELISA/ |
| | | | | 1P*F1 | D2- | 4*0 | | SSFDF | | | 132*1 | 2*0 | | AGI | | | | OCTET |
| | | | | 33 | 2*01 | 1 | | | | | | 1 | | NTLV | | | | |
| 1177 | Fab | LN | 6 | IgHV4_ | IgH | IgHJ | 10 | ARSRSGS | 272 | 15 | IgLV | IgLJ | 6 | SSY | 352 | 10 | ND | ELISA/ |
| | | | | 5N*F | D4- | 5- | | NSESRED | | | 132*1 | 1*0 | | VGS | | | | OCTET |
| | | | | 133 | 1*01 | 1*01 | | V | | | 5 | 1 | | GTYI | | | | |
| 1178 | Fab | LN | 6 | IgHV4_ | IgH | IgHJ | 0 | ARDSYK | 431 | 13 | IgLV | IgLJ | 2 | SSY | 361 | 10 | ND | ELISA/ |
| | | | | 11*59 | D1- | 3*0 | | DSPAFDF | | | 124*6 | 1*0 | | AGS | | | | OCTET |
| | | | | 280 | 8*01 | 1 | | | | | | 1 | | NTFI | | | | |
| 1180 | Fab | LN | 8 | IgHV5_ | IgH | IgHJ | 5 | AKDQTD | 432 | 17 | IgLV | IgLJ | 6 | SSY | 383 | 10 | YES | ELISA/ |
| | | | | 1F*F1 | D3- | 4*01 | | LDWLLY | | | 132*1 | 1*0 | | AGS | | | | OCTET |
| | | | | 34 | 2*01 | | | GGFDY | | | 5 | 1 | | NTYI | | | | |

TABLE 11
QxxDSS motif-containing bNAbs (″QxxDSS″ disclosed as SEQ ID NO: 20).

| | | | | SEQ ID | | SEQ ID |
|---|---|---|---|---|---|---|
| bNAb | VH | VL | CDRL3 (MT) | NO: | CDRL3 (iGL) | NO: |
| PGT121 | 4-59 | L3-21 | HIWDSRVPTKWV | 434 | QVWDSSSDHPWV | 445 |
| PGT122 | 4-59 | L3-21 | HIWDSRRPTNWV | 435 | QVWDSSSDHPWV | 445 |
| PGT123 | 4-59 | L3-21 | HIYDARGGTNWV | 436 | QVWDSSSDHPWV | 445 |
| 10-1074 | 4-59 | L3-21 | HMWDSRSGFSWS | 437 | QVWDSSSDHPWV | 445 |
| PGT124 | 4-59 | L3-21 | MWDSRSGFSWS | 438 | QVWDSSSDHPWV | 445 |
| BG18 | 4-4 | L3-25 | QSSDTSDSYKM | 439 | | |
| PGT125 | 4-39 | L2-8 | GSLVGNWDVI | 440 | SSYAGSNXXX | 446 |
| PGT126 | 4-39 | L2-8 | SSLVGNWDVI | 441 | SSYAGSNXXX | 446 |
| PGT127 | 4-39 | L2-8 | SSLVGNWDVI | 441 | SSYAGSNXXX | 446 |
| PGT128 | 4-39 | L2-8 | GSLVGNWDVI | 440 | SSYAGSNXXX | 446 |
| PGT130 | 4-39 | L2-8 | SSLFGRWDVV | 442 | SSYAGSNXXX | 446 |
| PGT131 | 4-39 | L2-8 | SSLSGRWDIV | 443 | SSYAGSNXXX | 446 |
| DH270.6 | 1-2 | L2-23 | SFGGSATVV | 444 | SYAGSSTVI | 447 |

TABLE 12
PCR Primers.
Heavy chain

| | | | | SEQ ID |
|---|---|---|---|---|
| | | Primer name | Primer sequence | NO: |
| 1st PCR | Forward | p1350 | ACAGGTGCCCACTCCCAGGTGCAG | 448 |
| | | p1351 | AAGGTGTCCAGTGTGARGTGCAG | 449 |
| | | p1352 | CCCAGATGGGTCCTGTCCCAGGTGCA | 450 |
| | | | G | |
| | | p1353 | CAAGGAGTCTGTTCCGAGGTGCAG | 451 |
| | | VH5 LEADER-A | TTCTCCAAGGAGTCTGT | 452 |
| | | VH3 LEADER-A | TAAAAGGTGTCCAGTGT | 453 |
| | | VH3 LEADER- | TAAGAGGTGTCCAGTGT | 454 |
| | | AB | | |
| | | VH3 LEADER-C | TAGAAGGTGTCCAGTGT | 455 |
| | | VH4 LEADER-D | ATGAAACATCTGTGGTTCTT | 456 |
| | | VH3 LEADER-E | TACAAGGTGTCCAGTGT | 457 |
| | | VH3 LEADER-F | TTAAAGCTGTCCAGTGT | 458 |
| | Reverse | 3′SalI.JH1/4/5 | GCTGAGGAGACGGTGACCAG | 459 |
| | | 3′SalI.JH2 | GCTGAGGAGATGGTGATTGGG | 460 |
| | | 3′SalI.JH3 | GCTGAAGAGACGGTGACCCTG | 461 |
| | | 3′SalI.JH6 | GCTGAGGAGACGGTGACGACG | 462 |
| 2nd | Forward | p1355 | CTAGTAGCAACTGCAACCGGTGTACA | 463 |
| PCR | | | TTCCCAGGTGCAGCTGGTGCAG | |
| | | p1356 | CTAGTAGCAACTGCAACCGGTGTACA | 464 |
| | | | TTCCGAGGTGCAGCTGGTGCAG | |
| | | p1357 | CTAGTAGCAACTGCAACCGGTGTACA | 465 |
| | | | TTCCCAGGTTCAGCTGGTGCAG | |
| | | p1358 | CTAGTAGCAACTGCAACCGGTGTACA | 466 |
| | | | TTCCCAGGTCCAGCTGGTACAG | |
| | | p1359 | CTAGTAGCAACTGCAACCGGTGTACA | 467 |
| | | | TTCTGAGGTGCAGCTGGTGGAG | |
| | | p1360 | CTAGTAGCAACTGCAACCGGTGTACA | 468 |
| | | | TTCTCAGGTGCAGCTGGTGGAG | |
| | | p1361 | CTAGTAGCAACTGCAACCGGTGTACA | 469 |
| | | | TTCTGAGGTGCAGCTGTTGGAG | |
| | | p1362 | CTAGTAGCAACTGCAACCGGTGTACA | 468 |
| | | | TTCTCAGGTGCAGCTGGTGGAG | |
| | | p1363 | CTAGTAGCAACTGCAACCGGTGTACA | 470 |
| | | | TTCTGAAGTGCAGCTGGTGGAG | |
| | | p1364 | CTAGTAGCAACTGCAACCGGTGTACA | 471 |
| | | | TTCCCAGGTGCAGCTGCAGGAG | |
| | | p1365 | CTAGTAGCAACTGCAACCGGTGTACA | 472 |
| | | | TTCCCAGGTGCAGCTACAGCAGTG | |
| | | p1366 | CTAGTAGCAACTGCAACCGGTGTACA | 473 |
| | | | TTCCCAGCTGCAGCTGCAGGAG | |
| | | p1367 | CTAGTAGCAACTGCAACCGGTGTACA | 474 |
| | | | TTCCCAGGTACAGCTGCAGAG | |
| | Reverse | p1370 | CCGATGGGCCCTTGGTCGACGCTGAG | 475 |
| | | | GAGACGGTGACCAG | |
| | | p1371 | CCGATGGGCCCTTGGTCGACGCTGAA | 476 |
| | | | GAGACGGTGACCATTG | |
| | | p1372 | CCGATGGGCCCTTGGTCGACGCTGAG | 475 |
| | | | GAGACGGTGACCAG | |
| | | p1373 | CCGATGGGCCCTTGGTCGACGCTGAG | 477 |
| | | | GAGACGGTGACCGTG | |
| Sequencing | | RM_FWD_T4_Seq | GTAGCAACTGCAACCGGTGT | 478 |
| Colony | Forward | Ab-sense | GCTTCGTTAGAACGCGGCTAC | 479 |
| PCR | Reverse | p1354 | GGAAGGTGTGCACGCCGCTGGTC | 480 |
| | Sequencing | Ab-sense | GCTTCGTTAGAACGCGGCTAC | 479 |

Light chain (λ)

| | | | | SEQ ID |
|---|---|---|---|---|
| | | Primer name | Primer sequence | NO: |
| 1st PCR | Forward | p1394 | GGTCCTGGGCCCAGTCTGTGCTG | 481 |
| | | p1395 | GGTCCTGGGCCCAGTCTGCCCTG | 482 |
| | | p1396 | GCTCTGTGACCTCCTATGAGCTG | 483 |
| | | p1397 | GGTCTCTCTCSCAGCYTGTGCTG | 484 |
| | | p1398 | GTTCTTGGGCCAATTTTATGCTG | 485 |
| | | p1399 | GGTCCAATTCYCAGGCTGTGGTG | 486 |
| | | p1400 | GAGTGGATTCTCAGACTGTGGTG | 487 |
| | Reverse | p1401 | CACCAGTGTGGCCTTGTTGGCTTG | 488 |
| 2nd PCR | Forward | p1402 | CTAGTAGCAACTGCAACCGGTTCCTG | 489 |
| | | | GGCCCAGTCTGTGCTGACKCAG | |
| | | p1403 | CTAGTAGCAACTGCAACCGGTTCCTG | 490 |
| | | | GGCCCAGTCTGCCCTGACTCAG | |
| | | p1404 | CTAGTAGCAACTGCAACCGGTTCTGT | 491 |
| | | | GACCTCCTATGAGCTGACWCAG | |
| | | p1405 | CTAGTAGCAACTGCAACCGGTTCTCT | 492 |
| | | | CTCSCAGCYTGTGCTGACTCA | |
| | | p1406 | CTAGTAGCAACTGCAACCGGTTCTTG | 493 |
| | | | GGCCAATTTTATGCTGACTCAG | |
| | | p1407 | CTAGTAGCAACTGCAACCGGTTCCAA | 494 |
| | | | TTCYCAGRCTGTGGTGACYCAG | |
| | Reverse | p1409 | GGCTTGAAGCTCCTCACTCGAGGGYG | 495 |
| | | | GGAACAGAGTG | |
| Sequencing | | p1409 | GGCTTGAAGCTCCTCACTCGAGGGYG | 495 |
| | | | GGAACAGAGTG | |
| Colony | Forward | Ab-sense | GCTTCGTTAGAACGCGGCTAC | 479 |
| PCR | Reverse | p1409 | GGCTTGAAGCTCCTCACTCGAGGGYG | 495 |
| | | | GGAACAGAGTG | |
| | Sequencing | Ab-sense | GCTTCGTTAGAACGCGGCTAC | 479 |

We claim:

1. A method of treating or preventing HIV infection in a subject in need thereof, comprising administering to the subject a therapeutically effective amount of a polypeptide having an amino acid sequence selected from the group consisting of SEQ ID NOs: 2, 4, 6, 8, 11, and 13.

2. The method of claim 1, further comprising administering to the subject a therapeutically effective amount of an anti-viral agent.

3. The method of claim 2, wherein the anti-viral agent comprises a transcriptase inhibitor, a non-nucleoside reverse transcriptase inhibitor, a fusion protein inhibitor, or helper T cells.

4. The method of claim 3, wherein the transcriptase inhibitor is selected from the group consisting of abacavir, AZT, didanosine, emtricitabine, lamivudine, stavudine, tenofovir, zalcitabine, and zidovudine.

5. The method of claim 3, wherein the non-nucleoside reverse transcriptase inhibitor is selected from the group consisting of delavirdine, efavirenz, nevirapine, protease inhibitors such as amprenavir, atazanavir, indinavir, lopinavir, nelfinavir, fosamprenavir, ritonavir, saquinavir, and tipranavir.

6. The method of claim 3, wherein the fusion protein inhibitor is enfuvirtide.

7. The method of claim 2, wherein the therapeutically effective amount of the polypeptide is administered to the subject before the anti-viral agent.

8. The method of claim 2, wherein the therapeutically effective amount of the polypeptide is administered to the subject after the anti-viral agent.
